(19) United States
(12) Patent Application Publication
(10) Pub. No.: US 2024/0317847 A1
(43) Pub. Date: Sep. 26, 2024

(54) INHIBITORS OF IL-11 OR IL-11Ra FOR USE IN THE TREATMENT OF ABNORMAL UTERINE BLEEDING

(71) Applicant: BAYER AKTIENGESELLSCHAFT, Leverkusen (DE)

(72) Inventors: Maik Stefan Wilhelm OBENDORF, Weimar (DE); Frank SACHER, Berlin (DE); Jörg MÜLLER, Berlin (DE); Ralf LESCHE, Berlin (DE); Christian VOTSMEIER, Paderborn (DE); Stephan MÄRSCH, Pähl (DE); Jan TEBBE, Köln (DE); Philipp ELLINGER, Solingen (DE); Patrick Michael SMITH, Leverkusen (DE); Jenny FITTING, Köln (DE); Katharina FILARSKY, Düsseldorf (DE); Mathias GEHRMANN, Köln (DE); Marcus KARLSTETTER, Bergisch Gladbach (DE); Ernst WEBER, Langenfeld (DE); Mark TRAUTWEIN, Wülfrath (DE)

(73) Assignee: BAYER AKTIENGESELLSCHAFT, Leverkusen (DE)

(21) Appl. No.: 18/277,573

(22) PCT Filed: Feb. 24, 2022
(86) PCT No.: PCT/EP2022/054612

§ 371 Date: Aug. 16, 2023

(30) Foreign Application Priority Data
Feb. 26, 2021 (EP) 21159569.9

(51) Int. Cl. C07K 16/24; A61K 39/00; A61P 7/04; A61P 15/02

(52) U.S. Cl. CPC C07K 16/244; A61P 7/04; A61P 15/02; A61K 2039/505; C07K 2317/21; C07K 2317/31; C07K 2317/565; C07K 2317/76; C07K 2317/92

(57) ABSTRACT

The present invention is related to agents capable of binding to and inhibiting or antagonizing the action of IL-11 and/or IL-11RA for the treatment and/or prevention of abnormal uterine bleeding, which comprises heavy menstrual bleeding, prolonged bleeding, altered bleeding pattern, dysmenorrhea, as well as of the underlying diseases leiomyoma and endometriosis and the use of the agent to inhibit menstruation. Furthermore, the invention provides novel IL-11 antibodies.

### FIELD OF THE INVENTION

The present invention provides agents in form of inhibitors and antagonists of interleukin-11 (IL-11) and/or interleukin-11 receptor alpha (IL-11RA) including allosteric inhibitors and antagonists for the treatment and/or prevention of abnormal uterine bleeding. The invention provides inhibitors or antagonists in the form of antibodies, fragments and derivatives thereof, antibody mimetics, nucleic acids, aptamers, or small molecules. The invention also provides assays and screening technologies to find such agents.

Furthermore, the present invention provides isolated antibodies or antigen-binding fragments thereof that bind to human interleukin-11 (IL-11), pharmaceutical compositions and combinations comprising said isolated antibodies or antigen-binding fragments thereof, and to the use of said isolated antibodies or antigen-binding fragments thereof for manufacturing a pharmaceutical composition for the treatment or prophylaxis of a disease, in particular in mammals, such as but not limited to abnormal uterine bleeding (AUB), such as heavy menstrual bleeding and menorrhagia as well as dysmenorrhea associated with AUB.

### BACKGROUND OF THE INVENTION

Abnormal uterine bleeding (AUB) may be diagnosed when a woman experiences a change in her menstrual blood loss (MBL), or the degree of MBL or vaginal bleeding pattern differs from that experienced by the age-matched general female population (National Collaborating Centre for Women's and Children's Health (NCCWCH): National Institute for Clinical Excellence (NICE) guidelines. CG44 heavy menstrual bleeding: full guideline. 24 Jan. 2007). Normal menstruation occurs at a cycle of 28±7 days, lasting 4±2 days with a mean MBL of 40±20 ml. AUB presents a spectrum of abnormal menstrual bleeding patterns that includes irregular, heavy or prolonged menstrual bleeding or an altered bleeding pattern.

AUB may be associated with ovulatory or anovulatory cycles. Terms in use are dysfunctional uterine bleeding (DUB), menorrhagia (abnormally heavy menstrual bleeding at regular intervals which may also be prolonged), metrorrhagia (uterine bleeding at irregular intervals, particularly between the expected menstrual periods), and metromenorrhagia (combination of both). AUB is one of the most frequent gynecological disorders observed by general practitioners and gynecologists.

Heavy menstrual bleeding (HMB) is widely defined in the medical literature as menstrual blood loss (MBL) of 80 ml or more per menstrual period (Hallberg & Nilsson 1964, Hallberg et al. (1966)). Within the meaning of the present invention, HMB is defined as menstrual blood loss of 60 ml or more per cycle, for example 60 to 80 ml per cycle, in particular more than 80 ml per cycle. According to NICE, HMB should be defined for clinical purposes as excessive menstrual blood loss which interferes with the woman's physical, emotional, social and material quality of life and which can occur alone or in combination with other symptoms. Any interventions should aim to improve quality of life measures. The global prevalence rate of HMB, based on 18 epidemiological studies, ranges from 4% to 52% (Fraser et al., 2009). The wide variation can be accounted for by different methods of assessment and population samples used by each study. Prevalence rates in studies that use subjective assessments have been found to be consistently higher, compared to 9-11% in studies that directly measured MBL. However, an estimated 30% of women suffering from HMB appear to be more representative (El-Hemaidi et al. (2007)). HMB is more prevalent among women at the extreme ends of the reproductive age spectrum (i.e. adolescent girls and women approaching or going through menopause) (Shapley et al. (2004)).

Underlying organic causes might be diagnosed, such as benign uterine neoplasia, especially cervical and endometrial polyps and leiomyoma, adenomyosis and malignancies of the cervix and endometrium. Actually, the most prominent cause of AUB and HMB is leiomyoma and a substantial proportion of women with symptomatic leiomyoma experience HMB.

Heavy menstrual bleeding is often the presenting symptom of an underlying bleeding disorder, such as hemophilia and von Willebrand's disease, platelet disorders/dysfunctions like Glantzmann's thrombasthenia and thrombocytopenia as well as plasminogen activator inhibitor 1 PAI-1 deficiency and can be the only bleeding symptom in women. Despite the large impact on women's health therapeutic approaches to AUB and HMB are still an unmet medical need.

Leiomyoma (also known as uterine fibroids or myoma) are the most common benign gynecological tumors of women of reproductive age. Approximately 5-10% of women of reproductive age have symptoms of uterine fibroids and requires treatment. Leiomyoma consist of muscle cells and other tissues that grow in and around the wall of the uterus or womb. They are frequently characterized by heavy menstrual bleeding, pain and bulk symptoms. The symptoms can range from mild to severe and have the potential to impact a woman's day-to-day life. Leiomyoma are among the leading causes of hospitalization for gynecological disorders and are a primary indication for hysterectomy. Hysterectomy is the only permanent treatment for leiomyoma appropriate for women not wishing to preserve their fertility or their uterus. Minimally invasive surgical interventions exist but are associated with a risk of recurrence and the need for additional interventions. Surgical interventions are associated with risk for complications. Current medical treatment options are limited to short-term reduction of symptoms. Evidence for sustained benefit of long-term use of current drugs is lacking and significant adverse events are associated with some treatments. The avoidance of surgery or invasive procedures for leiomyoma requires an effective long-term medical treatment option, which is still an unmet medical need.

Dysmenorrhea, also known as painful periods, or menstrual cramps, is pain during menstruation. Its usual onset occurs around the time that menstruation begins. Symptoms typically last less than three days. The pain is usually in the pelvis or lower abdomen. Other symptoms may include back pain, diarrhea, or nausea. (Osayande & Mehulic (2014)). It is the most common menstrual disorder with a prevalence varying depending on the study between 16% and 91% in women of reproductive age, with severe pain in 2%-29% (Ju et al. (2014)). Dysmenorrhea is often due to an underlying issue such as leiomyoma or endometriosis (secondary dysmenorrhea), the latter accounting the underlying reason for 70% of all dysmenorrhea patient (Janssen et al. (2013)). Furthermore, dysmenorrhea is more common among those women with heavy periods, irregular periods, or whose periods started before twelve years of age (Wikipedia: https://en.wikipedia.org/wiki/dysmenorrhea).

Prostaglandins and other inflammatory mediators are released during menstruation due to the destruction of the endometrial cells at the end of the menstrual cycle (Lethaby et al. (2013)). These factors cause the myometrium to contract (Wright & Solange (2003)). When the uterine muscles contract they constrict the blood supply to the tissue of the endometrium which in turn breaks down and dies further. In conclusion these contractions and the resulting temporary oxygen deprivation to nearby tissues are responsible for the pain or “cramps” experienced during menstruation.

Current treatment options are nonsteroidal anti-inflammatory drugs (NSAIDs) for relieving the pain of primary dysmenorrhea (Marjoribanks et al. (2015)). However, they can have side effects of nausea, dyspepsia, peptic ulcer, and diarrhea.

Other treatment options for dysmenorrhea are combined oral contraceptives. A systematic review of Wong et al. (2009) however, described limited evidence that combined oral contraceptives containing low doses or medium doses of estrogen reduce pain associated with dysmenorrhea. Progestin-only hormonal treatments might be more effective as often attenuation of menstruation is achieved (Power et al. (2007); Sachedina & Todd (2020)). However, continuous progestin-only application very often causes spotting with unpredictable time patterns limiting also quality of life and acceptance. Better and more accepted treatment options are necessary, also for those patients hesitant to hormonal treatments.

Endometriosis is a chronic gynecological disorder defined by the presence of endometrial tissue (lesions) outside the uterus e.g., in the peritoneal cavity which induces a chronic inflammatory reaction leading to chronic pelvic pain and infertility (Giudice & Kao (2004)). With a prevalence of approximately 5-10% of the female population endometriosis is estimated to affect 176 million women worldwide (Adamson et al. (2010)). Endometriosis can have a severe impact on women's lives. For many the symptoms are so severe that they are bedridden for an average of 18 days a year (Kjerulff et al. (1996)). Lesions are hormone-responsive and may haemorrhage during menstruation.

Women with endometriosis may suffer from chronic, cyclic pelvic pain (dysmenorrhea) and/or non-cyclic pelvic pain, have impaired fertility and experience pain during intercourse (dyspareunia). Of these, dysmenorrhea is often reported as the most common symptom (Sinaii et al. (2008)). Other symptoms may include gastrointestinal disturbances, urinary problems and fatigue. The most accepted theory for how endometriosis arises is that during menstruation endometrial tissue is shed through the fallopian tubes and implants on the peritoneal surfaces (Sampson (1927), Giudice (2010)). Prior to receiving a laparoscopic diagnosis women with endometriosis are commonly treated with non-steroidal anti-inflammatory drugs (NSAIDs), opioids and/or empirically with combined oral contraceptives (COCs) or progestins. The use of COCs is off-label while a few progestins have been licensed in some countries for this indication. Other hormone-based therapies involve gonadotropin releasing hormone (GnRH) analogues or antagonists. While hormone-based treatments can attenuate lesion growth they often have limited efficacy or potential adverse effects (DiVasta & Laufer (2013)). For safety issues there is no option for long-term continuous treatment with GnRH analogues or GnRH antagonists as well as not for currently used pain inhibitors as e.g., ibuprofen. Surgical removal of lesions is another option; however, symptoms typically recur in up to 75% of women within two years (Olive (2002)). The avoidance of surgery or invasive procedures for endometriosis requires an effective long-term medical treatment option which is still an unmet medical need.

There is an unmet medical need for the treatment of abnormal uterine bleeding such as heavy menstrual bleeding, menorrhagia and dysmenorrhea and the underlying diseases such as leiomyoma and endometriosis. The object of the present invention is to provide means which enable the treatment of the aforementioned diseases.

The physiological role of Interleukin-11 (IL-11) and IL-11RA signaling is discussed controversially. IL-11 (Interleukin 11 UniProtKB—P20809) also known as AGIF is described as a stromal cell-derived cytokine that belongs to a family of pleiotropic and redundant cytokines that use the Interleukin 6 Signal Transducer (IL6ST)/glycoprotein 130 (gp130) transducing subunit in their high affinity multisubunit receptor complexes (gp130 family of cytokines). IL-11 cytokine is shown to stimulate the T-cell-dependent development of immunoglobulin-producing B cells. It is also found to support the proliferation of hematopoietic stem cells and megakaryocyte progenitor cells and induces megakaryocyte maturation resulting in increased platelet production (Paul et al. (1990)). IL-11 also promotes the proliferation of hepatocytes in response to liver damage. Binding to its receptor IL-11RA or sIL-11RA and gp130 activates a signaling cascade that promotes cell proliferation (Harmegnies et al. (2003)). Signaling leads to the activation of intracellular protein kinases and the phosphorylation of ‘Signal transducer and activator of transcription 3’ (STAT3). For the IL11 gene alternatively spliced transcript variants encoding distinct isoforms have been found.

IL-11RA (interleukin-11 receptor alpha (A), UniProtKB—Q14626) also known as CRSDA is the receptor for interleukin-11, which is a member of the hematopoietic cytokine receptor family. Structurally this particular receptor is similar to ciliary neurotrophic factor receptor, since both contain an extracellular region with a 2-domain structure composed of an immunoglobulin-like domain and a cytokine receptor-like domain. The receptor systems for interleukin 6 (IL-6), leukemia inhibitory factor (LIF), oncostatin M (OSM), ciliary neurotrophic factor (CNTF), IL-11 and cardiotrophin 1 (CT1) can utilize IL6ST for initiating signal transmission. Multiple alternatively spliced transcript variants have been found for this gene. In rodents two orthologues for the human IL-11RA exist, IL-11RA1 and IL-11RA2. The membrane bound IL-11RA receptors can be cleaved resulting in the release of a soluble isoform: sIL-11RA. Contrary to the classical signaling of membrane-bound IL-11RA/IL-11/IL6ST (cis-signaling), the soluble IL-11RA can mediate the trans-signaling: after binding of IL-11 to the receptor the IL-11/sIL-11RA complex binds to cells expressing the membrane-bound IL6ST and the IL-11R/sIL-11RA/IL6ST complex can initiate intracellular signaling comparable to the classical membrane-bound IL-11RA. For trans-signaling no expression of IL-11RA is necessary in the target cell (Lokau et al., 2017).

Cork et al. 2002 described that IL-11 is produced in the endometrium by both stromal cells and epithelial cells and stromal cell production is increased during decidualization. Additionally, IL-11RA is present in the human endometrium, with little variation in receptor expression through the menstrual cycle. The patent application WO9603143A1 discloses that bleeding disorders like von Willebrand's disease, blood coagulation disorders or patients with unexplained prolongation of bleeding time should be treated with IL-11 cytokine. Ragni et al. 2011 reported on a clinical trial in women with mild von Willebrand disease which were treated with human recombinant IL-11. The treatment reduced menstrual bleeding severity in 71% of the subjects. Hence prior art describes that menstrual bleeding can be reduced by the application of IL-11. However, there is no evidence in the prior art suggesting treatment of abnormal uterine bleeding or menstruation by antagonization or inhibiting of IL-11 or IL-11RA.

It has been shown that among several other deregulated factors expression of IL-11 is upregulated in leiomyoma tissue (Luo et al. (2005)) and that its expression might be a relevant diagnostic marker for leiomyoma or a diagnostic marker for monitoring the treatment of leiomyoma (WO2005/098041A2). However, there was no indication in the literature that the inhibition or antagonization of IL-11 and/or IL-11RA could be a treatment option.

For endometriosis the data on expression of IL-11 and/or IL-11RA are controversial, one study described a deregulation of IL-11 and IL-11RA in the endometrium of infertile women with endometriosis in comparison to fertile women (Dimitriadis et al. (2006)). However, this finding was discussed with respect to etiopathogenesis of infertility but not for endometriosis itself. Furthermore, this finding could not be confirmed in a separate independent study (Mikolajczyk et al. (2006)). Another study described secreted IL-11 in the peritoneal fluid of women with endometriosis and controls; however, no correlation with endometriosis or disease stage of endometriosis (Gazvani et al. (2000)) was shown. Furthermore Gazvani et al. stated that there is no evidence to suggest a role of IL-11 in the pathogenesis of pelvic endometriosis.

Function blocking antibodies of IL-11 or recombinant proteins antagonizing IL-11 e.g., receptor-bodies derived from IL-11RA have already been described in the scientific and patent literature, however, not for treatment and/or prevention of abnormal uterine bleeding such as heavy menstrual bleeding, prolonged bleeding or altered bleeding pattern as well as dysmenorrhea, and of the underlying diseases leiomyoma and endometriosis and menstruation.

Examples for inhibitory receptor-bodies which function as antagonists of IL-11 signaling are described in the patent application WO 9959608. Examples for inhibitory antibodies which function as antagonists of IL-11 and IL-11 signaling are described in the patent applications WO2018109170, WO2018109174, WO2017103108, WO2019238882, and WO2019238884. Function blocking antibodies for human and mouse IL-11 are provided by commercial suppliers e.g., MAB218 and Mab418 from R&D Systems, Inc.

### SUMMARY OF THE INVENTION

One aspect of the present invention is the non-hormonal treatment, prevention or alleviation of abnormal uterine bleeding such as heavy menstrual bleeding, menorrhagia and dysmenorrhea in women with or without leiomyoma by inhibiting the action and signaling of Interleukin 11 (IL-11) or its receptor (IL-11RA). Another aspect of the invention is the non-hormonal treatment, prevention or alleviation of leiomyoma. Another aspect of the invention is the non-hormonal treatment, prevention or alleviation of endometriosis. Another aspect of the invention is the non-hormonal treatment, prevention or alleviation of dysmenorrhea. Another aspect of the invention is the non-hormonal prevention or alleviation of menstruation itself.

Furthermore, this invention is related to novel antibodies, or antigen-binding antibody fragments thereof, which display high affinity for human, mouse, rat, and/or marcaca IL-11 protein and which inhibit the IL-11 mediated signalling. Some IL-11 antibodies or antigen-binding antibody fragments thereof of this invention inhibit the interaction of IL-11 with IL-11Ra and the formation of IL-11/IL-11Ra/gp130 complex. Further antibodies or antigen-binding antibody fragments thereof of this invention inhibit the formation of IL-11/IL-11Ra/gp130 complex and do not inhibit the interaction of IL-11 with IL-11Ra. Furthermore, this invention relates to novel bispecific antibodies or antigen-binding antibody fragments thereof which display high affinity for human, mouse, rat, and/or marcaca IL-11 protein and which inhibit the IL-11 mediated signalling. In addition, antibodies, or antigen-binding antibody fragments thereof of this invention do not bind to IL-2Ra.

Highly preferred IL-11 antibodies of the invention are depicted in Table 9 and 10 and characterized by their structural features.

The invention is also related to polynucleotides encoding the antibodies of the invention, or antigen-binding fragments thereof, cells expressing the antibodies of the invention, or antigen-binding fragments thereof, methods for producing the antibodies of the invention, or antigen-binding fragments thereof. The invention is also related to isolated nucleic acid sequences, each of which can encode an aforementioned antibody or antigen-binding fragment thereof. Nucleic acids of the invention are suitable for recombinant production of antibodies or antigen-binding antibody fragments. Thus, the invention also relates to vectors and host cells containing a nucleic acid sequence of the invention.

The antibodies or antigen-binding antibody fragments thereof of this invention are suitable for treatment, prevention or alleviation of abnormal uterine bleeding such as heavy menstrual bleeding, menorrhagia and dysmenorrhea in women with or without leiomyoma, the treatment prevention or alleviation of leiomyoma and endometriosis. Furthermore, The antibodies or antigen-binding antibody fragments thereof of this invention are suitable for the prevention or alleviation of menstruation.

Compositions of the invention may be used for therapeutic or prophylactic applications. The invention, therefore, includes a pharmaceutical composition comprising an inventive antibody or antigen-binding fragment thereof and a pharmaceutically acceptable carrier or excipient therefore.

These and further objects are met with methods and means according to the independent claims of the present invention. The dependent claims are related to specific embodiments.

### DETAILED DESCRIPTION OF THE INVENTION

It is to be understood that this invention is not limited to the particular component parts or structural features of the devices or compositions described or process steps of the methods described as such devices and methods may vary. It is also to be understood that the terminology used herein is for purposes of describing particular embodiments only and is not intended to be limiting. The mere fact that certain measures are recited in mutually different dependent claims does not indicate that a combination of these measures cannot be used to advantage. Any reference signs in the claims should not be construed as limiting the scope. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment but with another, the skilled person would understand that does not necessarily mean that said feature is not meant to be disclosed with said another embodiment. The skilled person would understand that it is the gist of this application to disclose said feature also for the other embodiment, but that just for purposes of clarity and to keep the length of this specification manageable. It is further to be understood that the content of the prior art documents referred to herein is incorporated by reference, e.g., for enablement purposes, namely when e.g., a method is discussed details of which are described in said prior art document. This approach serves to keep the length of this specification manageable.

### Definitions

Unless otherwise defined, all scientific and technical terms used in the description, figures and claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and are not intended to be limiting.

Unless stated otherwise, the following terms used in this document, including the description and claims, have the definitions given below.

The terms “comprising”, “including”, “containing”, “having” etc. shall be read expansively or open-ended and without limitation. The term comprising when used in the specification includes “consisting of”.

Singular forms such as “a”, “an” or “the” include plural references unless the context clearly indicates otherwise. Thus, for example, reference to “a monoclonal antibody” includes a single monoclonal antibody as well as a plurality of monoclonal antibodies, either the same or different. Likewise reference to “cell” includes a single cell as well as a plurality of cells.

The expression “about” or “˜” as used herein refers to a value being within an acceptable error range for the particular value as determined by one of ordinary skill in the art, which will depend in part on how the value is measured or determined, i.e., on the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects as described herein. In this context “about” may refer to a range above and/or below of up to 10%. Wherever the term “about” is specified for a certain assay or embodiment, that definition prevails for the particular context.

The term “amino acid” or “amino acid residue” as used herein typically refers to a naturally-occurring amino acid. The one letter code is used herein to refer to the respective amino acid. As used herein, a “charged amino acid” is an amino acid which is negatively charged or positively charged. “Negatively charged amino acids” are aspartic acid (D) and glutamic acid (E). “Positively charged amino acids” are arginine (R) lysine (K) and histidine (H). “Polar amino acids” are all amino acids that form hydrogen bonds as donors or acceptors. These are all charged amino acids and asparagine (N), glutamine (Q), serine (S), threonine (T), tyrosine (Y) and cysteine (C). “Polar uncharged amino acids” are asparagine (N), glutamine (Q), serine (S), threonine (T), tyrosine (Y) and cysteine (C). “Amphiphatic amino acids” are tryptophan (W), tyrosine (Y) and methionine (M). “Aromatic amino acids” are phenylalanine (F), tyrosine (Y), and tryptophan (W). “Hydrophobic amino acids” are glycine (G), alanine (A), valine (V), leucine (L), isoleucine (I), proline (P), phenylalanine (F), methionine (M) and cysteine. “Small amino acids” are glycine (G), alanine (A), serine (S), proline (P), threonine (T), aspartic acid (D) and asparagine (N).

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Where generic reference is made to a gene or protein from a certain species such as mouse, the analogue from human shall likewise be meant, if not stated otherwise or obviously incompatible. This holds in particular in the context of biomarkers.

The term “isolated” when applied to a nucleic acid, polypeptide, protein or antibody, denotes that the nucleic acid, polypeptide, protein or antibody is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high-performance liquid chromatography. A protein, polypeptide or antibody that is the predominant species present in a preparation is substantially purified. In particular, an isolated gene is separated from open reading frames that flank the gene and encode a protein other than the gene of interest. An isolated polypeptide may however be immobilized, e.g., on beads or particles, e.g., via a suitable linker.

The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

As used herein, the term “synthetic”, with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

“Post-translational modification(s)” (PTM) refer to the covalent modification(s) of peptides or proteins, which are introduced following protein biosynthesis under natural conditions. The term includes without limitation glycosylation, phosphorylation, acylation, adenylation, farnesylation, ubiquitination, and sulfation. Post-translational modifications may influence the activity of peptides or proteins.

“Sequence identity” or “percent identity” is a number that describes how similar a query sequence is to a target sequence, more precisely how many characters in each sequence are identical after alignment. The most popular tool to calculate sequence identity is BLAST (basic local alignment search tool, https://blast.ncbi.nlm.nih.gov/), which performs comparisons between pairs of sequences, searching for regions of local similarity. Suitable alignment methods are known in the art, e.g., Needleman-Wunsch algorithm for global-global alignment, using BLOSUM62 matrix, with gap opening penalty of 11 and a gap extension penalty of 1. Afterwards, the pairs of aligned identical residues can be counted and then divided by the total length of the alignment (including gaps, internal as well as external) to arrive at the percent identity value.

For “percent similarity” or “sequence similarity” values, the same approach as for percent identity values can be used, except that what is counted, instead of pairs of identical residues, is the aligned residue pairs with BLOSUM62 values that are not negative (i.e., ≥0).

An “isotype control” is an antibody or fragment that does not bind a target but has the same class and type as the reference antibody or fragment recognizing the target.

An antibody or fragment is termed “cross-reactive” or “cross reactive” if the antibody or fragment binds an antigen from two or more different species, e.g., with a K_{D} value of 1.0 E-07 M or less, more preferably of less than 1.0 E-08 M, even more preferably less than 1.0 E-09 M even more preferably less than 1.0 E-10 M.

By the term “specifically binds”, “specific to/for” or “specifically recognizes” as used herein with respect to an antibody, is meant an antibody which recognizes a specific antigen, but does not substantially recognize or bind other molecules in a sample: An antibody characterized by substantial unspecific binding would lack therapeutic applicability, such that these embodiments are excluded. However, as known in the art, specific binding of an antibody or binder does not necessarily exclude an antibody or binder binding to further antigens/target molecules. An antibody that specifically binds to an antigen from one species may also bind to that antigen from one or more further species. Such cross-species reactivity does not itself alter the classification of an antibody as specific.

In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “off target binding” refers to the ability of an antibody to bind individual proteins different from the intended target, for example proteins of the targets' protein family. Off target binding may be evaluated using commercial assays known in the art such as the Retrogenix off target profiling assay. In brief, antibodies are tested on microarrays containing HEK293 cells individually expressing several thousand human membrane proteins and secreted proteins. Binding of the antibody to a potential off target has to be confirmed by FACS using cells overexpressing the potential off target.

The term “affinity” is a term of the art and describes the strength of binding between a binder, antibody or antibody fragment and a target. The “affinity” of antibodies and fragments thereof for a target can be determined using techniques well known in the art or described herein, for example by ELISA, isothermal titration calorimetry (ITC), surface plasmon resonance (SPR), flow cytometry or fluorescent polarization assays. Preferably the affinity is provided as dissociation constant K_{D}.

The “dissociation constant” or “K_{D}” or “KD” has molar units [M] and corresponds to the concentration of the binder/antibody at which half of the target proteins are occupied at equilibrium. The smaller the dissociation constant is, the higher is the affinity between the binder or antibody and its target.

“Half maximal effective concentration” or “EC50” or “EC₅₀” refers to the concentration of a drug, antibody, fragment, conjugate or molecule which induces a response halfway between the baseline and maximum after a specified incubation time. In the context of antibody binding, the EC50 thus reflects the antibody concentration needed for half-maximal binding. An EC50 can be determined if an inflection point can be determined by mathematical modeling (e.g., non-linear regression) of the dose-response curve describing the relationship between applied drug, antibody, fragment, conjugate or molecule concentration and signal. For example, if the dose-response curve follows a sigmoidal curve, an EC50 can be determined. Where the response is an inhibition, the EC50 is termed half maximal inhibitory concentration (IC50).

The term “antibody” (Ab) refers to an immunoglobulin molecule (e.g. without limitation human IgG1, IgG2, IgG3, IgG4, IgM, IgD, IgE, IgA1, IgA2, mouse IgG1, IgG2a, IgG2b, IgG2c, IgG3, IgA, IgD, IgE or IgM, rat IgG1, IgG2a, IgG2b, IgG2c, IgA, IgD, IgE or IgM, rabbit IgA1, IgA2, IgA3, IgE, IgG, IgM, goat IgA, IgE, IgG1, IgG2, IgE, IgM or chicken IgY) that specifically binds to, or is immunologically reactive with, a particular antigen. Antibodies or antibody fragments comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. As used herein, numbering of immunoglobulin amino acid residues is done according to the immunoglobulin amino acid residue numbering system of Kabat et al. The variable domains of native heavy and light chains each comprise four FR regions. The three CDRs in each chain are held together in close proximity by the FR regions and, with the CDRs from the other chain, contribute to the formation of the antigen binding site of antibodies, see Kabat, E. A., et al. “Sequences of Proteins of Immunological Interest (Natl. Inst. Health, Bethesda, MD), GPO Publ.” No 165-462 (1987). The term antibody as used herein also refers to antibody fragments, except where explicitly stated otherwise. Depending on the respective context, the term antibody may also refer to any proteinaceous binding molecule with immunoglobulin-like function.

The term “CDR” refers to the complementary determining region of the antibody. As known in the art complementarity-determining regions (CDRs) are part of the variable chains in antibodies and T cell receptors. A set of CDRs constitutes a paratope. CDRs are crucial to the diversity of antigen specificities. There are usually six CDRs that can collectively come into contact with the antigen. The CDRs of the light chain are LCDR1, LCDR2 and LCDR3. The CDRs of the heavy chain are termed HCDR1, HCDR2 and HCDR3. HCDR3 is the most variable complementary determining region (see, e.g., Chothia, Cyrus, and Arthur M. Lesk. “Canonical structures for the hypervariable regions of immunoglobulins.” Journal of molecular biology 196.4 (1987): 901-917; Kabat, E. A., et al. “Sequences of proteins of immunological interest. Bethesda, MD: US Department of Health and Human Services.” Public Health Service, National Institutes of Health (1991): 103-511.).

The “constant region” refers to the portion of the antibody molecule that confers effector functions. The heavy chain constant region can be selected from any of the five isotypes: alpha (α), delta (δ), epsilon (ε), gamma (g), or mu (μ).

The term “Fc domain”, “Fc region” or “Fc part” as used herein refers to a C-terminal region of an antibody heavy chain that contains at least a portion of the constant region. The term includes native sequence Fc regions and variant Fc regions. For example, a human IgG heavy chain Fc region may extend from Cys226, or from Pro230, to the carboxyl-terminus of the heavy chain.

Antibodies or binding fragments according to the current invention may have been modified to alter at least one constant region-mediated biological effector function. For example, in some embodiments, an antibody may be modified to reduce or enhance at least one constant region-mediated biological effector function relative to the unmodified antibody, e.g., reduced or improved binding to the Fc receptor (FcγR). FcγR binding may be reduced, e.g., by mutating the immunoglobulin constant region segment of the antibody at particular regions necessary for FcγR interactions (see, e.g., Canfield, Stephen M., and Sherie L. Morrison. “The binding affinity of human IgG for its high affinity Fc receptor is determined by multiple amino acids in the CH2 domain and is modulated by the hinge region.” The Journal of experimental medicine 173.6 (1991): 1483-1491; and Lund, John, et al. “Human Fc gamma RI and Fc gamma RII interact with distinct but overlapping sites on human IgG.” The Journal of Immunology 147.8 (1991): 2657-2662.). FcγR binding may be enhanced, e.g. by afucosylation. Reducing FcγR binding may also reduce other effector functions which rely on FcγR interactions, such as opsonization, phagocytosis and antigen-dependent cellular cytotoxicity (“ADCC”).

Furthermore, addressing the interaction of Fc with FcRn allows to modulate the half-life of antibodies in vivo. Abrogating the interaction by e.g., introduction of mutation H435A leads to an extremely short half-life, since the antibody is no longer protected from lysosomal degradation by FcRn recycling. In some preferred embodiments according to all aspects, the antibody according to the current invention comprises mutation H435A or has otherwise been engineered for a reduced half-life.

The terms “anti-IL-11 antibody” or “IL-11 antibody” and “an antibody that binds to IL-11” refer to an antibody that is capable of binding IL-11 with sufficient affinity such that the antibody is useful as a diagnostic and/or therapeutic agent in targeting IL-11. According to the invention, the antibodies preferably have a target affinity of less than 1.0 E-08 M (as KD value), more preferably less than 1.0 E-09 M, even more preferably less than 5.0 E-10 M, even more preferably less than 1.0 E-10 M, even more preferably less than 5.0 E-11 M, even more preferably less than 2.5 E-11 M, even more preferably less than 1.0 E-11 M. The KD values can be preferably determined by means of surface plasmon resonance spectroscopy, e.g., as described elsewhere herein. In certain embodiments, an IL-11 antibody binds to an epitope of IL-11 that is conserved among IL-11 from different species.

The terms “anti-IL-11Ra antibody” or “IL-11Ra antibody” and “an antibody that binds to IL-11Ra” refer to an antibody that is capable of binding IL-11Ra with sufficient affinity such that the antibody is useful as a diagnostic and/or therapeutic agent in targeting IL-1Ra. According to the invention, the antibodies preferably have a target affinity of at least 1.0 E-07 M (as KD value), more preferably of at least 1.0 E-08 M, even more preferably in the range from 1.0 E-09 M to 1.0 E-10 M. The KD values can be preferably determined by means of surface plasmon resonance spectroscopy or ELISA.

A “fragment” of an antibody as used herein is required to substantially retain the desired affinity of the full-length antibody. As such, suitable fragments of e.g., an anti-human IL-11 antibody will retain the ability to e.g., bind to human IL-11 receptor. Fragments of an antibody comprise a portion of a full-length antibody, generally the antigen binding or variable region thereof. Examples of antibody fragments include, but are not limited to, Fab, Fab′, F(ab′)2, and Fv fragments, single-chain antibody molecules, diabodies and domain antibodies, see Holt, Lucy J., et al. “Domain antibodies: proteins for therapy.” Trends in biotechnology 21.11 (2003): 484-490.

A “Fab fragment” contains the constant domain of the light chain and the first constant domain (CH2) of the heavy chain.

“Fab′ fragments” differ from Fab fragments by the addition of a few residues at the carboxyl terminus of the heavy chain CH2 domain including one or more cysteines from the antibody hinge region.

“F(ab′) fragments” are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of intact antibody, clear more rapidly from the circulation of animals, and may have less non-specific tissue binding than an intact antibody, see, e.g., Wahl, Richard L., Charles W. Parker, and Gordon W. Philpott. “Improved radioimaging and tumor localization with monoclonal F (ab′) 2.” Journal of nuclear medicine: official publication, Society of Nuclear Medicine 24.4 (1983): 316-325.

An “Fv fragment” is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Often, the six CDRs confer antigen binding specificity upon the antibody. However, in some instances even a single variable domain (or half of an Fv comprising only three CDRs specific for a target) may have the ability to recognize and bind the antigen, although at a lower affinity than the entire binding site.

“Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for antigen binding.

“Bispecific antibodies” are monoclonal antibodies that have binding specificities for at least two different epitopes on the same or different antigens. In the present disclosure, the binding specificities can be directed towards e.g., two different epitopes of IL-11. It is also possible that one of the binding specificities can be directed towards e.g., IL-11 the other can be for any other antigen, e.g., without limitation for a cell-surface protein, receptor, receptor subunit, tissue-specific antigen, virally derived protein, virally encoded envelope protein, bacterially derived protein, or bacterial surface protein.

“Derivatized antibodies” are typically modified by glycosylation, acetylation, pegylation, phosphorylation, sulfation, amidation, derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin, etc. Additionally, the derivative may contain one or more non-natural amino acids, e.g., using ambrx technology, see, e.g., Wolfson, Wendy. “Amber codon flashing ambrx augments proteins with unnatural amino acids.” Chemistry & biology 13.10 (2006): 1011-1012. Antibodies according to the current invention may be derivatized, e.g., glycosylated or sulfated.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining IL-11 or IL-11RA binding capacities.

Other antibody derivatives known to the skilled person are diabodies, camelid antibodies, nanobodies, domain antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g. antibody or fragments or derivatives linked to a toxin, a cytokine, a radioisotope or a label). These types are well described in literature and can be used by the skilled person on the basis of the present disclosure with adding further inventive activity.

As used herein, the term “antibody mimetic” relates to an organic molecule, most often a protein that specifically binds to a target protein, similar to an antibody, but is not structurally related to antibodies. Antibody mimetics are usually artificial peptides or proteins with a molar mass of about 3 to 20 kDa. The definition encompasses, inter alia, affibody molecules, affilins, affimers, affitins, alphabodies, anticalins, avimers, DARPins, fynomers, kunitz domain peptides, monobodies, and nanoCLAMPs

“Monoclonal antibodies” are substantially homogenous populations of antibodies binding a particular antigen. Monoclonal immunoglobulins may be obtained by methods well known to those skilled in the art (see for example, Kohler, Georges, and Cesar Milstein. “Continuous cultures of fused cells secreting antibody of predefined specificity.” nature 256.5517 (1975): 495-497., and U.S. Pat. No. 4,376,110). An immunoglobulin or immunoglobulin fragment with specific binding affinity can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms. The antibodies according to the current invention are preferably monoclonal.

“Humanized antibodies” contain CDR regions derived from a non-human species, such as mouse, that have, for example, been engrafted, along with any necessary framework back-mutations, into human sequence-derived V regions. Thus, for the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues. Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally comprises at least a portion of an immunoglobulin constant region (Fc), typically that of a human immunoglobulin. For further details see Jones, Peter T., et al. “Replacing the complementarity-determining regions in a human antibody with those from a mouse.” Nature 321.6069 (1986): 522-525; Riechmann, Lutz, et al. “Reshaping human antibodies for therapy.” Nature 332.6162 (1988): 323-327; and Presta, Leonard G. “Antibody engineering.” Current Opinion in Structural Biology 2.4 (1992): 593-596., each incorporated herein by reference.

Fully human antibodies (human antibodies) comprise human derived CDRs, i.e., CDRs of human origin. Preferably, a fully human antibody according to the current invention is an antibody having at least 90%, 91%, 92%, 93%, 94%, 95%, 96%, 97%, 98%, 99%, 99.5% or 100% sequence identity with the closest human VH germline gene (e.g., sequence extracted from recommended list and analyzed in IMGT/Domain-gap-align).

As accepted by usual nomenclature systems such as the INN species subsystem in force until 2017, fully human antibodies may comprise a low number of germline deviations compared with the closest human germline reference determined based on the IMGT database (http://www.imgt.org, Nov. 29, 2019). For example, a fully human antibody according to the current invention may comprise up to 1, 2, 3, 4, 5, 6, 7, 8, 9, 10, 12, 13, 14 or 15 germline deviations in the CDRs compared with the closest human germline reference. Fully human antibodies can be developed from human derived B cells by cloning techniques in combination with a cell enrichment or immortalization step. The majority of fully human antibodies in clinical use, however, were isolated either from immunized mice transgenic for the human IgG locus or from sophisticated combinatorial libraries by phage display (Brüggemann, Marianne, et al. “Human antibody production in transgenic animals.” Archivum immunologiae et therapiae experimentalis 63.2 (2015): 101-108; Carter, Paul J. “Potent antibody therapeutics by design.” Nature reviews immunology 6.5 (2006): 343-357; Frenzel, André, Thomas Schirrmann, and Michael Hust. “Phage display-derived human antibodies in clinical development and therapy.” MAbs. Vol. 8. No. 7. Taylor & Francis, 2016; Nelson, Aaron L., Eugen Dhimolea, and Janice M. Reichert. “Development trends for human monoclonal antibody therapeutics.” Nature reviews drug discovery 9.10 (2010): 767-774.).

Several techniques are available to generate fully human antibodies or to generate antibodies comprising human derived CDRs (cf. WO2008112640). Cambridge Antibody Technologies (CAT) and Dyax have obtained antibody cDNA sequences from peripheral B cells isolated from immunized humans and devised phage display libraries for the identification of human variable region sequences of a particular specificity. Briefly, the antibody variable region sequences are fused either with the Gene III or Gene VIII structure of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody having the desired isotype using established antibody engineering techniques. Antibodies constructed in accordance with this method are considered fully human antibodies (including the CDRs). In order to improve the immunoreactivity (antigen binding affinity and specificity) of the selected antibody, an in vitro maturation process can be introduced, including a combinatorial association of different heavy and light chains, deletion/addition/mutation at the CDR3 of the heavy and light chains (to mimic V-J, and V-D-J recombination), and random mutations (to mimic somatic hypermutation). An example of a “fully human” antibody generated by this method is the anti-tumor necrosis factor α antibody, Humira (adalimumab).

The term “polynucleotide” refers to a recombinantly or synthetically produced polymeric desoxyribonucleotide or analog thereof, or a modified polynucleotide. The term comprises double and single stranded DNA or RNA. The polynucleotide can be integrated e.g., into minicircles, plasmids, cosmids, minichromosomes, or artificial chromosomes. The polynucleotide can be isolated or integrated in another nucleic acid molecule, e.g., in an expression vector or chromosome of a eukaryotic host cell.

The term “vector”, as used herein, refers to a nucleic acid molecule capable of propagating a nucleic acid molecule to which it is linked. The term further comprises plasmids (non-viral) and viral vectors. Certain vectors are capable of directing the expression of nucleic acids or polynucleotides to which they are operatively linked. Such vectors are referred to herein as “expression vectors”. Expression vectors for eukaryotic use can be constructed by inserting a polynucleotide sequence encoding at least one protein of interest (POI) into a suitable vector backbone. The vector backbone can comprise the necessary elements to ensure maintenance of the vector and, if desirable, to provide amplification within the host. For viral vectors, e.g., lentiviral or retroviral vectors, further virus specific elements such as structural elements or other elements can be required and are well known in the art. These elements can be for instance provided in cis (on the same plasmid) or in trans (on a separate plasmid). Viral vectors may require helper viruses or packaging lines for large-scale transfection. Vectors may contain further elements such as e.g., enhancer elements (e.g., viral, eukaryotic), introns, and viral origins of plasmid replication for replication in mammalian cells. According to the current invention, expression vectors typically have a promoter sequence that drives expression of the POI. Expression of the POI and/or selective marker protein may be constitutive or regulated (e.g., inducible by addition or removal of small molecule inductors). Preferred regulatory sequences for mammalian host cell expression include viral elements that direct high levels of expression of a POI in mammalian cells, such as regulatory elements, promoters and/or enhancers derived from cytomegalovirus (CMV), Simian Virus 40 (SV40), adenovirus, (e.g., the adenovirus major late promoter Ad LP) or polyoma. For further description of viral regulatory elements, and sequences thereof, see e.g., U.S. Pat. Nos. 5,168,062, 4,510,245 and 4,968,615.

A “host cell” is a cell that is used to receive, maintain, reproduce and amplify a vector. A host cell also can be used to express the polypeptide, e.g., an antibody or fragment thereof encoded by the vector. The nucleic acid contained in the vector is replicated when the host cell divides, thereby amplifying the nucleic acids. Preferred host cells are mammalian cells, such as CHO cells or HEK cells.

“Linkers for polypeptides” may be attached through an amide linkage or any other functional residue. Linkers for polypeptides may be attached N-terminal or C-terminal of the polypeptide or may be attached via a reactive functional group or amino acid side chain. Polypeptides may be coupled for example to biotin, proteins such as human serum albumin (HSA), carrier proteins such as keyhole limpet hemocyanin (KLH), ovalbumin (OVA) or bovine serum albumin (BSA), fluorescent dyes, short amino acid sequences such as Flag tag, HA tag, Myc tag or His tag, reactive tags such as maleimides, iodoacetamides, alkyl halides, 3-mercaptopropyl or 4-azidobutyric acid, or to various further suitable moieties. Non-limiting examples for suitable linkers, e.g., for conjugation of polypeptides, include beta-alanine, 4-aminobutyric acid (GABA), (2-aminoethoxy) acetic acid (AEA), 5-aminovaleric acid (Ava), 6-aminohexanoic acid (Ahx), PEG2 spacer (8-amino-3,6-dioxaoctanoic acid), PEG3 spacer (12-amino-4,7,10-trioxadodecanoic acid), PEG4 spacer (15-amino-4,7,10,13-tetraoxapenta-decanoic acid), and Ttds (Trioxatridecan-succinamic acid). In some cases, the linker may be derived from a reactive moiety, e.g., maleimides, iodoacetamides, alkyl halides, 3-mercaptopropyl or 4-azidobutyric acid. In some cases, the linker may comprise polyethylene glycol (PEG), polypropylene glycol, polyoxyalkylenes, or copolymers of polyethylene glycol or polypropylene glycol.

“Treating” a disease in a subject or “treating” a subject having a disease refers to subjecting the subject to a pharmaceutical treatment, e.g., the administration of a drug, such that at least one symptom of the disease is decreased or prevented from worsening.

The terms “prevent”, “preventing”, “prevention” and the like refer to reducing the probability of developing a disease, disorder, or condition in a subject, who does not have, but is at risk of or susceptible to developing a disease, disorder, or condition.

The term “effective amount” or “therapeutically effective amount” are used interchangeably herein and refer to an amount sufficient to achieve a particular biological result or to modulate or ameliorate a symptom in a subject, or the time of onset of a symptom. For the treatment of heavy menstrual bleeding, abnormal uterine bleeding or heavy menstrual bleeding secondary to leiomyoma or endometriosis a typical effective amount is an amount that results in at least about 35%; usually by at least about 50%, preferably at least about 60%, or more preferably at least about 70% reduction of bleeding. An effective amount for a particular subject may vary depending on factors such as the condition being treated, the overall health of the subject, the method, route, and dose of administration and the severity of side effects. When in combination, an effective amount is in ratio to a combination of components and the effect is not limited to individual components alone.

“Pharmaceutical compositions” (also “therapeutic formulations”) of the antibody, fragment or conjugate can be prepared by mixing the antibody having the desired degree of purity with optional physiologically acceptable carriers, excipients or stabilizers, e.g. according to Remington's Pharmaceutical Sciences (18th ed.; Mack Pub. Co.: Eaton, Pa., 1990), e.g. in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween®, Pluronic® or polyethylene glycol (PEG).

Typical “subjects” according to the current invention include human and non-human subjects. Subjects can be mammals such as mice, rats, cats, dogs, primates and/or humans.

As used herein, the term “abnormal uterine bleeding” (AUB) is defined as a change in a woman's menstrual blood loss (MBL) or the degree of MBL or vaginal bleeding pattern which differs from that experienced by the age-matched general female population. Abnormal uterine bleeding includes heavy menstrual bleeding or menorrhagia often associated with dysmenorrhea.

As used herein, the term “Endometriosis” is defined as (i) endometriosis externa with endometrial tissue containing endometrial glands (endometrial epithelial cells) and endometrial stroma outside the uterus (endometriotic lesions), typically present within but not restricted to the peritoneal cavity and (ii) endometriosis interna, also termed adenomyosis with endometrial tissue containing endometrial glands (endometrial epithelial cells) and endometrial stroma within the myometrium.

As used herein, the term “Leiomyoma” (also known as uterine fibroids or myoma) are the most common benign gynecological tumors of women of reproductive age and is defined of muscle cells and other tissues that grow in and around the wall of the uterus, or womb.

As used herein, the term “Dysmenorrhea”, also known as painful periods, or menstrual cramps, is defined as pain during menstruation.

As used herein, the term “Interleukin 11”, “IL-11”, or “IL11”, also known as AIGF (adipogenesis inhibitory factor), refers to proteins with the UniProt IDs P20809 (Human), P47873 (Mouse), P20808 (Macaca fascicularis/Cynomolgus monkey), or Q99MF5 (Rat), as listed in Table 1. IL-11 is a cytokine that belongs to the IL-6-type cytokine family distinguished based on their use of the common co-receptor gp130. Human IL-11 is expressed in two isoforms (Table 1) and the human IL-11 gene is spliced at least into two major variants (Table 2). The NCBI reference identifier of the canonical interleukin 11 mRNA sequences of human, mouse, cynomolgus monkey and rat are shown in Table 2.

TABLE 1
IL-11 protein sequence identifiers

| | UniProt | | SEQ ID | |
|---|---|---|---|---|
| Isoform | Identifier | Species | NO | Remarks |
| 1 | P20809-1 | human | 1 | ‘canonical’ sequence (human IL-11); |
| | | | | including signal sequence aa 1-21 |
| 2 | P20809-2 | human | 2 | differs from canonical sequence as |
| | | | | follows: 1-79 missing. |
| 1 | P47873 | mouse | 16 | ‘canonical’ sequence (mouse IL-11) |
| | | | | including signal sequence aa 1-21 |
| 1 | P20808 | cynomolgus | 17 | ‘canonical’ sequence (cynomolgus |
| | | | | IL-11) including signal sequence aa |
| | | | | 1-21 |
| 1 | Q99MF5 | rat | 18 | ‘canonical’ sequence (rat IL-11) |
| | | | | including signal sequence aa 1-21 |

TABLE 2
IL-11 mRNA sequence identifiers

| | NCBI Reference | | SEQ ID | |
|---|---|---|---|---|
| Variant | Sequence | Species | NO | Remarks |
| 1 | NM_000641.4 | human | 5 | ‘full length’ sequence |
| 2 | NM_001267718.1 | human | 6 | lacks exon 2, an exon in the 5′ coding |
| | | | | region, resulting in a downstream in-frame |
| | | | | start codon compared to variant 1 |
| 1 | NM_008350.4 | mouse | 19 | ‘full length’ sequence |
| X1 | XM_045380640.1 | cynomolgus | 20 | ‘full length’ sequence |
| 1 | NM_133519.5 | rat | 21 | ‘full length’ sequence |

As used herein, the term “IL-11RA” or “IL-11Ra” (UniProt Q14626 (human) P70225 (mouse)) is a subunit of the interleukin 11 receptor and also known as CRSDA, Interleukin 11 receptor alpha subunit, interleukin 11 receptor subunit alpha. The interleukin 11 receptor is a type I cytokine receptor, binding interleukin 11. It is a heterodimer composed of an interleukin 11 receptor alpha subunit and the signal transducing subunit gp130. The membrane bound IL-11RA receptors can be cleaved resulting in the release of a soluble isoform sIL-11RA. IL-11RA is expressed in 2 isoforms (Table 3). The human IL-11RA gene is spliced in several variants, from which variant 3 is relevant for the encoded protein isoforms (Table 4). There are two murine IL-11Ra genes: Il11ra1 (homologue to human IL-11RA) and Il11ra2 (no homologue in human existing) and the encoded proteins are shown in Table 3. For Ilra1 three transcript variants exists, all coding for the same protein, only transcript variant 2 of Ilra1 is given in Table 4. For cynomolgus IL11RA no UniProt ID exists. Therefore, we used the NCBI reference protein ID (Table 3), showing the highest homology to human IL11Ra (Uniprot ID Q14626-1) as well as the corresponding cynomolgus IL11RA NCBI reference mRNA ID (Table 4).

TABLE 3
IL-11 receptor alpha protein sequence identifiers

| | UniProt/NCBI | | | SEQ ID | |
|---|---|---|---|---|---|
| Isoform | RefSeqIdentifier | Species | Alias | NO | Remarks |
| 1 | Q14626-1 | human | HCR1 | 3 | ‘canonical’ sequence (IL-11RA) |
| 2 | Q14626-2 | human | HCR2 | 4 | soluble form (sIL-11RA): differs |
| | | | | | from canonical sequence as |
| | | | | | follows: 391-422 missing |
| 1 | Q64385 | mouse | | 8 | encoded by the mice gene Il11ra1 |
| | | | | | which is homologue to human |
| 1 | P70225 | mouse | | 9 | encoded by the mice gene Il11ra2 |
| 1 | XP_045229354.1 | cynomolgus | | 22 | ‘full length’ sequence |
| 1 | Q99MF4 | rat | | 23 | ‘full length’ sequence |

TABLE 4
IL-11 receptor alpha nucleic acid sequence identifiers

| | NCBI Reference | | SEQ ID | |
|---|---|---|---|---|
| Variant | Sequence | Species | NO | Remarks |
| 3 | NM_001142784.3 | human | 7 | encodes the receptor protein |
| 2 | NM_001163401.1 | mouse | 10 | Il11ra1, transcript variant 2 cDNA |
| | NM_010550.3 | mouse | 11 | Il11ra2 cDNA |
| X4 | XM_045373419.1 | cynomolgus | 24 | ‘full length’ sequence |
| 1 | NM_139116.1 | rat | 25 | ‘full length’ sequence |

The term “gp130” refers to glycoprotein gp130 also known as Interleukin-6-Signal Transducer, IL6ST, CD130, CDW130, GP130, or IL-6RB (UniProt.P40189 (human); Q00560 (mouse)). Gp130 is a transmembrane protein and forms one subunit of the type I cytokine receptor within the IL-6 receptor family. It is often referred to as the common gp130 subunit and is important for signal transduction following cytokine engagement. As with other type I cytokine receptors, gp130 possesses a WSXWS amino acid motif that ensures correct protein folding and ligand binding. It interacts with Janus kinases to elicit an intracellular signal following receptor interaction with its ligand. Structurally, gp130 is composed of five fibronectin type-III domains and one immunoglobulin-like C2-type (immunoglobulin-like) domain in its extracellular portion. IL-11 binds to the IL-11Ra. The complex of these two proteins then associates with gp130. This complex of the 3 proteins then homodimerizes to form a hexameric complex which can produce downstream signals. gp130 has no intrinsic tyrosine kinase activity. Instead, it is phosphorylated on tyrosine residues after complexing with other proteins. The phosphorylation leads to association with JAK/Tyk tyrosine kinases and STAT protein transcription factors. In particular, STAT-3 is activated which leads to the activation of many downstream genes.

TABLE 5
UniProt/NCBI RefSeq identifier of interleukin 6 signal
transducer protein sequences of different species

| | UniProt/NCBI | | SEQ ID | |
|---|---|---|---|---|
| Isoform | RefSeqIdentifier | Species | NO | Remarks |
| 1 | P40189-1 | human | 12 | ‘canonical’ sequence (Interleukin-6 |
| | | | | receptor subunit beta) |
| 2 | P40189-2 | human | n.a. | The sequence of this isoform differs |
| | | | | from the canonical sequence as |
| | | | | follows: 325-329: RPSKA → NIASF |
| | | | | and 330-918: Missing |
| 3 | P40189-3 | human | 13 | The sequence of this isoform differs |
| | | | | from the canonical sequence as |
| | | | | follows: 423-483: Missing |
| 1 | Q6PDI9 | mouse | 26 | |
| X1 | XP_045249657.1 | cynomolgus | 27 | Longest of four isoforms (aa): 924 |
| 1 | P40190 | rat | 28 | |

TABLE 6
NCBI RefSeq identifier of interleukin 6 signal
transducer mRNA sequences of different species

| | NCBI Reference | | SEQ ID | |
|---|---|---|---|---|
| Isoform | Sequence | Species | NO | Remarks |
| 1 | NM_002184.4 | human | 14 | encodes the ‘canonical sequence’ of the |
| | | | | Interleukin-6 receptor subunit beta |
| 2 | NM_175767.3 | human | n.a. | encodes the isoform 3 of the Interleukin-6 |
| | | | | receptor subunit beta |
| 3 | NM_001190981.2 | human | 15 | encodes the isoform 3 of the Interleukin-6 |
| | | | | receptor subunit beta |
| | NM_010560.3 | mouse | 29 | |
| 1 | XM_045393722.1 | cynomolgus | 30 | |
| 1 | NM_001008725.3 | rat | 31 | |

As used herein, the term “IL-11 mediated signaling” refers to signal transduction that is initiated upon binding of IL-11 to IL-11RA and gp130, facilitating the formation of higher order structures involving dimers of gp130:IL-11:IL-11RA complexes. This permits e.g. gp130-associated downstream Janus kinases (JAK) activation, STAT-mediated transcriptional activities, activation of non-canonical MAPK/ERK-dependent signaling, and ERK-dependent transcriptional activities. Either soluble or membrane bound IL-11Ra can form a complex with IL-11. Therefore, it is possible that IL-11 binds to soluble IL-11Ra prior to binding cell surface gp130, which facilitates IL-11 mediated signaling in cells which express gp130 but not IL-11Ra (Lokau et al., 2016 Cell Reports 14, 1761-1773). Because IL-11Ra expression is only observed in a small number of cell types, whereas gp130 is expressed in a wide range of cell types the so-called IL-11 trans signaling might be the most common form of IL-11 mediated signaling.

The term, “IL-11 function blocking antibody”, “anti-IL-11 function blocking antibody” refers to an IL-11 antibody that inhibits the IL-11 mediated signal transduction.

Treatment and/or Prevention of Abnormal Uterine Bleeding

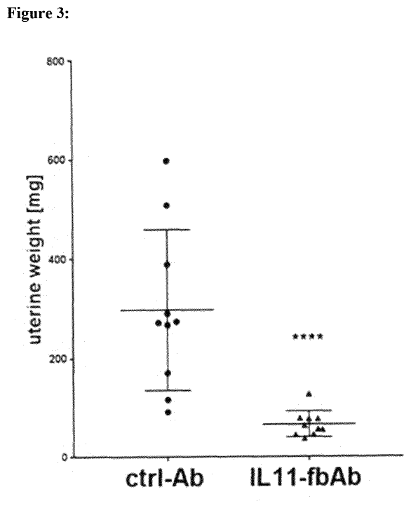

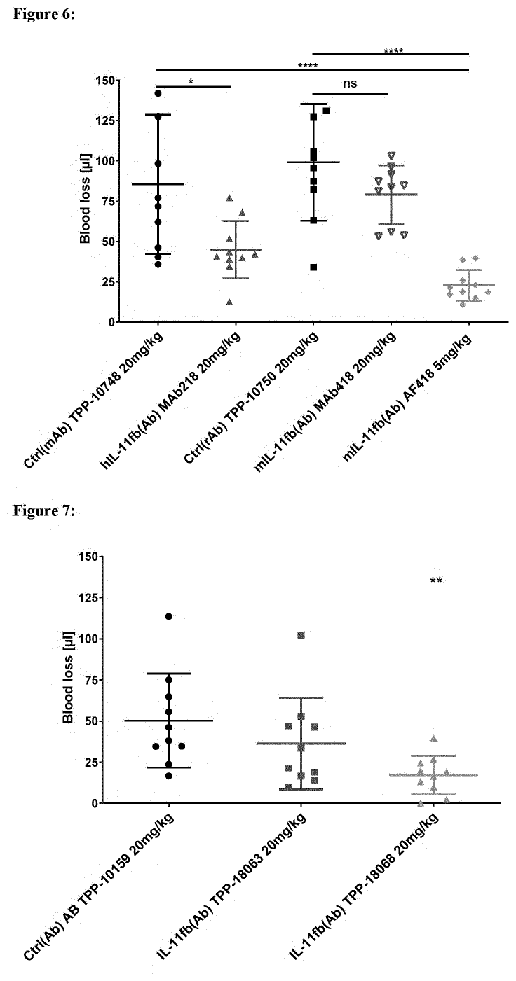

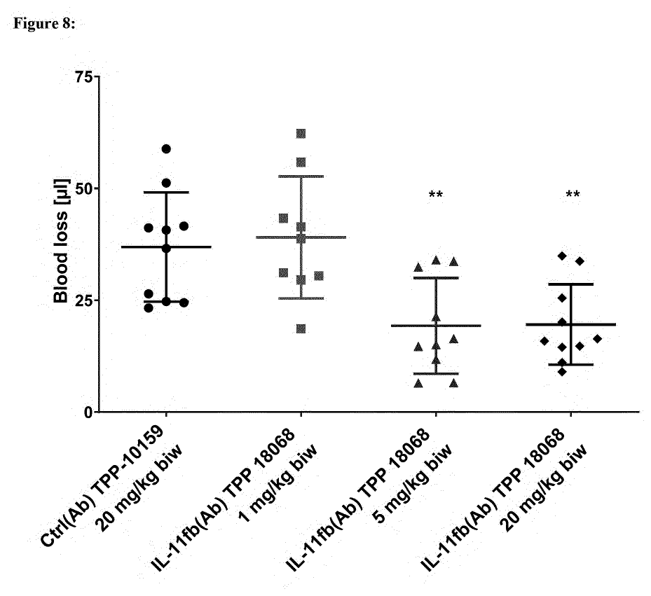

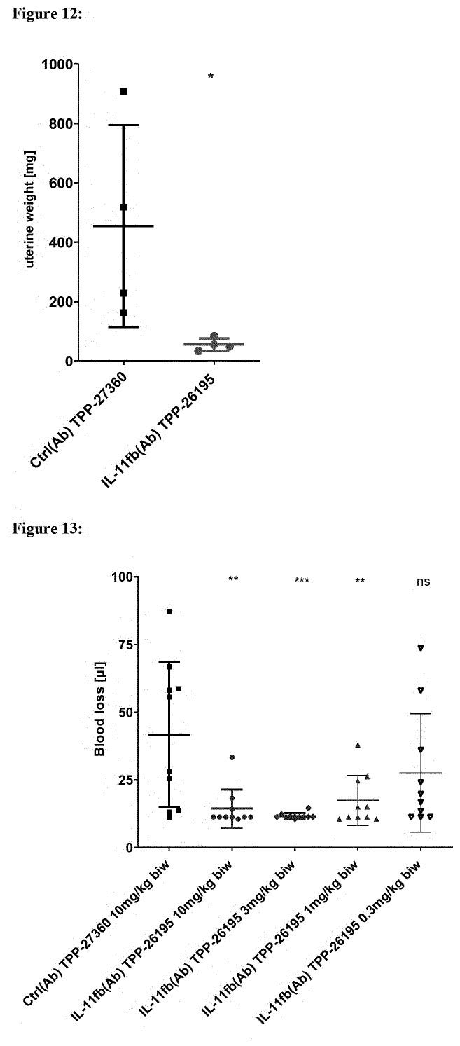

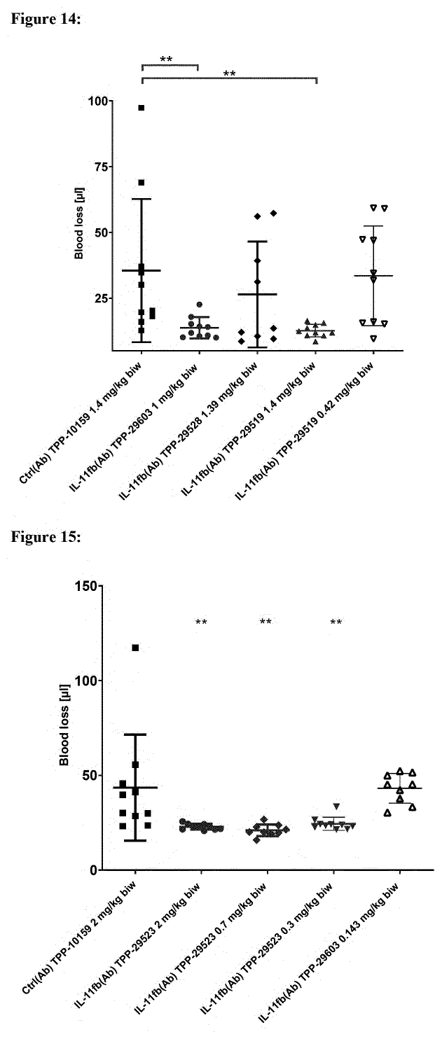

It has been found that the inhibition of the IL-11 signaling pathway by a function blocking IL-11 antibody showed very strong effects on both heavy menstrual bleeding (e.g., Example 1, FIG. 2; Example 5, FIG. 6; Example 6, FIG. 7; Example 7, FIG. 8; Example 10, FIG. 11; Example 11, FIG. 13; Example 12, FIG. 14; Example 13, FIG. 15) and uterus weight (e.g., Example 2, FIG. 3; Example 10, FIG. 12). These surprising results lead to the conclusion that binding to IL-11 and/or IL-11RA and inhibition or antagonizing IL-11 signaling attenuates menstruation and abnormal uterine bleeding including heavy menstrual bleeding.

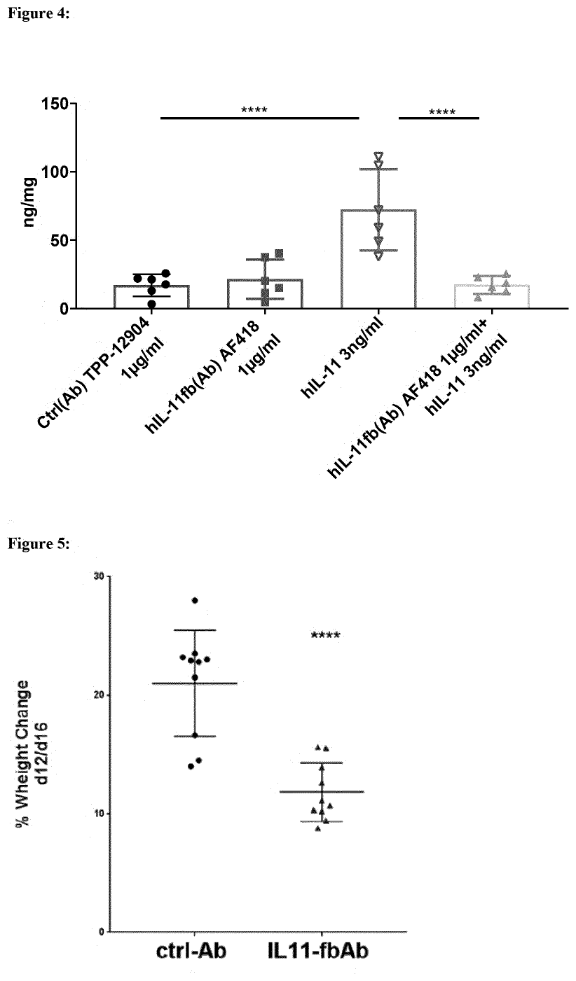

Furthermore, it has been found that IL-11 significantly induced VEGF-A secretion. This was completely inhibited by additional treatment with the IL-11 function blocking antibody. VEGF-A is a well-known proangiogenic mediator, which induction by IL-11 might lead to increased leiomyoma vascularization and enhanced leiomyoma growth (Example 3, FIG. 4).

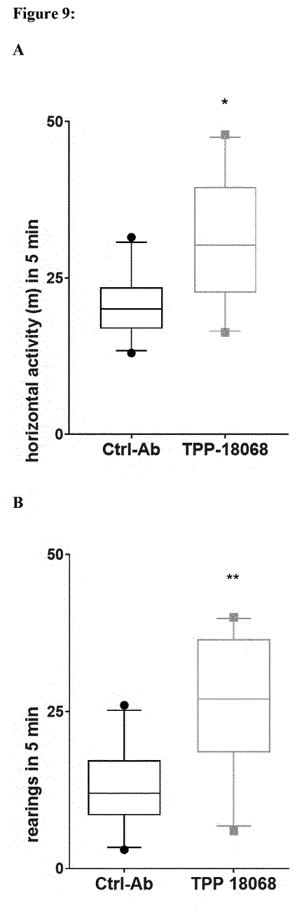

Additionally, it has been found that IL-11 antibody treated animals with HMB showed an attenuation of the bodyweight loss during menstruation compared to animals treated with a control antibody (Example 4, FIG. 5) and that IL-11 antibody treated animals showed increased exploratory behavior as seen in the travelled distance and in rearing compared to animals treated with a control antibody (Example 8; FIG. 9). That indicates that the treatment with an IL-11 function-blocking antibody results in increased activity and increased well-being of the treated animals with HMB during menstruation.

The inhibitory or antagonizing action of the IL-11 and/or IL-11RA agents resulting in attenuation of abnormal uterine bleeding such as heavy menstrual bleeding, menorrhagia, and dysmenorrhea as well as of the underlying diseases such as leiomyoma and endometriosis or menstruation itself is therefore unexpected and surprising.

In accordance with a first aspect, the present invention covers agents which are capable of binding to IL-11 and/or IL-11RA and inhibiting or antagonizing IL-11 mediated signaling for use in the treatment and/or prevention of abnormal uterine bleeding, dysmenorrhea, leiomyoma, or endometriosis.

In accordance with a further aspect, the present invention covers the use of agents which are capable of binding to IL-11 and/or IL-11RA and inhibiting or antagonizing IL-11 mediated signaling for the treatment and/or prevention of abnormal uterine bleeding, dysmenorrhea, leiomyoma, or endometriosis.

In accordance with a further aspect, the present invention covers the use of agents which are capable of binding to IL-11 and/or IL-11RA and inhibiting or antagonizing IL-11 mediated signaling in a method of treatment and/or prevention of abnormal uterine bleeding, dysmenorrhea, leiomyoma, or endometriosis.

In accordance with a further aspect, the present invention covers use of agents which are capable of binding to IL-11 and/or IL-11RA and inhibiting or antagonizing IL-11 mediated signaling for the preparation of a pharmaceutical composition, preferably a medicament, for the treatment and/or prevention of abnormal uterine bleeding, dysmenorrhea, leiomyoma, or endometriosis.

In accordance with a further aspect, the present invention covers a method of treatment and/or prevention of abnormal uterine bleeding, dysmenorrhea, leiomyoma, or endometriosis using an effective amount of agents which are capable of binding to IL-11 and IL-11RA and/or inhibiting or antagonizing IL-11 mediated signaling.

In accordance with an embodiment of all aspects of the invention covers agents which are capable of binding to IL-11 and/or IL-11RA and inhibiting or antagonizing IL-11 mediated signaling for use in the treatment and/or prevention of abnormal uterine bleeding, wherein abnormal uterine bleeding is heavy menstrual bleeding, prolonged bleeding or bleeding with altered bleeding pattern.

In accordance with another embodiment of all aspects, the present invention covers agents which are capable of binding to IL-11 and/or IL-11RA and inhibiting or antagonizing IL-11 mediated signaling for use in the treatment and/or prevention of abnormal uterine bleeding,
wherein abnormal uterine bleeding is heavy menstrual bleeding and wherein heavy menstrual bleeding is secondary to leiomyoma or endometriosis.

In accordance with another embodiment of all aspects, the present invention covers agents which are capable of binding to IL-11 and/or IL-11RA, and inhibiting or antagonizing IL-11 mediated signaling for use in the treatment and/or prevention of abnormal uterine bleeding,
wherein abnormal uterine bleeding is associated with dysmenorrhea.

In accordance with another embodiment of all aspects, the present invention covers agents which are capable of binding to IL-11 and/or IL-11RA and inhibiting or antagonizing IL-11 mediated signaling for use in the treatment and/or prevention of abnormal uterine bleeding,
wherein abnormal uterine bleeding is associated with dysmenorrhea secondary to uterine leiomyoma or endometriosis.

In accordance with a further aspect, the present invention covers use of agents which are capable of binding to IL-11 and/or IL-11RA and inhibiting or antagonizing IL-11 mediated signaling for inhibition or modulation of menstruation.

According to one embodiment of the present invention, the agent capable of binding to and inhibiting or antagonizing the action of IL-11 and/or IL-11RA is an allosteric inhibitor or antagonist. As used herein, the term “allosteric inhibitor” or “allosteric antagonist” relates to an agent that, by binding to an allosteric site of a target protein, alters the protein conformation in the active site of the target, and, consequently changes the shape of active site. Thus, the target e.g, a ligand, no longer remains able to bind to its specific receptors, or experiences a reduced ability to bind its receptors.

According to one embodiment of the present invention, the agent capable of binding to and inhibiting or antagonizing the action of IL-11 and/or IL-11RA is a monoclonal antibody or an IL-11 and/or IL-11RA-binding fragment or derivative thereof retaining IL-11 and/or IL-11RA binding capacities, or an antibody mimetic, which specifically binds to the IL-11 and/or IL-11RA protein.

### Agents

In accordance with another embodiment of all aspects, the present invention covers agents which are capable of binding to IL-11 or IL-11Ra and inhibiting or antagonizing IL-11 mediated signaling for the treatment and/or prevention of abnormal uterine bleeding, dysmenorrhea, leiomyoma, or endometriosis
wherein the agents are allosteric inhibitors or antagonists.

Allosteric inhibitors or antagonists include but are not limited to
IL-11 or IL-11Ra antibodies or IL-11 or IL-11Ra antibody fragments or derivatives thereof
nucleic acid molecules such as but not limited to siRNA (small interfering RNA) or a shRNA (short hairpin RNA)
aptamers that specifically binds to IL-11 or IL-11Ra
small molecules that specifically bind to IL-11 or IL-11Ra

### Antibodies, Antibody Fragments, Antibody-Mimetica or Derivatives Thereof According to the Present Invention

In accordance with another embodiment of all aspects, the present invention covers agents which are capable of binding to IL-11 and/or IL-11RA and inhibiting or antagonizing IL-11 mediated signaling for use in the treatment and/or prevention of abnormal uterine bleeding, wherein the agents are IL-11 and/or IL-11RA antibodies, IL-11 and/or IL-11RA antibody fragments, IL-11 and/or IL-11RA antibody-mimetica or derivatives thereof.

In accordance with another embodiment of all aspects, the present invention covers agents which are capable of binding to IL-11 and/or IL-11RA and inhibiting or antagonizing IL-11 mediated signaling for use in the treatment and/or prevention of abnormal uterine bleeding, wherein the agents is an IL-11 monoclonal antibody and wherein the monoclonal antibody binds to human IL-11 with a dissociation constant (K_{D})≤1.0 E-08 M, ≤1.0 E-09 M, ≤5.0 E-10 M, ≤1.0 E-10 M, ≤5.0 E-11 M, ≤2.5 E-11 M or ≤1.0 E-11 M.

IL-11 and/or IL-11RA antibodies have already been described in the scientific or patent literature already, yet not for use in the treatment of abnormal uterine bleeding, dysmenorrhea, leiomyoma, or endometriosis.

According to the invention IL-11 and/or IL-11RA antibodies include but are not limited to IL-11 and/or IL-11RA antibodies which are described in the scientific and patent literature e.g., recombinant antibody that specifically binds to IL-11 and/or IL-11RA.

Function blocking antibodies for human and mouse IL-11 and/or IL-11RA are provided by commercial suppliers. Examples of known anti-IL-11 antibodies include monoclonal antibody clone 6D9A (Abbiotec or Genetex), clone KT8 (Abbiotec or LS-bio), clone M3103F11 (BioLegend), clone 1F1 (Merck), clone 3C6 (Abnova Corporation), clone GF1 (LifeSpan Biosciences), clone 22616 (Thermo Fisher Scientific), clone 9T27 (Genetex), clone 12 (Thermo Fisher Scientific), unknown clone (LS-bio, #LS-C104441), clone 9 (Thermo Fisher Scientific), clone 13455 (Source BioScience) and clone 22626 (R & D Systems, used in Bockhorn et al. (2013); Monoclonal Mouse IgG2A; Catalog No. MAB218; R&D Systems, MN, USA). Examples of known anti-IL-11RA antibodies include monoclonal antibody clone 025 (Sino Biological), clone EPR5446 (Abcam), clone 473143 (R & D Systems), clones 8E2 and 8E4 described in US 2014/0219919 A1 and the monoclonal antibodies described in Blanc et al. (2000).

In accordance with another embodiment of all aspects, the present invention covers agents which are capable of binding to IL-11 and/or IL-11RA and inhibiting or antagonizing IL-11 mediated signaling for use in the treatment and/or prevention of abnormal uterine bleeding wherein the agent is an IL-11 and/or IL-11RA monoclonal antibody, or an IL-11 and/or IL-11RA-binding antibody fragment or derivative thereof retaining IL-11 and/or IL-11RA binding capacities, or an antibody mimetic, which specifically binds to the IL-11 and/or IL-11RA protein.

As discussed above, IL-11 and/or IL-11RA is sufficiently specified to enable a skilled person to make a monoclonal antibody there against. Routine methods encompass hybridoma, chimerization/humanization, phage display/transgenic mammals, and other antibody engineering technologies.

Methods for the production of a hybridoma cell are disclosed in Köhler & Milstein (1975). Essentially, e.g., a mouse is immunized with a human IL-11 and/or IL-11RA protein, following B-cell isolation and fusion with a myeloma cell.

Methods for the production and/or selection of chimeric or humanised mAbs are known in the art. Essentially, e.g., the protein sequences from a murine anti IL-11 and/or IL-11RA antibody which are not involved in IL-11 and/or IL-11RA binding are replaced by corresponding human sequences. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Methods for the production and/or selection of fully human mAbs are known in the art. These can involve the use of a transgenic animal which is immunized with human IL-11 and/or IL-11RA, or the use of a suitable display technique, like yeast display, phage display, B-cell display or ribosome display, where antibodies from a library are screened against human IL-11 and/or IL-11RA in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis.

IgG, scFv, Fab and/or F(ab)₂ are antibody formats well known to the skilled person. Related enabling techniques are available from the respective textbooks.

Modified antibody formats are for example bi- or trispecific antibody constructs antibody-based fusion proteins, immunoconjugates and the like. These types are well described in literature and can be used by the skilled person on the basis of the present disclosure, with adding further inventive activity.

Finding a suitable antibody, or fragment or derivative, that is capable of acting as an inhibitor or antagonist of IL-11 and/or IL-11RA, e.g., by binding to the IL-11RA, IL-11 or IL6ST interaction sites, is hence a matter of routine for the skilled person, based on the public availability of the amino acid sequences of the different IL-11 and/or IL-11RA isoforms.

Polyclonal antibodies against IL-11 and/or IL-11RA for scientific research are commercially available, e.g., from R&D Systems, Inc., emphasizing that the skilled person is capable of also making therapeutic antibodies against said targets.

### Nucleic Acids

According to one embodiment of the present invention, the agent capable of binding to and inhibiting or antagonizing the action of IL-11 and/or IL-11RA comprises a first nucleic acid molecule that specifically binds to a second nucleic acid molecule, wherein the second nucleic acid molecule encodes for IL-11 and/or IL-11RA protein.

Said second nucleic acid molecule can be an mRNA transcribed from the gene encoding for the IL-11 and/or IL-11RA protein. Said second nucleic is devoid of introns, but due to alternative splicing different mRNAs transcribed from the gene encoding for the IL-11 and/or IL-11RA protein can differ from one another. In such case, the first nucleic acid molecule can be a siRNA (small interfering RNA) or a shRNA (short hairpin RNA).

siRNAs are short artificial RNA molecules which can be chemically modified to enhance stability. Because siRNAs are double-stranded, the principle of the ‘sense’ and the ‘antisense’ strand also applies. The sense strands have a base sequence identical to that of the transcribed mRNA and the antisense strand has the complementary sequence. Technically, a siRNA molecule administered to a patient is bound by an intracellular enzyme called Argonaut to form a so-called RNA-induced silencing complex (RISC). The antisense strand of the siRNA guides RISC to the target mRNA, where the antisense strand hybridizes with the target mRNA, which is then cleaved by RISC. In such way, translation of the respective mRNA is interrupted. The RISC can then cleave further mRNAs. Delivery technologies are e.g., disclosed in Xu and Wang (2015). Finding a suitable sequence for the siRNA is a matter of routine for the skilled person, based on the public availability of the different mRNA isoforms of IL-11 and/or IL-11R.

shRNA is an artificial RNA molecule with a tight hairpin turn that can be used to silence target gene expression via RNA interference (RNAi). shRNA can be delivered to cells, e.g., by means of a plasmid or through viral or bacterial vectors. shRNA is an advantageous mediator of RNAi in that it has a relatively low rate of degradation and turnover. The respective plasmids comprise a suitable promoter to express the shRNA, like a polymerase III promoter such as U6 and H1 or a polymerase II promoter. Once the plasmid or vector has integrated into the host genome, the shRNA is transcribed in the nucleus. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC), after which the same silencing follows as in siRNA. Finding a suitable sequence for the shRNA is a matter of routine for the skilled person, based on the public availability of the different mRNA isoforms of IL-11 OR IL-11RA.

Said second nucleic acid molecule can also be a genomic DNA comprised in the gene encoding for IL-11 and/or IL-11RA protein. Said gene comprises several non-coding introns hence its sequence differs from the sequence of the mRNA or the cDNA disclosed herein.

In such case, the first nucleic acid molecule can be the guide RNA of a CRISPR Cas system (see e.g., Jinek et al. (2012)) which guide RNA comprises a target-specific crRNA (“small interfering CRISPR RNA”) capable of hybridizing with a genomic strand of the IL11 and/or IL11RA gene (or the first nucleic acid molecule can be the crRNA alone). The guide RNA/crRNA is capable of directing the Cas enzyme, which is an endonuclease, to the IL11 and/or IL11RA gene where the Cas enzyme carries out sequence specific strand breaks. By creating one or more double strand breaks the IL11 and/or IL11RA gene hence can be silenced. To use said system for in vivo gene silencing of IL11 and/or IL-11RA, e.g., in different cells of endometrial tissue a dedicated delivery technology is required which comprises a delivery vehicle such as lipid nanoparticles as for example discussed in Yin et al. (2016). Finding a suitable sequence for the crRNA comprised in the guide RNA is a matter of routine for the skilled person based on the public availability of the genomic sequence of IL-11 and/or IL-11RA gene.

In another embodiment, said first nucleic acid molecule can also the guide RNA of a CRISPR Cpf system (Zetsche et al. (2015)), which guide RNA comprises a target-specific crRNA (“small interfering CRISPR RNA”). Similar to CRISPR Cas the guide RNA is capable of directing the Cpf enzyme, which is an endonuclease, to the IL11 and/or IL11RA gene. As regards technical considerations e.g., delivery for in vivo applications and finding of the suitable sequence for the first nucleic acid molecule, similar aspects as with CRISPR Cas apply.

Further embodiments of the CRISPR technology are currently under development, with different endonucleases. However, all these approaches use a target-specific RNA (the guide RNA or crRNA as in CRISPR Cas) that hybridizes with a target sequence. In all these cases the target-specific RNA qualifies as the first nucleic acid molecule in the meaning of the preferred embodiment discussed herein. As regards technical considerations e.g., delivery for in vivo applications and finding of the suitable sequence for the first nucleic acid molecule similar aspects as with CRISPR Cas apply.

According to one embodiment of the present invention, the agent capable of binding to and inhibiting or antagonizing the action of IL-11 and/or IL-11RA is an aptamer that specifically binds to the IL-11 and/or IL-11RA protein.

Aptamers are oligonucleotides that have specific binding properties for a pre-determined target. They are obtained from a randomly synthesized library containing up to 10¹⁵ different sequences through a combinatorial process named SELEX (“Systematic Evolution of Ligands by EXponential enrichment”). Aptamer properties are dictated by their 3D shape resulting from intramolecular folding driven by their primary sequence. An aptamer 3D structure is exquisitely adapted to the recognition of its cognate target through hydrogen bonding, electrostatic and stacking interactions. Aptamers generally display high affinity (K_{d} about micromolar for small molecules and picomolar for proteins).

An overview on the technical repertoire to generate target specific aptamers is given e.g., in Blind and Blank (2015). Aptamers can also be delivered into the intracellular space as disclosed in Thiel & Giangrande (2010).

Finding a suitable aptamer that is capable of acting as an inhibitor or antagonist of IL-11 and/or IL-11RA e.g., by binding to its active center or an allosteric site, is hence a matter of routine for the skilled person, based on the public availability of the amino acid sequences of the different IL-11 and/or IL-11RA isoforms.

According to one embodiment of the present invention the agent capable of binding to and inhibiting or antagonizing the action of IL-11 and/or IL-11RA is a small molecule (SMol) that specifically binds to one or more isoforms of the IL-11 and/or IL-11RA protein.

Methods for the identification and/or selection and/or optimization of suitable inhibitory or antagonising small chemical molecules (SMol) are known in the art. These can involve but are not limited to methods such as binding assays or displacement assays of such SMols or SMol libraries to human or non-human or modified human or modified non-human IL-11 and/or IL-11RA e.g., by measuring e.g., Fluorescence Resonance Energy Transfer (FRET) or Time Resolved Fluorescence Resonance Energy Transfer (TR-FRET) in IL-11/IL-11RA recruitment.

All of these molecules have the potential to act as inhibitors or antagonists of IL-11 and/or IL-11RA for use in the treatment and/or prevention of abnormal uterine bleeding such as heavy menstrual bleeding or menorrhagia, dysmenorrhea, as well as of the underlying diseases leiomyoma and endometriosis and menstruation.

According to one embodiment of the present invention, the antagonist or inhibitor can be found by means of an IL-11/IL-11RA binding assay, inhibition assay, recruitment assay or activation assay.

According to one embodiment of the present invention, the IL-11 and/or IL-11RA protein to which the antibody, fragment or derivative, antibody mimetic, aptamer or small molecule binds comprises a sequence comprised in any of SEQ IDs No 1-4, 8, 9.

According to one embodiment of the present invention, the second nucleic acid molecule encoding the IL-11 and/or IL-11RA protein comprises a nucleotide sequence comprised in any of SEQ IDs No 5-7, 10, 11 or derivatives thereof.

### Pharmaceutical Composition

According to another aspect of the invention, the use of an agent capable of binding to IL-11 and/or IL-11RA and inhibiting or antagonizing the action of IL-11 and/or IL-11RA according to the above description (for the manufacture of a medicament) in the treatment of a human subject being diagnosed for or suffering from abnormal uterine bleeding, heavy menstrual bleeding dysmenorrhea, as well as of the underlying diseases leiomyoma and endometriosis and menstruation is provided.

According to another aspect of the invention, a pharmaceutical composition comprising an agent capable of binding to IL-11 and/or IL-11RA and inhibiting or antagonizing the action of IL-11 and/or IL-11RA according to the above description and one or more pharmaceutically acceptable excipients are provided.

The present invention further provides pharmaceutical compositions comprising an agent capable of binding to IL-11 and/or IL-11RA and inhibiting or antagonizing the action of IL-11 and/or IL-11RA according to the above description and at least one or more than one further active ingredient, especially for treatment and/or prophylaxis of the aforementioned disorders. Preferred examples of such further active ingredients include but are not limited to: selective oestrogen receptor modulators (SERMs), oestrogen receptor (ER) antagonists, aromatase inhibitors, 17β-HSD1 inhibitors, steroid sulphatase (STS) inhibitors, GnRH agonists and antagonists, kisspeptin receptor (KISSR) antagonists, selective androgen receptor modulators (SARMs), androgens, 5α-reductase inhibitors, selective progesterone receptor modulators (SPRMs), gestagens, antigestagens, oral contraceptives, inhibitors of mitogen-activated protein (MAP) kinases and inhibitors of the MAP kinases (Mkk3/6, Mek1/2, Erk1/2), inhibitors of the protein kinases B (PKBα/β/γ; Akt1/2/3), inhibitors of the phosphoinositide 3-kinases (PI3K), inhibitors of cyclin-dependent kinase (CDK1/2), inhibitors of the hypoxia-induced signaling pathway (HIF1alpha inhibitors, activators of prolylhydroxylases), histone deacetylase (HDAC) inhibitors, prostaglandin F receptor (FP) (PTGFR) antagonists, and non-steroidal inflammation inhibitors (NSAIDs).

For example, agents of the present invention can be combined with known antihyperproliferative, cytostatic or cytotoxic substances for treatment of cancers. In addition, inventive agents can also be used in combination with radiotherapy and/or surgical intervention.

Examples of suitable combination active ingredients include but are not limited to:

131I-chTNT, abarelix, abiraterone, aclarubicin, aldesleukin, alemtuzumab, alitretinoin, altretamine, aminoglutethimide, amrubicin, amsacrine, anastrozole, arglabin, arsentrioxidas, asparaginase, azacitidine, basiliximab, RDEA 119, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+oestrone, eculizumab, edrecolomab, elliptinium acetate, eltrombopag, endostatin, enocitabine, epirubicin, epitiostanol, epoetin alfa, epoetin beta, eptaplatin, eribulin, erlotinib, oestradiol, oestramustine, etoposide, everolimus, exemestane, fadrozole, filgrastim, fludarabine, fluorouracil, flutamide, formestane, fotemustine, fulvoestrant, gallium nitrate, ganirelix, gefitinib, gemcitabine, gemtuzumab, glutoxim, goserelin, histamine dihydrochloride, histrelin, hydroxycarbamide, I-125 pellets, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostan, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oxaliplatin, p53 gene therapy, paclitaxel, palifermin, palladium-103 pellets, pamidronic acid, panitumumab, pazopanib, pegaspargase, pEG-epoetin beta (methoxy PEG-epoetin beta), pegfilgrastim, peginterferon alfa-2b, pemetrexed, pentazocine, pentostatin, peplomycin, perfosfamid, picibanil, pirarubicin, plerixafor, plicamycin, poliglusam, polyoestradiol phosphate, polysaccharide-K, porfimer sodium, pralatrexate, prednimustine, procarbazine, quinagolide, radium-223 chloride, raloxifen, raltitrexed, ranimustine, razoxane, regorafenib, risedronic acid, rituximab, romidepsin, romiplostim, sargramostim, sipuleucel-T, sizofiran, sobuzoxan, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

The present invention preferably relates to medicaments comprising at least one agent which is capable of binding to IL-11 and/or IL-11RA and inhibiting or antagonizing IL-11 mediated signaling according to the above description and one or more of the following active ingredients, especially for treatment and/or prophylaxis of steroid receptor-dependent proliferative disorders:
LHRH (luteinizing hormone-releasing hormone) agonists,
LHRH (luteinizing hormone-releasing hormone) antagonists,
C(17,20)-lyase inhibitors,
type I 5-α-reductase inhibitors,
type II 5-α-reductase inhibitors,
mixed type I/II 5-α-reductase inhibitors,
α-radiation-emitting radiopharmaceuticals for treatment of bone metastases, for example radium-223 chloride,
cytostatics,
VEGF (Vascular Endothelial Growth Factor) kinase inhibitors,
antigestagens,
antioestrogens,
EGF antibodies,
oestrogens or
poly(ADP-ribose) polymerase I inhibitors, or
bi-specific T-cell engagers (BiTE) coupled to a cell surface protein, for example prostate-specific membrane antigen (PSMA).

In accordance with a further aspect, the present invention covers pharmaceutical combinations comprising:
(a) one or more agents which are capable of binding to IL-11 and/or IL-11RA and inhibiting or antagonizing IL-11 mediated signaling according to the above description and
(b) one or more further active ingredient.

Another aspect of the invention are methods for identifying an agent for use in the treatment and/or prevention of a patient suffering from, at risk of developing, abnormal uterine bleeding, which method comprises the screening of one or more test agents in a suitable assay.

According to one embodiment, such method further comprises a prior step of creation and/or provision of a library of test compounds.

In accordance with another embodiment of all aspects, the present invention covers methods for determining whether a human or animal subject is suitable of being treated with an agent, a pharmaceutical composition or a combination according to the above description, said method comprising
a. providing a tissue or liquid sample from said subject, and
b. determining whether or not said sample is characterized by expression or overexpression of IL-11 and/or IL-11RA.

According to further aspect of the invention, a method for treating or preventing abnormal uterine bleeding is provided, comprising administering to a subject in need thereof an effective amount of the agent capable of inhibiting or antagonizing the action of IL-11 and/or IL-11RA the pharmaceutical composition according or the combination according to the above description.

In accordance with another embodiment of all aspects, the present invention covers methods for determining whether a human or animal subject is suitable of being treated with an agent, a pharmaceutical composition or a combination according to the above description, said method comprising
a. providing a tissue or liquid sample from said subject, and
b. determining whether or not said sample is characterized by expression or overexpression of IL-11, wherein said expression IL-11 is determined
   i. on an mRNA level (e.g., RT-PCR, in situ PCR and/or Fluorescence in situ hybridization (FISH),
   ii. on a protein level (e.g., with Immunohistochemistry, Immunoblot, ELISA, and the like).

According to another aspect of the invention, a companion diagnostic for use in a method according to the above description is provided, wherein the companion diagnostic comprises at least one agent which is selected from the group consisting of a nucleic acid probe or primer capable of hybridizing to a nucleic acid (DNA or RNA) that encodes an IL-11 and/or IL-11RA protein
a. an antibody that is capable of binding to an IL-11 and/or IL-11RA protein, and/or
b. an aptamer that is capable of binding to an IL-11 and/or IL-11RA protein.

### SCREENING and TARGET Inhibition Assay

Methods for the identification and/or selection and/or optimization of suitable agents capable of binding to and inhibiting or antagonizing the action of IL-11 and/or IL-11RA are known in the art. These can involve but are not limited to methods such as binding assays of such agents capable of inhibiting or antagonizing the action of IL-11 and/or IL-11RA to human or non-human or modified human or modified non-human IL-11 and/or IL-11RA. There are numerous types of ligand binding assays known to the skilled person, both radioactive and non-radioactive assays. Binding of an agent can be measured by using e.g., immobilized IL-11 or IL-11RA and labelled agents e.g., by radioactive labelling with an radioactive isotope or adding e.g. fluorescence moieties, in case of peptide agents further tags can be added as e.g. Fc (fragment crystallisable region of an immunoglobulin)-tag or Avi-tag (Streptavidin-tag). In addition, binding assays can involve but are not limited to methods such as displacement assays as inhibition or antagonization of recruitment of the natural or an artificial binding partner such as e.g., recruitment of human or non-human IL-11 or derivatives thereof on human or non-human IL-11RA or derivatives thereof. Recruitment and inhibition of recruitment can be measured by the skilled person by several methods e.g., measuring e.g., Fluorescence Resonance Energy Transfer (FRET) or Time Resolved Fluorescence Resonance Energy Transfer (TR-FRET) in such IL-11/IL-11RA recruitment. In one embodiment the inhibition and/or antagonization of IL6ST recruitment to IL-11 or IL-11RA or IL-11/IL-11RA complex can be measured. In another embodiment binding or inhibition/antagonization of recruitment can be measured by cellular assays in human or non-human cells, which naturally express IL-11RA (or IL-11RA and IL6ST) or in cells expressing recombinant human or non-human IL-11RA (or IL-11RA and IL6ST) or derivatives thereof.

According to one embodiment of the present invention, the antagonist or inhibitor can be found by means of an IL-11 and/or IL-11RA inhibition assay or activation assay. In one embodiment, such methods may include but are not limited to cellular assays measuring inhibition or antagonization of IL-11 signaling by analysing downstream markers of the IL-11/IL-11RA signaling pathways, such as recruitment of Janus kinases (1) or Signal Transducer and Activator of Transcription proteins (STATs) to the IL-11/IL-11RA/IL6ST signaling complex e.g. with the commercially available assay ‘Phospho-STAT3 (Tyr705) Assay Base Kit’ from MSD (mesoscale) or a Stat3 dependent reporter assay, e.g. ‘STAT3 Reporter Assay By Luciferase’ from Biocompare. In another embodiment phosphorylation of STAT3 or MEK/ERK kinases can be measured by e.g., ELISA techniques in human or non-human cells which naturally express IL-11RA and IL6ST or in cells expressing recombinant human or non-human IL-11RA (or IL-11RA and IL6ST) or derivatives thereof.

### Novel Therapeutic Antibodies for IL-11 and Medical Uses Thereof

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are capable of reducing blood loss during abnormal uterine bleeding.

The reduction of blood loss during menstruation by an isolated antibody or antigen-binding fragment thereof can be analysed in a murine HMB model as described in example 1.

In accordance with another embodiment of all aspects, the present invention covers isolated antibodies or antigen-binding fragments thereof wherein said isolated antibody or antigen-binding fragment thereof reduces blood loss during menstruation by at least about 35%, or by at least about 50%, or preferably by at least about 60%, or more preferably by at least about 70% reduction of bleeding.

Isolated antibodies or antigen-binding fragments thereof capable of binding to IL-11 and inhibiting IL-11 mediated signaling are capable of reducing uterine differentiation as described in example 2. Uterine differentiation as induced in the described animal model of uterine bleeding leads to menstruation and uterine bleeding after removal of progesterone.

Reduction of the uterine weight at day 12 of the described murine HMB model can be interpretated as predictive for the reduction of uterine bleeding as measured between day 12 and day 15 in the murine HMB model as described in example 2.

Heavy menstrual bleeding or menstruation is often characterized by reduced well-being. Avoiding or attenuation of menstruation and heavy menstrual bleeding is an effective treatment for primary and secondary dysmenorrhea. Isolated antibodies or antigen-binding fragments thereof capable of binding to IL-11 and inhibiting IL-11 mediated signaling are capable of attenuating signs of reduced well-being as reduction of body weight loss during menstruation and heavy menstrual bleeding as described in example 4 or attenuating the reduction of explorative behavior at the time of menstruation and heavy menstrual bleeding as shown in example 8.

Another surrogate marker for reduced bleeding in the murine HMB model is the attenuation of IL-11 mediated Stat3 phosphorylation downstream of the activated receptor complex in differentiated uteri at day 12 as shown in example 9.

In accordance with another aspect, the present invention covers isolated antibodies or antigen-binding fragments thereof capable of inhibiting the secretion of VEGF-A by fibroid tissue.

The inhibition of the secretion of VEGF-A by fibroid tissue by an isolated antibody or antigen-binding fragment thereof can be analysed as described in example 3.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof binds to human IL-11 with a dissociation constant (K_{D})≤1.0 E-08 M, ≤1.0 E-09 M, ≤5.0 E-10 M, ≤1.0 E-10 M, ≤5.0 E-11 M, ≤2.5 E-11 M or ≤1.0 E-11 M.

The binding of an isolated antibody or antigen-binding fragment thereof to IL-11 can be analysed by Surface plasmon resonance (SPR) from the Biacore system, as described in example 16, or by ELISA methods as described in Examples 23, and 24. Other methodologies to determine the binding of an isolated antibody or antigen-binding fragment thereof to IL-11 include, but are not limited to, using electro-chemiluminescence method (ELC) via mesoscale discovery (MSD), Luminex xMAP® platform, Immuno-PCR (Lasseter H C et al., 2020, Cytokine X; 28; 2), radioimmunoassays (RIA), fluorescence immunoassays (FIA), thermal shift assays, LC-MS detection, and Bio-layer interferometry (BLI) from Octet system, and kinetic exclusion assay technology, e.g., KinExA® (Sapidyne Instruments, Inc., Boise, ID). KinExA offers a platform that allows the measurement of equilibrium binding affinity and kinetics using unmodified molecules in solution phase. This is accomplished by using a solid-phase immobilized molecule to probe for free concentration of one interaction component after allowing sufficient time to reach equilibrium (affinity measurements), or under pre-equilibrium conditions (kinetics) (Darling R J et al. (2004). ASSAY and Drug Development Technologies. 2 (6): 647-657).

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof inhibits human IL-11 mediated signaling with an IC₅₀ of ≤100 nM, ≤50 nM, ≤25 nM, ≤10 nM, ≤1 nM, ≤0.5 nM or ≤0.1 nM.

An in vitro IL-11 function blocking assay can be an assay as described in example 17. In addition, further in vitro IL-11 function blocking assays are described in the literature. IL-11 signaling and inhibition of its signaling by function blocking antibodies can be measured and quantified by the downstream phosphorylation of STAT3 by analysing the amount of phosphorylated STAT3 in comparison to total STAT3 in western blot based assays after induction of the downstream signaling by recombinant or induced IL-11 in primary cells or a cell line expressing the IL-11Ra and gp130 either intrinsically or after transient or stable transfection of DNA encoding the respective receptors and resulting in expression of these in the transfected cells. Suitable primary cells are e.g., PBMCs of healthy donors as described by Sumida et al., 2015. After induction with recombinant IL-11 (e.g., 10 ng/ml) total STAT3, phosphorylated STAT3 (pSTAT3), and a control protein as e.g. alpha-tubulin protein can be evaluated by immunoblotting using specific antibodies.

In addition an in vitro IL-11 function blocking assay can measure and quantify the cell proliferation induced by e.g. human or murine IL-11 in the T11 mouse plasmacytoma cell line in a dose-dependent manner as described by R&D Systems (https://www.rndsystems.com/products/human-il-11-antibody_af-218-na or Nordan, R. P. et al. (1987) J. Immunol. 139:813). Proliferation elicited by recombinant IL-11 (e.g., by 1 ng/mL) can be inhibited by function blocking antibodies.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof inhibits the interaction of IL-11 with IL-11Ra and wherein said isolated antibody or antigen-binding fragment thereof inhibits the formation of IL-11/IL-11Ra/gp130 complex.

In accordance with another embodiment of all aspects, the present invention covers isolated antibodies or antigen-binding fragments thereof capable of binding to IL-11 and inhibiting IL-11 mediated signaling, wherein said isolated antibodies or antigen-binding fragments thereof inhibits the interaction of IL-11 with IL-11Ra with an IC₅₀≤1000 nM, ≤100 nM, ≤10 nM as determined by use of a Di-complex ELISA and wherein said isolated antibodies or antigen-binding fragments thereof inhibit the formation of IL-11/IL-11Ra/gp130 complex with an IC₅₀≤1000 nM, ≤100 nM, ≤10 nM as determined by use of a Tri-complex ELISA.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof inhibits the interaction of IL-11 with IL-11Ra and wherein said isolated antibody or antigen-binding fragment thereof inhibits the formation of IL-11/IL-11Ra/gp130 complex.

In accordance with another embodiment of all aspects, the present invention covers isolated antibodies or antigen-binding fragments thereof capable of binding to IL-11 and inhibiting IL-11 mediated signaling, wherein said isolated antibodies or antigen-binding fragments thereof inhibit the formation of IL-11/IL-11Ra/gp130 complex with an IC₅₀≤1000 nM, ≤100 nM, ≤10 nM as determined by use of a Tri-complex ELISA and wherein said isolated antibodies or antigen-binding fragments thereof does not inhibit the interaction of IL-11 with IL-11Ra as determined by use of Di-complex ELISA.

The inhibition of IL-11 signaling can be either due to blocking of the interaction site of IL-11 with IL-11Ra or due to the inhibition of the activation of gp130 upon interaction of IL-11:IL-11Ra complex with cell membrane bound gp130. The IL-11:IL-11Ra complexed can comprise soluble or membrane bound IL-11Ra.

The inhibition of the interaction of IL-11 with soluble or membrane bound IL-11Ra by an antibody or antigen-binding fragment thereof can be analysed by various analytical methods. Methods include, but are not limited to e.g., ELISA, FACS, electro-chemiluminescence method (ELC) via mesoscale discovery (MSD), Luminex xMAP® platform, Immuno-PCR (Lasseter H C et al., 2020, Cytokine X; 28; 2), radioimmunoassays (RIA), fluorescence immunoassays (FIA), thermal shift assays, LC-MS detection, Bio-layer interferometry (BLI) from Octet system, and kinetic exclusion assay technology, e.g., KinExA®. One preferred assay format is an ELISA where soluble IL-11Ra binds to immobilized IL-11 in the presence or absence of IL-11 antibodies and the formation of the IL-11:IL-11Ra complex can be detected by a labelled detection reagent e.g., an antibody-enzyme conjugate binding to IL-11Ra. Antibodies interfering with IL-11Ra:IL11 complex formation will reduce the final readout signal in the assay. In order to facilitate binding of the detection reagent, IL-11Ra can be employed as fusion-protein, in which IL-11Ra has been fused by recombinant DNA technology to e.g., the Fc part of an antibody or to a small peptide sequence, particularly suited for recognition by detection reagents, e.g., cMyc-tag, His-tag, or other tags. IL-11Ra can also be chemically modified e.g., biotinylated, in order to allow detection by a reagent specifically recognising the chemical modification e.g., streptavidin-horseradish-peroxidase. ELISA formats employing IL-11Ra-Fc fusion-proteins are described in Example 18. In order to allow to test for inhibition of IL-11:IL-Ra complex formation by human IgGs as well as murine IgGs, two different IL11Ra-Fc fusion proteins were employed. Canine IL-11Ra fused to human Fc (Sino Biological; #70078-D02H) were used when murine IgGs were tested and mouse Il-11Ra fused to mouse Fc (R&D System, #7405-MR) were used when human IgGs were tested. It is apparent, that due to high IL-11Ra homology between species, canine IL-11Ra can be used for complex formation with either human (SEQ ID NO: 1, aa 22-199; e.g., Invigate, e.g., lot #C121021-19) or murine IL-11 (SEQ ID NO: 16; aa 22-199, e.g., Invigate, e.g., lot #C210819-09) and murine Il-11Ra can be used for complex formation with either human (SEQ ID NO: 1; aa 22-199, e.g., Invigate, e.g., lot #C121021-19) or murine Il-11 (SEQ ID NO: 16; aa 22-199, e.g., Invigate, e.g., lot #C210819-09). A schematic drawing of the Di-complex assay formats is shown in FIG. 17 (a, b).

The specific inhibition of the interaction of IL-11:IL-11Ra complex with gp130 by an IL-11 binding antibody or antigen-binding fragment thereof, can be analyzed by the combination of an assay testing for the inhibition of the formation of IL-11:IL-11Ra di-complexes, as described above, and an assay testing for the inhibition of the formation of IL-11Ra:IL-11:gp130 tri-complexes. If an antibody interferes with IL-11Ra:IL11:gp130 tri-complex formation in a tri-complex assay but not with IL-11:IL-11Ra di-complex formation in a di-complex assay, it is concluded, that the antibody binds to an epitope on IL-11 relevant for interaction of IL-11 with gp130 but not relevant for interaction of IL-11 with IL-11Ra. In contrast, an antibody that interferes with IL-11:IL-11Ra complex formation in a di-complex assay is expected to interfere with IL-11Ra:IL-11:gp130 complex formation in a tri-complex assay as well. Importantly, since the antibody interferes with di-complex formation, it is concluded, that the antibody binds to an epitope on IL-11 relevant for the interaction of IL-11 with IL-11Ra.

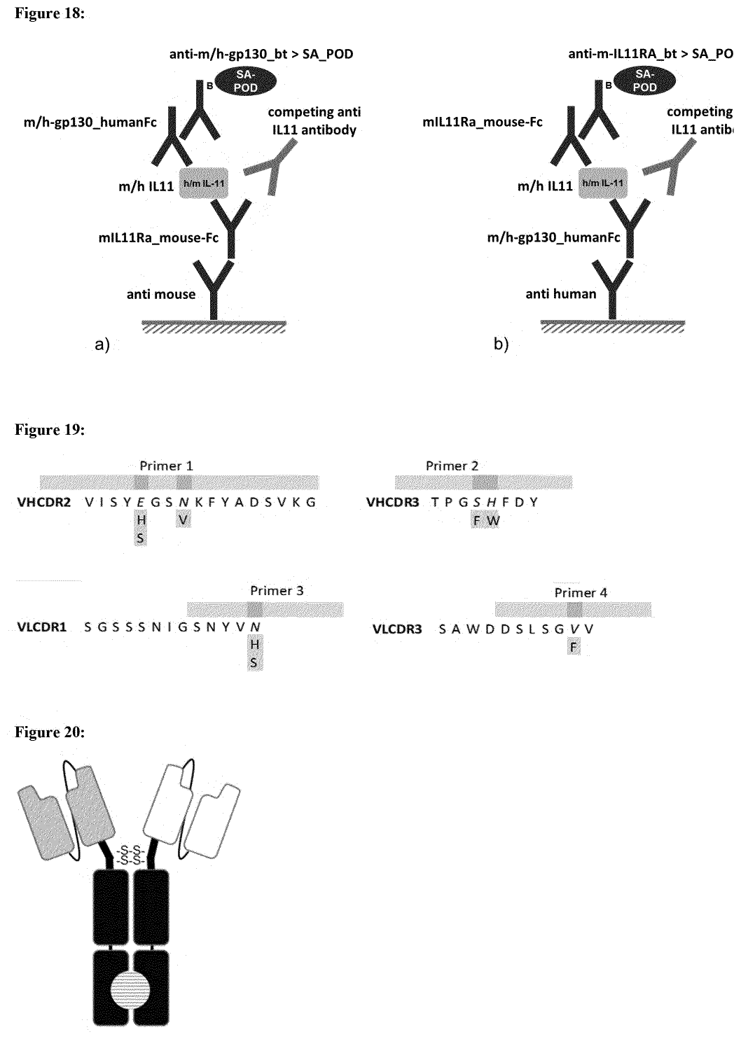

The inhibition of the interaction of an IL-11:IL-11Ra complex with soluble or membrane bound gp130 by an antibody or antigen-binding fragment thereof can be analysed by various analytical methods. Methods include, but are not limited to e.g., ELISA, FACS, electro-chemiluminescence method (ELC) via mesoscale discovery (MSD), Luminex xMAP® platform, Immuno-PCR (Lasseter H C et al., 2020, Cytokine X; 28; 2), radioimmunoassays (RIA), fluorescence immunoassays (FIA), thermal shift assays, LC-MS detection, Bio-layer interferometry (BLI) from Octet system, and homogeneous time resolved fluorescence (HTRF) assays One preferred assay format is an ELISA where soluble IL-11Ra-Fc (e.g., R&D Systems, #7405-MR), human IL-11 (e.g., Invigate, e.g., lot #C121021-19) or murine IL-11 (e.g., Invigate, e.g., lot #C210819-09), and murine (e.g., R&D Systems, #468-MG) or human gp130-Fc (e.g., R&D Systems, #671-GP) are mixed in the presence or absence of IL-11 antibody. Formed tri-complexes of IL-11Ra:IL-11:gp130 are captured by immobilized anti mouse Fc or anti human Fc capture reagents, depending on whether human or murine IL-11 antibodies are tested for inhibition of tri-complex formation. Finally, captured tri-complexes are detected by a biotinylated antibody directed against either human or mouse gp130 in assays testing human IL-11 IgGs for inhibition of tri-complex formation. When mouse IL-11 IgGs are tested for inhibition of tri-complex formation, a biotinylated antibody directed against murine IL-11Ra is used as detection reagent. In both assay formats, for human and mouse IgGs, the presence of biotinylated detection reagent is made visible by streptavidin-POD reagent and substrate. The readout signal will be lower if the tested antibody inhibits tri-complex formation. A schematic drawing of the Tri-complex assay formats is shown in FIG. 18 (a, b).

Antibodies TPP-16478, TPP-18068, TPP-27159, TPP-29386, TPP-29528, TPP-29536 show activity in both, the di-complex and tri-complex ELISA. In addition, competitor antibodies TPP-23552 and TPP-23580 as well as all tested commercially available antibodies with at least some functional activity, were active in both, di-complex and tri-complex ELISA, as well.

None of the tested commercially available antibodies nor the competitor antibodies TPP-23552 and TPP-23580 do show activity in the Tri-complex ELISA and but not in the Di-complex ELISA. In contrast, antibodies TPP-18087, TPP-29519, TPP-29520, TPP-29521, TPP-29522, TPP-29523, TPP-29680, TPP-30000, TPP-30001, TPP-30002, TPP-30003, TPP-31325 and TPP-31385 do show activity in the Tri-complex ELISA and but not in the Di-complex ELISA, indicating, that these antibodies recognize a novel epitope on IL-11 relevant for interaction with gp130 but not with IL-11Ra.

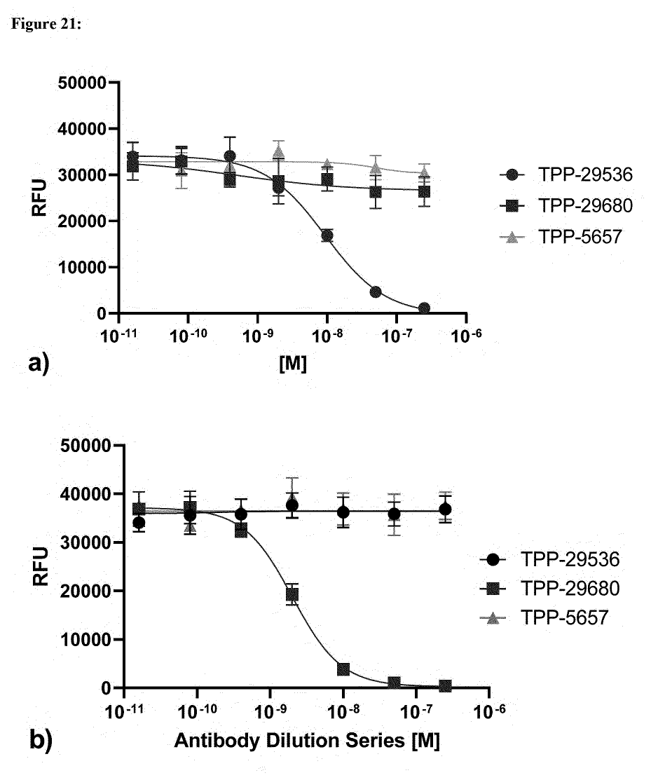

Additionally, it could be shown in example 39 that, increasing concentrations of competing antibody TPP-29536, a derivative of TPP-18068 (active in both di-complex and tri-complex ELISA), completely block binding of TPP-18068 to IL-11, while TPP-29680, a derivative of TPP-18087 (active in tri-complex ELISA but not in di-complex ELISA), has no effect (FIG. 21 a). Furthermore, increasing concentrations of competing antibody TPP-29680, completely block binding of TPP-18087 to IL-11, while TPP-29536 has no effect (FIG. 21 b). These data indicate that the epitopes of TPP-18068 (active in both di-complex and tri-complex ELISA) and its derivatives are distinct and not overlapping with those of TPP-18087 (active in tri-complex ELISA but not in di-complex ELISA) and its derivatives.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are bispecific antibodies or antigen-binding fragments thereof capable of binding to two different IL-11 epitopes.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are bispecific antibodies or antigen-binding fragment thereof capable of binding to two different IL-11 epitopes and wherein said isolated antibodies or antigen-binding fragments thereof inhibit the binding of IL-11 to IL-11Ra and wherein said isolated antibodies or antigen-binding fragments thereof inhibit the formation of IL-11/IL-11Ra/gp130 complex.

The different activity of isolated antibodies or antigen-binding fragments thereof of the present invention in the Di-complex ELISA and Tri-complex ELISA indicates these antibodies or antigen-binding fragments thereof form two classes of IL-11 antibodies that bind to different IL-11 epitopes. Therefore, antibodies of each class can be combined to a bispecific antibody that bind to two different IL-11 epitopes. Such bispecific antibodies comprise the CDR's of an antibody that inhibits the interaction of IL-11 with IL-11Ra and the CDR's of an antibody that inhibits the interaction of IL-11:IL-11Ra complex with gp130. Examples for such antibodies are TPP-20489, TPP-26195, TPP-29603 and TPP-29697.

In accordance with another embodiment of all aspects, the present invention covers isolated antibodies or antigen-binding fragments thereof capable of binding to IL-11 and inhibiting IL-11 mediated signaling, wherein said isolated antibodies or antigen-binding fragments thereof is a bispecific antibody or antigen-binding fragment thereof capable of binding to two different IL-11 epitopes and wherein the bispecific antibody or antigen-binding fragment thereof comprises a first single chain fragments (scFv) comprising a first binding site for a first IL-11 epitope and a second single chain fragments (scFv) comprising a second binding site for a second IL-11 epitope. Each scFv fragment can be fused to a separate Fc domain (e.g., IgG Fc domain) via a linker such as a peptide linker e.g., GG GGSGGGGSGG GGSG (e.g., SEQ ID NO: 74, aa 240-256). One Fc-domain can comprise a knop mutation and the other Fc domain can comprise a corresponding hole mutation. (see also FIG. 20). Upon expression of both chains in a host cell, bispecific antibodies are formed, which can be purified by conventional methods from the culture supernatant.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are bispecific antibody or antigen-binding fragment thereof capable of binding to two different IL-11 epitopes and wherein the bispecific antibodies or antigen-binding fragments thereof comprise
i) a first chain comprising a heavy chain antigen-binding region that comprises an H-CDR1 comprising SEQ ID NO: 33, an H-CDR2 comprising SEQ ID NO: 34, and an H-CDR3 comprising SEQ ID NO: 35 and a light chain antigen-binding region that comprises an L-CDR1 comprising SEQ ID NO: 37, an L-CDR2 comprising SEQ ID NO: 38, and an L-CDR3 comprising SEQ ID NO:39 and
   a second chain comprising a heavy chain antigen-binding region that comprises an H-CDR1 comprising SEQ ID NO: 55, an H-CDR2 comprising SEQ ID NO: 56, and an H-CDR3 comprising SEQ ID NO: 57 and a light chain antigen-binding region that comprises an L-CDR1 comprising SEQ ID NO: 59, an L-CDR2 comprising SEQ ID NO: 60, and an L-CDR3 comprising SEQ ID NO:61; or
ii) a first chain comprising a heavy chain antigen-binding region that comprises an H-CDR1 comprising SEQ ID NO: 121, an H-CDR2 comprising SEQ ID NO: 122, and an H-CDR3 comprising SEQ ID NO: 123 and a light chain antigen-binding region that comprises an L-CDR1 comprising SEQ ID NO: 125, an L-CDR2 comprising SEQ ID NO: 126, and an L-CDR3 comprising SEQ ID NO:127 and
   a second chain comprising a heavy chain antigen-binding region that comprises an H-CDR1 comprising SEQ ID NO: 107, an H-CDR2 comprising SEQ ID NO: 108, and an H-CDR3 comprising SEQ ID NO: 109 and a light chain antigen-binding region that comprises an L-CDR1 comprising SEQ ID NO: 111, an L-CDR2 comprising SEQ ID NO: 112, and an L-CDR3 comprising SEQ ID NO:113.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are bispecific antibody or antigen-binding fragment thereof capable of binding to two different IL-11 epitopes and wherein the bispecific antibodies or antigen-binding fragments thereof comprise
i) a first chain comprising a variable heavy chain domain comprising SEQ ID NO: 32 and a variable light chain domain comprising SEQ ID NO: 36 and
   a second chain comprising a variable heavy chain domain comprising SEQ ID NO: 54 and a variable light chain domain comprising SEQ ID NO: 58; or
ii) a first chain comprising a variable heavy chain domain comprising SEQ ID NO: 120 and a variable light chain domain comprising SEQ ID NO: 124 and
   a second chain comprising a variable heavy chain domain comprising SEQ ID NO: 106 and a variable light chain domain comprising SEQ ID NO: 110.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof are bispecific antibody or antigen-binding fragment thereof capable of binding to two different IL-11 epitopes and wherein the bispecific antibodies or antigen-binding fragments thereof comprise
i) a first chain comprising SEQ ID NO: 74 and a second chain comprising SEQ ID NO: 75; or
ii) a first chain comprising SEQ ID NO: 132 and a second chain comprising SEQ ID NO: 133.

In accordance with another embodiment of all aspects, the present invention covers isolated antibodies or antigen-binding fragments thereof capable of binding to IL-11 and inhibiting IL-11 mediated signaling, wherein said human IL-11 is of the sequence of SEQ ID NO: 1, aa 22-199; wherein said human IL-11Ra is of the sequence of SEQ ID NO: 3 and/or wherein said human gp130 is sequence SEQ ID 12.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof cross-react with mouse, rat, and cynomolgus IL-11, particularly having an affinity to cynomolgus IL-11 that is less than 100-fold, less than 30-fold, less than 15-fold or less than 5-fold different to that to human IL-11.

In accordance with another embodiment of all aspects, the present invention covers isolated antibodies or antigen-binding fragments thereof capable of binding to IL-11 and inhibiting IL-11 mediated signaling, wherein said IL-11 is human IL-11 in particular human IL-11 of the sequence of SEQ ID NO 1, aa 22-199.

In accordance with another embodiment of all aspects, the present invention covers isolated antibodies or antigen-binding fragments thereof capable of binding to IL-11 and inhibiting IL-11 mediated signaling, wherein said IL-11 is murine IL-11, in particular murine IL-11 of the sequence of SEQ ID NO: 16, aa 22-199.

In accordance with another embodiment of all aspects, the present invention covers isolated antibodies or antigen-binding fragments thereof capable of binding to IL-11 and inhibiting IL-11 mediated signaling, wherein said IL-11 is cynomolgus IL-11, in particular cynomolgus IL-11 of the sequence of SEQ ID NO: 17, aa 22-199.

The binding of an isolated antibody or antigen-binding fragment thereof to IL-11 can be analysed by Surface plasmon resonance (SPR) from the Biacore system, as described in example 16, or by ELISA methods as described in Examples 23, and 24. Other methodologies to determine the binding of an isolated antibody or antigen-binding fragment thereof to IL-11 include, but are not limited to, using electro-chemiluminescence method (ELC) via mesoscale discovery (MSD), Luminex xMAP® platform, Immuno-PCR (Lasseter H C et al., 2020, Cytokine X; 28; 2), radioimmunoassays (RIA), fluorescence immunoassays (FIA), thermal shift assays, LC-MS detection, and Bio-layer interferometry (BLI) from Octet system, and kinetic exclusion assay technology, e.g., KinExA® (Sapidyne Instruments, Inc., Boise, ID). KinExA offers a platform that allows the measurement of equilibrium binding affinity and kinetics using unmodified molecules in solution phase. This is accomplished by using a solid-phase immobilized molecule to probe for free concentration of one interaction component after allowing sufficient time to reach equilibrium (affinity measurements), or under pre-equilibrium conditions (kinetics) (Darling R J et al. (2004). ASSAY and Drug Development Technologies. 2 (6): 647-657).

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof does not bind to IL-2Ra.

As shown in example 20 and 26 some IL-11 antibodies show cross-reactivity with IL-2 receptor alpha (IL-2Ra or IL2Ra). However, antibodies according to the invention, in particular TPP-29603, TPP-29697, TPP-18087, TPP-29536, TPP-29528, TPP-29519, TPP-29520, TPP-29521, TPP-29522, TPP-29523, and TPP-23580 do not bind to IL2Ra (see Example 33).

The isolated antibodies or antigen-binding fragments according to the present invention may exhibit any combination of the above described characteristics.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof comprise
i. a heavy chain antigen-binding region that comprises an H-CDR1 comprising SEQ ID NO: 33, an H-CDR2 comprising SEQ ID NO: 34, and an H-CDR3 comprising SEQ ID NO: 35 and a light chain antigen-binding region that comprises an L-CDR1 comprising SEQ ID NO: 37, an L-CDR2 comprising SEQ ID NO: 38, and an L-CDR3 comprising SEQ ID NO:39; or
ii. a heavy chain antigen-binding region that comprises an H-CDR1 comprising SEQ ID NO: 43, an H-CDR2 comprising SEQ ID NO: 44, and an H-CDR3 comprising SEQ ID NO: 45 and a light chain antigen-binding region that comprises an L-CDR1 comprising SEQ ID NO: 47, an L-CDR2 comprising SEQ ID NO: 48, and an L-CDR3 comprising SEQ ID NO:49; or
iii. a heavy chain antigen-binding region that comprises an H-CDR1 comprising SEQ ID NO: 55, an H-CDR2 comprising SEQ ID NO: 56, and an H-CDR3 comprising SEQ ID NO: 57 and a light chain antigen-binding region that comprises an L-CDR1 comprising SEQ ID NO: 59, an L-CDR2 comprising SEQ ID NO: 60, and an L-CDR3 comprising SEQ ID NO:61; or
iv. a heavy chain antigen-binding region that comprises an H-CDR1 comprising SEQ ID NO: 65, an H-CDR2 comprising SEQ ID NO: 66, and an H-CDR3 comprising SEQ ID NO: 67 and a light chain antigen-binding region that comprises an L-CDR1 comprising SEQ ID NO: 69, an L-CDR2 comprising SEQ ID NO: 70, and an L-CDR3 comprising SEQ ID NO:71; or
v. a heavy chain antigen-binding region that comprises an H-CDR1 comprising SEQ ID NO: 83, an H-CDR2 comprising SEQ ID NO: 84, and an H-CDR3 comprising SEQ ID NO: 85 and a light chain antigen-binding region that comprises an L-CDR1 comprising SEQ ID NO: 87, an L-CDR2 comprising SEQ ID NO: 88, and an L-CDR3 comprising SEQ ID NO:89; or
vi. a heavy chain antigen-binding region that comprises an H-CDR1 comprising SEQ ID NO: 107, an H-CDR2 comprising SEQ ID NO: 108, and an H-CDR3 comprising SEQ ID NO: 109 and a light chain antigen-binding region that comprises an L-CDR1 comprising SEQ ID NO: 111, an L-CDR2 comprising SEQ ID NO: 112, and an L-CDR3 comprising SEQ ID NO:113; or
vii. a heavy chain antigen-binding region that comprises an H-CDR1 comprising SEQ ID NO: 121, an H-CDR2 comprising SEQ ID NO: 122, and an H-CDR3 comprising SEQ ID NO: 123 and a light chain antigen-binding region that comprises an L-CDR1 comprising SEQ ID NO: 125, an L-CDR2 comprising SEQ ID NO: 126, and an L-CDR3 comprising SEQ ID NO:127; or
viii. a heavy chain antigen-binding region that comprises an H-CDR1 comprising SEQ ID NO: 137, an H-CDR2 comprising SEQ ID NO: 138, and an H-CDR3 comprising SEQ ID NO: 139 and a light chain antigen-binding region that comprises an L-CDR1 comprising SEQ ID NO: 141, an L-CDR2 comprising SEQ ID NO: 142, and an L-CDR3 comprising SEQ ID NO:143; or
ix. a heavy chain antigen-binding region that comprises an H-CDR1 comprising SEQ ID NO: 149, an H-CDR2 comprising SEQ ID NO: 150, and an H-CDR3 comprising SEQ ID NO: 151 and a light chain antigen-binding region that comprises an L-CDR1 comprising SEQ ID NO: 153, an L-CDR2 comprising SEQ ID NO: 154, and an L-CDR3 comprising SEQ ID NO:155; or
x. a heavy chain antigen-binding region that comprises an H-CDR1 comprising SEQ ID NO: 161, an H-CDR2 comprising SEQ ID NO: 162, and an H-CDR3 comprising SEQ ID NO: 163 and a light chain antigen-binding region that comprises an L-CDR1 comprising SEQ ID NO: 165, an L-CDR2 comprising SEQ ID NO: 166, and an L-CDR3 comprising SEQ ID NO:167; or
xi. a heavy chain antigen-binding region that comprises an H-CDR1 comprising SEQ ID NO: 173, an H-CDR2 comprising SEQ ID NO: 174, and an H-CDR3 comprising SEQ ID NO: 175 and a light chain antigen-binding region that comprises an L-CDR1 comprising SEQ ID NO: 177, an L-CDR2 comprising SEQ ID NO: 178, and an L-CDR3 comprising SEQ ID NO:179; or
xii. a heavy chain antigen-binding region that comprises an H-CDR1 comprising SEQ ID NO: 185, an H-CDR2 comprising SEQ ID NO: 186, and an H-CDR3 comprising SEQ ID NO: 187 and a light chain antigen-binding region that comprises an L-CDR1 comprising SEQ ID NO: 189, an L-CDR2 comprising SEQ ID NO: 190, and an L-CDR3 comprising SEQ ID NO:191.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof comprise
i. a variable heavy chain domain comprising SEQ ID NO: 32 and a variable light chain domain comprising SEQ ID NO: 36; or
ii. a variable heavy chain domain comprising SEQ ID NO: 42 and a variable light chain domain comprising SEQ ID NO: 46; or
iii. a variable heavy chain domain comprising SEQ ID NO: 54 and a variable light chain domain comprising SEQ ID NO: 58; or
iv. a variable heavy chain domain comprising SEQ ID NO: 64 and a variable light chain domain comprising SEQ ID NO: 68; or
v. a variable heavy chain domain comprising SEQ ID NO: 82 and a variable light chain domain comprising SEQ ID NO: 86; or
vi. a variable heavy chain domain comprising SEQ ID NO: 106 and a variable light chain domain comprising SEQ ID NO: 110; or
vii. a variable heavy chain domain comprising SEQ ID NO: 120 and a variable light chain domain comprising SEQ ID NO: 124; or
viii. a variable heavy chain domain comprising SEQ ID NO: 136 and a variable light chain domain comprising SEQ ID NO: 140; or
ix. a variable heavy chain domain comprising SEQ ID NO: 148 and a variable light chain domain comprising SEQ ID NO: 152; or
x. a variable heavy chain domain comprising SEQ ID NO: 160 and a variable light chain domain comprising SEQ ID NO: 164; or
xi. a variable heavy chain domain comprising SEQ ID NO: 172 and a variable light chain domain comprising SEQ ID NO: 176; or
xii. a variable heavy chain domain comprising SEQ ID NO: 184 and a variable light chain domain comprising SEQ ID NO: 188.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof comprise
i. a heavy chain comprising SEQ ID NO: 40 and a light chain comprising SEQ ID NO: 41; or
ii. a heavy chain comprising SEQ ID NO: 90 and a light chain comprising SEQ ID NO: 91; or
iii. a heavy chain comprising SEQ ID NO: 92 and a light chain comprising SEQ ID NO: 33; or
iv. a heavy chain comprising SEQ ID NO: 116 and a light chain comprising SEQ ID NO: 117; or
v. a heavy chain comprising SEQ ID NO: 130 and a light chain comprising SEQ ID NO: 131; or
vi. a heavy chain comprising SEQ ID NO: 146 and a light chain comprising SEQ ID NO: 147; or
vii. a heavy chain comprising SEQ ID NO: 158 and a light chain comprising SEQ ID NO: 159; or
viii. a heavy chain comprising SEQ ID NO: 170 and a light chain comprising SEQ ID NO: 171; or
ix. a heavy chain comprising SEQ ID NO: 182 and a light chain comprising SEQ ID NO: 183; or
x. a heavy chain comprising SEQ ID NO: 192 and a light chain comprising SEQ ID NO: 193; or
xi. a heavy chain comprising SEQ ID NO: 194 and a light chain comprising SEQ ID NO: 195; or
xii. a heavy chain comprising SEQ ID NO: 196 and a light chain comprising SEQ ID NO: 197.

In accordance with another embodiment of all aspects, the present invention covers isolated antibodies or antigen-binding fragments thereof capable of binding to IL-11 and inhibiting IL-11 mediated signaling, wherein said antibodies or antigen-binding fragments thereof are monoclonal antibodies or antigen-binding fragments.

In accordance with another embodiment of all aspects, the present invention covers isolated antibodies or antigen-binding fragments thereof capable of binding to IL-11 and inhibiting IL-11 mediated signaling, wherein said antibodies or antigen-binding fragments thereof are IgG antibody, in particular an IgG1 or an IgG4 antibody.

In accordance with another embodiment of all aspects, the present invention covers isolated antibodies or antigen-binding fragments thereof capable of binding to IL-11 and inhibiting IL-11 mediated signaling, wherein said antigen-binding fragments are scFv, Fab, Fab′ fragments or F(ab′)2 fragments.

In accordance with another embodiment of all aspects, the present invention covers isolated antibodies or antigen-binding fragments thereof capable of binding to IL-11 and inhibiting IL-11 mediated signaling, wherein said antibodies or antigen-binding fragments thereof are human, humanized or chimeric antibodies or antigen-binding fragments thereof, more particularly fully human antibodies or antigen-binding fragments thereof.

In accordance with another aspect, the present invention covers isolated antibodies or antigen-binding fragments thereof capable of binding to IL-11 and inhibiting IL-11 mediated signaling, wherein said isolated antibodies or antigen-binding fragments thereof compete with the isolated antibody or antigen-binding fragment according to the present invention for binding to IL-11.

In accordance with another embodiment of all aspects, the present invention covers isolated antibodies or antigen-binding fragments thereof capable of binding to IL-11 and inhibiting IL-11 mediated signaling, wherein said antibodies or antigen-binding fragments thereof are monospecific antibodies or multi-specific antibodies which bind to IL-11 and at least one further antigen, such as bispecific, trispecific or tetraspecific antibodies.

In accordance with another embodiment of all aspects, the present invention covers isolated antibodies or antigen-binding fragments thereof capable of binding to IL-11 and inhibiting IL-11 mediated signaling, wherein said antibodies or antigen-binding fragments thereof compete with the isolated antibodies or antigen-binding fragments thereof according to the present invention for binding to IL-11.

TABLE 7
Brief description of commercially available antibodies:

| Catalogue # | Company | Clone | Isotype | Host | Di-/Tri-complex ELISA |
|---|---|---|---|---|---|
| MAB218 | R&D Systems | 22626 | IgG2a | Mouse | Active in Di-complex ELISA |
| | | | | | and Tri-complex ELISA |
| MAB418 | R&D Systems | 188520 | IgG2a | Rat | Not tested |
| AF-418 | R&D Systems | NA | IgG | Goat | Not tested |
| GTX34009 | Genetex | 6D9A1 | IgG1 | Mouse | No activity |
| GTX52814 | Genetex | 9T27 | IgG2 | Mouse | Active in Di-complex ELISA |
| | | | | | and Tri-complex ELISA |
| LS-C104441 | LS-bio | unknown | IgG1 | Mouse | Active in Di-complex ELISA |
| | | | | | and Tri-complex ELISA |
| LS-C193526 | LS-bio | KT8 | IgG1 | Mouse | Active in Di-complex ELISA |
| | | | | | and Tri-complex ELISA |
| MA5-30696 | Thermo Fisher | 12 | IgG1 | Mouse | No activity |
| | Scientific | | | | |
| MA5-30695 | Thermo Fisher | 9 | IgG1 | Mouse | No activity |
| | Scientific | | | | |
| MA5-23711 | Thermo Fisher | 22616 | IgG2b | Mouse | Active in Di-complex ELISA |
| | Scientific | | | | and Tri-complex ELISA |

TABLE 8
Brief description of antibodies:

| Antibody | Isotype | Di-/Tri-complex ELISA | Description |
|---|---|---|---|
| TPP-12904 | | Not applicable | Isotype control |
| TPP-10159 | mIgG1, Lambda | Not applicable | Isotype control |
| TPP-10748 | mIgG2a, Lambda | Not applicable | Isotype control |
| TPP-10750 | rIgG2a, Lambda | Not applicable | Isotype control |
| TPP-27360 | scFv-mIgG1 | Not applicable | Isotype control |
| TPP-16478 | hIgG1, Lambda | Active in Di-complex ELISA | |
| | | and Tri-complex ELISA | |
| TPP-18063 | mIgG1, Lambda | Active in Di-complex ELISA | |
| | | and Tri-complex ELISA | |
| TPP-18068 | mIgG1, Lambda | Active in Di-complex ELISA | |
| | | and Tri-complex ELISA | |
| TPP-18087 | mIgG1, Lambda | Active in Tri-complex ELISA | |
| | | and but not in Di-complex | |
| | | ELISA | |
| TPP-19528 | mIgG1, Lambda | Active in Di-complex ELISA | |
| | | and Tri-complex ELISA | |
| TPP-23552 | hIgG1, Kappa | Active in Di-complex ELISA | BSN 3C6 |
| | | and Tri-complex ELISA | WO2019238882 |
| TPP-23580 | hIgG1, Kappa | Active in Di-complex ELISA | BSN 3C6 |
| | | and Tri-complex ELISA | WO2019238882 |
| TPP-27159 | hIgG1, Lambda | Active in Di-complex ELISA | |
| | | and Tri-complex ELISA | |
| TPP-29386 | hIgG1, Lambda | Active in Di-complex ELISA | |
| | | and Tri-complex ELISA | |
| TPP-29519 | mIgG1, Lambda | Active in Tri-complex ELISA | |
| | | and but not in Di-complex | |
| | | ELISA | |
| TPP-29520 | mIgG1, Lambda | Active in Tri-complex ELISA | |
| | | and but not in Di-complex | |
| | | ELISA | |
| TPP-29521 | mIgG1, Lambda | Active in Tri-complex ELISA | |
| | | and but not in Di-complex | |
| | | ELISA | |
| TPP-29522 | mIgG1, Lambda | Active in Tri-complex ELISA | |
| | | and but not in Di-complex | |
| | | ELISA | |
| TPP-29523 | mIgG1, Lambda | Active in Tri-complex ELISA | |
| | | and but not in Di-complex | |
| | | ELISA | |
| TPP-29528 | mIgG1, Lambda | Active in Di-complex ELISA | |
| | | and Tri-complex ELISA | |
| TPP-29536 | hIgG-lambda-HS | Active in Di-complex ELISA | |
| | | and Tri-complex ELISA | |
| TPP-29680 | hIgG1, Lambda | Active in Tri-complex ELISA | |
| | | and but not in Di-complex | |
| | | ELISA | |
| TPP-30000 | hIgG1, Lambda | Active in Tri-complex ELISA | |
| | | and but not in Di-complex | |
| | | ELISA | |
| TPP-30001 | hIgG1, Lambda | Active in Tri-complex ELISA | |
| | | and but not in Di-complex | |
| | | ELISA | |
| TPP-30002 | hIgG1, Lambda | Active in Tri-complex ELISA | |
| | | and but not in Di-complex | |
| | | ELISA | |
| TPP-30003 | hIgG1, Lambda | Active in Tri-complex ELISA | |
| | | and but not in Di-complex | |
| | | ELISA | |
| TPP-31325 | hIgG4-SPLE, | Active in Tri-complex ELISA | |
| | Lambda | and but not in Di-complex | |
| | | ELISA | |
| TPP-31385 | hIgG4-SPLE, | Active in Tri-complex ELISA | |
| | Lambda | and but not in Di-complex | |
| | | ELISA | |
| TPP-31391 | hIgG4-SPLE, | Active in Di-complex ELISA | |
| | Lambda | and Tri-complex ELISA | |
| TPP-26195 | scFv-kih-mIgG1 | Active in Di-complex ELISA | Bispecific comprising |
| | | and Tri-complex ELISA | CDR's of TPP-18068 |
| | | | and TPP-18087 |
| TPP-20489 | scFv-kih-hIgG1 | Active in Di-complex ELISA | Bispecific comprising |
| | | and Tri-complex ELISA | CDR's of TPP-18068 |
| | | | and TPP-18087 |
| TPP-29697 | scFv-kih-hIgG1 | Active in Di-complex ELISA | Bispecific comprising |
| | | and Tri-complex ELISA | CDR's of TPP-29528 |
| | | | and TPP-29519 |
| TPP-29603 | scFv-kih-mIgG1 | Active in Di-complex ELISA | Bispecific comprising |
| | | and Tri-complex ELISA | CDR's of TPP-29528 |
| | | | and TPP-29519 |

The sequence listing provided with the application via electronic filing is included herein in its entirety. SEQ ID NO:1 to SEQ ID NO: 31 relates to IL-11, IL-11Ra and interleukin-6 signal transducer (see Table 1-6). SEQ ID NO: 32 to SEQ ID NO: 75 and SEQ ID NO: 80 to SEQ ID NO: 197 relate to inventive antibodies (see Table 9-12). SEQ ID NO: 76 to SEQ ID NO: 79 relate to competitor antibodies (see Table 9).

Amino acid sequences of preferred monospecific antibodies according to the present invention are listed in Table 8 and Amino acid sequences of preferred bispecific antibodies according to the present invention are listed in Table 9.

TABLE 9
Amino acid sequences of preferred monospecific antibodies according to the present invention

| | | | | | | | | | SEQ | SEQ |
|---|---|---|---|---|---|---|---|---|---|---|
| | SEQ | SEQ | SEQ | SEQ | SEQ | SEQ | SEQ | SEQ | ID NO: | ID NO: |
| | ID NO: | ID NO: | ID NO: | ID NO: | ID NO: | ID NO: | ID NO: | ID NO: | Heavy | Light |
| Antibody | VH | HCDR1 | HCDR2 | HCDR3 | VL | LCDR1 | LCDR2 | LCDR3 | Chain | Chain |
| TPP-16478 | 32 | 33 | 34 | 35 | 36 | 37 | 38 | 39 | 40 | 41 |
| TPP-18063 | 42 | 43 | 44 | 45 | 46 | 47 | 48 | 49 | 50 | 51 |
| TPP-18068 | 32 | 33 | 34 | 35 | 36 | 37 | 38 | 39 | 52 | 53 |
| TPP-18087 | 54 | 55 | 56 | 57 | 58 | 59 | 60 | 61 | 62 | 63 |
| TPP-19528 | 64 | 65 | 66 | 67 | 68 | 69 | 70 | 71 | 72 | 73 |
| TPP-23552 | | | | | | | | | 76 | 77 |
| competitor | | | | | | | | | | |
| AB | | | | | | | | | | |
| TPP-23580 | | | | | | | | | 78 | 79 |
| competitor | | | | | | | | | | |
| AB | | | | | | | | | | |
| TPP-27159 | 82 | 83 | 84 | 85 | 86 | 87 | 88 | 89 | 90 | 91 |
| TPP-29386 | 106 | 107 | 108 | 109 | 110 | 111 | 112 | 113 | 92 | 93 |
| TPP-29519 | 120 | 121 | 122 | 123 | 124 | 125 | 126 | 127 | 94 | 95 |
| TPP-29520 | 136 | 137 | 138 | 139 | 140 | 141 | 142 | 143 | 96 | 97 |
| TPP-29521 | 148 | 149 | 150 | 151 | 152 | 153 | 154 | 155 | 98 | 99 |
| TPP-29522 | 160 | 161 | 162 | 163 | 164 | 165 | 166 | 167 | 100 | 101 |
| TPP-29523 | 172 | 173 | 174 | 175 | 176 | 177 | 178 | 179 | 102 | 103 |
| TPP-29528 | 106 | 107 | 108 | 109 | 110 | 111 | 112 | 113 | 104 | 105 |
| TPP-29536 | 106 | 107 | 108 | 109 | 110 | 111 | 112 | 113 | 116 | 117 |
| TPP-29680 | 120 | 121 | 122 | 123 | 124 | 125 | 126 | 127 | 130 | 131 |
| TPP-30000 | 136 | 137 | 138 | 139 | 140 | 141 | 142 | 143 | 146 | 147 |
| TPP-30001 | 148 | 149 | 150 | 151 | 152 | 153 | 154 | 155 | 158 | 159 |
| TPP-30002 | 160 | 161 | 162 | 163 | 164 | 165 | 166 | 167 | 170 | 171 |
| TPP-30003 | 172 | 173 | 174 | 175 | 176 | 177 | 178 | 179 | 182 | 183 |
| TPP-31325 | 184 | 185 | 186 | 187 | 188 | 189 | 190 | 191 | 192 | 193 |
| TPP-31385 | 54 | 55 | 56 | 57 | 58 | 59 | 60 | 61 | 194 | 195 |
| TPP-31391 | 64 | 65 | 66 | 67 | 68 | 69 | 70 | 71 | 196 | 197 |

TABLE 10
Amino acid sequences of preferred bispecific antibodies according to the present invention

| | SEQ | SEQ | SEQ | SEQ | SEQ | SEQ | SEQ | SEQ | SEQ | SEQ |
|---|---|---|---|---|---|---|---|---|---|---|
| | ID NO: | ID NO: | ID NO: | ID NO: | ID NO: | ID NO: | ID NO: | ID NO: | ID NO: | ID NO: |
| Antibody | VH | HCDR1 | HCDR2 | HCDR3 | VL | LCDR1 | LCDR2 | LCDR3 | Chain 1 | Chain 2 |
| TPP-20489 | 32 | 33 | 34 | 35 | 36 | 37 | 38 | 39 | 74 | |
| Chain 1 | | | | | | | | | | |
| TPP-20489 | 54 | 55 | 56 | 57 | 58 | 59 | 60 | 61 | | 75 |
| Chain 2 | | | | | | | | | | |
| TPP-26195 | 32 | 33 | 34 | 35 | 36 | 37 | 38 | 39 | 80 | |
| Chain 1 | | | | | | | | | | |
| TPP-26195 | 54 | 55 | 56 | 57 | 58 | 59 | 60 | 61 | | 81 |
| Chain 2 | | | | | | | | | | |
| TPP-29603 | 120 | 121 | 122 | 123 | 124 | 125 | 126 | 127 | 118 | |
| Chain 1 | | | | | | | | | | |
| TPP-29603 | 106 | 107 | 108 | 109 | 110 | 111 | 112 | 113 | | 119 |
| Chain 2 | | | | | | | | | | |
| TPP-29697 | 120 | 121 | 122 | 123 | 124 | 125 | 126 | 127 | 132 | |
| Chain 1 | | | | | | | | | | |
| TPP-29697 | 106 | 107 | 108 | 109 | 110 | 111 | 112 | 113 | | 133 |
| Chain 2 | | | | | | | | | | |

Nucleic acid sequences of preferred antibodies according to the present invention are listed in Table 11.

TABLE 11
Nucleic acid sequences of preferred antibodies
according to the present invention

| | SEQ ID NO: | SEQ ID NO: |
|---|---|---|
| Antibody | Heavy Chain | Light Chain |
| TPP-29536 | 114 | 115 |
| TPP-29680 | 128 | 129 |
| TPP-29697 | 134 | 135 |
| | Chain 1 | Chain 2 |
| TPP-30000 | 144 | 145 |
| TPP-30001 | 156 | 157 |
| TPP-30002 | 168 | 169 |
| TPP-30003 | 180 | 181 |

### Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to IL-11 fall within the scope of the present invention.

A variant can include, for example, an antibody that has at least one altered complementary determining region (CDR) (hyper-variable) and/or framework (FR) (variable) domain/position, vis-à-vis a peptide sequence disclosed herein.

By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected as shown in Table 8. The skilled worker can use the data in Table 8 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g., by synthesizing a collection of DNA molecules using trinucleotide mutagenesis (TRIM) technology (Virnekäs B. et al., Nucl. Acids Res. 1994, 22: 5600.). Antibodies or antigen-binding fragments thereof include molecules with modifications/variations including but not limited to e.g., modifications leading to altered half-life (e.g., modification of the Fc part or attachment of further molecules such as PEG), altered binding affinity or altered ADCC or CDC activity.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

### Glycosylation Variants

Where the antibody comprises an Fc region, the carbohydrate attached thereto may be altered. Native antibodies produced by mammalian cells typically comprise a branched, biantennary oligosaccharide that is generally attached by an N-linkage to Asn297 using Kabat EU numbering of the CH2 domain of the Fc region; see, e.g., Wright et al. Trends Biotechnol. 15: 26-32 (1997).

In certain embodiments, an antibody provided herein is altered to increase or decrease the extent to which the antibody is glycosylated. Addition or deletion of glycosylation sites to an antibody may be conveniently accomplished by altering the expression system (e.g. host cell) and/or by altering the amino acid sequence such that one or more glycosylation sites is created or removed.

In one embodiment of this invention, aglycosyl antibodies having decreased effector function or antibody derivatives are prepared by expression in a prokaryotic host. Suitable prokaryotic hosts for include but are not limited to E. coli, Bacillus subtilis, Salmonella typhimurium and various species within the genera Pseudomonas, Streptomyces, and Staphylococcus.

In one embodiment, antibody variants are provided having decreased effector function, which are characterized by a modification at the conserved N-linked site in the CH2 domains of the Fc portion of said antibody. In one embodiment of present invention, the modification comprises a mutation at the heavy chain glycosylation site to prevent glycosylation at the site. Thus, in one preferred embodiment of this invention, the aglycosyl antibodies or antibody derivatives are prepared by mutation of the heavy chain glycosylation site, —i.e., mutation of N297 using Kabat EU numbering and expressed in an appropriate host cell.

In another embodiment of the present invention, aglycosyl antibodies or antibody derivatives have decreased effector function, wherein the modification at the conserved N-linked site in the CH2 domains of the Fc portion of said antibody or antibody derivative comprises the removal of the CH2 domain glycans, —i.e., deglycosylation. These aglycosyl antibodies may be generated by conventional methods and then deglycosylated enzymatically. Methods for enzymatic deglycosylation of antibodies are well known in the art (e.g. Winkelhake & Nicolson (1976), J Biol Chem. 251(4):1074-80).

In another embodiment of this invention, deglycosylation may be achieved using the glycosylation inhibitor tunicamycin (Nose & Wigzell (1983), Proc Natl Acad Sci USA, 80(21):6632-6). That is, the modification is the prevention of glycosylation at the conserved N-linked site in the CH2 domains of the Fc portion of said antibody.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function.

Examples of publications related to “defucosylated” or “fucose-deficient” antibody variants include Okazaki et al. J Mol. Biol. 336: 1239-1249 (2004); Yamane-Ohnuki et al. Biotech. Bioeng. 87: 614 (2004).

Examples of cell lines capable of producing defucosylated antibodies include Lec13 CHO cells deficient in protein fucosylation (Ripka et al. Arch. Biochem. Biophys. 249:533-545 (1986); and WO 2004/056312), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006)).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546.

Antibody variants with at least one galactose residue in the oligosaccharide attached to the Fc region are also provided. Such antibody variants may have improved CDC function. Such antibody variants are described, e.g., in WO1997/30087; WO1998/58964; and WO1999/22764.

### FC Region Variants

In certain embodiments, one or more amino acid modifications (e.g. a substitution) may be introduced into the Fc region of an antibody (e.g., a human IgG1, IgG2, IgG3 or IgG4 Fc region) provided herein, thereby generating an Fc region variant.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. In some embodiments, alterations are made in the Fc region that result in altered (i.e., either improved or diminished) C1q binding and/or Complement Dependent Cytotoxicity (CDC).

In some embodiments, the isolated antibodies or antigen-binding fragments according to the present invention comprise silencing mutations in the Fc-part of the antibody. Such silencing mutations in the Fc-part of the antibody are for e.g., but not limited to E233P, L234V, L235A, ΔG236, D265G, A327Q, or A330S or more preferably E233P, L234V, L235A, ΔG236, D265G, A327Q, and A330S (Durben et al., 2015, Mol Ther. 2015 April; 23(4):648-55 and EP2794658). Further Fc engineering examples include human IgG4 variants L235E or F234A/L235A and the human IgG1 variant L234A/L235A (“LALA”; Xu et al., Cell Immunol 2000 Feb. 25; 200(1):16-26). Another early approach intended to reduce effector function was to mutate the glycosylation site at N297 with mutations such as N297A, N297Q, and N297G (“aglycosylation”; Bolt et al., Eur J Immunol. 1993 February; 23(2):403-11; Tao and Morrison, J Immunol. 1989 Oct. 15; 143(8):2595-601; Walker et al., Biochem J. 1989 Apr. 15; 259(2):347-53; Leabman et al., MAbs November-December 2013; 5(6):896-903). Another variation is a cross-subclass approach to reduce effector function as exemplified by the approved anti-C5 therapeutic eculizumab, which carries CH1 and hinger region from IgG2 but carries CH2 and CH3 from IgG4. Other examples include L234F/L235E/P331S in human IgG1 (“FES”; Oganesyan et al., Acta Crystallogr D Biol Crystallogr. 2008 June; 64(Pt 6):700-4), P329G/L234A/L235A in human IgG1 (“PG-LALA”; Schlothauer et al., Protein Eng Des Sel 2016 October; 29(10):457-466), “IgG1sigma” (L234A/L235A/G237A/P238S/H268A/A330S/P331S, Tam et al., Antibodies (Basel) 2017 Sep. 1; 6(3):12), “IgG1-NNAS” (S298N/T299A/Y300S, Zhou et al., MAbs January-December 2020; 12(1):1814583)(numbering according to Eu nomenclature; Edelman et al., Proc Natl Acad Sci USA. 1969 May; 63(1): 78-85; Kabat et al., 1991, Sequences of Proteins of Immunological Interest, 5th Edition. U.S. Department of Health and Human Services, Public Health Service, National Institutes of Health, NIH Publication No. 91-3242).

In some embodiments, the isolated bispecific antibodies or antigen-binding fragments according to the present invention comprise corresponding knob/hole (knob-into-hole) mutations in the Fc-part of the antibody. The knob-into-hole approach is an effective way to produce bispecific antibodies by driving heterodimerization with mutations in the CH3 domain of each half antibody, such as but not limited to mutating several CH3 amino acid residues, i.e., threonine (T) 366 to tryptophan (W) for the “knob” half antibody, and threonine (T) 366 to serine (S), leucine (L) 368 to alanine (A), and tyrosine (Y) 407 to valine (V) for the “hole” half antibody.

In certain embodiments, the invention contemplates an antibody variant that possesses an increased or decreased half-live. Antibodies with increased half-lives and improved binding to the neonatal Fc receptor (FcRn), which is responsible for the transfer of maternal IgGs to the fetus (Guyer et al., J Immunol. 117:587 (1976) and Kim et al., J Immunol. 24:249 (1994)), are described in US2005/0014934 (Hinton et al.). Those antibodies comprise an Fc region with one or more substitutions therein which improve binding of the Fc region to FcRn. For example, but not limited to, the mutation “YTE” (M252Y/S254T/T256E) and equivalent mutations, have been shown to significantly extend the half-life by more efficient recycling from endosomes in both pre-clinical species as well as humans. Abrogating the interaction between FcRn and the Fc part of the antibody, by e.g., H435A leads to an extremely short half-life, since the antibody is no longer protected from lysosomal degradation by FcRn recycling.

In accordance with a further aspect, the present invention covers antibody conjugates, comprising the isolated antibodies or antigen binding fragments according to the present invention.

### Antibody Generation

An antibody of the invention may be derived from a recombinant antibody library that is based on amino acid sequences that have been isolated from the antibodies of a large number of healthy volunteers e.g., using the n-CoDeR® technology the fully human CDRs are recombined into new antibody molecules (Carlson & Söderlind, Expert Rev Mol Diagn. 2001 May; 1(1):102-8). Or alternatively for example antibody libraries as the fully human antibody phage display library described in Hoet R M et al., Nat Biotechnol 2005; 23(3):344-8) can be used to isolate IL-11-specific antibodies. Antibodies or antibody fragments isolated from human antibody libraries are considered human antibodies or human antibody fragments herein.

Human antibodies may be further prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. For example, immunization of genetically engineered mice inter alia immunization of hMAb mice (e.g., VelocImmune Mouse® or XENOMOUSE®) may be performed.

Further antibodies may be generated using the hybridoma technology (for example see Köhler and Milstein Nature. 1975 Aug. 7; 256(5517):495-7), resulting in for example murine, rat, or rabbit antibodies which can be converted into chimeric or humanized antibodies. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall' Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Examples are provided for the generation of antibodies using a recombinant antibody library.

### DNA Molecules According to the Present Invention

In accordance with a further aspect, the present invention covers isolated nucleic acid sequences which encode the antibodies or antigen-binding fragments thereof according to the present invention.

In accordance with a further aspect, the present invention covers vectors comprising a nucleic acid sequence according to the present invention.

The present invention also relates to an isolated nucleic acid sequence that encodes the antibody or antigen-binding fragment according to the present invention. The isolated nucleic acid sequence encoding the antibody or antigen-binding fragment according to the present invention can for instance be produced by techniques described in Sambrook et al., 1989, and Ausubel et al., 1989, or alternatively, by chemically synthesis. (e.g. techniques described in Oligonucleotide Synthesis (1984, Gait, ed., IRL Press, Oxford)). The DNA sequences used for the antibodies expressed are given in Table 2. These sequences are optimized in certain cases for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non stringent conditions favor hybridization of molecules displaying a lesser degree of structural relatedness. Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent DNA Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs.

Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209 217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single stranded DNA bacteriophage vehicle. Single-stranded DNA is isolated and hybridized with an oligonucleotide containing the desired nucleotide alteration(s). The complementary strand is synthesized, and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

In accordance with a further aspect, the present invention covers vectors comprising a nucleic acid sequence according to the present invention.

In accordance with a further aspect, the present invention covers isolated cells expressing the antibodies or antigen-binding fragments thereof according to the present invention comprising the nucleic acid according to the present invention or the vector according to the present invention.

In accordance with another embodiment of all aspects, the present invention covers isolated cells expressing the antibodies or antigen-binding fragments thereof according to the present invention comprising the nucleic acid according to the present invention or the vector according to the present invention wherein said cell is a prokaryotic or a eukaryotic cell.

In accordance with a further aspect, the present invention covers methods of producing the isolated antibodies or antigen-binding fragments according to the present invention comprising culturing of the cell according to the present invention and optionally purification of said antibodies or antigen-binding fragments.

The present invention further provides recombinant DNA constructs comprising one or more of the nucleotide sequences according to the present invention. The recombinant constructs of the present invention can be used in connection with a vector, such as a plasmid, phagemid, phage or viral vector, into which a DNA molecule encoding an antibody of the invention or antigen-binding fragment thereof or variant thereof is inserted.

Thus, in one aspect, the present invention relates to a vector comprising a nucleic acid sequence according to the present invention.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g., bacteria, examples for eukaryotic hosts cells are yeasts, insects and insect cells, plants and plant cells, transgenic animals, or mammalian cells. Introduction of the recombinant construct into the host cell can be carried out using standard techniques such as calcium phosphate transfection, DEAE dextran mediated transfection, electroporation, transduction or phage infection.

In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Thus, in a further aspect, the present invention relates to an isolated cell expressing the antibody or antigen-binding fragment according to the present invention and/or comprising the nucleic acid according to the present invention or the vector according to the present invention.

The isolated cell can be virtually any cell for which expression vectors are available. The isolated cell can for example a higher eukaryotic host cell, such as a mammalian cell, a lower eukaryotic host cell, such as a yeast cell, and may be a prokaryotic cell, such as a bacterial cell.

In a further aspect, the present invention relates to a method of producing the isolated antibody or antigen-binding fragment according to the present invention comprising culturing of the cell according to the present invention. In particular embodiments, the cell according to the present invention is cultivated under suitable conditions for antibody expression and the antibody or antigen-binding fragment is recovered. In particular embodiments, the antibody or antigen-binding fragment is purified, particularly to at least 95% homogeneity by weight.

### Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g., inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10(2):169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resistance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-K1SV [including dhfr-CHO cells, described in Urlaub and Chasin, (1980) Proc. Natl. Acad. Sci. USA 77:4216-4220 and Urlaub et al., Cell. 1983 June; 33(2):405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109(4):1007-15], NS0 myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-K1SV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Purification

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

### Therapeutic Methods

Therapeutic methods involve administering to a subject in need of treatment a therapeutically effective amount of an antibody or an antigen-binding fragment thereof or a variant thereof contemplated by the invention. A “therapeutically effective” amount hereby is defined as the amount of an antibody or antigen-binding fragment thereof that is of sufficient quantity to reduce blood loss during heavy menstrual bleeding, abnormal uterine bleeding or heavy menstrual bleeding secondary to leiomyoma or endometriosis—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

In accordance with a further aspect, the present invention covers isolated antibodies or antigen-binding fragments according the present invention or to a conjugate comprising the isolated antibody or antigen-binding fragment according the present invention or to a pharmaceutical composition comprising the isolated antibody or antigen-binding fragment according the present invention for use in the treatment or prophylaxis of diseases.

The isolated antibodies or antigen-binding fragments according to the present invention can be used as a therapeutic or a diagnostic tool in a variety of IL-11 associated disorders and/or diseases associated with abnormal uterine bleeding.

In accordance with a further aspect, the present invention covers the use of isolated antibodies or antigen-binding fragments according the present invention or antibody conjugates according to the present invention for use as a diagnostic agent.

In accordance with a further aspect, the present invention covers the use of isolated antibodies or antigen-binding fragments according the present invention for use in the treatment and/or prevention of abnormal uterine bleeding, dysmenorrhea, leiomyoma, or endometriosis.

In accordance with a further aspect, the present invention covers the use of isolated antibodies or antigen-binding fragments according the present invention for the treatment and/or prevention of abnormal uterine bleeding, dysmenorrhea, leiomyoma, or endometriosis.

In accordance with a further aspect, the present invention covers the use of isolated antibody or antigen-binding fragment according the present invention in a method of treatment and/or prevention of abnormal uterine bleeding, dysmenorrhea, leiomyoma, or endometriosis.

In accordance with a further aspect, the present invention covers use of isolated antibody or antigen-binding fragment according the present invention for the preparation of a pharmaceutical composition, preferably a medicament, for the treatment and/or prevention of abnormal uterine bleeding, dysmenorrhea, leiomyoma, or endometriosis.

In accordance with a further aspect, the present invention covers a method of treatment and/or prevention of abnormal uterine bleeding, dysmenorrhea, leiomyoma, or endometriosis using an effective amount of isolated antibody or antigen-binding fragment according the present invention.

In accordance with an embodiment of all aspects of the invention covers isolated antibody or antigen-binding fragment according the present invention for use in the treatment and/or prevention of abnormal uterine bleeding,
wherein abnormal uterine bleeding is heavy menstrual bleeding, prolonged bleeding or bleeding with altered bleeding pattern.

In accordance with another embodiment of all aspects, the present invention covers isolated antibody or antigen-binding fragment according the present invention for use in the treatment and/or prevention of abnormal uterine bleeding,
wherein abnormal uterine bleeding is heavy menstrual bleeding and wherein heavy menstrual bleeding is secondary to leiomyoma or endometriosis.

In accordance with another embodiment of all aspects, the present invention covers isolated antibody or antigen-binding fragment according the present invention for use in the treatment and/or prevention of abnormal uterine bleeding,
wherein abnormal uterine bleeding is associated with dysmenorrhea.

In accordance with another embodiment of all aspects, the present invention covers isolated antibody or antigen-binding fragment according the present invention for use in the treatment and/or prevention of abnormal uterine bleeding,
wherein abnormal uterine bleeding is associated with dysmenorrhea secondary to uterine leiomyoma or endometriosis.

In accordance with a further aspect, the present invention covers use of isolated antibodies or antigen-binding fragments according to the present invention or conjugates according to the present invention or the pharmaceutical composition according to the present invention for inhibition or modulation of menstruation.

The antibodies or the antigen-binding fragments according to the present invention or variants thereof might be co-administered with known medications, and in some instances the antibody or antigen-binding fragment thereof might itself be modified. For example, an antibody or an antigen-binding fragment thereof or a variant thereof could be conjugated to a drug or to another peptide or protein to potentially further increase efficacy.

Antibodies of the present invention or antigen-binding fragments thereof or variants thereof may be administered as the sole pharmaceutical agent or in combination with one or more additional therapeutic agents where the combination causes no unacceptable adverse effects.

Thus, in a further aspect, the present invention relates to the isolated antibodies or antigen-binding fragments according to the present invention or conjugates according to the present invention or pharmaceutical compositions according to the present invention for use in simultaneous, separate, or sequential combination with one or more further therapeutically active compounds.

Preferred examples of such further active compounds include but are not limited to: selective oestrogen receptor modulators (SERMs), oestrogen receptor (ER) antagonists, aromatase inhibitors, 17f-HSD1 inhibitors, steroid sulphatase (STS) inhibitors, GnRH agonists and antagonists, kisspeptin receptor (KISSR) antagonists, selective androgen receptor modulators (SARMs), androgens, 5α-reductase inhibitors, selective progesterone receptor modulators (SPRMs), gestagens, antigestagens, oral contraceptives, inhibitors of mitogen-activated protein (MAP) kinases and inhibitors of the MAP kinases (Mkk3/6, Mek1/2, Erk1/2), inhibitors of the protein kinases B (PKBα/β/γ; Akt1/2/3), inhibitors of the phosphoinositide 3-kinases (PI3K), inhibitors of cyclin-dependent kinase (CDK1/2), inhibitors of the hypoxia-induced signaling pathway (HIF1alpha inhibitors, activators of prolylhydroxylases), histone deacetylase (HDAC) inhibitors, prostaglandin F receptor (FP) (PTGFR) antagonists, and non-steroidal inflammation inhibitors (NSAIDs).

For example, antibodies or fragments thereof of the present invention can be combined with known antihyperproliferative, cytostatic or cytotoxic substances for treatment of cancers. In addition, inventive agents can also be used in combination with radiotherapy and/or surgical intervention.

Examples of suitable combination active ingredients include but are not limited to:

131I-chTNT, abarelix, abiraterone, aclarubicin, aldesleukin, alemtuzumab, alitretinoin, altretamine, aminoglutethimide, amrubicin, amsacrine, anastrozole, arglabin, arsentrioxidas, asparaginase, azacitidine, basiliximab, RDEA 119, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+oestrone, eculizumab, edrecolomab, elliptinium acetate, eltrombopag, endostatin, enocitabine, epirubicin, epitiostanol, epoetin alfa, epoetin beta, eptaplatin, eribulin, erlotinib, oestradiol, oestramustine, etoposide, everolimus, exemestane, fadrozole, filgrastim, fludarabine, fluorouracil, flutamide, formestane, fotemustine, fulvoestrant, gallium nitrate, ganirelix, gefitinib, gemcitabine, gemtuzumab, glutoxim, goserelin, histamine dihydrochloride, histrelin, hydroxycarbamide, I-125 pellets, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostan, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oxaliplatin, p53 gene therapy, paclitaxel, palifermin, palladium-103 pellets, pamidronic acid, panitumumab, pazopanib, pegaspargase, pEG-epoetin beta (methoxy PEG-epoetin beta), pegfilgrastim, peginterferon alfa-2b, pemetrexed, pentazocine, pentostatin, peplomycin, perfosfamid, picibanil, pirarubicin, plerixafor, plicamycin, poliglusam, polyoestradiol phosphate, polysaccharide-K, porfimer sodium, pralatrexate, prednimustine, procarbazine, quinagolide, radium-223 chloride, raloxifen, raltitrexed, ranimustine, razoxane, regorafenib, risedronic acid, rituximab, romidepsin, romiplostim, sargramostim, sipuleucel-T, sizofiran, sobuzoxan, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

The present invention preferably relates to medicaments comprising at least one antibody or antibody fragment thereof according to the present invention and one or more of the following active ingredients, especially for treatment and/or prophylaxis of steroid receptor-dependent proliferative disorders:
LHRH (luteinizing hormone-releasing hormone) agonists,
LHRH (luteinizing hormone-releasing hormone) antagonists,
C(17,20)-lyase inhibitors,
type I 5-α-reductase inhibitors,
type II 5-α-reductase inhibitors,
mixed type I/II 5-α-reductase inhibitors,
α-radiation-emitting radiopharmaceuticals for treatment of bone metastases, for example radium-223 chloride,
cytostatics,
VEGF (Vascular Endothelial Growth Factor) kinase inhibitors,
antigestagens,
antioestrogens,
EGF antibodies,
oestrogens or
poly(ADP-ribose) polymerase I inhibitors, or
bi-specific T-cell engagers (BiTE) coupled to a cell surface protein, for example prostate-specific membrane antigen (PSMA).

In accordance with a further aspect, the present invention covers isolated antibodies or antigen-binding fragments thereof according the present invention or conjugates comprising the isolated antibody or antigen-binding fragment according the present invention or pharmaceutical compositions comprising the isolated antibody or antigen-binding fragment according the present invention for use in simultaneous, separate, or sequential combination with one or more further therapeutically active compounds.

Combination therapy includes administration of a single pharmaceutical dosage formulation which comprises an antibody or antigen-binding fragment according to the present invention or a variant thereof and one or more additional therapeutic agents, as well as administration of an antibody or antigen-binding fragment according to the present invention and each additional therapeutic agent in its own separate pharmaceutical dosage formulation. For example, an antibody of the invention or an antigen-binding fragment thereof or a variant thereof and a therapeutic agent may be administered to the patient together in a single liquid composition, or each agent may be administered in separate dosage formulation.

Where separate dosage formulations are used, the antibody or antigen-binding fragment according to the present invention or the variant thereof and one or more additional therapeutic agents may be administered at essentially the same time (e.g., concurrently) or at separately staggered times (e.g., sequentially).

The antibodies or the antigen-binding fragments thereof according to the present invention or variants thereof might be used in combination with surgical interventions, such as but not limited to myomectomies, uterine artery embolization or laparoscopic or conventional surgery of endometriotic lesions especially for treatment after such surgical interventions.

### Diagnostic Methods

In accordance with a further aspect, the present invention covers isolated antibodies or antigen-binding fragments thereof according the present invention or conjugates comprising the isolated antibody or antigen-binding fragment according the present invention for use as a diagnostic agent.

Furthermore, the antibodies or antigen-binding fragments according to the present invention may be utilized, as such or in compositions, in research and diagnostics, or as analytical reference standards, and the like. IL-11 antibodies or antigen-binding fragments thereof can be used for detecting the presence of IL-11.

### Pharmaceutical Compositions and Administration

In accordance with a further aspect, the present invention covers pharmaceutical compositions comprising isolated antibodies or antigen-binding fragments according to present invention or antibody conjugates according to the present invention and optionally one or more pharmaceutically acceptable excipients.

To treat any of the foregoing disorders, pharmaceutical compositions for use in accordance with the present invention may be formulated in any conventional manner using one or more physiologically acceptable carriers, excipients, or auxiliaries. Further details on techniques for formulation and administration may be found in the latest edition of Remington's Pharmaceutical Sciences (Ed. Maack Publishing Co, Easton, Pa.).

The antibody or antigen-binding fragment according to the present invention can be administered by any suitable means, which can vary, depending on the type of disorder being treated. Possible administration routes include oral, parenteral, and topical administration. Methods of parenteral delivery include but are not limited to intra-arterial, intramuscular, subcutaneous, intramedullary, intrathecal, intraventricular, intravenous, intraperitoneal, intraocular, or intranasal administration. In addition, the antibody or antigen-binding fragment according to the present invention may be administered by pulse infusion, with, e.g., declining doses of the antibody. Preferably, administration is by injection, most preferably intravenous or subcutaneous injection, depending in part on whether the administration is brief or prolonged. The amount to be administered will depend on a variety of factors such as the clinical symptoms, weight of the individual, whether other drugs are administered, and the like. The skilled artisan will recognize that the route of administration will vary depending on the disorder or condition to be treated.

The pharmaceutical composition according to the present invention comprises the antibody or antigen-binding fragment according to the present invention alone or in combination with at least one other agent, such as a stabilizing compound. The antibody or antigen-binding fragment thereof according to the present invention may be administered in any sterile, biocompatible pharmaceutical carrier, including, but not limited to, saline, buffered saline, dextrose, and water. In particular embodiments, the pharmaceutical composition according to the present invention may comprise one or more further pharmaceutically active compounds, in particular one or more further pharmaceutically active compounds that are suitable to treat IL-11 associated disorders and/or disorders associated with abnormal uterine bleeding. Any of these agents can be administered to a patient alone, or in combination with other agents or drugs, in pharmaceutical compositions where it is mixed with excipient(s) or pharmaceutically acceptable carriers. In particular embodiments, the pharmaceutically acceptable carrier is pharmaceutically inert.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from coin, wheat, rice, potato, or other plants; cellulose such as methyl-cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine or phosphate or Tris, 0.1%-2% sucrose and/or 2%-7% mannitol at a pH range of 4.5 to 7.5 optionally comprising additional substances like polysorbate that is combined with buffer prior to use.

After pharmaceutical compositions comprising a compound of the invention formulated in an acceptable carrier have been prepared, they can be placed in an appropriate container and labeled for treatment of an indicated condition. For administration of IL-11 antibodies or antigen-binding fragment thereof, such labeling would include amount, frequency and method of administration.

### Therapeutically Effective Dose

Pharmaceutical compositions suitable for use according to the present invention include compositions wherein the active ingredients are contained in an effective amount to achieve the intended purpose, e.g., treatment of a particular disease state characterized by ischemic events due to partial or complete vessel occlusion.

The determination of an effective dose is well within the capability of those skilled in the art. Determining a therapeutically effective amount of the novel antibody of this invention or an antigen-binding fragment thereof or a variant thereof, largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

For any compound, the therapeutically effective dose can be estimated initially either in cell culture assays, or in animal models, usually mice, rabbits, dogs, pigs or monkeys. The animal model is also used to achieve a desirable concentration range and route of administration. Such information can then be used to determine useful doses and routes for administration in humans.

A therapeutically effective dose refers to that amount of antibody or antigen-binding fragment thereof, that ameliorates the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions that exhibit large therapeutic indices are preferred. The data obtained from cell culture assays and animal studies are used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, age, weight and gender of the patient; diet, time and frequency of administration, drug combination(s), reaction sensitivities, and tolerance/response to therapy. Long acting pharmaceutical compositions might be administered for example every 3 to 4 days, every week, once every two weeks, or once every three weeks, depending on half-life and clearance rate of the particular formulation.

Normal dosage amounts may vary from 0.1 to 100,000 micrograms, up to a total dose of about 10 g, depending upon the route of administration. Guidance as to particular dosages and methods of delivery is provided in the literature. See U.S. Pat. Nos. 4,657,760; 5,206,344; or 5,225,212.

### Kits

In accordance with a further aspect, the present invention kit comprising isolated antibodies or antigen-binding fragments according to present invention or antibody conjugates according to the present invention or pharmaceutical compositions according to the invention and instructions for use.

In particular embodiments, the kits comprise one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

### EXAMPLES AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope. All amino acid sequences disclosed herein are shown from N-terminus to C-terminus; all nucleic acid sequences disclosed herein are shown 5′->3′.

### Examples

The experiments shown herein clearly support IL-11 and/or IL-11RA as a target whose inhibition including allosteric inhibition provides a therapy option for abnormal uterine bleeding such as heavy menstrual bleeding, prolonged bleeding or altered bleeding pattern, as well as for leiomyoma, endometriosis or menstruation and dysmenorrhea associated with AUB.

### Example 1: Blood Reduction in Murine HMB Model Treated with AF418

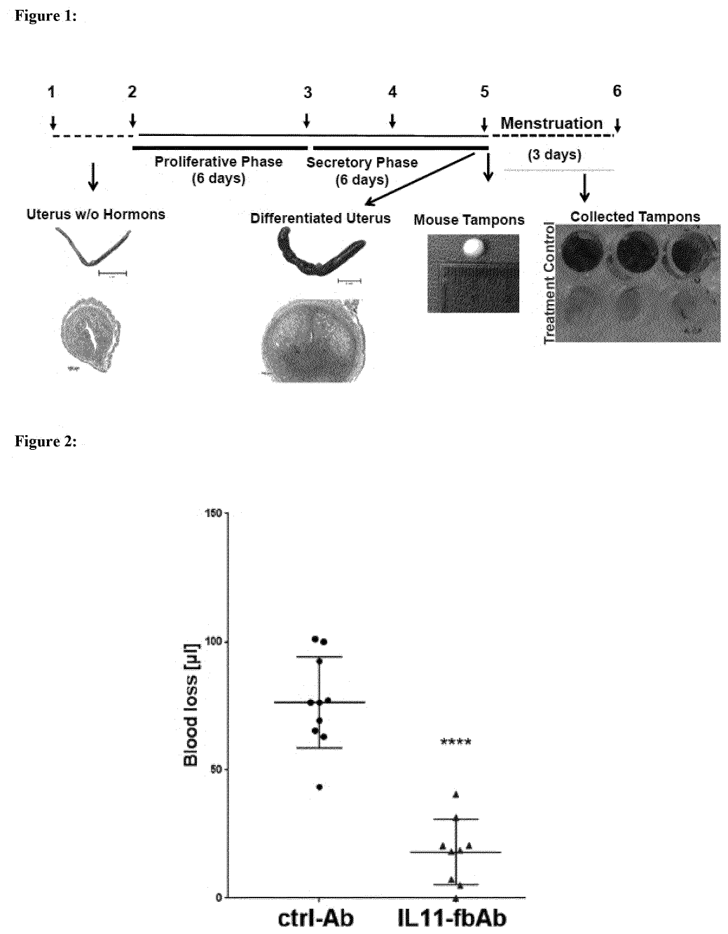

In a mouse model of heavy menstrual bleeding the effect of function blocking IL-11 antibody on menstruation was tested. Artificial menstrual cycle phases of ovariectomized mice were induced by estradiol (E2) and progesterone (P4) supplementation/withdraw schedule including the induction of decidualization with intra-uterine oil application as described e.g., in Menning (2012) and outlined in FIG. 1. Briefly, starting six days after ovariectomy female Scid beige mice [Charles River Sulzfeld, Deutschland] received 9 mg/kg twice weekly subcutaneous (s.c.) injections of control antibody or an IL-11 function blocking polyclonal antibody [AF-418-NA from R&D Systems, Inc.] for a period of 16 days. One week after ovariectomy female Scid beige mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

Results are shown in FIG. 2. A significant reduction (two tailed t-test, p<0.0001) of blood loss in the function blocking antibody treated group AF418 (blood loss 17.9 μl+/−12.7 μl SD) could be determined in comparison to the control antibody group TPP-12904 (blood loss 76.4 μl+/−17.9 μl SD).

### Example 2: Reduction of Uterine Weight in Murine HMB Model Treated with AF418

In a Mouse model of menstruation, effect of function blocking IL-11 antibody [AF-418-NA from R&D Systems, Inc.] on uterine weight was tested. Artificial menstrual cycle phases of ovariectomized mice were induced by estradiol (E2) and progesterone (P4) supplementation/withdrawal schedule. Decidualisation of the tissue was further induced with intra-uterine oil application as described elsewhere (e.g., in Menning, 2012) and outlined in FIG. 1. Briefly, starting six days after ovariectomy female Scid beige mice received 9 mg/kg twice weekly subcutaneous (s.c.) injections of control antibody or IL-11 function blocking antibody for a period of 16 days. One week after ovariectomy female Scid beige mice received s.c. injections of 100 ng 17alpha-estradiol (E2) in ethanol/arachis oil (1:9) on three consecutive days (FIG. 1). After a three day break a progesterone (P4) releasing silastic tube (0.5 mg P4/d) was implanted s.c. into the back of mice followed by further applications of 5 ng E2 on three consecutive days. Concomitant with the last E2 treatment 50 μl sesame oil were injected into one uterus horn. Four days later the P4 implant was removed to initiate P4 withdrawal. Mice were sacrificed four days later at the end of the experiment (day 15) and uteri were weighed and harvested for further analysis.

Results of effects on uterine weight are shown in FIG. 3. A significant reduction of uterine weight in the function blocking antibody treated group could be determined in comparison to the control antibody [AB-108-C from R&D Systems, Inc.] (70.1 mg+/−25.5 mg SD (AF418) versus 297.9 mg+/−162.1 mg SD (AB-108-C (TPP12904))).

Inhibiting the IL-11/IL-11RA signaling pathway by the function blocking IL-11 antibody [AF-418-NA from R&D Systems, Inc.] showed very strong effects on both heavy menstrual bleeding (Example 1, FIG. 2) and uterus weight (Example 2, FIG. 3). Therefore, these surprising results lead to the conclusion that IL-11/IL-11RA function blocking strongly attenuates menstruation. Accordingly, to the retrograde menstruation model of endometriosis (Sampson, 1927) retrograde menstruation is a prerequisite in the etiopathogenesis of endometriosis. In addition, retrograde menstruation is not the exception but occurs normally in menstruation. However, it has been shown and suspected that heavy menstruation shows also increased retrograde menstruation and is therefore associated with a higher risk for developing endometriosis (for review see Laux-Biehlmann et al., 2015). Beside the importance in the etiopathogenesis of endometriosis retrograde menstruation contributes to peritoneal inflammation, pain and inflammatory pain. It is conceivable that reduction or complete inhibition of menstruation would strongly reduce also retrograde menstruation and thereby endometriosis and endometriosis associated symptoms. In addition, it is well-known for endometriosis that active lesions (e.g., red lesions) in the ectopic endometrium in the peritoneal cavity are also regulated by the menstrual cycle. This ectopic tissue sheds endometrium and shows intraperitoneal bleeding (Burney & Lathi, 2009). Thereby the ‘intraperitoneal menstruation’ further contributes to peritoneal inflammation, pain and inflammatory pain in addition to the retrograde menstruation (Laux-Biehlmann et al., 2015). ‘Intraperitoneal menstruation’ may further disseminate new ectopic lesions within the peritoneal cavity. Retrograde menstruation and ‘intraperitoneal menstruation’ would be strongly prevented by inhibiting IL-11/IL-11RA signaling. Subsequently the establishment of new lesions in the peritoneum would be attenuated as well as endometriosis related peritoneal inflammation and inflammatory pain. Indeed, the currently available short term therapeutic interventions by gonadotropin releasing hormone (GnRH) analogues or antagonist also abolish menstruation which contributes to their overall beneficial effects. However, the strong side effects on e.g., bone metabolism prohibit long-term treatment. In opposite antagonists or inhibitors of IL-11/IL-11RA would not have such restrictions.

### Example 3: Inhibition of VEGF-A Secretion in Primary Human Fibroid Slice Assay Treated with AF418

In a Primary Human Fibroid Slice Assay effects of IL-11 [218-IL from R&D Systems, Inc.] and function blocking IL-11 antibody [AF-418-NA from R&D Systems, Inc.] on mediators of angiogenesis were tested. Induction of angiogenesis increases vascularization which might contribute to enhanced leiomyoma growth. Primary human fibroid tissue from a 41 year old patient was sliced with a Krumdick tissue slicer in 0.4 mm slices (diameter 5 mm) and washed in PBS. Slices were overnight incubated in cell culture media w/o L-Tryp containing 5% fetal calf serum (FCS). Slices were further incubated in 48 well plates in 400 μl media for 24 hours with IL-11 [218-IL from R&D Systems, Inc.], control antibody [AB-108-C from R&D Systems, Inc.] or IL-11 function blocking antibody [AF-418-NA from R&D Systems, Inc.].

Results are shown in FIG. 4. IL-11 significantly induced secretion of VEGF-A (72.2 ng/mg protein+/−29.8 ng/mg protein SD), which was completely inhibited by additional treatment with the IL-11 function blocking antibody [AF-418-NA from R&D Systems, Inc.](17.3 ng/mg protein+/−6.4 ng/mg protein SD). VEGF-A is a well-known proangiogenic mediator which induction by IL-11 might lead to increased leiomyoma vascularization and enhanced leiomyoma growth. Statistical test: Ordinary one-way ANOVA with Bonferroni correction for multiple comparison (**** p<0.0001).

### Example 4: Animal Weight in Murine HMB Model Treated with AF418

As pointed out in Examples 1 and 2 inhibiting the IL-11 signaling pathway by the function blocking IL-11 antibody showed marked effects on both heavy menstrual bleeding (FIG. 2) and uterus weight (FIG. 3). Therefore, these results lead to the conclusion that IL-11 function blocking strongly attenuates menstruation and causes amenorrhea. For the expert it is known that avoiding or attenuation of menstruation and induction of amenorrhea is an effective treatment for primary and secondary dysmenorrhea as continues application of progestins demonstrated (Power et al., 2007; Sachedina & Todd, 2020). Therefore, agents capable of binding to and inhibiting or antagonizing the action of IL-11 will be an effective treatment for primary and secondary dysmenorrhea. To verify this hypothesis the animal weights before start of the menstruation (day 12) and at the end of the bleeding period (day 15) were measured in the heavy menstrual bleeding model (FIG. 1) as a marker for well-being.

As shown in FIG. 5 the animal weights of the AF414 IL-11 antagonizing antibody treated animals showed an attenuation of the loss of bodyweight (12.1%+/−2.4% SD) in comparison to the TPP-12904 control-antibody treated animals (21%+/−4.5% SD. Statistical test: two tailed t test, **** p<0.0001).

### Example 5: Blood Reduction in Murine HMB Model Treated with AF418 and MAB218

In a mouse model of heavy menstrual bleeding the effects of commercially available function blocking IL-11 antibodies on menstruation were tested. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Scid beige mice [Charles River Sulzfeld, Deutschland] received 20 mg/kg twice weekly subcutaneous (s.c.) injections of an IL-11 function blocking monoclonal antibody [Mab-218 from R&D Systems, Inc.] or the mouse IgG2A control antibody [TPP-10748], an IL-11 function blocking monoclonal antibody [Mab-418 from R&D Systems, Inc.] or its rat IgG2A control antibody [TPP-10750], or 5 mg/kg twice weekly subcutaneous (s.c.) injections of an IL-11 function blocking polyclonal antibody [AF-418-NA from R&D Systems, Inc.] for a period of 16 days. One week after ovariectomy female Scid beige mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

Results are shown in FIG. 6., heavy menstrual bleeding (HMB) is significantly attenuated in the MAb218 human IL-11 function blocking antibody treated group (45 μl+/−18 μl SD vs 86 μl+/−43 μl SD of ctrl(mAb) TPP-10748; p<0.05) but not in the Mab418 mouse IL-11 function blocking antibody treated group (79 μl+/−18 μl SD vs 99 μl+/−36 μl SD of ctrl(rAb) TPP-10750) in Scid beige mice. The polyclonal AF-418 mouse IL-11 function blocking antibody showed again strong inhibition (23 μl+/−10 μl SD vs 99 μl+/−36 μl SD of ctrl(rAb) TPP-10750; p<0.0001). Significance was tested by one-way ANOVA with Bonferroni correction for multiple comparison.

### Example 6: Blood Reduction in Murine HMB Model Treated with TPP-18068 and TPP-18063

In a mouse model of heavy menstrual bleeding the effects of function blocking IL-1 antibodies on menstruation were tested. These antibodies were identified by antibody panning and showed different in vitro activities as for example binding potency on recombinant IL-11. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/cAnN mice [Janvier] received 20 mg/kg twice weekly subcutaneous (s.c.) injections of the IL-11 function blocking monoclonal antibodies TPP-18063 or TPP-18068 or a mouse IgG1 control antibody [TPP-10159] for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 7 shows inhibition of menstrual bleeding in Balb/c mice by monoclonal antibodies, with significant inhibition of menstrual bleeding by IL-11 function blocking antibody TPP-18068 (blood loss 17 μl+/−12 μl SD) but not IL-11 function blocking antibody TPP-18063 (blood loss 36 μl+/−28 μl SD) in comparison to the isotype control antibody TPP-10159 (blood loss 50 μl+/−29 μl SD). Significance was tested by one-way ANOVA with Bonferroni correction for multiple comparison (** p<0.01)

### Example 7: Dose Dependent Blood Reduction in Murine HMB Model Treated with TPP-18068

Example 7 shows the dose dependent effects on menstrual bleeding of the active IL-11 function blocking antibody TPP-18068 in comparison to an isotype control antibody (TPP-10159). Artificial menstrual cycle phases of ovariectomized mice were induced by estradiol (E2) and progesterone (P4) supplementation/withdraw schedule including the induction of decidualisation with intra-uterine oil application as described e.g. in Menning (2012) and outlined in FIG. 1 and Example 1. Briefly, starting six days after ovariectomy female Balb/cAnN mice [Janvier] received 20 mg/kg, 5 mg/kg or 1 mg/kg twice weekly subcutaneous (s.c.) injections of the IL-11 function blocking monoclonal antibodies TPP-18068 in comparison to 20 mg/kg of an IgG control antibody [TPP-10159] for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 8 shows dose dependent inhibition of menstrual bleeding in Heavy Menstrual Bleeding Model in Balb/c mice by the IL-11 function blocking antibody TPP-18068, with no inhibition by 1 mg/kg of TPP-18068 (blood loss 39 μl+/−14 μl SD), but significant inhibition by 5 mg/kg (blood loss 19 μl+/−11 μl SD) or 20 mg/kg (blood loss 19 μl+/−9 μl SD) vs 20 mg/kg control antibody TPP-10159 (blood loss 37 μl+/−12 μl SD) twice weekly s.c. injections. Significance was tested by one-way ANOVA with Bonferroni correction for multiple comparison (** p<0.01)

### Example 8: Animal Activity of Murine HMB Model Treated with TPP-18068

As pointed out in Examples 1 and 2 inhibiting the IL-11 signaling pathway by a function blocking IL-11 antibody showed marked effects on both heavy menstrual bleeding (FIG. 2) and uterus weight (FIG. 3). Therefore, these results lead to the conclusion that IL-11 function blocking strongly attenuates menstruation and reduces bleeding or even causes amenorrhea. For the expert it is known that avoiding or attenuation of menstruation and induction of amenorrhea is an effective treatment for primary and secondary dysmenorrhea as continues application of progestins demonstrated (Power et al., 2007; Sachedina & Todd, 2020). Therefore, agents capable of binding to and inhibiting or antagonizing the action of IL-11 will be an effective treatment for primary and secondary dysmenorrhea. In addition to the measurements of body weight as described in example 4 measurements of well-being can be also addressed in primary behavioral tests evaluating locomotion and exploration in an open field analyses (Murbach et al., 2014). Animals are exposed to a new environment in an open field setting. Horizontal and vertical activity is monitored in an ActiMoT (TSE systems). Animals with less stress, pain or better well-being are more active.

In a mouse model of heavy menstrual bleeding the effects of function blocking IL-11 antibodies on locomotion and exploration in an open field analyses were evaluated. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/c mice [Janvier Labs] received 20 mg/kg twice weekly subcutaneous (s.c.) injections of the IL-11 function blocking monoclonal antibodies TPP-18068 or an IgG control antibody [TPP-10159] for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. Two days after P4 withdrawal horizontal and vertical activity was monitored in an ActiMoT (TSE systems) during a period of five minutes.

As shown in FIG. 9 animals treated with the Il-11 antagonizing antibody TPP-18068 were more active. They showed increased exploratory behavior as seen in the travelled distance (horizontal activity in meter per 5 minutes) (30.6m/5 min+/−11.6m/5 min SD; two-tailed t test: *p<0.05) and in rearing (vertical activity in numbers of rearing within 5 minutes) (26.4/5 min+/−12.2/5 min; two-tailed t test: **p<0.01, respectively) which both were significantly increased in comparison to TPP-10159 control Ab treated animals (horizontal activity 19.1m/5 min+/−3.6m/5 min; rearing 12.5/5 min+/−7.0/5 min), indicating less stress, less pain or in general increased well-being.

### Example 9: Stat3 Phosphorylation in Decidualized Uterine Horns of the Animals (Murine HMB Model) Treated with TPP-18068

IL-11 binds to the IL-11 receptor. The dimer binds to Gp130 which is responsible for the further downstream signaling. Signaling leads to the activation of intracellular protein kinases and the phosphorylation of ‘Signal transducer and activator of transcription 3’ (STAT3) (Harmegnies et al. (2003)). Activation of IL-11 in the differentiation of the endometrium increases phosphorylation of STAT3. This was analyzed in a modified mouse model of heavy menstrual bleeding leading to differentiation of the murine endometrium. Artificial menstrual cycle phases of ovariectomized mice were induced by estradiol (E2) and progesterone (P4) supplementation/withdraw schedule including the induction of decidualisation with intra-uterine oil application in only one horn of the murine uterus as described e.g., in Menning (2012) and outlined in FIG. 1 and Example 1. Briefly, starting six days after ovariectomy female Balb/cAnN mice [Janvier] received 20 mg/kg twice weekly subcutaneous (s.c.) injections of the IL-11 function blocking monoclonal antibodies TPP-18063 or TPP-18068 or an IgG control antibody [TPP-10159] for a period of 13 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the uteri were removed, weighted and the horns were separated. Immediately after separation fragments of the decidualized uterine horns and the not decidualized uterine horns were separately transferred in small tubes containing lysis puffer (cisbio cat #64KL1FDF) with Phospho and Total protein blocking reagent (cisbio cat #64KB1AAD) and snap frozen in liquid nitrogen. For sample preparation tissues were thawed on ice and homogenized. Protein concentrations were determined to generate 2 mg/ml protein dilutions using Lysis/blocking buffer. Subsequently phosphorylated and total Stat3 were measured accordingly to manufacturers protocol by using Phospho-STAT3 (Tyr705) cellular kit #62AT3PEG and Total STAT3 cellular kit #64NT3PEG from cisbio.

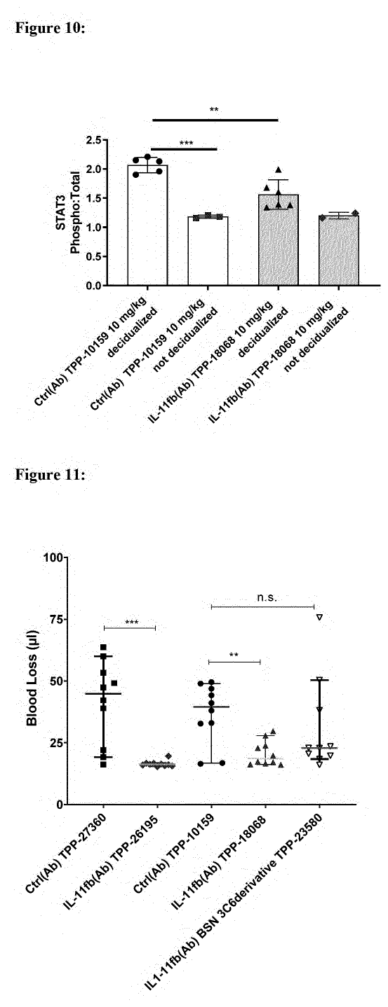

FIG. 10 shows the effect of uterine endometrial differentiation on uterine Stat3 phosphorylation and inhibition of this signaling by IL-11 blockage. Stat3 phosphorylation is significantly increased on day 12 in the differentiated uterine horns of the animals (relative value 2.07+/0.13 SD) treated with control antibody TPP-10159 in comparison to the undifferentiated horns (relative value 1.18+/−0.03 SD) treated with the control antibody TPP-10159. IL-11 function blocking antibody TPP-18068 reduces in comparison to the control antibody TPP-10159 (relative value 2.07+/0.13 SD) Stat3 phosphorylation in decidualized uterine horns (relative value 1.56+/−0.25 SD in TPP-18068 treated animals vs relative value 2.07+/0.13 SD in TPP-10159 treated animals) but not in the not decidualized uterine horns on d12 in the HMB model (relative value 1.2+/−0.04 in TPP-18068 treated animals vs relative value 1.18+/0.03 SD in TPP-10159 treated animals). Significance was determined by one-way ANOVA with Bonferroni correction for multiple comparison (** p<0.01; *** p<0.001).

### Example 10: Blood Reduction and Uterine Weight Reduction in Murine HMB Model Treated with TPP-18068 or TPP-26195

The IL-11 function blocking antibody TPP-18068 shows dose dependent significant effects on attenuation of heavy menstrual bleeding, intrauterine IL-11 signaling and signs of wellbeing on mice in the murine heavy menstrual bleeding model, as shown in examples 6-9. As a classical IgG antibody this antibody has two identical binding sites for IL-11 binding. By combination with a binding site for a different epitope the biparatopic IL-11 function blocking antibody TPP-26195 was generated as shown in EXAMPLE 34. The effect of the monospecific TPP-18068 and the derived biparatopic IL-11 function blocking antibody TPP-26195 on menstrual bleeding was tested in the murine heavy menstrual bleeding model in Balb/cAnN mice [Janvier] in comparison to their respective control IgG-antibodies and in addition the mouse IgG1 version of an IL-11 antibody described in WO2019238882 as IL-11 function blocking and named here BSN 3C6 2.2-2.1-mIgG1Kappa (TPP-23580)

In addition, the uteries were prepared at day 12 of the heavy menstrual bleeding model from four animals each treated with the IL-11 function blocking biparatopic antibody TPP-26195 or the control antibody. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/cAnN mice [Janvier] received 15 mg/kg twice weekly subcutaneous (s.c.) injections of the IL-11 function blocking monoclonal antibodies TPP-18068, TPP-26195 in comparison to 15 mg/kg of their respective IgG control antibodies [TPP-10159 and TPP-27360] for a period of 16 days. In addition, the mouse IgG1 version of an IL-11 antibody described in WO2019238882 as IL-11 function blocking and named here BSN 3C6 2.2-2.1-mIgG1Kappa (TPP-23580) was tested. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 11 shows the significant attenuations of menstrual bleeding by the IL-11 function blocking antibody TPP-18068 (blood loss 20.7 μl+/−5.6 μl SD; ** p<0.01) and by the derived biparatopic IL-11 function blocking antibody TPP-26195 (blood loss 16.4 μl+/−1.2 μl SD; *** p<0.001) in Balb/c mice in comparison to their control IgG-antibodies TPP-10159 (blood loss 36.8 μl+/−12.2 SD) or TPP-27360 (blood loss 41.2 μl+/−17.0 μl), respectively with s.c. dosing of 15 mg/kg twice weekly. The mouse IgG1 version of an IL-11 antibody described in WO2019238882 as IL-11 function blocking and named here BSN 3C6 2.2-2.1-mIgG1Kappa (TPP-23580) showed no significant effects on heavy menstrual bleeding at the selected s.c. dose of 15 mg/kg twice weekly (blood loss 30.8 μl+/−19 μl SD) in comparison to the control IgG anti body TPP-10159 (blood loss 36.8 μl+/−12.2 μl SD). Significance was determined by two-tailed Mann-Whitney test.

FIG. 12 shows the attenuation of uterine differentiation by the biparatopic IL-11 function blocking antibody TPP-26195 in Balb/c mice in comparison to the control IgG-antibody as measured by the uterine weight on day 12 of the murine heavy menstrual bleeding model (n=4). The biparatopic antibody TPP-26195 strongly inhibits endometrial differentiation as indicated by the strongly reduced uterine weight (55.6 mg+/−20.8 mg SD) in comparison to the control-antibody TPP-27360 treated group (454.8 mg+/−339.8 mg SD). Significance was determined by one-tailed t-test: * p<0.05.

### Example 11: Dose-Dependent Blood Reduction in Murine HMB Model Treated with TPP-26195

In a mouse model of heavy menstrual bleeding the effects of the biparatopic IL-11 function blocking antibody TPP-26195 on menstruation was tested dose dependently in comparison to its mouse IgG1 control antibody. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/cAnN mice [Janvier] received 10 mg/kg or 3 mg/kg or 1 mg/kg or 0.3 mg/kg twice weekly subcutaneous (s.c.) injections of the IL-11 function blocking biparatopic antibody TPP-26195 or 10 mg/kg twice weekly of its mouse IgG1 control antibody [TPP-27360] for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 13 shows the dose dependent attenuation of menstrual bleeding by the biparatopic IL-11 function blocking antibody TPP-26195 in Balb/c mice with twice weekly s.c. dosing of 10 mg/kg (blood loss 14.4 μl+/−7.0 μl SD; ** p<0.01), 3 mg/kg (blood loss 11.7 μl+/−1.1 μl SD; *** p<0.001), 1 mg/kg (blood loss 17.4 μl+/−9.2 μl SD; ** p<0.0l) and 0.3 mg/kg (blood loss 27.5 μl+/−21.9 μl SD) in comparison to the group treated twice weekly with 10 mg/kg control IgG-antibody TPP-27360 (blood loss 41.8 μl+/−26.8 μl SD) with significant inhibition at 10, 3 or 1 mg/kg twice weekly s.c. dosing. Significance was calculated by one-way ANOVA with Bonferroni correction for multiple comparison.

### Example 12: Blood Reduction in Murine HMB Model Treated with TPP-29603, TPP-29528 and TPP-29519

In a mouse model of heavy menstrual bleeding the effects of the biparatopic IL-11 function blocking antibody TPP-29603 or the IL-11 function blocking antibodies TPP-29528 or TPP-29519 on menstruation were tested in comparison to a mouse IgG1 control antibody (TPP-10159). Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/cAnN mice [Janvier] received equimolar twice weekly subcutaneous (s.c.) injections of the IL-11 function blocking biparatopic antibody TPP-29603 (1 mg/kg) or the IL-11 function blocking antibodies TPP-29528 (1.39 mg/kg) or TPP-29519 (1.4 mg/kg), respectively or a lower dose of the antibody TPP-29519 (0.42 mg/kg) twice weekly in comparison to 1.4 mg/kg twice weekly of the mouse IgG1 control antibody [TPP-10159] for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 14 shows the attenuation of menstrual bleeding by equimolar dosing of the biparatopic IL-11 function blocking antibody TPP-29603 (blood loss 13.8 μl+/−4.1 μl SD) and the antibodies TPP-29528 (blood loss 26.5 μl+/−20.2 μl SD) and TPP-29519 (blood loss 12.7 μl+/−2.4 μl SD) in Balb/c mice in comparison to the control IgG-antibody TPP-10159 (blood loss 35.5 μl+/−27.2 μl SD). In addition, also a lower dose of the antibody TPP-29519 is shown (blood loss 33.5 μl+/−19.0 μl SD). The experiment demonstrates significant inhibition of menstrual bleeding by the IL-11 function blocking antibodies TPP-29603 and TPP-29519 at the equimolar doses. Significance was calculated by one-way ANOVA with Dunnett's correction for multiple comparison (log normalized values): ** P<0.01.

### Example 13: Dose Dependent Blood Reduction in Murine HMB Model Treated with TPP-29523

In a mouse model of heavy menstrual bleeding the effects of the IL-11 function blocking antibody TPP-29523 on menstruation was tested dose dependently in comparison to its mouse IgG1 control antibody (TPP-10159) and a very low dose of the biparatopic IL-11 function blocking antibody TPP-29603. Artificial menstrual cycle phases of ovariectomized mice were induced by estradiol (E2) and progesterone (P4) supplementation/withdraw schedule including the induction of decidualisation with intra-uterine oil application as described e.g., in Menning (2012) and outlined in FIG. 1 and Example 1. Briefly, starting six days after ovariectomy female Balb/cAnN mice [Janvier] received 2 mg/kg or 0.7 mg/kg or 0.3 mg/kg twice weekly subcutaneous (s.c.) injections of the IL-11 function blocking antibody TPP-29523 or 2 mg/kg of its mouse IgG1 control antibody TPP-10159 or 0.143 mg/kg of the biparatopic IL-11 function blocking antibody TPP-29603 twice weekly for a period of 16 days. One week after ovariectomy female Balb/c mice received subcutaneous (s.c.) injections of 100 ng 17alpha-estradiol (E2) in ethanol/arachis oil (1:9) on three consecutive days (FIG. 1). After a three day break a progesterone (P4) releasing silastic tube (0.5 mg P4/day) was implanted s.c. into the back of mice followed by further applications of 5 ng E2 on three consecutive days. Concomitant with the last E2 treatment 50 μl sesame oil were injected into one uterus horn to induce decidualization. Four days later the P4 implant was removed to initiate P4 withdrawal. To quantify the total amount of blood loss tampon-like cotton pads (4-4.8 mm diameter, Roeko, Coltene/Whaledent, Altstatten, Switzerland) were inserted into the vagina of mice at P4 withdrawal. Tampons were changed twice daily and collected for each mouse separately for three days. Blood volume was quantified by the alkaline hematine method reported elsewhere (Hallberg & Nilsson, 1964). Briefly, tampons were left to dry at room temperature. Heme chromogens were dissolved in 1000 μl 5% NaOH (w/v) and rotated over night at room temperature. Optical density of the eluate was measured in an ELISA Reader at a wavelength of 546 nm. Blood volume contained in cotton swabs was calculated based on a regression curve of standards prepared from venous blood.

FIG. 15 shows the dose dependent attenuation of menstrual bleeding by the IL-11 function blocking antibody TPP-29523 in Balb/c mice at twice weekly s.c. dosing of 2 mg/kg (blood loss 22.9 μl+/−1.5 μl SD), 0.7 mg/kg (blood loss 21.0 μl+/−3.1 μl SD), and 0.3 mg/kg (blood loss 24.5 μl+/−3.5 μl SD) in comparison to the control IgG-antibody TPP-10159 of 2 mg/kg dosing (blood loss 43.5.8 μl+/−27.9 μl SD). In addition, twice weekly s.c. dosing of 0.143 mg/kg of the biparatopic IL-11 function blocking antibody TPP-29603 was tested (blood loss 43.11 μl+/−7.9 μl SD). The data demonstrate significant inhibition (** p<0.01) of menstrual bleeding by the IL-11 function blocking antibody TPP-29523 already at all tested twice weekly dosing. Significance was calculated by one-way ANOVA with Dunnett's post-hoc test for multiple comparison.

### Example 14-20 Show Generation and Characterization of TPP-18068, TPP-18087, and TPP-19528

### Example 14: Generation of TPP-18068, TPP-18087 and TPP-19528 from BioInvent Antibody Libraries

A fully human antibody phage display library (BioInvent n-CoDeR Fab lambda library) was used to isolate human monoclonal antibodies by selection against soluble biotinylated IL11 antigens. IL-11 was used from commercial sources: human IL-11 (Invigate, e.g., lot #C121021-19), murine IL-11 (Invigate, e.g., lot #C210819-09), cynomolgus IL-11 (Invigate, e.g., lot #C290621-19). Antigens were biotinylated using a Sulfo-NHS-LC-Biotin kit (Thermo Scientific™; CatNo. A39257). Free biotin was removed from the reactions by dialysis against the appropriate buffer.

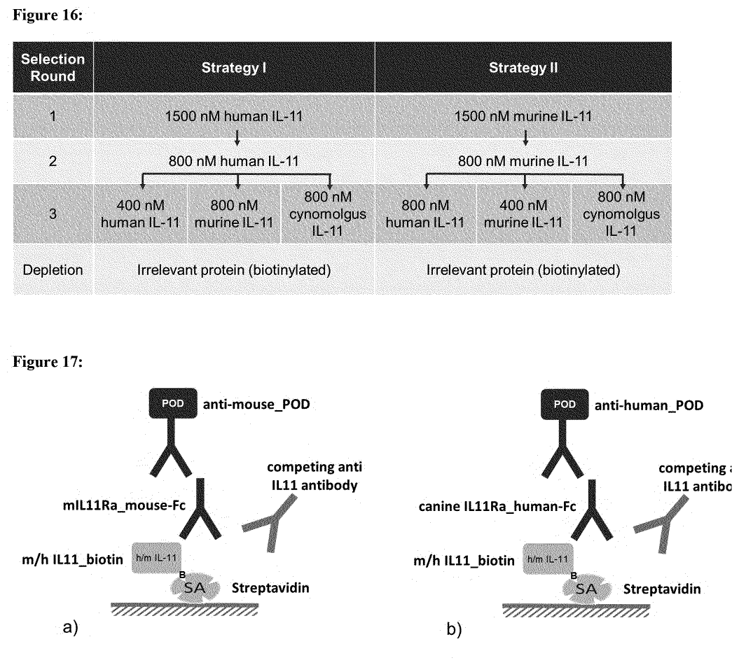

For the panning procedure, which is schematically shown in FIG. 16, the following protocol was applied: Streptavidin-coupled Dynabeads M-280 (Invitrogen™) were coated for one hour at room temperature (RT) with the biotinylated antigen (1 tube) and the biotinylated off-target (3 tubes), respectively. Dynabeads were washed and subsequently blocked for 1 h at RT with end-over-end rotation. For depletion of off-target binders the blocked phage library was added to the blocked off-target loaded Dynabeads and incubated for 10 min at room temperature with end-over-end rotation. This depletion step was repeated 2 times. The depleted phage library was added to the blocked target loaded Dynabeads and incubated for 60 min at RT with end-over-end rotation. After stringent washing (3× in blocking buffer and 9× in PBS (150 mM NaCl; 8 mM Na2HPO4; 1.5 mM KH2PO4; adjusted to pH=7.4-7.6) with 0.05% Tween-20) Dynabeads with Fab-phages binding specifically to the coated target were directly used to infect Escherichia coli strain HB101. Subsequently the phages are amplified in Escherichia coli strain HB101 using M13KO7 Helper Phage (Invitrogen™). In the following selection rounds the target concentration was decreased to augment the selection pressure for high affinity binders. During panning of this library, two different selection strategies were carried out in order to identify antibodies showing cross-reactivity against human, mouse and/or cynomolgus IL-11 In the first strategy, the human target was used in the first two rounds of panning, whereas in the second strategy, the murine target was used in the first two rounds of panning. In both strategies, the amplified phages of the second rounds were then panned in parallel against the human, murine and cynomolgus IL-11 in a third round.

For a first qualitative assessment, 88 Fab-phage clones from each clone pool were randomly picked and inoculated into a flatbottom 96-well plate filled with 100 μl LB media supplemented with 100 μg/ml Ampicillin and 1% Glucose. After overnight cultivation at 37° C., a new flatbottom 96-well plate filled with 100 μl LB media supplemented with 100 μl Ampicillin was inoculated with 5 μl of the overnight culture. The inoculated plate was incubated for 3 hours at 37° C. After the 3 hours 100 μl LB media supplemented with 100 μl/ml Ampicillin, 200 μM Isopropyl-β-D-thiogalactoside (IPTG) and M13KO7 helper phage (6×10⁹ plaque forming units/ml) was added, and plates further incubated at 37° C. for 30 minutes followed by incubation at 30° C. overnight. Next day the supernatant, including the expressed Fab-phage molecules, was tested for binding to the respective target used before for panning (human, mouse and cynomolgus IL-11). A “binder” has been defined as a Fab-phage molecule showing in an ELISA assay (see below) at least a signal intensity of the average signal intensity of non-binding control Fab-phage molecules plus 10 times the standard deviation (average+10 ×standard deviation of non-target binding Fab-phage). The ELISA assay was carried out as follows: 384-well streptavidin plates (Greiner) were coated over night at 4° C. with the biotinylated human or mouse IL-11 (30 μl of a 1 μg/ml PBS solution) or the biotinylated non-target control protein (30 μl of a 1 μg/ml PBS solution). Coating solution was discarded, plates washed using phosphate buffered saline (PBS) including 0.05% Tween (PBST) and subsequently blocked for 1 hour using 3% fat free milk powder solved in PBST. After a single washing step using PBST 30 μl/well of the Fab-phage containing supernatant from the expression cultures were added und plates were incubated for one hour at room temperature (RT). After another single washing step using PBST, 30 μl of a 1/5000 in PBST diluted anti-M13-antibody conjugated to horseradish peroxidase (GE Healthcare) were added to each well and subsequently incubated for one hour at RT. A last single washing step using PBST was followed by the addition of 30 μl substrate solution (Amplex Red, Invitrogen) After a 15 min incubation step in the dark at RT the fluorescence signals were measured in a fluorescence microplate reader (Tecan) using excitation of 535 nm and emission detection at 590 nm.

Plasmid-DNA from the 6 Pools of the third round which showed significant hit rates was extracted and purified. The gene-III was then cut out from the plasmid by means of a restriction enzyme, the plasmid was ligated again and transformed into E. coli TOP10. By removing the gene-III, the expression format is changed from Fab-phage to soluble Fabs. The soluble Fabs were produced as follows: Single bacterial colonies were picked from the transformation plates and inoculated in 384-well flat bottom plates filled with 50 μL LB media supplemented with 100 μg/ml Ampicillin and 1% glucose followed by an incubation step at 37° C. overnight. The next day 2 μl/well of overnight culture was transferred into a new 384-well flat bottom expression plate prefilled with 50 μl/well LB media supplemented with 100 μg/ml Ampicillin and 200 μM IPTG. The expression plates were incubated over night at 30° C. Next day 10 μl of the Fab expression culture was tested in an ELISA based High-Throughput-Screen (HTS-ELISA) on human and murine IL-11, respectively. The structure and the implementation of the HTS-ELISA is like the previously described Fab-phage ELISA with the following changes. As blocking reagent Smart Block (Candor) was used and as detection antibody an anti-c-myc antibody horseradish peroxidase conjugate (Bethyl).

### Example 15: Recombinant DNA Constructs and Expression, Purification and Quantification of and Full-Length Antibodies

From the panning campaign, two potential antibody candidates were selected for further characterization. The corresponding Fab fragments were reformatted to full-length murine IgG1 molecules. Heavy and light chains were cloned into pTT5 vector system (National Research Council Canada, NRC file 11266). and expressed in HEK293 cells using standard transient transfection procedures. In brief, for small-scale transfection, the HEK293 cells were diluted to 0.5×10⁶ cells/mL with F17 medium (Invitrogen, #A13835-01) supplemented with 10 mL/L of 10% Pluronic F68 (Invitrogen; #24040-32) solution to a total volume of 22.5 mL in 125 mL polycarbonate Erlenmeyer shaker flasks (Corning, #CLS431143), two days before transfection. The cell density at the day of transfection should be at 1.7×10⁶ cells/mL. At the day of transfection, 25 μg DNA were thawed in 1.25 mL transfection medium (F17 medium+Pluoronic as above) and 50 μl Polyethylenimine (Polysciences, #23966) at 1 mg/mL, i.e., 50 μg, were added to another 1.25 ml transfection medium. Both solutions were vortexed and subsequently, the Polyethylenimine (PEI) solution transferred to the DNA solution. The mixture was vortexed again and incubated for 15 min at room temperature. After the DNA-PEI mixture was added to the cells and the flask immediately swirled, the cells were incubated at 370 at 5% C02 in a humidified incubator for five days.

Supernatants were collected and antibodies were purified by Protein A chromatography followed by size-exclusion chromatography. After harvest, the supernatant was concentrated and filtered (pore size 0.2 μm) and loaded onto a HiTrap MabSelect SuRe column (Cytiva). The column was equilibrated in DPBS, pH 7.4. coupled to an Äkta Pure 25 system (Cytiva). The column was washed with 10 CV and elution was performed with 6 CV elution buffer (50 mM acetic acid+50 mM NaCl, pH 3.0). Peak fractions were pooled and neutralized using 3 M Tris pH 9.0. The pool was concentrated (Vivaflow 200 30 kDa membrane [Hydrosart 30 kDa]) and filtered through a 0.2 μm filter. The capture step pool was analyzed by analytical SEC (SEC sample was diluted with DPBS pH 7.4 to 2 mg/ml). Next, a preparative SEC run was performed using a Superdex 200 SEC column (Cytiva) coupled to an Äkta Pure 25 system. The SEC column was equilibrated using 2 CV DPBS, pH 7.4, sample was loaded, and the column was eluted with 1.5 CV DPBS, pH 7.4. Peak fractions were pooled. The pool was concentrated and sterile filtered (pore size 0.2 μm). The final concentration of the antibody solution was determined using absorbance at 280 nm using a Nanodrop UV spectrophotometer (Thermo). The antibodies were aliquoted, snap-frozen in liquid nitrogen and stored at −80° C.

Finally, the resulting antibodies TPP-18063, TPP-18068, TPP18087 and TPP-19528 were obtained.

### Example 16: Binding Affinity of TPP-18063, TPP-18068, TPP18087 and TPP-19528 Analyzed by SPR

SPR-based experiments to determine the binding affinity of TPP-18068, TPP-18087, TPP-19528 as well as of commercially available IL-11 antibody TPP-14250 (R&D Systems, #MAB218) were run on a Biacore T200 instrument (GE Healthcare) at 25° C. using assay buffer HBS EP+, 300 mM NaCl, 1 mg/ml BSA, 0.05% NaN3. Antibodies were captured via anti-mouse Fc IgGs (“Mouse antibody capture kit, Order No. BR100383, Cytiva) covalently amine coupled to a Series S CM5 sensor chip (Cytiva). The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human IL-11 (Invigate, e.g., lot #C121021-19), and murine IL-11 (Invigate, e.g., lot #C210819-09) were used as analytes. First, a single concentration binding experiment using 200 nM IL-11 was performed to determine a rough affinity. Based on that experiment an IL-11 concentration range was chosen to optimally determine kinetics and affinity of anti-IL-11 antibodies in multicycle kinetics mode. The sensor surface was regenerated with glycine pH 1.7 after each antigen injection. Obtained sensorgrams were double referenced (subtraction of reference flow cell signal and buffer injection) and were fitted to a 1:1 Langmuir binding model to derive kinetic data using the Biacore T200 Evaluation software or the Biacore Insight software. Results are shown in Table E1.

TABLE E1
Binding affinity of TPP-18063, TPP-18068,
TPP18087, TPP-19528 and TPP-14250

| Antibody | K_{D} [M]: Human IL11 | K_{D} [M]: Mouse IL11 |
|---|---|---|
| TPP-14250 | 1.9E−08 | 3.8E−09 |
| TPP-18063 | 5.3E−08 | 1.0E−08 |
| TPP-18068 | 4.8E−08 | 7.2E−10 |
| TPP-18087 | 5.3E−08 | 4.2E−08 |
| TPP-19528 | 1.1E−08 | 9.0E−09 |

### Example 17: Inhibition of IL-11 Mediated Signaling by TPP-18063, TPP-18068, TPP18087 and TPP-19528 by Use of Reporter Gene Assay

The blocking activity of TPP-18063, TPP-18068, TPP-18087, TPP-19528, commercial IL-11 antibody TPP-14250 (MAB218) and isotype control TPP-10159 for human IL-11 and mouse IL-11 mediated Stat3 signaling were measured in a reporter gene assay. Briefly, corresponding human IL11RA (SEQ ID NO: 7) or mouse Il11 ra (SEQ ID NO: 10) expression plasmid, previously generated by recombinant DNA technology, was co-transfected with reporter gene plasmid (pNL[NlucP/SIE/Hygro] Vector, Promega #CS189701) into HEK 293F cells. 10000 transfected cells per well were seeded in flat-bottom 384-well plates (Corning, #354660), and then incubated overnight at 37° C., 5% CO₂. At the next day, a serial dilution from 4E-7 to 5.5E-10 [M] (final assay dilution) of TPP-18068, TPP-18087, TPP-19528, TPP-14250, and TPP-10159 were prepared in cell culture medium, containing a fixed concentration of 0.78 [nM] human IL-11 (Invigate, e.g., lot #C121021-19) or 3.13 [nM] murine IL-11 (Invigate, e.g., lot #C210819-09). The fixed human/murine IL11 concentration should be an equal to the EC40 value of a human/murine IL dose response cure in the reporter gene assay and had been determined beforehand. The mixture of antibody/antigen was incubated for 30 min at RT. As a negative control, cells were also incubated in the absence of antibody and IL11. Thereafter, the culture medium above the cells was discarded, 25 μl per well of the mixtures were added and incubated with the cells for 5 hours at 37° C. Thereafter, 25 μl per well of Nano-Luc substrate (Promega, N1120) diluted (1:50) in Nano-Luc buffer (Promega, N1120) was added to the plate. The plate was incubated for 3 min at room temperature in the dark and then the luminescence was measured by a microtiter plate reader. Signals from dose responses were used to calculate IC50 values by GraphPad Prism (GraphPad Software, San Diego). Efficacy was calculated as [(signal measured in the absence of antibody−signal measured at the highest antibody concentration)×100] divided by (signal in the absence of antibody−signal measured in the absence of IL11). Results are shown in Table E2.

TABLE E2
Inhibition of IL-11 mediated signaling by TPP-
18063, TPP-18068, TPP18087 and TPP-19528

| | hIL-11 | | mIL-11 | |
|---|---|---|---|---|
| Antibody | IC50 [M] | Efficacy (%)* | IC50 [M] | Efficacy (%)* |
| TPP-14250 | 2.41E−10 | 27.1 | 1.42E−07 | 69.1 |
| TPP-18063 | 1.1E−07 | 54.3 | 9.5E−08 | 60.0 |
| TPP-18068 | 4.83E−09 | 20.7 | 2.67E−09 | 84.1 |
| TPP-18087 | 1.07E−07 | 51.5 | 3.31E−07 | 49.0 |
| TPP-19528 | 1.5E−08 | 65.6 | 9.6E−09 | 80.7 |
| TPP-10159 | NM | 0 | NM | 9.4 |

NM = Not Meaningful (=no value reported if efficacy <20% or no IC₅₀ value could be calculated);
*Efficacy was calculated as [(signal measured in the absence of antibody − signal measured at the highest antibody concentration) × 100] divided by (signal in the absence of antibody − signal measured in the absence of IL11).

### Example 18: Inhibition of the Interaction of IL-11 with IL-11Ra by TPP-18063, TPP-18068, TPP18087, and TPP-19528 Analysed in Di-Complex ELISA

In the Di-complex ELISA format anti IL-11 antibodies are tested for the ability to interfere with the formation of complexes consisting of immobilized murine IL-11 (Invigate, e.g., lot #C210819-09) or human IL-11 (Invigate, e.g., lot #C121021-19), and murine IL11RA-Fc fusion proteins or canine IL11RA-human-Fc fusion protein (FIG. 17a, b).

In order to test the ability of murine IL-11 antibodies to interfere with the complex formation of immobilized murine or human IL-11 with canine IL11RA-human-Fc fusion protein the assay is performed as described below with canine IL11RA-Fc (Sino Biological, #70078-D02H) and anti-human-POD (Sigma, #A0170) allowing the detection.

In order to test the ability of human IL-11 antibodies to interfere with the complex formation of immobilized murine or human IL-11 with murine IL11RA-Fc fusion protein the assay is performed as described below, with the exception that murine IL11RA-Fc fusion protein (R&D System, #7405-MR) is used instead of the canine IL11RA-Fc fusion protein, allowing the detection with anti-murine IgG (Fc specific)-Peroxidase (Jackson, #715-035-) instead of anti-human POD.

The assay is performed using streptavidin coated microtiter plates (Greiner bio-one, #781997). In a first step, 10 nM biotinylated mouse IL-11 (Invigate, Germany, e.g., lot #C210819-09) or human IL-11 (Invigate, Germany, e.g., lot #C121021-19) is coated in 25 μl PBS on the plate at 4° C. overnight. Next day, the plate is washed three times with 50 μl PBST and then blocked with 50 μl SmartBlock (Candor, #113125) for one hour at room temperature (RT). After additional three washes with PBST, 25 μl of a mixture of the murine anti IL-11 antibody and recombinant canine IL11RA-Fc fusion protein (Sino Biological, #70078-D02H) in PBS-T, 10% Smartblock are added simultaneously to the plate and incubated for one hour at RT. In order to generate a dose response, the antibody is titrated from 1E-07 to 1.6E-10 [M] against 2 nM canine IL11RA-Fc., which is approximately the EC50 of a dose response titration of the receptor binding to immobilized IL-11 in the absence of antibody. After three additional washes with PBS-T, 25 μl anti-human-POD (Sigma, #A0170) in PBS-T, 10% Smartblock is added and incubated for 1 hour at RT. After a final three washes with PBS-T, 25 μl AmplexRed (Fisher Scientific, A12222), 1/1000 in PBS, 0,003% H₂O₂, is added as substrate and incubated for 15 min at RT in the dark. Then, the fluorescence is measured with a plate reader at 535/595 nm. Signals from dose responses were used to calculate IC50 values (GraphPad Prism). Efficacy was calculated as [(signal measured at lowest antibody concentration−signal measured at the highest antibody concentration)×100] divided by (signal measured at lowest antibody concentration−signal measured in the absence of IL11Ra). Results are shown in Table E3.

TABLE E3
Inhibition of the interaction of human IL-11
with human IL-11Ra by TPP-18068, TPP18087
and TPP-19528 analyzed in Di-complex ELISA

| | hIL11 | | mIL11 | |
|---|---|---|---|---|
| Antibody** | IC50 [M] | Efficacy (%)* | IC50 [M] | Efficacy (%)* |
| TPP-14250 | 7.4E−09 | 67.2 | 1.3E−09 | 92.3 |
| TPP-18063 | 5.4E−08 | 42.0 | 1.2E−08 | 68.9 |
| TPP-18068 | NM | 19.6 | 2.5E−09 | 72.7 |
| TPP-18087 | NM | 10.7 | NM | 13.4 |
| TPP-19528 | 3.0E−09 | 57.0 | 2.4E−08 | 79.0 |
| TPP-10159 | NM | 10.3 | NM | 10.2 |

NM = Not Meaningful (=no value reported if efficacy <20% or no IC₅₀ value could be calculated);
*Efficacy was calculated as [(signal measured at lowest antibody concentration − signal measured at the highest antibody concentration) × 100] divided by (signal measured at lowest antibody concentration − signal measured in the absence of IL11Ra).
**murine IgG1-IL-11 antibody

### Example 19: Inhibition of the Formation of IL-11/IL-11Ra/Gp130 Complex by TPP-18063, TPP-18068, TPP-18087 and TPP-19528 Analysed in Tri-Complex ELISA

In the Tri-complex ELISA format IL-11 antibodies are tested for their ability to inhibit the formation of a complex consisting of murine or human gp130-Fc fusion protein, with murine or human IL-11, and a murine IL11RA-Fc fusion protein. The assay format was adapted according to FIG. 18 (a, b), depending on whether murine or human IL-11 IgG antibodies were tested.

The assay was performed using High Binding microtiter plates (Greiner, #781077). In a first step, 25 μl of goat anti human IgG (Sigma, #12136), diluted 1:1000 in coating buffer (Candor, #121125) was coated on the plate at 4° C. overnight. Next day, the plate was washed one time with 50 μl PBST and then blocked with 50 μl SmartBlock (Candor, #113125) for one hour at room temperature (RT). At the same time, a mixture of a concentration range from 2.5E-07 [M] to 4.1E-10 [M] of the murine IL-11 antibody, and of a fixed concentration of human IL11 (e.g. Invigate, Germany [8 nM], e.g., lot #C121021-19) respectively murine IL-11 (e.g. Invigate, Germany [8 nM], e.g., lot #C210819-09), human gp130-human-Fc (R&D Systems, #671-GP, [2 NM]) respectively murine gp130-human-Fc (R&D Systems, #468-MG, [2 nM]) and the recombinant murine IL11RA-murine-Fc fusion protein (R&D System, #7405-MR, [2 nM]) in PBS-T, 10% SmartBlock, were preincubated in a 96 well microtiter plate (Nunc, #267334) for one hour at room temperature. After three additional washes of the coated and blocked microtiter plate with PBS-T, 25 μl of each mixture were added and the assay plate was incubated for one hour at room temperature. After three additional washes, anti-mouse-gp130 biotinylated antibody (R&D, #BAF468) or anti-human gp130 biotinylated antibody (R&D, #BAF228), both 1:1000 in 10% SmartBlock PBS-T, was added and incubated for 1 hour at RT. After three additional washes, a Streptavidin-Peroxidase conjugate (Sigma, #S5512) 1:1000 in SmartBlock PBS-T was added and incubated for one hour at room temperature. After a final three washes, AmplexRed (Fisher Scientific, A12222), 1/1000 in PBS, 0,003% H₂O₂, was added as substrate and incubated for 15 min at RT in the dark. Then, the fluorescence was measured with a plate reader at 535/595 nm. Signals from dose responses were used to calculate IC50 values using GraphPad Prism software. Efficacy was calculated as [(signal measured at lowest antibody concentration−signal measured at the highest antibody concentration)×100] divided by (signal measured at lowest antibody concentration−signal measured in the absence of antibody at an IL11 concentration of 8.2E-11 [M]). Results are shown in Table E4.

TABLE E4
Inhibition of the formation of human IL-11/IL-
11Ra/gp130 complex by TPP-18068, TPP-18087,
ad TPP-19528 analysed in Tri-complex ELISA

| | hIL11 | | mIL11 | |
|---|---|---|---|---|
| Antibody | IC50 [M] | Efficacy (%)* | IC50 [M] | Efficacy (%)* |
| TPP-14250 | 3.8E−08 | 67.2 | 1.7E−08 | 60.9 |
| TPP-18063 | 9.2E−08 | 60.6 | 1.7E−07 | 36.3 |
| TPP-18068 | NM | 7.3 | 6.6E−08 | 37.1 |
| TPP-18087 | 5.5E−08 | 60.4 | NM | 5.5 |
| TPP-19528 | 2.0E−08 | 57.0 | 1.4E−08 | 76.6 |
| TPP-10159 | NM | 13.7 | NM | 12.3 |

NM = Not Meaningful (=no value reported if efficacy <20% or no IC₅₀ value could be calculated);
*Efficacy was calculated as [(signal measured at lowest antibody concentration − signal measured at the highest antibody concentration) × 100] divided by (signal measured at lowest antibody concentration − signal measured in the absence of antibody at an IL11 concentration of 8.2E−11 [M]).

### Example 20: Binding of TPP-18068, TPP-18087 and TPP-19528 to IL2Ra Analyzed by ELSA

We tested the binding of TPP-18068, TPP-18087 and TPP-19528 to a panel of proteins and found specific binding of TPP-18068 towards human IL2Ra, which was further analyzed in a human IL2Ra direct binding ELISA. IL2RA (Peprotech, Germany, #200-02RC) was coated at 1 or 0 μg/ml (buffer only control) in 30 μl coating buffer (Candor, #113500) per well on a 384 well microtiter plate (Maxisorb, Nunc, #460518) over night at 4-8° C. After two washes with 50 μl/well washing buffer (PBS+0.05% Tween, pH 7.4), the plates were incubated for two hours at room temperature with 50 μl/well SmartBlock (Candor, #113500). After blocking, the plates were washed three times with washing buffer. Then, a dilution series of the respective antibody (1E-7 [M] down to 2E-13 [M]) in PBS+0.05% Tween, 10% SmartBlock were added in 30 μl to the microtiter plate and incubated for 90 min at room temperature. After three additional washes, 30 μl anti mouse IgG HRP-conjugated antibody (Biotechne, HAF007), diluted 1:1000 in PBS+0.05% Tween (pH 7.4), were added per well and incubated for one hour. After three additional washes, a mixture of Amplex red (Invitrogen, #12222, 10 mM stock solution in DMSO) 1:1000 diluted in PBS and H2O2 (30%, Merck, #107209)) diluted 1:10000 in PBS were added as substrate with 30 μl per well and incubated for 20 min in the dark. Relative fluorescence was measured with a microtiter plate reader at 535/590 nm.

The ratio of the signals obtained at an antibody concentration of 3.3E-7 [M] for TPP-18068 versus the isotype control TPP-10159 was calculated as 256, indicating a strong and significant binding of TPP-18068 to hIL2Ra, while TPP-18087 and TPP-19528 showed only ratios of 2.4, respectively of 0.8, indicating no significant binding.

### Example 21-24 Show Germlining, Sequence Optimization (PTM Removal and Affinity Maturation of TPP-18087

### Example 21: 1st Round Germlining (Single Mutation) and PTM Removal of TPP-18087

Antibody TPP-18087 was subjected to lead optimization procedures aiming to (i) optimize its affinity, (ii) increase its functional efficiency, (iii) reduce the risk for sequence-based immunogenicity and (iv) improve compatibility with downstream development processes.

### Germlining Design, PTM Removal Design and Plasmid Construction

The sequence of the parental antibody was aligned to the human germline sequence repertoire of IMGT (Lefranc 2003). The human germline sequence with the least number of amino acid differences at counterpart positions in framework and in CDRs with respect to TPP-18087 was selected as the germlining template. VL sequence of TPP-18087 shares the highest level of identity with human germline genes: hIGLV1-47*01 germline gene. VH sequence of TPP-18087 shares the highest level of identity with human germline gene: hIGHV3-30*03 germline gene. Single back mutation on non-human germline position was selected to constitute 1^{st} round germlining variants.

The sequence of TPP-18087 was scanned for the following types of critical post-translational modification (PTM) sites: asparagine deamidation (Asn-Gly and Asn-Ser) in CDRs, aspartate isomerization (Asp-Gly) in CDRs, unpaired Cys in CDRs and in frameworks, and N-linked glycosylation sites (Asn-Xxx-Ser/Thr in which Xxx can be any amino acid except for Pro) in CDRs and in frameworks. TPP-18087 has one Asn-Gly (NG) site in VL and one Asp-Gly (DG) site in VH. To remove NG site, three single mutations were performed: asparagine was converted to glutamine or serine, while glycine was converted to alanine. To remove DG site, three single mutations were performed: aspartate acid was converted to glutamate acid, while glycine was converted to alanine or serine. For TPP-18087, two PTM hot spots were identified for TPP-18087: one Asn-Gly (NG) site in VL and one Asp-Gly (DG) site in VH. NG site was mutated to QG, SG and NA. DG site was mutated to EG, DA and DS. The two PTM risk sites were probed combinatorically using all combinations of WT and single mutations

Plasmids of the chimeric antibodies were codon optimized for mammalian expression and then synthesized at Genewiz (South Plainfield, NJ, USA). V-genes of parental antibody and germlining variants were cloned into human IgG expression vectors (WuXi, China) to generate human IgG constructs of a desired isotype (VH domain was fused with human IgG4 SPLE variant, and VL domain was fused with human Ig lambda CL domain). Each construct contains the same constant segments, but confers a different VH or VL domain, as well as different site mutations.

### Antibody Production

The plasmids containing VH and VL gene were co-transfected into Expi293F cells. Cells were cultured for 5 days and the supernatant was collected for one-step protein purification using Protein A column (GE Healthcare, Cat. 175438). The protein concentration from the elution was determined by A280/Extinction coefficient using Nanodrop 2000. Thirty-six antibodies, including one chimeric antibody, twenty germlining antibody variants and fifteen PTM antibody variants were purified, analyzed by SDS-PAGE and HPLC-SEC, and then were stored at −80° C. or used in the subsequent assays.

### Affinity to Human/Mouse IL-11 by SPR

Antibody binding affinity to hIL-11 (Invigate, Germany, e.g., lot #C121021-19) and mIL-11 (Invigate, Germany, e.g., lot #C210819-09) was detected using Biacore 8K. Each antibody was captured on an anti-human IgG Fc antibody immobilized CM5 sensor chip (GE). hIL-11 and mIL-11 at different concentrations (0, 5, 15.82 and 50 nM) in 1×HBS-EP+ (pH 7.4) (GE healthcare) were injected over the sensor chip at a flow rate of 30 μL/min for an association phase of 120 s, followed by 200 s dissociation phase. The chip was then regenerated by 10 mM Glycine, pH 1.5 after each binding cycle.

The sensorgrams of blank surface and buffer channel were subtracted from the test sensorgrams. The experimental data was fitted by 1:1 model using Langmiur analysis. Molecular weight of 19.3 kDa was used to calculate the molar concentration of human and mouse IL-11. SPR results of 1st round germlining and PTM mutants are shown in Table E5.

TABLE E5
SPR results of 1^{st} round germlining and
PTM removal mutants for human/mouse IL-11

| | K_{D} [M]: | K_{D} [M]: | Mutation | Mutation |
|---|---|---|---|---|
| Antibody | hIL-11 | mIL-11 | Heavy chain | Light chain |
| TPP-18087 | 5.2E−09 | 1.2E−08 | VH-WT | VL-WT |
| TPP-31312 | 9.2E−09 | 1.3E−08 | VH-WT | VL-WT |
| TPP-31277 | 9.4E−09 | 1.2E−08 | VH-WT | VL-N31S |
| TPP-31278 | 2.0E−09 | 1.2E−09 | VH-WT | VL-A33Y |
| TPP-31279 | no binding | 1.4E−07 | VH-WT | VL-N35Y |
| TPP-31280 | 6.0E−07 | no binding | VH-WT | VL-Y51R |
| TPP-31281 | 1.7E−07 | 1.3E−07 | VH-WT | VL-D52N |
| TPP-31282 | 1.3E−08 | 1.9E−08 | VH-WT | VL-D53N* |
| TPP-31283 | 4.8E−08 | 4.1E−08 | VH-WT | VL-L54Q |
| TPP-31284 | 1.1E−08 | 1.5E−08 | VH-WT | VL-L55R |
| TPP-31285 | 7.6E−09 | 1.8E−08 | VH-WT | VL-S90A |
| TPP-31286 | 9.1E−09 | 1.2E−08 | VH-E1Q | VL-WT |
| TPP-31287 | 9.3E−09 | 1.3E−08 | VH-L5V | VL-WT |
| TPP-31288 | 1.1E−08 | 1.5E−08 | VH-L11V | VL-WT |
| TPP-31289 | 9.6E−09 | 1.4E−08 | VH-G16R | VL-WT |
| TPP-31290 | 2.9E−07 | 3.0E−07 | VH-H31S | VL-WT |
| TPP-31291 | no binding | no binding | VH-D33G | VL-WT |
| TPP-31292 | 1.1E−08 | 1.9E−08 | VH-N35H* | VL-WT |
| TPP-31293 | 4.8E−09 | 1.2E−08 | VH-L37V | VL-WT |
| TPP-31294 | 1.1E−08 | 1.7E−08 | VH-G49A | VL-WT |
| TPP-31295 | 2.0E−08 | 3.3E−08 | VH-F59Y* | VL-WT |
| TPP-31296 | 9.4E−09 | 1.2E−08 | VH-F63S | VL-WT |
| TPP-31312 | 9.2E−09 | 1.3E−08 | VH-WT | VL-WT |
| TPP-31297 | 8.7E−09 | 1.4E−08 | VH-WT | VL-N97Q |
| TPP-31298 | 9.3E−09 | 1.3E−08 | VH-WT | VL-N97S |
| TPP-31299 | 1.8E−08 | 1.6E−08 | VH-WT | VL-G98A |
| TPP-31300 | 3.8E−09 | 3.0E−09 | VH-D54E | VL-WT |
| TPP-31301 | 3.6E−09 | 2.9E−09 | VH-D54E | VL-N97Q |
| TPP-31302 | 3.8E−09 | 3.2E−09 | VH-D54E | VL-N97S |
| TPP-31303 | 6.9E−09 | 3.6E−09 | VH-D54E | VL-G98A |
| TPP-31304 | 1.5E−08 | 2.3E−08 | VH-G55A | VL-WT |
| TPP-31305 | 1.5E−08 | 2.5E−08 | VH-G55A | VL-N97Q |
| TPP-31306 | 1.5E−08 | 2.3E−08 | VH-G55A | VL-N97S |
| TPP-31307 | 3.1E−08 | 2.8E−08 | VH-G55A | VL-G98A |
| TPP-31308 | 4.0E−08 | 4.7E−08 | VH-G55S | VL-WT |
| TPP-31309 | 4.1E−08 | 5.3E−08 | VH-G55S | VL-N97Q |
| TPP-31310 | 4.0E−08 | 4.3E−08 | VH-G55S | VL-N97S |
| TPP-31311 | 8.7E−08 | 5.8E−08 | VH-G55S | VL-G98A |

*Consecutive numbering of amino acid residues was used
Underscore = improved binding affinity to human/mouse antigen;
italic = comparable binding;
bold = significantly decreased binding;
asterisk (*) = slightly decreased binding affinity (decreased by 1.5~2.5 fold), compared with WT antibody

The germlining mutants TPP-31277 to TPP-31296 were classified in to four categories in terms of their changes in human and mouse IL-11 binding affinity. The mutants highlighted by underscore showed improved binding affinity to human/mouse antigen compared with WT antibody.

The mutants highlighted in italic showed comparable binding affinity to human/mouse antigen compared with WT antibody. The mutants highlighted in bold showed significantly decreased binding affinity to human/mouse antigen compared with WT antibody. The mutants labeled with asterisk (*) showed slightly decreased binding affinity (decreased by 1.5-2.5 fold), compared with WT antibody.

For 1^{st} round PTM removal mutants TPP-31297 to TPP-31311, TPP-31300 (VH-D54E) is the best clone on binding affinity to human/mouse antigen in 15 variants of PTM removal according to the SPR result (Table E5). The mutation site of TPP-31298 clone is VL-N97S, which is also a germ-line mutation site, and this clone shows comparable binding affinity to human/mouse antigen compared with TPP-31312 (Table E5). TPP-31302 clone which include two mutation sites VH-D54E/VL-N97S showed improved binding to human and mouse IL11, so the two mutation sites VH-D54E/VL-N97S were involved in 2^{nd} round CDR germ-lining.

### Human and Mouse IL-11 RGA Assay

The purified antibodies were tested in human and mouse IL-11 RGA assay as described in Example 17 with the exception, that the RGA assay was performed twice. The results of human and mouse IL-11 RGA assay for 1^{st} round germlining mutants and PTM removal mutants are summarized in Table E6. All the mutants showed consistent trend in terms of changes in SPR binding affinity and RGA potency.

TABLE E6
RGA results of 1^{st} round germlining and
PTM removal mutants to human/mouse IL-11

| | IC50 (nM): hIL-11 | | IC50 (nM): mIL-11 | | Mutation site | |
|---|---|---|---|---|---|---|
| Antibody | RGA 1 | RGA 2 | RGA 1 | RGA 2 | Heavy chain | Light chain |
| TPP-18087 | 93 | 12 | 231 | 21 | NA | |
| TPP-31312 | 171 | 80 | 75 | 20 | VH-WT | VL-WT |
| TPP-31277 | 211 | 47 | 43 | 27 | VH-WT | VL-N31S |
| TPP-31278 | 22 | 2.36 | 0.13 | 0.08 | VH-WT | VL-A33Y |
| TPP-31279 | >500 | >500 | 196 | >500 | VH-WT | VL-N35Y |
| TPP-31280 | >500 | NM | >500 | NM | VH-WT | VL-Y51R |
| TPP-31281 | >500 | NM | >500 | NM | VH-WT | VL-D52N |
| TPP-31282 | 345 | 287 | 70 | 28 | VH-WT | VL-D53N* |
| TPP-31283 | >500 | 200 | 247 | 118 | VH-WT | VL-L54Q |
| TPP-31284 | 101 | 131 | 65 | 17 | VH-WT | VL-L55R |
| TPP-31285 | 50 | 16 | 111 | 23 | VH-WT | VL-S90A |
| TPP-31286 | 59 | 37 | 18 | 13 | VH-E1Q | VL-WT |
| TPP-31287 | 127 | 56 | 41 | 15 | VH-L5V | VL-WT |
| TPP-31288 | 67 | 141 | 47 | 23 | VH-L11V | VL-WT |
| TPP-31289 | 61 | 76 | 27 | 23 | VH-G16R | VL-WT |
| TPP-31290 | >500 | NM | >500 | NM | VH-H31S | VL-WT |
| TPP-31291 | >500 | NM | >500 | NM | VH-D33G | VL-WT |
| TPP-31292 | 234 | 107 | 52 | 39 | VH-N35H* | VL-WT |
| TPP-31293 | 58 | 13 | 31 | 13 | VH-L37V | VL-WT |
| TPP-31294 | 96 | 38 | 41 | 28 | VH-G49A | VL-WT |
| TPP-31295 | 364 | 92 | 60 | 37 | VH-F59Y* | VL-WT |
| TPP-31296 | 46 | 158 | 20 | 22 | VH-F63S | VL-WT |
| TPP-18087 | 111 | 12 | 204 | 21 | VH-WT | VL-WT |
| TPP-31312 | 163 | 80 | 31 | 20 | VH-WT | VL-N97Q |
| TPP-31297 | 181 | 22 | 37 | 7.93 | VH-WT | VL-N97S |
| TPP-31298 | 135 | 33 | 21 | 6.22 | VH-WT | VL-G98A |
| TPP-31299 | 238 | 114 | 44 | 14 | VH-D54E | VL-WT |
| TPP-31300 | 1.09 | 4.75 | 3.42 | 0.81 | VH-D54E | VL-N97Q |
| TPP-31301 | 18.9 | 9.71 | 4.43 | 1.77 | VH-D54E | VL-N97S |
| TPP-31302 | 14.5 | 7.13 | 3.49 | 1.17 | VH-D54E | VL-G98A |
| TPP-31303 | 9.08 | 11 | 4.38 | 2.49 | VH-G55A | VL-WT |
| TPP-31304 | 104 | 35 | 42 | 21 | VH-G55A | VL-N97Q |
| TPP-31305 | 182 | 47 | 30 | 29 | VH-G55A | VL-N97S |
| TPP-31306 | 126 | 23 | 57 | 60 | VH-G55A | VL-G98A |
| TPP-31307 | >500 | 126 | 45 | 37 | VH-G55S | VL-WT |
| TPP-31308 | >500 | >500 | 63 | 170 | VH-G55S | VL-N97Q |
| TPP-31309 | 261 | 122 | 108 | 68 | VH-G55S | VL-N97S |
| TPP-31310 | >500 | >500 | 144 | 75 | VH-G55S | VL-G98A |
| TPP-31311 | >500 | 34 | 76 | 127 | VH-G55S | VL-G98A |

NM = Not Meaningful (=no value reported if efficacy <20% or no IC₅₀ value could be calculated);
*Consecutive numbering of amino acid residues was used;
Underscore = improved binding affinity to human/mouse antigen;
italic = comparable binding;
bold = significantly decreased binding;
asterisk (*) = slightly decreased binding affinity (decreased by 1.5~2.5 fold),
compared with WT antibody

### Example 22: 2^{nd} Round Germlining (Combined Mutation) of TPP-18087

### Germlining Design, PTM Removal Design and Plasmid Construction

Based on the results of 1^{st} round germlining, 12 germline single-site mutants VH-E1Q, VH-L5V, VH-L11V, VH-G16R, VH-N35H, VH-L37V, VH-G49A, VH-F63S, VL-N31S, VL-A33Y, VL-L55R and VL-S90A, did show SPR binding affinity <1.9E-08 M and RGA potency (mean)<1.7E-07 M. These 12 mutated positions were combined to generate the template for 2^{nd} round germlining, named as fundamental backbone (FB). To evaluate the effect of every germlining mutation on the activity of FB, 12 single position back-mutation variants upwards FB were constituted (FB-1). Another two mutants (VH-F59Y and VL-D53N) showed slightly decrease in SPR binding activity and RGA potency. These two sites were probed combinatorically as well. Thus, 16 mutants in total, which utilized PTM polished backbone (VH-D54E VL-N97S), were selected to constitute 2^{nd} round germlining variants. DNA for the 2^{nd} round germlining antibody variants were codon optimized for mammalian expression. and then synthesized at Genewiz (South Plainfield, NJ, USA). Genes encoding the variable region of parental antibody and germlining variants were cloned into human IgG expression vectors (WuXi, China) to generate human IgG constructs of a desired isotype (VH domain was fused with human IgG4 SPLE variant, and VL domain was fused with human Ig lambda CL domain).

### Affinity to Human/Mouse IL-11 by SPR

16 AB were produced as described in Example 21 and binding of purified AB to human and mouse IL-11 was analyzed using SPR as described in Example 21. SPR results of 2^{nd} round germlining mutants are summarized in Table E7.

TABLE E7
SPR assay results of 2^{nd} round germlining
mutants for human/mouse IL-11

| | K_{D} [M]: | K_{D} [M]: | |
|---|---|---|---|
| Antibody | hIL-11 | mIL-11 | Mutation site |
| TPP-31312 | 5.3E−09 | 9.3E−09 | WT |
| TPP-31313 | 8.9E−10 | 6.4E−10 | FB |
| TPP-31314 | 8.7E−10 | 5.9E−10 | FB − 1, back mutation |
| | | | VH-Q1E |
| TPP-31315 | 8.7E−10 | 6.5E−10 | FB − 1, back mutation |
| | | | VH-V5L |
| TPP-31316 | 8.5E−10 | 6.2E−10 | FB − 1, back mutation |
| | | | VH-V11L |
| TPP-31317 | 8.3E−10 | 5.8E−10 | FB − 1, back mutation |
| | | | VH-R16G |
| TPP-31318 | 8.9E−10 | 3.0E−10 | FB − 1, back mutation |
| | | | VH-H35N |
| TPP-31319 | 8.3E−10 | 5.9E−10 | FB − 1, back mutation |
| | | | VH-V37L |
| TPP-31320 | 9.5E−10 | 5.6E−10 | FB − 1, back mutation |
| | | | VH-A49G |
| TPP-31321 | 8.5E−10 | 6.7E−10 | FB − 1, back mutation |
| | | | VH-S63F |
| TPP-31322 | 8.1E−10 | 6.9E−10 | FB − 1, back mutation |
| | | | VL-S31N |
| TPP-31323 | 2.4E−09 | 1.7E−09 | FB − 1, back mutation |
| | | | VL-Y33A |
| TPP-31324 | 7.9E−10 | 5.0E−10 | FB − 1, back mutation |
| | | | VL-R55L |
| TPP-31325 | 5.9E−10 | 3.5E−10 | FB − 1, back mutation |
| | | | VL-A90S |
| TPP-31326 | 1.7E−09 | 1.2E−09 | FB + 1, plus VH-F59Y |
| TPP-31327 | 1.0E−09 | 8.5E−10 | FB + 1, plus VL-D53N |
| TPP-31328 | 1.8E−09 | 1.8E−09 | FB + 2, plus VH-F59Y |
| | | | & VL-D53N |

* Consecutive numbering of amino acid residues was used

TPP-31325 (FB with a back-mutation VL-A90S) showed the highest binding affinity against hIL-11 and mIL-11, thus was involved in 2^{nd} round affinity optimization as the backbone template.

### Human and Mouse IL-11 RGA Assay

Human and mouse IL-11 RGA assay was performed using purified 2^{nd} round germlining mutant antibodies as described in Example 21, with the exception that only one RGA run was performed, and results are summarized in Table E8.

TABLE E8
RGA results of 2^{nd} round germlining
mutants to human/mouse IL-11

| | IC50 (nM): | IC50 (nM): | |
|---|---|---|---|
| Antibody | hIL-11 | mIL-11 | Mutation site |
| TPP-31312 | 8.9 | 9.8 | WT |
| TPP-31313 | 5.4 | 1.6 | FB |
| TPP-31314 | 2.4 | 1.2 | FB − 1, back mutation |
| | | | VH-Q1E |
| TPP-31315 | 2.9 | 1.7 | FB − 1, back mutation |
| | | | VH-V5L |
| TPP-31316 | 3.2 | 1.1 | FB − 1, back mutation |
| | | | VH-V11L |
| TPP-31317 | 2.9 | 1.5 | FB − 1, back mutation |
| | | | VH-R16G |
| TPP-31318 | 3.8 | 1.2 | FB − 1, back mutation |
| | | | VH-H35N |
| TPP-31319 | 2.3 | 1.6 | FB − 1, back mutation |
| | | | VH-V37L |
| TPP-31320 | 2.8 | 1.1 | FB − 1, back mutation |
| | | | VH-A49G |
| TPP-31321 | 2.8 | 1.0 | FB − 1, back mutation |
| | | | VH-S63F |
| TPP-31322 | 1.7 | 1.4 | FB − 1, back mutation |
| | | | VL-S31N |
| TPP-31323 | 7.1 | 3.5 | FB − 1, back mutation |
| | | | VL-Y33A |
| TPP-31324 | 2.9 | 1.0 | FB − 1, back mutation |
| | | | VL-R55L |
| TPP-31325 | 1.2 | 0.6 | FB − 1, back mutation |
| | | | VL-A90S |
| TPP-31326 | 3.6 | 3.3 | FB + 1, plus VH-F59Y |
| TPP-31327 | 3.1 | 2.1 | FB + 1, plus VL-D53N |
| TPP-31328 | 7.9 | 2.1 | FB + 2, plus VH-F59Y |
| | | | & VL-D53N |

### Example 23: 1^{st} Round Affinity Maturation (Single Amino Acid Mutations) of TPP-18087

### Affinity Optimization Design and Mutant Construction

Antibody TPP-18087 was subjected to lead optimization procedures aiming to optimize its affinity and to increase its functional efficiency. Each amino acid of six complementary-determining regions (CDRs) of parental TPP-18087 clone was individually mutated to all 20 amino acids using a site-directed mutagenesis method. DNA primers containing a NNS codon encoding 20 amino acids were used to introduce mutation to each targeted CDR position. The degenerate primers were used in site-directed mutagenesis reactions. Briefly, each degenerate primer was phosphorylated. The PCR condition was, 94° C. for 2 minutes, (94° C. for 30 seconds, 55° C. for 30 seconds, 72° C. for 5 minutes), 16 cycles, 72° C. for 10 minutes. PCR products were purified and then transformed into BL21 for production of scFv fragments containing a c-Myc-tag followed by a His-tag. Periplasmatic extracts were used for further characterization.

### 1^{st} Round Screening ELISA

1^{st} round screening ELISA was set up as following: Individual wells of a 96-well Maxisorp Immunoplate were coated with 100 μl of 0.25 μg/ml Goat anti-c-myc antibody in coating buffer (PBS, pH 7.4) overnight at 4° C. Next day, the plate was washed three times with 300 μl washing buffer (PBS-T) and blocked with 200 μl of 1% casein in PBS for 1 hour at 25° C. After three additional washes, periplasmic extracts (PE) of the TPP-18087 library scFv were diluted with 1% casein in PBS/0.05% Tween 20 with volume ratio of 1:1 and 100 μl/well were added to the plate for 1 hour incubation at 25° C. At the same time, 0.5 μg/ml of hIL-11 or mIL-11 antigen and 1.875 μg/ml of IL11 antibody TPP-31391 were pre-mixed for 1 hour at 25° C. TPP-31391 contains variable domains, which are active in the Di-complex ELISA and which do not compete for IL-11 binding with antibody TPP-18087. After three washes of the assay plate with 300 μl washing buffer, 100 μl of the hIL-11/TPP-31391 or mIL 1/TPP-31391 mixture was added to wells for 1 hour incubation at 25° C. After three additional washes, this was followed by incubation with 100 μl/well anti-hFc-IgG-horseradish peroxidase (HRP) conjugate (1:5000 in PBS-T) for 1 hour at 25° C. After six final washes with 300 μl washing buffer, HRP activity was detected with tetra-methyl-benzidine (TMB) substrate and the reaction was quenched with 2M HCl. Plates were read at 450 nM.

187 out of 5368 clones subjected to the screening ELISA showed 5-6-fold increase in ELISA OD450 signal compared with wild type antibody TPP-18087 expressed as scFv antibody. DNA sequencing of the 187 clones revealed 24 clones with unique sequences at 12 CDR amino acid positions were. These clones were further confirmed by scFv capture ELISA.

scFv Capture ELISA

The EC50 values of 24 hits from TPP-18087 library primary screening were determined by use of scFv capture ELISA. In brief, 100 μl of 1 μg/ml of IL-11 monoclonal antibody TPP-31391 in PBS, pH 7.4, was added into individual wells of a 96-well Maxisorp Immunoplate and the assay plate incubated overnight at 4° C. Next day, plate was blocked with 1% casein for 1 hour at 25° C. 2 μg/ml hIL-11 or mIL-11 then were added to plate and incubated for 1 hour at 25° C. Then Periplasmic extracts of the 24 hits from TPP-18087 library primary screening were 3.16-fold serial diluted with 1% casein in PBS/0.05% Tween 20, added to the plates and incubated for 1 hour at 25° C. This was followed by incubation with goat anti-c-myc HRP conjugate in PBS-T for 1 hour at 25° C. HRP activity was detected with TMB substrate and the reaction was quenched with 2M HCl. Plates were read at 450 nm and EC50 values determined with GraphPad Prism.

Eight mutants that showed both binding improvement to hIL-11 and mIL-11 using scFv capture ELISA were selected for the combinatory library design (FIG. 19).

### Example 24: 2^{nd} Round Affinity Maturation (Combinatorial Mutation)

### Combinatorial Library Construction

Affinity improved mutations identified in primary screening and shown in FIG. 19 were used in combinatorial library construction.

The CJ236 strain with final germline TPP-31325 (after PTM removal and 2^{nd} round germlining) plasmid was used for generating uracilated single-strand DNA (ssDNA) which were used as a template of TPP-18087 combinatorial library construction. Briefly, primers encoding all identified mutations at specific CDR position along with the wild type amino acid were synthesized and mixed at equi-molar ratio. For primers containing more than one mutation site, primers containing all possible combination of mutations within the primer region were synthesized and mixed at equi-molar ratio. To construct the combinatorial library, Kunkel reaction was performed. Briefly, the mixture was heated to 85° C. for 5 minutes then cooldowned from 64° C. to 55° C. for over 1 hour. Thereafter, T4 ligase and T4 DNA polymerase were added and mixed. And then incubated for 1.5 hours at 37° C. Typically, 200 ng of the combinatorial library DNA was electroporated into BL21 for production of scFv fragments containing a c-Myc-tag followed by a His-tag.

2^{nd} Round Screening Capture scFv ELISA

The constructed library was screened to identify combination of mutations that produce synergistic effect in binding improvement. A total of 1848 clones were screened with biotinylated hIL-11 and mIL-11 by capture scFv ELISA. In brief, wells of a 96-well Maxisorp Immunoplate were coated with 0.2 μg/ml of Goat anti-c-myc antibody in coating buffer PBS at pH 7.4 overnight at 4° C. Next day, the plate was washed three times with 300 μl washing buffer (PBS-T) and then blocked with 200 μl 1% casein in PBS for 1 hour at 25° C. After three additional washes, the Periplasm (PE) of the combinatorial mutated scFv clones were diluted with 1% casein in 0.05% Tween 20 according to 1:1 volume ratio, then 100 μl were added to the plate and incubated for one hour at 25° C. After three washes, 0.25 μg/ml biotinylated hIL-11 or mIL-11 was added to the well and incubated for 1 hour at 25° C. After three washes, this was followed by incubation with SA-horseradish peroxydase (HRP) conjugate for 1 hour at 25° C. After six final washes, HRP activity was detected with tetra-methyl-benzidine (TMB) substrate and the reaction quenched with 2M HCl. Plates were read at 450 nM. Clones exhibiting an optical density (OD) signal at 450 nm greater than 2-fold compared to the non-mutated final germline scFv variant TPP-31466 were sequenced and further confirmed by SPR.

Combinatorial scFv Mutant Library Confirmation by koff Ranking SPR

The k_{d} (1/s) of 20 scFv hits binding to hIL-11 and mIL-11 was determined using Biacore 8K. Each scFv antibody from periplasmic extract was captured on CM5 sensor chip via a pre-immobilized mixture of anti-his antibody and anti-c-Myc antibody. 5 nM hIL-11 or mIL-11 in 1×HBS-EP+ (pH 7.4) buffer was injected over the sensor chip at a flow rate of 30 uL/min for an association phase of 120 s, followed by 600 s dissociation phase. The chip was then regenerated by 10 mM Glycine, pH 1.5 after each binding cycle. The sensorgrams of reference channel and buffer channel were subtracted from the test sensorgrams. The experimental data was fitted by 1:1 model using Langmiur analysis. Molecular weight of 19.3 kDa was used to calculate the molar concentration of analyte. kₐ, k_{d} and K_{D} values from SPR experiments are shown for TPP-31466, which is the scFv format of the final germline variant TPP-31325, as well as the scFv hits TPP-31413 to TPP-31432 in Table E9.

TABLE E9
SPR results for 20 screening hit from 2^{nd} affinity maturation

| | human IL-11 | | | mouse IL-11 | | |
|---|---|---|---|---|---|---|
| Antibody | kₐ (1/Ms) | k_{d} (1/s) | K_{D} (M) | kₐ (1/Ms) | k_{d} (1/s) | K_{D} (M) |
| TPP-31466 | 9.7E+06 | 4.2E−03 | 4.3E−10 | 7.4E+06 | 2.5E−03 | 3.3E−10 |
| TPP-31426 | 7.9E+06 | 1.6E−04 | 2.0E−11 | 8.5E+06 | 1.4E−04 | 1.6E−11 |
| TPP-31424 | 5.1E+06 | 1.8E−04 | 3.5E−11 | 5.4E+06 | 1.5E−04 | 2.8E−11 |
| TPP-31414 | 5.2E+06 | 2.1E−04 | 4.1E−11 | 5.1E+06 | 1.8E−04 | 3.6E−11 |
| TPP-31428 | 5.3E+06 | 2.3E−04 | 4.4E−11 | 6.1E+06 | 2.0E−04 | 3.2E−11 |
| TPP-31416 | 7.0E+06 | 2.4E−04 | 3.4E−11 | 5.6E+06 | 3.5E−04 | 6.3E−11 |
| TPP-31415 | 6.8E+06 | 2.4E−04 | 3.6E−11 | 5.7E+06 | 2.5E−04 | 4.4E−11 |
| TPP-31420 | 4.3E+06 | 2.6E−04 | 6.1E−11 | 4.4E+06 | 2.3E−04 | 5.2E−11 |
| TPP-31431 | 5.4E+06 | 2.8E−04 | 5.1E−11 | 5.5E+06 | 2.5E−04 | 4.6E−11 |
| TPP-31418 | 8.7E+06 | 2.9E−04 | 3.3E−11 | 7.7E+06 | 2.1E−04 | 2.7E−11 |
| TPP-31423 | 6.1E+06 | 2.9E−04 | 4.8E−11 | 5.4E+06 | 2.5E−04 | 4.7E−11 |
| TPP-31419 | 4.7E+06 | 2.9E−04 | 6.3E−11 | 4.6E+06 | 2.8E−04 | 6.0E−11 |
| TPP-31425 | 3.7E+06 | 3.0E−04 | 8.0E−11 | 4.5E+06 | 2.7E−04 | 6.1E−11 |
| TPP-31417 | 5.8E+06 | 3.0E−04 | 5.2E−11 | 5.4E+06 | 2.2E−04 | 4.1E−11 |
| TPP-31429 | 5.9E+06 | 3.0E−04 | 5.1E−11 | 6.4E+06 | 2.8E−04 | 4.4E−11 |
| TPP-31432 | 4.8E+06 | 3.1E−04 | 6.5E−11 | 5.3E+06 | 2.7E−04 | 5.0E−11 |
| TPP-31430 | 5.0E+06 | 3.6E−04 | 7.3E−11 | 4.8E+06 | 3.5E−04 | 7.2E−11 |
| TPP-31413 | 4.9E+06 | 3.7E−04 | 7.4E−11 | 4.7E+06 | 3.2E−04 | 6.8E−11 |
| TPP-31421 | 5.1E+06 | 7.6E−04 | 1.5E−10 | 4.7E+06 | 2.8E−04 | 6.0E−11 |
| TPP-31422 | 5.4E+06 | 9.9E−04 | 1.8E−10 | 4.7E+06 | 1.8E−04 | 3.8E−11 |
| TPP-31427 | 6.0E+06 | 1.6E−03 | 2.6E−10 | 4.8E+06 | 1.8E−04 | 3.7E−11 |

### IgG Conversion

Genes encoding the variable regions of five scFv from 2^{nd} round affinity maturation were cloned into murine IgG1 expression vector pTT5 (VH domain was fused with murine IgG1 variant, and VL domain was fused with murine Ig lambda CL domain) to generate murine IgG1 expression vectors for TPP-29519, TPP-29520, TPP-29521, TPP-29522 and TPP-29523. In addition, the five scFv were cloned into human expression vectors to generate human IgG constructs of a desired isotype for TPP-29680, TPP-30000, TPP-30001, TPP-30002 and TPP-30003 (VH domain was fused with human IgG1 HS variant, in which Fc-silencing mutations were introduced (E233P/L234V/L235A/DG236/D265G/A327Q/A330), and VL domain was fused with human Ig lambda CL domain).

### Example 25-27 Show Germlining, Sequence Optimization (PTM Removal) and Affinity Maturation of TPP-18068

### Example 25: Germlining and PTM Removal of TPP-18068

Antibody TPP-18068 was subjected to lead optimization procedures aiming to (i) optimize its affinity, (ii) increase its functional efficiency, (iii) reduce the risk for sequence-based immunogenicity and (iv) improve compatibility with downstream development processes.

For the germlining and sequence optimization process of TPP-18068 (mIgG1) respectively TPP-16478 (hIgG1) the closest germline families for light and heavy chain were selected and scrutinized for potential CMC relevant residues. Deviations from closest human germlines in CDR regions and FW regions and potential CMC relevant residues in CDR regions were adjusted by site directed mutagenesis following standard molecular protocols or purchased from DNA synthesis providers. The generated DNA constructs were expressed by transient transfection of mammalian cells and purified as described in Example 15. Antibodies were then tested for human IL-11 and murine IL-11 binding by SPR as described in Example 16. Measured K_{D} values are shown in Table E10.

TABLE E10
SPR results for 1^{st} round germlining
and PTM removal of TPP-18068

| Antibody | K_{D} [M]: hIL-11 | K_{D} [M]: mIL-11 | Mut VH | Mut VL |
|---|---|---|---|---|
| TPP-16478 | 6.6E−09 | 8.3E−10 | WT | WT |
| TPP-19841 | 6.3E−09 | 7.9E−10 | WT | T23S |
| TPP-19842 | 7.3E−09 | 6.6E−10 | WT | A31S |
| TPP-19843 | 1.7E−08 | 7.5E−09 | WT | Y33N |
| TPP-19844 | No binding | 1.1E−08 | WT | D34Y |
| TPP-19845 | 2.0E−08 | 5.0E−09 | WT | H36Y |
| TPP-19846 | No binding | 1.8E−08 | WT | D34T |
| TPP-19847 | 9.0E−09 | 2.1E−09 | WT | H36N |
| TPP-19848 | 5.2E−08 | 2.4E−09 | WT | E55Q |
| TPP-19849 | 5.9E−09 | 6.8E−10 | WT | R81Q |
| TPP-19850 | 6.5E−09 | 1.6E−09 | WT | N98S |
| TPP-19861 | 1.7E−08 | 2.8E−08 | G33A | WT |
| TPP-19862 | 1.9E−08 | 4.7E−09 | H35S | WT |
| TPP-19863 | 4.7E−09 | 6.4E−10 | V50A | WT |
| TPP-19864 | 1.3E−08 | No binding | Y53G | WT |
| TPP-19865 | 2.5E−08 | 3.6E−09 | D54S | WT |
| TPP-19866 | 8.5E−09 | 1.4E−09 | S56G | WT |
| TPP-19867 | 2.7E−08 | 3.4E−09 | Y57S | WT |
| TPP-19868 | 6.2E−09 | 7.2E−10 | K58T | WT |
| TPP-19869 | No binding | 9.3E−09 | A98K | WT |
| TPP-19870 | No binding | 6.5E−09 | V100Y | WT |
| TPP-19871 | No binding | 4.7E−08 | P101F | WT |
| TPP-19872 | 5.5E−09 | 4.7E−10 | S49A | WT |
| TPP-19873 | 2.1E−08 | 4.0E−09 | Y57N | WT |
| TPP-19874 | 9.9E−08 | 9.9E−09 | A98R | WT |

* Consecutive numbering of amino acid residues was used

For the 2^{nd} round of germlining, single reversions were then combined on DNA level, expressed and purified as described in Example 15, and tested by SPR as described in Example 16. Measured K_{D} values are shown in Table E11.

TABLE E11
SPR results for 2^{nd} round germlining
and PTM removal of TPP-18068

| | K_{D} [M]: | K_{D} [M]: | | |
|---|---|---|---|---|
| Antibody | hIL-11 | mIL-11 | Mut VH | Mut VL |
| TPP-16478 | 7.3E−09 | 6.0E−10 | WT | WT |
| TPP-21150 | 5.6E−09 | 1.4E−09 | WT | T23S, A31S, N98S |
| TPP-21151 | no binding | 1.2E−07 | WT | T23S, A31S, S52D, |
| | | | | N98S |
| TPP-21153 | 1.0E−08 | 3.6E−09 | WT | T23S, A31S, E55Q, |
| | | | | N98S |
| TPP-21157 | 1.3E−08 | 7.3E−08 | WT | T23S, A31S, E55Q, |
| | | | | N98S, P100V |
| TPP-21158 | 4.7E−08 | 1.3E−08 | S49A, A98R | WT |
| TPP-27154 | 5.4E−09 | 1.3E−09 | WT | N98S |
| TPP-27155 | 5.8E−09 | 6.5E−10 | WT | T23S, A31S |
| TPP-27156 | 6.6E−09 | 4.5E−10 | S49A | WT |
| TPP-27157 | 5.1E−09 | 1.1E−09 | S49A | N98S |
| TPP-27158 | 5.9E−09 | 4.0E−10 | S49A | T23S, A31S |
| TPP-27159 | 5.4E−09 | 1.1E−09 | S49A | T23S, A31S, N98S |
| TPP-27160 | 1.8E−07 | 5.6E−08 | S49A | T23S, A31S, S52D, |
| | | | | N98S |
| TPP-27162 | 7.4E−09 | 3.2E−09 | S49A | T23S, A31S, E55Q, |
| | | | | N98S |
| TPP-27163 | 1.2E−08 | 2.8E−08 | S49A | T23S, A31S, E55Q, |
| | | | | N98S, P100V |
| TPP-27164 | 5.7E−08 | 2.4E−08 | S49A, A98 | N98S |
| TPP-27165 | 4.9E−08 | 1.4E−08 | S49A, A98 | T23S, A31S |
| TPP-27167 | no binding | 8.1E−09 | S49A, A98 | T23S, A31S, S52D, |
| | | | | N98S |
| TPP-27169 | 1.0E−07 | 4.1E−08 | S49A, A98 | T23S, A31S, E55Q, |
| | | | | N98S |
| TPP-27170 | no binding | 8.4E−09 | S49A, A98 | T23S, A31S, E55Q, |
| | | | | N98S, P100V |

* Consecutive numbering of amino acid residues was used

In addition, the variants of 2^{nd} round germlining and PTM removal were tested in the human and murine IL-11 reporter gene assay, as describe in Example 17, with the exception, that a fixed concentration of 1.9 [nM] human IL-11 (Invigate, e.g., lot #C121021-19) and 5.6 [nM] murine IL-11 (Invigate, e.g., lot #C210819-09) were used. Results are shown in Table E12.

TABLE E12
RGA results for 2^{nd} round germlining and PTM removal of TPP-18068

| | human IL-11 | | mouse IL-11 | | Mutation Site | |
|---|---|---|---|---|---|---|
| Antibody | IC50 [M] | Efficacy (%) | IC50 [M] | Efficacy (%) | VH | VL |
| TPP-16478 | 5.6E−09 | 51.4 | 9.6E−10 | 90.8 | WT | WT |
| TPP-21150 | 1.2E−08 | 46.9 | 1.6E−09 | 92.2 | WT | T23S, A31S, N98S |
| TPP-21151 | NM | 19.4 | 5.5E−08 | 65.3 | WT | T23S, A31S, S52D, N98S |
| TPP-21153 | 2.6E−08 | 34.0 | 3.4E−09 | 81.2 | WT | T23S, A31S, E55Q, N98S |
| TPP-21157 | NM | 13.4 | 4.4E−08 | 59.2 | WT | T23S, A31S, E55Q, N98S, P100V |
| TPP-21158 | NM | 19.4 | 2.4E−09 | 73.2 | S49A, A98R | WT |
| TPP-27154 | 2.4E−08 | 58.7 | 1.8E−09 | 92.9 | WT | N98S |
| TPP-27155 | 2.0E−08 | 56.5 | 1.1E−09 | 94.3 | WT | T23S, A31S |
| TPP-27156 | 1.5E−08 | 50.4 | 1.2E−09 | 90.6 | S49A | WT |
| TPP-27157 | 1.3E−08 | 51.2 | 1.4E−09 | 86.4 | S49A | N98S |
| TPP-27158 | 2.0E−08 | 55.8 | 1.4E−09 | 89.9 | S49A | T23S, A31S |
| TPP-27159 | 1.9E−08 | 43.2 | 1.9E−09 | 82.4 | S49A | T23S, A31S, N98S |
| TPP-27160 | NM | −2.8 | 1.0E−08 | 63.8 | S49A | T23S, A31S, S52D, N98S |
| TPP-27162 | 9.1E−08 | 36.0 | 3.3E−09 | 77.6 | S49A | T23S, A31S, E55Q, N98S |
| TPP-27163 | NM | 2.9 | 5.2E−08 | 52.3 | S49A | T23S, A31S, E55Q, N98S, P100V |
| TPP-27164 | NM | 13.9 | 4.1E−08 | 63.2 | S49A, A98 | N98S |
| TPP-27165 | NM | 17.5 | 4.2E−09 | 75.1 | S49A, A98 | T23S, A31S |
| TPP-27167 | NM | 2.5 | 2.1E−09 | 29.3 | S49A, A98 | T23S, A31S, S52D, N98S |
| TPP-27169 | NM | 4.0 | 2.0E−09 | 50.2 | S49A, A98 | T23S, A31S, E55Q, N98S |
| TPP-27170 | NM | 0.3 | NM | 41.9 | S49A, A98 | T23S, A31S, E55Q, N98S, P100V |

NM = Not Meaningful (=no value reported if efficacy <20% or no IC₅₀ value could be calculated)

Analysis of SPR and RGA results led to the final germlined and sequence optimized molecule TPP-27159. TPP-27159 carries in comparison to TPP-18068 the reversions T23S, A31S, and N98S in the variable region of the light chain and S49A in the heavy chain.

### Example 26: 1^{st} Round of Affinity and Potency Maturation

### Affinity Optimization Design and Mutant Construction

Affinity maturation was done by a first single mutation gathering round followed by recombination of the most affinity- and potency-increasing amino acid exchanges in a germlined and sequence optimized antibody backbone.

For mutation gathering NNK (N=A or G or C or T, K=G or T) randomizations at the following individual amino acid positions were generated by site directed mutagenesis using synthetic oligonucleotides including NNK for codon-diversification. For TPP-18068 the following regions were analyzed for their effect on affinity and potency: SYGMH (residues 31 to 35 of VH SEQ ID NO: 32), VISYDGSYKYYADSVKG (residues 50 to 66 of VH SEQ ID NO: 32), GVPDY (residues 99 to 103 of VH SEQ ID NO:32), TGSSSNIGAGYDVH (residues 23 to 36 of VL SEQ ID NO:36), SNNERPS (residues 52 to 58 of VL SEQ ID NO: 36), and AAWDDSLNGPV (residues 91 to 101 of VL SEQ ID NO: 36).

The resulting single NNK libraries were sequenced and about 800 single amino acid exchange variants of TPP-18068 were identified.

### Expression of Mutated Variants of the Parent Clone TPP-18068 in a μ-Scale Format

To test the mutated variants of the parent clone in an ELISA based assay setup, generated DNA pTT5 constructs of these variants were expressed by transient transfection of mammalian cells. To transfect cells 7.5 μl each of heavy chain (HC) and light chain (LC) coding plasmids (25 μg/ml each diluted in Opti-MEM [Invitrogen, #11058-02] cell culture media, Gibco) are pipetted in each well of a 96-well polystyrene round bottom plate (ThermoFisher Scientific, #268152) followed by an addition of 15 μl/well Transfectin (ThermoFisher Scientific, cat #12347019) (1:12.5 dilution in Opti-MEM, (ThermoFisher Scientific, cat #12347019)) and an incubation phase at RT for 20 minutes. Human embryonic kidney (HEK) cells were diluted in FreeStyle F17 media (Gibco) supplemented with 2 mM GlutaMAX (ThermoFisher Scientific, Cat #35050061) and 0.1% Pluronic F-68 (ThermoFisher Scientific, Cat #35050061) to a cell count of 1E6 cells/ml. 380 μl/well of the diluted cells was transferred into a 1 ml deep well plate (Hj-Bioanalytik, #750289). Subsequently 20 μl of the DNA mixture from the round bottom plate was added to the cells in the deep well plate followed by a four-hour incubation step (37° C., 5% C02, 70% humidity, 700 rounds per minute shaking). After the incubation Penicillin (final concentration: 100U/ml) Streptomycin (final concentration: 100 μg/ml) and Gelatin Peptone N3 (OrganoTechnie, #19554, final concentration: 0.25%) were added to each well. An incubation period of 5 days followed, after which the plates were centrifuged (5 min, 1000×g) and the supernatant containing the expressed antibodies was removed and stored at 4° C. for further testing.

### Assay Set-Up for Testing of Anti-IL-11 Lead Optimized Variants

The following ELISA based assay setup was used to test the mutated variants of the parent clone for binding to their target. 384-well plates were coated over night at 4° C. with 20 μl/well of a 1 μg/ml anti-mouse IgG (Fc specific) solution (coating buffer: Candor, anti-mouse IgG: Sigma #M3534). Next day, the plates were washed three times with 50 μl/well PBST and subsequently blocked for one hour at room temperature using 50 μl 100% smart block (Candor) followed by again three wash steps like described before. After that, 20 μl/well of the to be tested mutated variants (1 μg/ml in 10% smart block in PBST) or of the parental control TPP-18068 were added and plates incubated for one hour at room temperature. Plates were washed again three times before adding 25 μl/well of a biotinylated IL-11 solution (0.5 nM human IL-11 (Invigate, e.g., lot #C121021-19); 0.3 nM murine IL-11 (Invigate e.g., lot #C210819-09)) and incubating the plates for one hour at room temperature. This was followed by three washing steps and subsequently adding of 25 μl/well of Streptavidin-horseradish peroxidase conjugate (Sigma, 1:1000 dilution in 10% smart block in PBST). A last triple washing step using PBST was followed by the addition of 30 μl substrate solution (Amplex Red, Invitrogen). After a 15 min incubation step in the dark at room temperature the fluorescence signals were measured in a fluorescence microplate reader (Envision) using excitation of 535 nm and emission detection at 590 nm. The ratio between the OD value obtained for each mutant variant and the OD of TPP-16478 WT antibody, which had been expressed as part of the library, were calculated. High ratios indicated improved binding to human respectively mouse IL-11.

### Assay Set-Up for Testing of IL-2Rα Binding

The following ELISA based assay setup was used to test the mutated variants of the parent clone for specific binding to IL-2Rα (Peprotech, Germany, #200-02RC). IL-2Rα was coated at 0.5 μg/ml in 30 μl coating buffer (Candor, #113500) per well on a 384 well microtiter plate (Maxisorb, Nunc, #460518) over night at 4° C. After three washes with 50 μl/well washing buffer PBST, the plates were incubated for one hour at room temperature with 50 μl/well SmartBlock (Candor, #113500). After blocking, the plates were washed three times with washing buffer. 30 μl/well (4 μg/ml in PBS+0.05% Tween, 10% SmartBlock) of the antibodies to be tested were added, followed by a 60 minute incubation phase at room temperature. Then, after three additional washes, 30 μl anti mouse IgG HRP-conjugated antibody (Sigma, A0168, diluted 1:1000 in PBST), were added per well and incubated for one hour. A last triple washing step using PBST was followed by the addition of 30 μl substrate solution (Amplex Red, Invitrogen). After a 15 min incubation step in the dark at room temperature the fluorescence signals were measured in a fluorescence microplate reader (Envision) using excitation of 535 nm and emission detection at 590 nm. The ratio between the OD value obtained for each mutant variant and the OD of an isotype control antibody, which had been expressed as negative control as part of the library, were calculated. Small ratios indicated loss of IL-2Ra binding.

Expression and Characterization of Selected Hits from 1^{st} Affinity Maturation and IL2Ra Binding

42 hits from the IL-11 and EL-2Ra screening analysis were selected, expressed by transient transfection of mammalian cells and purified as described in Example 15. Antibodies were then tested for human IL-11 and murine IL-11 binding by SPR as described in Example 16. Measured K_{D} values are shown in Table E13.

TABLE E13
SPR results for 42 selected hits from
1^{st} round affinity maturation screening

| | KD [M]: | KD [M]: | | |
|---|---|---|---|---|
| Antibody | hIL-11 | mIL-11 | Mut VH | Mut VL |
| TPP-18068 | 1.2E−08 | 8.6E−10 | WT | WT |
| TPP-26290 | 8.6E−09 | 5.4E−10 | WT | Y33M |
| TPP-26291 | 9.7E−09 | 9.4E−10 | WT | D95P |
| TPP-26292 | 1.3E−08 | 4.8E−10 | WT | D95E |
| TPP-26293 | 1.1E−08 | 2.6E−10 | WT | G32W |
| TPP-26294 | 1.1E−08 | 8.2E−10 | WT | G32A |
| TPP-26295 | 9.8E−09 | 6.5E−10 | WT | D95H |
| TPP-26296 | 1.4E−08 | 7.1E−10 | WT | D95N |
| TPP-26297 | 1.3E−08 | 8.9E−10 | WT | D94Q |
| TPP-26298 | 1.3E−08 | 7.3E−10 | WT | T23L |
| TPP-26299 | 1.1E−08 | 6.3E−10 | WT | N98Y |
| TPP-26300 | 9.2E−09 | 4.5E−10 | WT | D95S |
| TPP-26301 | no binding | 1.0E−09 | WT | G32D |
| TPP-26302 | 1.3E−08 | 7.0E−10 | WT | A91S |
| TPP-26303 | 1.3E−08 | 6.5E−10 | WT | S58E |
| TPP-26304 | 1.2E−08 | 6.4E−10 | WT | G30W |
| TPP-26306 | 1.2E−08 | 6.5E−10 | WT | D95Q |
| TPP-26307 | 1.6E−08 | 1.6E−09 | WT | D95I |
| TPP-26308 | 1.7E−08 | 1.3E−09 | WT | D95V |
| TPP-26309 | 8.2E−09 | 8.0E−10 | WT | D95F |
| TPP-26310 | 1.3E−08 | 6.6E−10 | WT | S58W |
| TPP-26311 | no binding | 3.3E−09 | WT | V35H |
| TPP-26312 | 1.0E−08 | 8.0E−10 | WT | Y33L |
| TPP-26313 | 9.1E−09 | 4.6E−10 | WT | G32Y |
| TPP-26314 | 8.3E−09 | 5.6E−10 | WT | D95W |
| TPP-26315 | 1.4E−08 | 9.5E−10 | WT | G32H |
| TPP-26316 | 8.4E−09 | 7.2E−10 | WT | D95M |
| TPP-26317 | 8.1E−09 | 9.0E−10 | WT | G32E |
| TPP-26318 | 1.4E−08 | 8.1E−09 | WT | D34F |
| TPP-26319 | 1.2E−08 | 1.4E−09 | WT | R56K |
| TPP-26320 | 1.3E−08 | 8.3E−10 | WT | N54E |
| TPP-26321 | 9.3E−09 | 9.4E−10 | WT | D95L |
| TPP-26323 | 1.0E−08 | 5.8E−10 | WT | D95G |
| TPP-26324 | 1.3E−08 | 1.2E−09 | A61R | WT |
| TPP-26325 | 6.4E−09 | no binding | S31W | WT |
| TPP-26326 | 9.3E−09 | 1.2E−09 | S31Y | WT |
| TPP-26327 | no binding | 1.3E−09 | S52R | WT |
| TPP-26328 | 1.2E−08 | 1.3E−09 | G99T | WT |
| TPP-26329 | 1.4E−08 | 9.2E−10 | Y32W | WT |
| TPP-26330 | 9.5E−09 | 1.7E−09 | S31F | WT |
| TPP-26331 | 1.5E−08 | 9.5E−10 | M34G | WT |
| TPP-26332 | no binding | 1.5E−09 | G33K | WT |
| TPP-26333 | 1.1E−08 | no binding | M34F | WT |

In addition, 42 hits were tested for inhibition of human IL-11 and murine IL-11 mediated Stat3 signaling by the reporter gene assays as described in Example 17. Potency (IC50 [M]) and efficacy (%) were calculated as described in Example 17 and values are shown in Table E14.

TABLE E 14
RGA results for 42 selected hits from 1^{st} round affinity maturation screening

| | human IL-11 | | mouse IL-11 | | Mutation Site | |
|---|---|---|---|---|---|---|
| Antibody | IC50 [M] | Efficacy (%) | IC50 [M] | Efficacy (%) | VH | VL |
| TPP-18068 | 6.7E−09 | 60.3 | 1.0E−09 | 93.1 | WT | WT |
| TPP-26290 | 4.7E−09 | 75.9 | 9.7E−10 | 95.1 | WT | Y33M |
| TPP-26291 | 4.1E−09 | 68.9 | 1.6E−09 | 92.1 | WT | D95P |
| TPP-26292 | 5.6E−09 | 74.0 | 9.4E−10 | 94.7 | WT | D95E |
| TPP-26293 | 2.8E−09 | 73.7 | 9.5E−10 | 96.9 | WT | G32W |
| TPP-26294 | 9.8E−09 | 69.4 | 1.5E−09 | 97.9 | WT | G32A |
| TPP-26295 | 1.3E−08 | 69.2 | 2.1E−09 | 93.9 | WT | D95H |
| TPP-26296 | 2.0E−08 | 71.0 | 2.9E−09 | 93.1 | WT | D95N |
| TPP-26297 | 3.7E−09 | 57.1 | 1.8E−09 | 93.5 | WT | D94Q |
| TPP-26298 | 4.1E−09 | 62.9 | 1.5E−09 | 96.3 | WT | T23L |
| TPP-26299 | 3.1E−09 | 70.1 | 9.4E−10 | 96.9 | WT | N98Y |
| TPP-26300 | 3.3E−09 | 75.5 | 1.1E−09 | 94.9 | WT | D95S |
| TPP-26301 | 2.6E−09 | 76.1 | 1.5E−09 | 91.8 | WT | G32D |
| TPP-26302 | 2.2E−08 | 65.8 | 1.1E−09 | 94.3 | WT | A91S |
| TPP-26303 | 2.2E−08 | 70.2 | 2.3E−09 | 95.7 | WT | S58E |
| TPP-26304 | 5.9E−09 | 70.4 | 1.2E−09 | 95.2 | WT | G30W |
| TPP-26306 | 2.2E−09 | 70.0 | 1.5E−09 | 96.4 | WT | D95Q |
| TPP-26307 | 5.3E−09 | 52.7 | 1.9E−09 | 92.3 | WT | D95I |
| TPP-26308 | 5.9E−09 | 63.6 | 1.3E−09 | 91.6 | WT | D95V |
| TPP-26309 | 1.5E−09 | 63.9 | 9.4E−10 | 92.2 | WT | D95F |
| TPP-26310 | 4.5E−09 | 59.5 | 1.1E−09 | 94.3 | WT | S58W |
| TPP-26311 | 1.9E−09 | 38.1 | 2.2E−09 | 86.2 | WT | V35H |
| TPP-26312 | 3.8E−09 | 68.6 | 1.2E−09 | 87.3 | WT | Y33L |
| TPP-26313 | 1.7E−09 | 76.1 | 6.6E−10 | 96.2 | WT | G32Y |
| TPP-26314 | 1.1E−09 | 69.3 | 8.0E−10 | 94.4 | WT | D95W |
| TPP-26315 | 1.7E−09 | 68.3 | 1.5E−09 | 91.5 | WT | G32H |
| TPP-26316 | 1.6E−09 | 69.4 | 1.0E−09 | 90.4 | WT | D95M |
| TPP-26317 | 9.9E−10 | 69.9 | 1.2E−09 | 93.2 | WT | G32E |
| TPP-26318 | 2.4E−09 | 45.9 | 4.7E−09 | 82.3 | WT | D34F |
| TPP-26319 | 7.4E−09 | 48.6 | 1.5E−09 | 91.1 | WT | R56K |
| TPP-26320 | 7.4E−09 | 70.3 | 1.5E−09 | 100.3 | WT | N54E |
| TPP-26321 | 2.1E−09 | 71.3 | 1.1E−09 | 93.5 | WT | D95L |
| TPP-26323 | 2.0E−09 | 57.6 | 9.2E−10 | 95.9 | WT | D95G |
| TPP-26324 | 1.5E−09 | 59.0 | 1.3E−09 | 92.4 | A61R | WT |
| TPP-26325 | 1.7E−09 | 81.4 | 1.3E−09 | 93.0 | S31W | WT |
| TPP-26326 | 5.4E−09 | 72.0 | 2.0E−09 | 83.0 | S31Y | WT |
| TPP-26327 | NM | 15.0 | 4.7E−07 | 38.0 | S52R | WT |
| TPP-26328 | NM | 18.6 | 6.4E−08 | 70.8 | G99T | WT |
| TPP-26329 | 2.6E−09 | 67.1 | 1.0E−09 | 95.5 | Y32W | WT |
| TPP-26330 | 3.4E−09 | 64.8 | 1.3E−09 | 88.2 | S31F | WT |
| TPP-26331 | 5.4E−09 | 54.9 | 1.5E−09 | 90.9 | M34G | WT |
| TPP-26332 | NM | 0.8 | NM | 0.9 | G33K | WT |
| TPP-26333 | 1.3E−08 | 64.7 | 1.2E−09 | 94.1 | M34F | WT |

NM = Not Meaningful (=no value reported if efficacy <20% or no IC₅₀ value could be calculated)

IL-2Ra Binding of Selected 42 Antibodies from 1^{st} Affinity Maturation

Next, the 42 antibodies were subjected to an IL2Ra binding ELISA as described in Example 20. Ratios of IL-2Ra binding relative to isotype control were calculated and are shown in Table E15.

TABLE E15
IL2Ra binding results for 42 selected hits from
1^{st} round affinity maturation screening

| | hIL2RA ELISA Binding: |
|---|---|
| Antibody | Ratio to Isotpye |
| TPP-18068 | 398 |
| TPP-26290 | 52 |
| TPP-26291 | 334 |
| TPP-26292 | 557 |
| TPP-26293 | 384 |
| TPP-26294 | 275 |
| TPP-26295 | 385 |
| TPP-26296 | 379 |
| TPP-26297 | 183 |
| TPP-26298 | 474 |
| TPP-26299 | 96 |
| TPP-26300 | 447 |
| TPP-26301 | 15 |
| TPP-26302 | 522 |
| TPP-26303 | 481 |
| TPP-26304 | 436 |
| TPP-26306 | 232 |
| TPP-26307 | 176 |
| TPP-26308 | 188 |
| TPP-26309 | 234 |
| TPP-26310 | 231 |
| TPP-26311 | 292 |
| TPP-26312 | 62 |
| TPP-26313 | 195 |
| TPP-26314 | 455 |
| TPP-26315 | 205 |
| TPP-26316 | 221 |
| TPP-26317 | 31 |
| TPP-26318 | 267 |
| TPP-26319 | 235 |
| TPP-26320 | 260 |
| TPP-26321 | 182 |
| TPP-26323 | 256 |
| TPP-26324 | 247 |
| TPP-26325 | 542 |
| TPP-26326 | 516 |
| TPP-26327 | 11 |
| TPP-26328 | 27 |
| TPP-26329 | 247 |
| TPP-26330 | 264 |
| TPP-26331 | 107 |
| TPP-26332 | 19 |
| TPP-26333 | 436 |

Compared to Wild Type antibody TPP-18068, several mutants, e.g., TPP-26301, TPP-26317, TPP-26327, TPP-26328 and TPP-26332 exhibit strongly reduced IL2RA binding.

### Example 27: 2^{nd} Round Affinity Maturation (Combinatorial Mutation)

For the final recombination library of TPP-18068 twelve single substitution variants that were shown in the NNK library screening step to exhibit improved affinity, functional efficiency or reduced IL-2Ra binding were selected. VL mutations G30W, G32Y, G32D, Y33L, N54E, A91S, D95F, D95P, D95S and S98Y and mutations M34G, V50L in the VH region were combined in one recombination library in the final germline TPP-27159 (continuous amino acid nomenclature, reference is TPP-27159 as defined by SEQ ID NOs: XX-VH and XX-VL).

For TPP-27159 oligonucleotides were generated to introduce selected mutations or the corresponding wild type amino acid at each selected position. Library construction was performed using sequential rounds of standard overlap extension PCR (Sambrook J, Russell DW (2001) Molecular cloning: a laboratory manual, 3rd edn. Sambrook).

More than 800 unique combinatorial amino acid exchange variants of TPP-27159 were generated in that way, expressed by transient transfection of mammalian cells as described in Example 26, and resulting expression supernatants screened for murine and human IL-11 binding and IL-2Ra binding as described in Example 26.

18 hits with highest human and mouse IL-11 binding but lowest IL-2Ra binding were selected for small scale expression and purification as described in Example 15. Antibodies were then tested for human IL-11 and murine IL-11 binding by SPR as described in Example 16. Measured K_{D} values are shown in Table E16.

TABLE E16
SPR results for 18 human IgG1 hits after 2^{nd} round affinity maturation

| | K_{D} [M]: | K_{D} [M]: | K_{D} [M]: | K_{D} [M]: | | |
|---|---|---|---|---|---|---|
| TPP-ID | hIL-11 | mIL-11 | ratIL-11 | cynoL-11 | Mut VH | Mut VL |
| TPP-16478 | 2.2E−09 | 6.2E−10 | 3.3E−10 | 1.8E−09 | WT | WT |
| TPP-29381 | 6.2E−10 | 5.7E−10 | 3.0E−10 | 4.3E−10 | WT | G32D, A91S, D95S |
| TPP-29382 | 4.4E−10 | 5.6E−10 | 2.0E−10 | 4.6E−10 | WT | G32D, Y33L, D95F, S98Y |
| TPP-29383 | 1.1E−09 | 6.2E−10 | 3.1E−10 | 1.3E−09 | WT | G32D, Y33L, N54E, D95P, S98Y |
| TPP-29384 | 9.8E−10 | 3.0E−10 | 1.8E−10 | 8.6E−10 | WT | N54E, D95P |
| TPP-29386 | 2.7E−10 | 3.0E−10 | 1.5E−10 | 3.1E−10 | WT | G32D, N54E, A91S, D95S |
| TPP-29389 | 2.3E−10 | 1.2E−10 | 8.3E−11 | 9.1E−10 | WT | G32Y, N54E, D95P, S98Y |
| TPP-29391 | 3.6E−10 | 1.7E−10 | 7.1E−11 | 6.9E−10 | WT | G32D, D95S, S98Y |
| TPP-29392 | 9.2E−10 | 1.1E−09 | 9.5E−10 | 8.5E−10 | WT | G30W, G32D, N54E, D95S |
| TPP-29393 | 6.6E−10 | 3.9E−10 | 1.8E−10 | 7.2E−10 | WT | G32D, Y33L, N54E, S98Y |
| TPP-29394 | 1.2E−09 | 6.5E−10 | 3.7E−10 | 6.7E−10 | WT | G32D, D95F, S98Y |
| TPP-29396 | 7.8E−10 | 7.0E−10 | 4.4E−10 | 9.2E−10 | WT | G30W, G32D, N54E, D95F, S98Y |
| TPP-29397 | 3.5E−10 | 1.1E−10 | 5.7E−11 | 8.0E−10 | WT | G30W, N54E, D95S, S98Y |
| TPP-29399 | 5.6E−10 | 9.3E−10 | 5.3E−10 | 5.7E−10 | WT | G32D, D95S |
| TPP-29400 | 5.0E−10 | 9.6E−10 | 7.5E−10 | 3.5E−10 | M34G, V50L | G32D, D95F |
| TPP-29401 | 6.9E−10 | 1.1E−10 | 5.8E−11 | 2.3E−10 | M34G | G32Y, N54E |
| TPP-29402 | 1.9E−10 | 4.5E−11 | 1.8E−11 | 3.2E−10 | M34G, V50L | Y33L, N54E, D95S, S98Y |
| TPP-29403 | 7.7E−10 | 1.8E−10 | 1.1E−10 | 2.6E−10 | M34G, V50L | G32Y |
| TPP-29404 | 2.0E−10 | 9.1E−11 | 6.7E−11 | 1.4E−10 | M34G, V50L | G32Y, D95S |

In addition, 18 hits were tested for human IL-11 and murine IL-11 binding by reporter gene assay as described in Example 17. Potency (IC50 [M]) and efficacy (%) were calculated as described in Example 17. Results are shown in Table E17.

TABLE E17
RGA results for 18 human IgG1 hits after 2^{nd} round affinity maturation

| | human IL-11 | | mouse IL-11 | | Mutation Site | |
|---|---|---|---|---|---|---|
| Antibody | IC50 [M] | Efficacy (%) | IC50 [M] | Efficacy (%) | VH | VL |
| TPP-16478 | 1.6E−08 | 50.9 | 2.4E−10 | 89.0 | WT | WT |
| TPP-29381 | 4.8E−10 | 75.3 | 4.5E−10 | 94.8 | WT | G32D, A91S, D95S |
| TPP-29382 | 6.4E−10 | 81.4 | 2.6E−10 | 87.5 | WT | G32D, Y33L, D95F, S98Y |
| TPP-29383 | 1.6E−09 | 86.0 | 5.7E−10 | 94.2 | WT | G32D, Y33L, N54E, D95P, S98Y |
| TPP-29384 | 2.8E−09 | 81.9 | 3.3E−10 | 97.2 | WT | N54E, D95P |
| TPP-29386 | 6.4E−10 | 88.9 | 1.2E−10 | 97.9 | WT | G32D, N54E, A91S, D95S |
| TPP-29389 | 8.4E−10 | 87.3 | 2.9E−10 | 97.4 | WT | G32Y, N54E, D95P, S98Y |
| TPP-29391 | 5.4E−10 | 84.0 | NM | 96.9 | WT | G32D, D95S, S98Y |
| TPP-29392 | 1.2E−09 | 80.2 | NM | 89.3 | WT | G30W, G32D, N54E, D95S |
| TPP-29393 | 6.1E−10 | 87.3 | 2.4E−10 | 96.2 | WT | G32D, Y33L, N54E, S98Y |
| TPP-29394 | 1.7E−09 | 89.8 | 4.8E−10 | 93.9 | WT | G32D, D95F, S98Y |
| TPP-29396 | 6.5E−10 | 78.9 | 3.9E−10 | 96.1 | WT | G30W, G32D, N54E, D95F, S98Y |
| TPP-29397 | 5.3E−10 | 71.0 | 3.1E−10 | 93.8 | WT | G30W, N54E, D95S, S98Y |
| TPP-29399 | 7.3E−10 | 70.2 | NM | 90.0 | WT | G32D, D95S |
| TPP-29400 | 4.6E−10 | 59.6 | NM | 87.3 | M34G, V50L | G32D, D95F |
| TPP-29401 | 1.3E−09 | 83.3 | 1.5E−10 | 96.9 | M34G | G32Y, N54E |
| TPP-29402 | 5.3E−10 | 80.7 | 1.9E−10 | 95.5 | M34G, V50L | Y33L, N54E, D95S, S98Y |
| TPP-29403 | 1.7E−09 | 82.4 | 2.7E−10 | 94.3 | M34G, V50L | G32Y |
| TPP-29404 | 7.2E−10 | 71.8 | 1.9E−10 | 91.8 | M34G, V50L | G32Y, D95S |

NM = Not Meaningful (=no value reported if efficacy <20% or no IC₅₀ value could be calculated)

Based on the result in these assays, mutants were compared against the Wild Type antibody TPP-16478 and either categorized as ‘improved’ or ‘non-improved’ and the final candidate TPP-29386 (hIgG1) SEQ ID NO: 92, Heavy Chain and SEQ ID NO: 93, Light Chain, was selected.

### IgG Conversion

Genes encoding the variable region of TPP-29386 from 2^{nd} round affinity maturation were cloned into murine IgG1 expression vector pTT5 (VH domain was fused with murine IgG1 variant, and VL domain was fused with murine Ig lambda CL domain) to generate a murine IgG1 expression vector for TPP-29528. In addition, genes encoding the variable region of TPP-29386 were cloned into a human expression vector pTT5 to generate a human IgG construct of a desired isotype for TPP-29536 (VH domain was fused with human IgG1 HS variant, in which Fc-silencing mutations were introduced (E233P/L234V/L235A/DG236/D265G/A327Q/A330), and VL domain was fused with human Ig lambda CL domain).

### Example 28-31 Describe the In Vitro Characterization of IgG Converted Antibodies Resulting from Optimization of TPP-18068 and TPP-18087

### Example 28: Antibody Production

For TPP-29519, TPP-29520, TPP-29521, TPP-29522, TPP-29523, TPP-29528, TPP-29680, TPP-30000, TPP-30001, TPP-30002, TPP-30003, and TPP-29536, the plasmids containing respective VH and VL genes were co-transfected into Expi293F cells. Cells were cultured for 5 days and the supernatants were collected for protein purification using Protein A column (GE Healthcare, Cat. 175438). The protein concentration from the elution was determined by A280/Extinction coefficient using Nanodrop 2000. The purified antibodies were analyzed by SDS-PAGE and HPLC-SEC, and then were stored at −80° C.

### Example 29: Affinity to Human/Mouse/Cyno and Rat IL-11 by SPR

To assess the binding kinetics and affinity of anti-IL-11 antibodies as well as their species cross-reactivity profile, binding assays were conducted using surface plasmon resonance (SPR). Binding assays were performed on a Biacore T200 instrument or on a Biacore 8K+ instrument (Cytiva) at 37° C. using assay buffer HBS EP+, 300 mM NaCl, 1 mg/ml BSA, 0.05% NaN3. Antibodies were captured either via anti-human Fc IgGs (“Human antibody capture kit”, Order No. BR100839, Cytiva) or via anti-mouse Fc IgGs (“Mouse antibody capture kit, Order No. BR100383, Cytiva) covalently amine coupled to a Series S CM5 sensor chip (Cytiva) depending on the isotype. The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human, mouse, cynomolgus or rat IL-11 was used as analyte at several dilutions within the concentration range from 0.05-200 nM. For binding assays with only one concentration 200 nM IL-11 was used. The sensor surface was regenerated with glycine pH 1.7 after each antigen injection. Obtained sensorgrams were double referenced (subtraction of reference flow cell signal and buffer injection) and were fitted to a 1:1 Langmuir binding model to derive kinetic data using the Biacore T200 Evaluation software or the Biacore Insight software. Results are shown in Table E18. If no value is reported, there was either no binding or very low binding and multiphasic binding behavior. Values below 1E-12 were considered to be below the dynamic range of the instrument and set to <1E-12.

TABLE E18
SPR results for IgG converted antibodies resulting from optimization
of TPP-18068 and TPP-18087 in comparison to parental (TPP-18068
and TPP-18087) and competitor (TPP-23552 and TPP-23580) antibodies

| Antibody | SPR: K_{D} [M] hIL11 | SPR: K_{D} [M] mIL11 | SPR: K_{D} [M] cIL11 | SPR: K_{D} [M] rIL11 |
|---|---|---|---|---|
| TPP-18068 | No binding | 4.1E−10 | 5.0E−09 | 5.8E−10 |
| TPP-18087 | No binding | 4.1E−08 | No binding | |
| TPP-29536 | 1.8E−10 | 1.3E−10 | 2.4E−10 | 2.5E−10 |
| TPP-29680 | 1.6E−12 | <1E−12 | 3.5E−10 | 9.0E−12 |
| TPP-30000 | 1.8E−11 | 3.9E−11 | 3.2E−08 | 2.1E−10 |
| TPP-30001 | 6.4E−11 | 5.5E−11 | No binding | 1.1E−09 |
| TPP-30002 | 7.4E−12 | 1.3E−11 | 1.0E−08 | 1.8E−10 |
| TPP-30003 | <1E−12 | <1E−12 | 4.9E−10 | 6.7E−12 |
| TPP-23552 | 1.4E−09 | 1.8E−09 | No binding | 3.2E−09 |
| TPP-29528 | 3.4E−10 | 2.3E−10 | 2.0E−10 | 3.3E−10 |
| TPP-29519 | 1.0E−11 | 3.5E−12 | 6.1E−10 | 2.3E−11 |
| TPP-29520 | 9.0E−11 | 1.1E−10 | No binding | 2.9E−10 |
| TPP-29521 | 1.8E−10 | 9.8E−11 | No binding | 2.1E−09 |
| TPP-29522 | 3.4E−11 | 2.9E−11 | 2.4E−08 | 3.4E−10 |
| TPP-29523 | 2.6E−12 | 4.0E−12 | 1.1E−09 | 2.0E−11 |
| TPP-23580 | 2.1E−09 | 2.4E−09 | No binding | 4.1E−09 |

### Example 30: Human, Mouse, Cyno and Rat IL-11 Reporter Gene Assay

Stat3 Reporter HEK293 cells (BPS Biosciences, #79800-P) were grown in Growth Medium 1N (BPS Biosciences, #79801). Cells were seeded at day 1 in a volume of 50 μl containing 40.000 cell per well in white 384er cell culture plates (BD BioCoat™ Poly-D-Lysine 384-well, #354660) in Growth Medium 1N.

At the same day, lipofection was done by, depending on the IL-11 species to be tested, diluting the human receptor plasmid (PJF-2813; TPP-14223; SEQ ID: no) or the mouse receptor plasmid (PJF-2809-1; TPP-14189; SEQ ID: no) or the rat receptor plasmid (PJF2811; TPP-14222; SEQ ID: no) to 0.2 μg/ml in Optimem (Invitrogen, #11058-02). Next, LF2000 (Invitrogen, 11668-027) was diluted 1:50 in Optimem. The respective diluted plasmid and diluted LF2000 were mixed in a 1:1 ratio. After an incubation for 20 min at room temperature, per well 25 μl of complex mix were added to the cells and the plate were incubated over night at 37° C., 5% CO₂.

The next day, human IL-11 (Invigate, e.g., lot #C121021-19), mouse IL-11 (Invigate, e.g., lot #C210819-09), cynomolgus IL-11 (Invigate, lot #C290621-19), or rat IL-11 (Invigate, e.g., lot #C301019-19), was diluted to a constant final concentration of 6 nM in Growth Medium 1N. In addition, a final assay serial dilution of the anti IL11 antibodies to be tested was prepared in Growth Medium 1N, starting from 100 [nM] by a factor of 3 down to 0.137 [nM], including a negative control without any antibody. IL11 and the antibody serial dilution were mixed in a 1:1 ratio and incubated for 30 min at room temperature in a microtiter plate. Then, the cell culture medium above the cells were discarded and 25 μl/well of the mix added to the cells. Thereafter, the plate was incubated for five hours at 37° C. at 5% CO₂. Finally, 25 μL/well of luciferase substrate Bright Glo (Promega; #E2620) was added to the cells and the plate was incubated for 3 min at room temperature in the dark, before luminescence was measured on a plate reader. Signals from dose responses were used to calculate efficacy and IC50 values as described in Example 17. Results are shown in Table E19.

TABLE E19
RGA results for IgG converted antibodies resulting from optimization of TPP-18068 and TPP-18087 in comparison
to parental (TPP-18068 and TPP-18087) and competitor (TPP-23552 and TPP-23580) antibodies

| | hIL11 | | mIL11 | | cIL11 | | rIL11 | |
|---|---|---|---|---|---|---|---|---|
| Antibody | IC50 | Efficacy (%) | IC50 | Efficacy (%) | IC50 | Efficacy (%) | IC50 | Efficacy (%) |
| TPP-18068 | 6.5E−09 | 46.1 | 1.2E−09 | 93.6 | 4.1E−09 | 70.0 | 1.8E−09 | 94.0 |
| TPP-18087 | NM | 47.8 | 1.6E−08 | 59.4 | NM | 47.7 | NM | 31.8 |
| TPP-29536 | 1.2E−09 | 85.1 | 1.4E−09 | 97.2 | 1.0E−09 | 89.7 | 1.3E−09 | 90.9 |
| TPP-29680 | 1.5E−09 | 98.4 | 6.6E−10 | 96.2 | 2.9E−10 | 97.5 | 1.1E−09 | 98.5 |
| TPP-30000 | 1.8E−09 | 97.8 | 6.6E−10 | 97.9 | NM | 64.6 | 1.4E−09 | 100.1 |
| TPP-30001 | 1.3E−09 | 96.5 | 9.3E−10 | 101.0 | 5.6E−09 | 56.6 | 2.1E−09 | 98.9 |
| TPP-30002 | 1.2E−09 | 96.5 | NM | 97.2 | NM | 72.8 | 1.1E−09 | 99.1 |
| TPP-30003 | 1.8E−09 | 100.4 | 7.5E−10 | 97.1 | NM | 97.1 | 7.2E−10 | 99.5 |
| TPP-23552 | 3.7E−09 | 76.7 | 8.1E−09 | 90.9 | NM | 52.4 | 8.8E−09 | 81.3 |
| TPP-29528 | NM | 81.2 | 8.9E−10 | 96.9 | 4.4E−10 | 87.8 | 1.8E−09 | 89.9 |
| TPP-29519 | 1.8E−09 | 100.1 | 1.2E−09 | 101.2 | 8.9E−10 | 99.2 | 1.0E−09 | 99.7 |
| TPP-29520 | 1.7E−09 | 99.7 | 9.1E−10 | 100.2 | NM | 58.6 | 1.3E−09 | 101.2 |
| TPP-29521 | 1.9E−09 | 99.2 | 7.6E−10 | 103.0 | NM | 60.0 | 1.8E−09 | 98.9 |
| TPP-29522 | 1.6E−09 | 98.6 | 7.8E−10 | 100.8 | NM | 52.0 | 1.6E−09 | 100.7 |
| TPP-29523 | 1.6E−09 | 100.3 | 1.4E−09 | 100.7 | 6.8E−10 | 97.2 | 1.4E−09 | 100.7 |
| TPP-23580 | NM | 64.0 | 7.9E−09 | 88.9 | NM | 21.9 | 1.4E−08 | 62.2 |

NM = Not Meaningful (=no value reported if efficacy <20% or no IC₅₀ value could be calculated)

### Example 31: Inhibition of the Interaction of IL-11 with IL-11Ra by IgG Converted Antibodies Analysed in Di-Complex ELSA

IgG converted antibodies were subjected to the experimental assay procedure described in Example 18. IC50 [M], respectively efficacy (%) were calculated accordingly and results are shown in Table E20.

TABLE E20
Inhibition of the interaction of human IL-11 with human IL-11Ra
by IgG converted antibodies analyzed in Di-complex ELISA

| | hIL11 | | mIL11 | |
|---|---|---|---|---|
| Antibody | IC50 [M] | Efficacy (%) | IC50 [M] | Efficacy (%) |
| TPP-18068 | 1.2E−08 | 17.4 | 2.9E−09 | 64.2 |
| TPP-18087 | NM | 0.0 | NM | 2.4 |
| TPP-29536 | 8.8E−10 | 37.6 | 5.5E−10 | 42.2 |
| TPP-29680 | NM | 12.2 | NM | 8.6 |
| TPP-30000 | NM | 0.0 | NM | 3.5 |
| TPP-30001 | NM | 5.2 | NM | 10.1 |
| TPP-30002 | NM | 3.5 | NM | −3.3 |
| TPP-30003 | NM | 8.4 | NM | 4.1 |
| TPP-23552 | 2.9E−09 | 97.2 | 3.3E−09 | 98.4 |
| TPP-29528 | NM | 36.3 | 2.6E−09 | 43.4 |
| TPP-29519 | NM | 6.8 | NM | 12.8 |
| TPP-29520 | NM | 0.3 | NM | 1.1 |
| TPP-29521 | NM | 1.7 | NM | 12.1 |
| TPP-29522 | NM | 0.0 | NM | 3.6 |
| TPP-29523 | NM | 2.0 | NM | 4.8 |
| TPP-23580 | 1.0E−08 | 93.3 | 6.8E−09 | 93.0 |
| TPP-14250 | 6.8E−09 | 75.7 | 3.3E−09 | 91.6 |

NM = Not Meaningful (=no value reported if efficacy <20% or no IC₅₀ value could be calculated)

TPP-18068, TPP-29536, TPP-23552, TPP-29528, TPP-23580 and TPP-14250 show activity in the Di-complex ELISA, while TPP-18087, TPP-29680, TPP-30000, TPP-30001, TPP-30002, TPP-30003, TPP-29519, TPP-29520, TPP-29521, TPP-29522 and TPP-29523 do not.

### Example 32: Inhibition of the Formation of IL-11/IL-11Ra/Gp130 Complex by IgG Converted Antibodies Analysed in Tri-Complex ELISA

IgG converted antibodies were subjected to the experimental assay procedure described in Example 19 and IC50 [M], respectively efficacy (%) were calculated accordingly. Results are shown in Table E21.

### Table E21: Inhibition of the Formation of Human IL-11/IL-11Ra/Gp130 Complexes by IgG Converted Antibodies Analyzed in Tri-Complex

| | hIL11 | | mIL11 | |
|---|---|---|---|---|
| Antibody | IC50 [M] | Efficacy (%) | IC50 [M] | Efficacy (%) |
| TPP-18068 | 1.7E−06 | 84.9 | 3.9E−09 | 84.9 |
| TPP-18087 | 1.5E−09 | 25.0 | 3.3E−08 | 25.0 |
| TPP-29536 | 1.0E−09 | 98.7 | 6.4E−10 | 102.8 |
| TPP-29680 | 7.0E−09 | 78.8 | 1.1E−10 | 100.7 |
| TPP-30000 | 5.7E−09 | 86.0 | 1.2E−10 | 98.8 |
| TPP-30001 | 1.1E−08 | 81.0 | 1.1E−10 | 99.7 |
| TPP-30002 | 4.9E−09 | 86.2 | 7.3E−11 | 102.4 |
| TPP-30003 | 7.4E−09 | 85.4 | 1.0E−10 | 101.7 |
| TPP-23552 | 6.8E−09 | 90.6 | 2.3E−08 | 90.7 |
| TPP-29528 | 1.0E−09 | 94.7 | 6.0E−10 | 94.7 |
| TPP-29519 | 2.4E−09 | 81.3 | 1.4E−10 | 81.3 |
| TPP-29520 | 1.3E−09 | 81.8 | 1.3E−10 | 81.8 |
| TPP-29521 | 1.8E−09 | 85.7 | 9.2E−11 | 85.7 |
| TPP-29522 | 2.0E−09 | 85.4 | 1.1E−10 | 85.4 |
| TPP-29523 | 2.1E−09 | 84.3 | 1.2E−10 | 84.3 |
| TPP-23580 | 3.4E−09 | 45.8 | 3.1E−08 | 45.8 |
| TPP-14250 | 1.5E−08 | 81.1 | 8.0E−09 | 81.1 |

All tested antibodies are active in the tri-complex ELISA.

### Example 33: Binding of Optimized and IgG Converted Antibodies to IL2Ra

IgG converted antibodies TPP-were tested for binding to IL2Ra as described in Example 20. The ratios of the signals obtained at an antibody concentration of 3.3E-7 [M] for IgG converted antibody versus the isotype control TPP-10159 were calculated and are shown in Table E22.

TABLE E22
Ratio of IL-2R binding by IgG converted antibody
versus binding by isotype control antibody

| | Antibody/Isotype Control Ratio |
|---|---|
| Antibody | of IL-2Ra Binding Signals |
| TPP-18068 | 377.3 |
| TPP-18087 | 0.6 |
| TPP-29536 | 1.1 |
| TPP-29528 | 24.2 |
| TPP-29519 | 0.7 |
| TPP-29520 | 0.6 |
| TPP-29521 | 0.7 |
| TPP-29522 | 0.7 |
| TPP-29523 | 1.2 |
| TPP-23580 | 2.2 |

All tested antibodies do not bind to IL2RA with the exception of TPP-18068.

### Example 34: Generation of Bispecific IL-11 Antibodies

The Di- and Tri-complex ELISA data indicate that TPP-18068 and TPP-18087 bind to two different epitopes on IL-11. In order to generate antibodies capable of binding to both epitopes a bispecific scFv-kih construct consisting of one arm of each, TPP-18068 and TPP-18087, was generated. Therefore, the variable regions of TPP-18068 and TPP-18087 were transferred to a scFv format by using standard recombinant DNA techniques (Sambrook, J. et al. eds., MOLECULAR CLONING: A LABORATORY MANUAL (2d Ed. 1989) Cold Spring Harbor Laboratory Press, NY. Vols. 1-3). The VH sequences were linked by a 15 amino acid GGGGS GGGGSGGGGS (e.g., SEQ ID NO: 74, aa 114-129) linker to the respective VL sequence. scFv resulting from TPP-18068 was fused to a human IgG Fc domain containing the knob mutation and scFv resulting from TPP-18087 was fused to a human IgG Fc domain containing the hole mutations via the sequence GG GGSGGGGSGG GGSG (e.g., SEQ ID NO: 74, aa 240-256), (FIG. 20). The resulting constructs were both cloned into the vector pTT5 for expression in HEK293-6E cells using standard transient transfection procedures and resulting human IgG antibody TPP-20489 was purified from the cell culture supernatant via Protein-A and size exclusion chromatography as described in Example 15. For the respective murine variant TPP-26195 murine IgG Fc domains with knob and hole mutations were used.

In a similar way TPP-29603 and TPP-29697 were generated by combining anti-LI 1 scFvs derived from TPP-29528 (optimized antibody based on TPP-18086) and TPP-29519 (optimized antibody based on TPP-18087). TPP-29603 has a murine IgG1 format with knob-into-hole mutations, while TPP-29697 has a human IgG1 format in which the Fc contains silencing mutations (E233P/L234V/L235A/DG236/D265G/A327Q/A330S mutations) next to knob-into-hole mutations.

### Example 33: Affinity of Bispecific Antibodies by SPR

To assess the binding kinetics and affinity of anti-IL-11 bispecific antibodies as well as their species cross-reactivity profile, binding assays were conducted using surface plasmon resonance (SPR). Binding assays were performed on a Biacore T200 instrument or on a Biacore 8K+ instrument (Cytiva) at 25° C. for TPP-26195 and TPP-20489 and at 37° C. for TPP-29603 and TPP-29697, using assay buffer HBS EP+, 300 mM NaCl, 1 mg/ml BSA, 0.05% NaN3. Antibodies were captured either via anti-human Fc IgGs (“Human antibody capture kit”, Order No. BR100839, Cytiva) or via anti-mouse Fc IgGs (“Mouse antibody capture kit, Order No. BR100383, Cytiva) covalently amine coupled to a Series S CM5 sensor chip (Cytiva) depending on the isotype. The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human, mouse, cynomolgus or rat IL-11 was used as analyte at several dilutions within the concentration range from 0.05-200 nM. For binding assays with only one concentration 200 nM IL-11 was used. The sensor surface was regenerated with glycine pH 1.7 after each antigen injection. Obtained sensorgrams were double referenced (subtraction of reference flow cell signal and buffer injection) and were fitted to a 1:1 Langmuir binding model to derive kinetic data using the Biacore T200 Evaluation software or the Biacore Insight software.

Results for TPP-26195 and TPP-20489 are shown in Tables E23, and for TPP-29603 and TPP-29697 in Table E24. If no value is reported, there was either no binding or very low binding and multiphasic binding behavior.

TABLE E23
SPR results for bispecific antibodies generated from non-optimized parental
antibodies TPP-18068 and TPP-18087. SPR experiments were performed at 25° C.

| Antibody | SPR: K_{D} [M] hIL11 | SPR: K_{D} [M] mIL11 | SPR: K_{D} [M] cIL11 | SPR: K_{D} [M] rIL11 |
|---|---|---|---|---|
| TPP-18068 | 2.50E−08 | 3.50E−10 | 3.80E−09 | 2.90E−10 |
| TPP-18087* | 1.30E−08 | 3.30E−08 | 5.60E−08 | 3.40E−08 |
| TPP-26195 | 8.30E−11 | 4.60E−11 | 7.40E−11 | 4.50E−11 |
| TPP-20489 | 1.10E−11 | 5.00E−12 | 2.50E−11 | 2.60E−12 |

*Determined in steady state

TABLE E24
SPR results for bispecific antibodies generated from optimized antibodies
TPP-29528 and TPP-29519. SPR experiments were performed at 37° C.

| Antibody | SPR: K_{D} [M] hIL11 | SPR: K_{D} [M] mIL11 | SPR: K_{D} [M] cIL11 | SPR: K_{D} [M] rIL11 |
|---|---|---|---|---|
| TPP-18068 | No binding | 4.1E−10 | 5.0E−09 | 5.8E−10 |
| TPP-18087 | No binding | 4.1E−08 | No binding | No binding |
| TPP-29536 | 1.8E−10 | 1.3E−10 | 2.4E−10 | 2.5E−10 |
| TPP-29680 | 1.6E−12 | <1E−12 | 3.5E−10 | 9.0E−12 |
| TPP-29697 | <1E−12 | <1E−12 | <1E−12 | <1E−12 |
| TPP-29528 | 3.4E−10 | 2.3E−10 | 2.0E−10 | 3.3E−10 |
| TPP-29519 | 1.0E−11 | 3.5E−12 | 6.1E−10 | 2.3E−11 |
| TPP-29603 | 1.9E−12 | 1.7E−12 | 2.2E−12 | 3.4E−12 |

### Example 35: Reporter Gene Assay for Human/Mouse IL-11 Blocking by Bispecific IL-11 Antibodies

The non-optimized bispecific antibodies TPP-20489 and TPP-26195 were tested in the Stat3 reporter gene assay described in Example 17 for inhibition of IL-11 mediated signaling in comparison to prior art antibodies TPP-14250, TPP23580 and TPP-23552. Potency values (IC50 [M]) were calculated using GraphPad Prism. Efficacy were calculated as described in Example 17. Results are shown in Table E25.

TABLE E25
RGA results for bispecific antibodies TPP-20489
and TPP-26195 generated from non-optimized parental
antibodies TPP-18068 and TPP-18087

| | hIL11 | | mIL11 | |
|---|---|---|---|---|
| Antibody | IC50 [M] | Efficacy (%) | IC50 [M] | Efficacy (%) |
| TPP-18068 | 4.8E−09 | 20.7 | 2.7E−09 | 84.1 |
| TPP-18087 | 1.1E−07 | 51.5 | 3.3E−07 | 49 |
| TPP-20489 | 3.1E−10 | 102 | 1.0E−09 | 93.9 |
| TPP-26195 | 6.5E−10 | 88.5 | 1.8E−09 | 96.9 |
| TPP-14250 | 2.4E−10 | 27.1 | 1.4E−07 | 69.1 |
| TPP-23580 | 8.7E−09 | 68.6 | 2.8E−08 | 46.7 |
| TPP-23552 | 1.8E−08 | 87.9 | 2.2E−08 | 76 |

Finally, the optimized bispecific antibodies TPP-29603 and TPP-29697 were tested in the Stat3 reporter gene assay described in Example 17 for inhibition of IL-11 mediated signaling, with the exception, that cynomolgus IL-11 (Invigate, e.g., lot #C290621-19) and rat IL-11 (Invigate, e.g., lot #C301019-19),) were tested as well, and all antigens were used at 6 [nM] in the assay. For comparison, prior art antibodies TPP23580 and TPP-23552 were included in the assay. Results are shown in Table E26.

TABLE E26
RGA results for bispecific antibodies TPP-29603 and TPP-29697 generated
from optimized parental antibodies TPP-29528 and TPP-29519.

| | hIL11 | | mIL11 | | cIL11 | | rIL11 | |
|---|---|---|---|---|---|---|---|---|
| Antibody | IC50 | Efficacy (%) | IC50 | Efficacy (%) | IC50 | Efficacy (%) | IC50 | Efficacy (%) |
| TPP-18068 | 6.5E−09 | 46.1 | 1.2E−09 | 93.6 | 4.1E−09 | 70.0 | 1.8E−09 | 94.0 |
| TPP-18087 | NM | 47.8 | 1.6E−08 | 59.4 | NM | 47.7 | NM | 31.8 |
| TPP-29528 | NM | 81.2 | 8.9E−10 | 96.9 | 4.4E−10 | 87.8 | 1.8E−09 | 89.9 |
| TPP-29519 | 1.8E−09 | 100.1 | 1.2E−09 | 101.2 | 8.9E−10 | 99.2 | 1.0E−09 | 99.7 |
| TPP-29603 | 4.1E−09 | 101.0 | 2.0E−09 | 101.7 | 1.7E−09 | 101.3 | 2.0E−09 | 99.9 |
| TPP-29536 | 1.2E−09 | 85.1 | 1.4E−09 | 97.2 | 1.0E−09 | 89.7 | 1.3E−09 | 90.9 |
| TPP-29680 | 1.5E−09 | 98.4 | 6.6E−10 | 96.2 | 2.9E−10 | 97.5 | 1.1E−09 | 98.5 |
| TPP-29697 | 2.0E−09 | 99.1 | 1.4E−09 | 99.6 | 7.5E−10 | 100.5 | ~1.2e−09 | 97.6 |
| TPP-23552 | 3.7E−09 | 76.7 | 8.1E−09 | 90.9 | NM | 52.4 | 8.8E−09 | 81.3 |
| TPP-23580 | NM | 64.0 | 7.9E−09 | 88.9 | NM | 21.9 | 1.4E−08 | 62.2 |

NM = Not Meaningful (=no value reported if efficacy <20% or no IC₅₀ value could be calculated)

### Example 35: Inhibition of the Interaction of IL-11 with IL-11Ra by Bispecific IL-11 Antibodies Analysed in Di-Complex ELSA

Bispecific antibodies were subjected to the experimental assay procedure described in Example 18 and IC50 [M], respectively efficacy (%) were calculated accordingly. Results are shown in Table E27.

TABLE E27
Inhibition of the interaction of human IL-11 with human IL-
11Ra by bispecific antibodies analyzed in Di-complex ELISA

| | hIL11 | | mIL11 | |
|---|---|---|---|---|
| Antibody | IC50 [M] | Efficacy (%) | IC50 [M] | Efficacy (%) |
| TPP-20489 | NM | 13.3 | 3.5E−08 | 45.6 |
| TPP-26195 | 6.3E−09 | 28.6 | 4.1E−09 | 59.7 |
| TPP-29697 | 1.6E−09 | 46.7 | 1.4E−09 | 52.3 |
| TPP-29603 | 1.7E−09 | 35.6 | 3.9E−09 | 53.3 |

NM = Not Meaningful (=no value reported if efficacy <20% or no IC₅₀ value could be calculated)

### Example 36: Inhibition of the Formation of IL-11/IL-11Ra/Gp130 Complex by Bispecific Antibodies Analysed in Tri-Complex ELISA

Bispecific antibodies were subjected to the experimental assay procedure described in Example 19 and IC50 [M], respectively efficacy (%) were calculated accordingly. Results are shown in Table E28

TABLE E 28
Inhibition of the formation of human IL-11/IL-11Ra/gp130 complexes
by bispecific antibodies analyzed in Tri-complex ELISA

| | hIL11 | | mIL11 | |
|---|---|---|---|---|
| Antibody | IC50 [M] | Efficacy (%) | IC50 [M] | Efficacy (%) |
| TPP-20489 | 6.28E−09 | 104.7 | 1.81E−09 | 94.0 |
| TPP-26195 | 1.28E−09 | 98.0 | 2.58E−10 | 98.0 |
| TPP-29697 | 1.60E−09 | 104.1 | 6.22E−11 | 104.5 |
| TPP-29603 | 6.96E−10 | 98.5 | 1.70E−10 | 98.5 |

### Example 37: Binding of Bispecific IL-11 Antibodies to IL2Ra

Bispecific antibodies were subjected to the experimental assay procedure described in Example 20. Ratios of the signals generated at an antibody concentration of 1E-07 [M] by the respective bispecific antibody versus those of an isotype control antibody were calculated and are shown in Table E29. The bispecific antibodies show no binding to IL-2 Ra.

TABLE E29
Ratio of IL-2R binding by bispecific antibodies
versus binding by isotype control antibody

| | Ratio IL-2Ra Binding Signal |
|---|---|
| Antibody | Antibody/Isotype Control |
| TPP-26195 | 146.4 |
| TPP-20489 | 851.9 |
| TPP-29603 | 1.9 |
| TPP-29697 | 0.8 |

TPP-29603 and TPP-29697 do not bind to IL2Ra.

### Example 38: Characterization of Additional Commercially Available Antibodies by SPR, RGA, Di-Complex and Tri-Complex ELISA

Commercially available antibodies (see Table E30) described to recognized human IL-11 were analysed in SPR, RGA and ELISA assay according to the experimental procedures described in examples 16, 17 and 18.

TABLE E30
List of additional commercially available anti IL-11 antibodies
tested in SPR, RGA, Di-Complex and Tri-Complex ELISA

| Cat # | Company | Applications | Reactivity | Antigen | Isotype | Clone |
|---|---|---|---|---|---|---|
| GTX34009 | Genetex | WB | Human | IL11 | IgG1 | 6D9A1 |
| GTX52814 | Genetex | WB, | Human | IL11 | IgG2 | 9T27 |
| | | Neutralizing/Inhibition | | | | |
| LS-C104441 | LS-bio | WB, ELISA | Human | IL11 | IgG1 | |
| MA5-30696 | Thermo Fisher | ELISA | Human | IL11 | IgG1 | 12 |
| | Scientific | | | | | |
| MA5-30695 | Thermo Fisher | ELISA | Human | IL11 | IgG1 | 9 |
| | Scientific | | | | | |
| MA5-23711 | Thermo Fisher | WB, ELISA | Human | IL11 | IgG2b | 22616 |
| | Scientific | | | | | |
| LS-C193526 | LS-bio | WB, ELISA | Human | IL11 | IgG1 | KT8 |

Commercially available antibodies listed in Table E30 were subjected to SPR as described in example 16, with the exception, that human, respectively mouse IL-11 were measured only at a single concentration of 200 [nM]. Results are shown in Table E31.

TABLE E31
Commercially available antibodies tested by SPR

| Antibody Cat # | SPR: hIL-11 KD [M] | SPR: mIL-11 KD [M] |
|---|---|---|
| GTX34009 | No binding | No binding |
| GTX52814 | 3.3E−09 | 2.0E−09 |
| LS-C104441 | 3.9E−09 | 2.2E−09 |
| MA5-30696 | No binding | No binding |
| MA5-30695 | No binding | No binding |
| MA5-23711 | 3.6E−09 | 2.0E−09 |
| LS-C193526 | 6.3E−09 | 6.2E−09 |

Only four of the tested commercial antibodies showed binding to human or mouse IL-11 in SPR (GTX52814, LS-C104441, MA5-23711, and LS-C193526).

Commercially available antibodies listed in Table E30 were subjected to RGA as described in example 17. Results are shown in Table E32.

TABLE E32
Commercially available antibodies tested by RGA

| | hIL-11 | | mIL-11 | |
|---|---|---|---|---|
| Cat # | IC50 [M] | Efficacy (%)* | IC50 [M] | Efficacy (%)* |
| GTX34009 | NM | 21.3 | NM | 0.0 |
| GTX52814 | NM | 5.1 | NM | 75.5 |
| LS-C104441 | NM | 42.6 | 3.9E−09 | 88.0 |
| MA5-30696 | NM | 4.9 | NM | 17.2 |
| MA5-30695 | NM | 23.9 | NM | 15.6 |
| MA5-23711 | NM | 19.9 | NM | 47.6 |
| LS-C193526 | 5.0E−08 | 52.7 | 5.6E−09 | 88.3 |

NM = Not Meaningful (=no value reported if efficacy <20% or no IC₅₀ value could be calculated)

Commercially available antibodies listed in Table E30 were subjected to Di-complex ELISA as described in example 18. Results are shown in Table E33.

TABLE E33
Commercially available antibodies tested by Di-complex ELISA

| | human IL-11 | | mouse IL-11 | |
|---|---|---|---|---|
| Antibody | IC50 [M] | Efficacy (%) | IC50 [M] | Efficacy (%) |
| GTX34009 | NM | 10.3 | NM | 11.7 |
| GTX52814 | 1.9E−09 | 74.9 | 4.7E−09 | 89.9 |
| LS-C104441 | 5.3E−09 | 71.5 | 3.5E−09 | 91.2 |
| MA5-30696 | NM | 8.1 | NM | 3.1 |
| MA5-30695 | NM | 0.0 | NM | 0.0 |
| MA5-23711 | 6.0E−08 | 51.1 | 2.9E−08 | 70.5 |
| LS-C193526 | 1.0E−08 | 90.1 | 6.5E−09 | 96.2 |

NM = Not Meaningful (=no value reported if efficacy <20% or no IC₅₀ value could be calculated)

Commercially available antibodies listed in Table E30 were subjected to Tri-Complex as described in example 19. Results are shown in Table E34.

TABLE E34
Commercially available antibodies tested by Tri-Complex ELISA

| | human IL-11 | | mouse IL-11 | |
|---|---|---|---|---|
| Antibody | IC50 [M] | Efficacy (%) | IC50 [M] | Efficacy (%) |
| GTX34009 | NM | 1.1 | NM | 0.0 |
| GTX52814 | 1.4E−08 | 36.9 | 1.7E−08 | 105.9 |
| LS-C104441 | 2.0E−08 | 65.6 | 3.8E−09 | 98.4 |
| MA5-30696 | NM | 4.7 | NM | 4.3 |
| MA5-30695 | NM | 0.0 | NM | 1.5 |
| MA5-23711 | 1.9E−08 | 34.1 | NM | 62.3 |
| LS-C193526 | 1.0E−08 | 78.5 | 6.6E−09 | 90.3 |

NM = Not Meaningful (=no value reported if efficacy <20% or no IC₅₀ value could be calculated)

Commercial antibodies, which were tested in all assays, were either inactive in SPR and RGA assay (GTX34009, MA5-30696, and MA5-30695) or were active in SPR, RGA, Di-complex ELISA and Tri-complex ELISA (GTX52814, LS-C104441, MA5-23711, LS-C193526). None of the tested commercial antibodies were active in the Tri-complex ELISA but not in the Di-complex ELISA as described for antibodies according to the invention.

### Example 39: Binding of TPP-18068 or TPP-18087 to IL-11 in the Presence of Increasing Concentrations of Affinity Matured Derivatives Thereof

25 μl/well of 20 [nM] biotinylated human IL-11 (Invigate, e.g., lot #C121021-19) in coating buffer pH 9.6 (Candor, #121125) were added to a streptavidin coated 384 well microtiter plate (Greiner bio-one, #781997) and incubated for one hour at room temperature. After three washes with 50 μl/well PBS-t, the plate was blocked with 50 μl/well SmartBlock solution (Candor, #113125) and incubated again for one hour at room temperature. After three additional washes with PBS-T, a dilution series of human IgGs, ie., TPP-29536, TPP-29680 or isotype control antibody TPP-5657 in 10% SmartBlock in PBS-T, ranging from 2.5E-07 [M] to 1.6E-11 [M], were added in 25 μl/well in the presence of a constant concentration of 1E-08 [M] of murine IgGs TPP-18068 or TPP18087 to the plate and incubated for one hour at room temperature. After three additional washes, 25 μl anti-mouse-POD (Jackson, #715-035-150), diluted 1:5000 in 10% SmartBlock PBS-T were added to the plate and incubated for one hour at room temperature. After three final washes, 25 μl AmplexRed solution (Fisher Scientific, #VXA12222), 1:1000 diluted in PBS, 0.003% H₂O₂ were added and incubated for 15 min at room temperature in the dark. Relative fluorescence was measured with a plate reader at 535/595 nm and signals were transferred to GraphPad Prism for analysis.

As shown in FIG. 21 a), increasing concentrations of competing antibody TPP-29536, a derivative of TPP-18068 (active in both di-complex and tri-complex ELISA), completely block binding of TPP-18068 to IL-11, while TPP-29680, a derivative of TPP-18087 (active in tri-complex ELISA but not in di-complex ELISA), has no effect. Furthermore, increasing concentrations of competing antibody TPP-29680, completely block binding of TPP-18087 to IL-11, while TPP-29536 has no effect (FIG. 21 b). These data indicate that the epitopes of TPP-18068 (active in both di-complex and tri-complex ELISA) and its derivatives are distinct and not overlapping with those of TPP-18087 (active in tri-complex ELISA but not in di-complex ELISA) and its derivatives.

### FIGURES

FIG. 1 describes the mouse model of heavy menstrual bleeding. It shows that progesterone (P4) withdrawal in ovariectomized and estrogen and progesterone substituted mice with induced endometrial differentiation mimics menstruation and menstrual uterine bleeding as described in Example 1. Blood loss was monitored by analyzing blood content in tampons. Further effects on menstruation were monitored by analyzing uterus weight or histology or expression of tissue degradation markers in comparison to e.g., proliferation markers. 1) Removal of Ovary; 2) Estrogen Substitution; 3) Progesterone/Estrogen Substitution; 4) Intrauterine Application of Oil; 5) Progesterone Withdrawal (on day (d) 12); 6) Autopsy (on day 16).

FIG. 2 shows the results of the experiments of Example 1: It demonstrates the effect of a function blocking polyclonal IL-11 antibody (IL11-fbAb AF418; triangles) vs. isotype control antibody (ctrl-Ab; TPP-12904; circles) on menstruation in a mouse model of HMB. The blood loss [μL] is significantly (two tailed t test, **** p<0.0001) attenuated in the IL-11 antibody treated group in comparison to the isotype control antibody group.

FIG. 3 shows the results of the experiments of Example 2: It demonstrates the effect of a function blocking IL-11 antibody (polyclonal IL11-fbAb AF418; triangles) vs. isotype control antibody (ctrl-Ab; TPP-12904; circles) on uterus weight in a mouse model of HMB. The uterine weight [mg] is significantly (two tailed t test, *** t<0.001) reduced by IL-11 function blocking antibody in comparison to the control antibody.

FIG. 4 shows the result of Example 3: The effect of 3 ng/ml human IL-11 (hIL-11 [218-IL from R&D Systems, Inc.]), open triangles) and 1 μg/ml human IL-11 function blocking antibody (hIL11-fbAb AF418; squares) vs. isotype control antibody (ctrl-Ab; TPP-12904; circles) on secretion of Vascular Endothelial Growth Factor (VEGF; [ng VEGF/mg tissue) in a Primary Human Fibroid Slice Assay. VEGF, a key mediator of angiogenesis IL-11 is shown. The significantly induced secretion of the angiogenic mediator VEGF-A by hIL-11 was completely inhibited by additional treatment with the IL-11 function blocking antibody AF418 (closed triangles). Statistical test: one-way ANOVA, ****p<0.0001.

FIG. 5 shows the results of the experiments of Example 4: Effect of a function blocking IL-11 antibody (IL11-fbAb; AF418; triangles) vs. isotype control antibody (ctrl-Ab; TPP-12904; circles) during menstruation on change in body weight in percent (%; d12/d16) in a mouse model of HMB. The body weight loss is significantly (two tailed t test, **** p<0.0001) attenuated in the IL-11 antibody treated group in comparison to the isotype control antibody treated group.

FIG. 6: shows the result of the experiments of Example 5: Effects of commercially available Monoclonal IL-11 Function Blocking Antibodies Mab-218 (closed triangles) in comparison to its IgG control antibody TPP-10748 (circles) and Mab-418 (open triangles) in comparison to its IgG control antibody TPP-10750 (squares) on Menstruation in ScidBeige mice, in comparison to the Polyclonal Function Blocking Antibody AF418 (diamonds). Significance was tested by one-way ANOVA with Bonferroni correction for multiple comparison (* p<0.05; **** p<0.0001)

FIG. 7: Potency dependent inhibition of menstrual bleeding in Scid beige mice by monoclonal antibodies. Significant effects of IL-11 function blocking antibody TPP-18068 (triangles) but not IL-11 function blocking antibody TPP-18063 (squares) in comparison to the isotype control antibody TPP-10159 (circles) on menstrual blood loss. Significance was tested by one-way ANOVA with Bonferroni correction for multiple comparison (** p<0.01)

FIG. 8: Dose dependent inhibition of menstrual bleeding in Heavy Menstrual Bleeding Model in Balb/c mice with no inhibition by 1 mg/kg of TPP-18068 (squares), but significant inhibition by 5 mg/kg (triangles) or 20 mg/kg (diamonds) vs 20 mg/kg control antibody TPP-10159 (circles). Significance was tested by one-way ANOVA with Bonferroni correction for multiple comparison (** p<0.01)

FIG. 9: Effect of a function blocking IL-11 antibody (TPP-18068, circles) during menstruation on spontaneous activity measured by travelled distance (horizontal activity) and rearing (vertical activity) in comparison to control-antibody TPP-TPP10159 (squares) A: horizontal activity as travelled distance in meter in 5 minutes; B: vertical activity (rearing) numbers in 5 minutes. Animals treated with IL-11 antagonizing antibody TPP-18068 were more active (two-tailed t test: *p<0.05; **p<0.01, respectively) in A and B.

FIG. 10: IL-11 function blocking antibody TPP-18068 reduces Stat3 phosphorylation in decidualized (triangles) but not in the not decidualized (diamonds) uterus horns on d12 in HMB model in comparison to the control antibody TPP-10159 in the decidualized (circles) and not decidualized uterus horns (squares). Statistical test: One-way ANOVA with Bonferroni correction for multiple comparison ** p<0.01; *** p<0.001.

FIG. 11: Attenuation of menstrual bleeding in Balb/c mice by monoclonal antibodies. TPP-18068 (closed triangles), TPP-26195 (diamonds) and TPP-23580 open triangles). In comparison to their respective control IgG-antibodies TPP-27360 (squares) and TPP-10159 (circles). TPP-18068 and TPP-26195 but not TPP-23580 showed significant effects on heavy menstrual bleeding at the dose of 15 mg/kg twice weekly. Statistical test: Two-way Mann-Whitney test, *** p<0.001; ** p<0.01; ns=not significant

FIG. 12 shows the attenuation of uterine differentiation by the biparatopic IL-11 function blocking antibody TPP-26195 (circles) in Balb/c mice at the dose of 15 mg/kg twice weekly in comparison to the control IgG-antibody (squares) as measured by the uterine weight on day 12 of the murine heavy menstrual bleeding model. Statistical test: one-tailed t test, *p<0.05

FIG. 13 shows the dose dependent attenuation of menstrual bleeding by the biparatopic IL-11 function blocking antibody TPP-26195 in Balb/c mice with doses of 10 mg/kg (circles), 3 mg/kg diamonds), 1 mg/kg (closed triangles) and 0.3 mg/kg (open triangles) twice weekly in comparison to 10 mg/kg of the control IgG-antibody TPP-27360 (squares) demonstrating significant inhibition by this IL-11 function blocking antibody already at 1 mg/kg twice weekly dosing (p<0.01). Statistical test: One-way ANOVA with Bonferroni correction for multiple comparison, ** p<0.01; *** p<0.001; ns=not significant

FIG. 14 shows the attenuation of menstrual bleeding by equimolar dosing of the biparatopic IL-11 function blocking antibody TPP-29603 (circles) and the antibodies TPP-29528 (diamonds) and TPP-29519 (closed triangles) in Balb/c mice in comparison to the control IgG-antibody TPP-10159 (squares). In addition, also a lower dose of the antibody TPP-29519 (open triangles) is shown. The experiment demonstrates significant inhibition of menstrual bleeding especially by the IL-11 function blocking antibodies TPP-29603 and TPP-29519 at the equimolar dose. Significance test: One-way ANOVA with Dunnett's post-hoc test for multiple comparison (log normalized values), ** p<0.01

FIG. 15 shows the dose dependent attenuation of menstrual bleeding by the IL-11 function blocking antibody TPP-29523 in Balb/c mice at twice weekly s.c. dosing of 2 mg/kg (circles), 0.7 mg/kg (diamonds), and 0.3 mg/kg (closed triangles) in comparison to 2 mg/kg of the control IgG-antibody TPP-10159 (squares) and 0.143 mg/kg dosing of the biparatopic IL-11 function blocking antibody TPP-29603 (open triangles) demonstrating significant inhibition by the IL-11 function blocking antibody TPP-29523 at all tested doses (** p<0.01). Statistical test: One way ANOVA with Dunnett's post-hoc test for multiple comparison,

FIG. 16: Panning strategy for lead finding: 2 major strategies for selections on biotinylated antigens are depicted. Prior to each round of selection a depletion step on an irrelevant biotinylated protein was included.

FIG. 17: Schematic diagram of the Di-complex ELISA for testing human anti IL-11 antibodies (a) or murine anti IL-11 antibodies (b). Panel a) shows the Di-complex assay principle when the competing IL-11 antibody has a human IgG format. First, biotinylated human or murine IL-11 is captured by immobilized streptavidin on the surface of a microtiter plate. A murine IL-11Ra-mouse-Fc fusionprotein will bind to the captured biotinylated IL-11, unless the competing antibody binds to IL-11 in way, that the binding site of IL-11Ra gets blocked. The relative amount of murine IL-11Ra-mouse-Fc bound to IL-11 is detected with an anti-mouse-POD reagent and substrate. Panel b) shows the Di-complex assay principle when the competing IL-11 antibody has a murine IgG format. First, biotinylated human or murine IL-11 is captured by immobilized streptavidin on the surface of a microtiter plate. A canine IL-11Ra-human-Fc fusionprotein will bind to the captured biotinylated IL-11, unless the competing antibody binds to IL-11 in way, that the binding site of IL-11Ra gets blocked. The relative amount of canine IL-11Ra-mouse-Fc bound to IL-11 is detected with an anti-mouse-POD reagent and substrate.

FIG. 18: Schematic diagram of the Tri-complex assay format for testing human anti IL-11 antibodies (a) or murine anti IL-11 antibodies (b). Panel a) shows the Tri-complex assay principle when the competing IL-11 antibody has a human IgG format. First, anti-mouse Fc antibodies are coated as capture reagent on the surface of a microtiter plate. Next, a mixture of mouse IL-11Ra-mouse-Fc fusionproteins, mouse or human IL-11, mouse or human gp130-human-Fc fusionprotein and competing IL-11 antibody is added to the plate. Depending on whether mouse or human IL-11 is tested, the species matched mouse or human gp130 fusionprotein is used. Mouse-IL-11Ra, mouse or human IL-11 and mouse or human gp130 will form a tri-molecular complex, that will be captured by the coated anti mouse Fc antibody and detected by a labelled anti mouse or human gp130 antibody (labelled via biotinylation and streptavidin-POD). However, in the presence of a competing antibody, that binds to mouse respectively human IL-11 in a way that either the interaction between mouse IL-11Ra and IL-11 or the interaction between gp130 and IL-11 is blocked, no tri-molecular complex will be generated, and the signal generated by streptavidin peroxidase will be diminished. Of note, the human Fc tag in the gp130 fusionproteins is not used in this assay format.

Panel b) shows the Tri-complex assay principle when the competing IL-11 antibody has a murine IgG format. First, anti-human Fc antibodies are coated as capture reagent on the surface of a microtiter plate. Next, a mixture of mouse IL-11Ra-mouse-Fc fusionproteins, mouse or human IL-11, mouse or human gp130-human-Fc fusionprotein and competing IL-11 antibody is added to the plate. Depending on whether mouse or human IL-11 is tested, the species matched mouse or human gp130-human-Fc fusionprotein is used. Mouse-IL-11Ra, mouse or human IL-11 and mouse or human gp130 will form a tri-molecular complex, that will be captured by the coated anti human Fc antibody and detected by a labelled anti mouse Il-11Ra antibody (labelling via biotinylation and streptavidin-POD). However, in the presence of a competing antibody, that binds to mouse respectively human IL-11 in a way that either the interaction between mouse IL-11Ra and IL-11 or the interaction between gp130 and IL-11 is blocked, no tri-molecular complex will be generated, and the signal generated by streptavidin peroxidase will be diminished. Of note, the mouse Fc tag in the mouse Il-11Ra fusionprotein is not used in this assay format.

FIG. 19: Illustration of combinatorial library design for 2nd round affinity maturation TPP-18087. Selected mutations are shown below the sequences of VHCDR2, VHCDR3, VLCDR1 and VLCDR3.

FIG. 20: Generic makeup of bispecific antibodies as generated in Example 34. A variable region of a first antibody, e.g., TPP-18068, is transferred to a scFv, which is fused to a human IgG Fc domain containing the knob mutation. A variable region of a second antibody, e.g., TPP-18087, is transferred to a scFv, which is fused to a human IgG Fc domain containing the hole mutations. Upon expression of both chains in a host cell, bispecific antibodies are formed, which can be purified by conventional methods from the culture supernatant.

FIG. 21: Lack of competition between an IL-ii antibody active in Di- and Tri-complex ELISA and an IL-11 antibody active in Tri-complex but not in Di-complex ELISA. Panel a) shows the relative fluorescence generated by binding of TPP-18068 to human IL-11, coated on a microtiter plate and subsequent detection via anti-mouse Fc antibody-POD conjugate. In the presence of an increasing amount of TPP-29536, an affinity matured and human Fc containing derivative of TPP-18068, the signal is diminished and completely blocked at the highest antibody concentration. TPP-18068 and TPP-29536 are active in the di- and tri-complex ELISA. In contrast, an increasing concentration of TPP-29680, an affinity matured and human Fc containing derivative of TPP-18087, does not lead to a significant decrease in signal intensity. TPP-18087 and TPP-29680 are active in the tri-complex but not in the di-complex ELISA. Panel b) shows the relative fluorescence generated by binding of TPP-18087 to human IL-11, coated on a microtiter plate and subsequent detection via anti-mouse Fc antibody-POD conjugate. In the presence of an increasing amount of TPP-29680, the signal is diminished and completely blocked at the highest antibody concentration. In contrast, an increasing concentration of TPP-29536, does not lead to a significant decrease in signal intensity.

Sequence Listing
Sequences of IL-11, Il-11Ra, and gp130

| No | Type | Sequence |
|---|---|---|
| 1 | IL-11 | MNCVCRLVLVVLSLWPDTAVAPGPPPGPPRVSPDPRAELDSTVLLT |
| | AA sequence | RSLLADTRQLAAQLRDKFPADGDHNLDSLPTLAMSAGALGALQLP |
| | Isoform 1 | GVLTRLRADLLSYLRHVQWLRRAGGSSLKTLEPELGTLQARLDRL |
| | | LRRLQLLMSRLALPQPPPDPPAPPLAPPSSAWGGIRAAHAILGGLHL |
| | | TLDWAVRGLLLLKTRL |
| 2 | IL-11 | MSAGALGALQLPGVLTRLRADLLSYLRHVQWLRRAGGSSL |
| | AA sequence | KTLEPELGTLQARLDRLLRRLQLLMSRLALPQPPPDPPAPPL |
| | Isoform 2 | APPSSAWGGIRAAHAILGGLHLTLDWAVRGLLLLKTRL |
| 3 | IL-11RA | MSSSCSGLSRVLVAVATALVSASSPCPQAWGPPGVQYGQP |
| | AA sequence | GRSVKLCCPGVTAGDPVSWFRDGEPKLLQGPDSGLGHELV |
| | Isoform 1 | LAQADSTDEGTYICQTLDGALGGTVTLQLGYPPARPVVSCQ |
| | | AADYENFSCTWSPSQISGLPTRYLTSYRKKTVLGADSQRRS |
| | | PSTGPWPCPQDPLGAARCVVHGAEFWSQYRINVTEVNPLG |
| | | ASTRLLDVSLQSILRPDPPQGLRVESVPGYPRRLRASWTYPA |
| | | SWPCQPHFLLKFRLQYRPAQHPAWSTVEPAGLEEVITDAVA |
| | | GLPHAVRVSARDFLDAGTWSTWSPEAWGTPSTGTIPKEIPA |
| | | WGQLHTQPEVEPQVDSPAPPRPSLQPHPRLLDHRDSVEQVA |
| | | VLASLGILSFLGLVAGALALGLWLRLRRGGKDGSPKPGFLA |
| | | SVIPVDRRPGAPNL |
| 4 | IL-11RA | MSSSCSGLSRVLVAVATALVSASSPCPQAWGPPGVQYGQP |
| | AA sequence | GRSVKLCCPGVTAGDPVSWFRDGEPKLLQGPDSGLGHELV |
| | Isoform 2 | LAQADSTDEGTYICQTLDGALGGTVTLQLGYPPARPVVSCQ |
| | | AADYENFSCTWSPSQISGLPTRYLTSYRKKTVLGADSQRRS |
| | | PSTGPWPCPQDPLGAARCVVHGAEFWSQYRINVTEVNPLG |
| | | ASTRLLDVSLQSILRPDPPQGLRVESVPGYPRRLRASWTYPA |
| | | SWPCQPHFLLKFRLQYRPAQHPAWSTVEPAGLEEVITDAVA |
| | | GLPHAVRVSARDFLDAGTWSTWSPEAWGTPSTGTIPKEIPA |
| | | WGQLHTQPEVEPQVDSPAPPRPSLQPHPRLLDHRDSVEQVA |
| | | VLASLGILSFLGLVAGALALGLW |
| 5 | IL11 | ACTGCCGCGGCCCTGCTGCTCAGGGCACATGCCTCCCCTCCCCAGGCCGCGGCCCA |
| | cDNA sequence | GCTGACCCTCGGGGCTCCCCCGGCAGCGGACAGGGAAGGGTTAAAGGCCCCCGGC |
| | Isoform 1 | TCCCTGCCCCCTGCCCTGGGGAACCCCTGGCCCTGTGGGGACATGAACTGTGTTTG |
| | | CCGCCTGGTCCTGGTCGTGCTGAGCCTGTGGCCAGATACAGCTGTCGCCCCTGGGC |
| | | CACCACCTGGCCCCCCTCGAGTTTCCCCAGACCCTCGGGCCGAGCTGGACAGCACC |
| | | GTGCTCCTGACCCGCTCTCTCCTGGCGGACACGCGGCAGCTGGCTGCACAGCTGAG |
| | | GGACAAATTCCCAGCTGACGGGGACCACAACCTGGATTCCCTGCCCACCCTGGCCA |
| | | TGAGTGCGGGGGCACTGGGAGCTCTACAGCTCCCAGGTGTGCTGACAAGGCTGCG |
| | | AGCGGACCTACTGTCCTACCTGCGGCACGTGCAGTGGCTGCGCCGGGCAGGTGGCT |
| | | CTTCCCTGAAGACCCTGGAGCCCGAGCTGGGCACCCTGCAGGCCCGACTGGACCGG |
| | | CTGCTGCGCCGGCTGCAGCTCCTGATGTCCCGCCTGGCCCTGCCCCAGCCACCCCC |
| | | GGACCCGCCGGCGCCCCCGCTGGCGCCCCCCTCCTCAGCCTGGGGGGGCATCAGGG |
| | | CCGCCCACGCCATCCTGGGGGGGCTGCACCTGACACTTGACTGGGCCGTGAGGGG |
| | | ACTGCTGCTGCTGAAGACTCGGCTGTGACCCGGGGCCCAAAGCCACCACCGTCCTT |
| | | CCAAAGCCAGATCTTATTTATTTATTTATTTCAGTACTGGGGGCGAAACAGCCAGG |
| | | TGATCCCCCCGCCATTATCTCCCCCTAGTTAGAGACAGTCCTTCCGTGAGGCCTGGG |
| | | GGGCATCTGTGCCTTATTTATACTTATTTATTTCAGGAGCAGGGGTGGGAGGCAGG |
| | | TGGACTCCTGGGTCCCCGAGGAGGAGGGGACTGGGGTCCCGGATTCTTGGGTCTCC |
| | | AAGAAGTCTGTCCACAGACTTCTGCCCTGGCTCTTCCCCATCTAGGCCTGGGCAGG |
| | | AACATATATTATTTATTTAAGCAATTACTTTTCATGTTGGGGTGGGGACGGAGGGG |
| | | AAAGGGAAGCCTGGGTTTTTGTACAAAAATGTGAGAAACCTTTGTGAGACAGAGA |
| | | ACAGGGAATTAAATGTGTCATACATATCCACTTGAGGGCGATTTGTCTGAGAGCTG |
| | | GGGCTGGATGCTTGGGTAACTGGGGCAGGGCAGGTGGAGGGGAGACCTCCATTCA |
| | | GGTGGAGGTCCCGAGTGGGCGGGGCAGCGACTGGGAGATGGGTCGGTCACCCAGA |
| | | CAGCTCTGTGGAGGCAGGGTCTGAGCCTTGCCTGGGGCCCCGCACTGCATAGGGCC |
| | | TTTTGTTTGTTTTTTGAGATGGAGTCTCGCTCTGTTGCCTAGGCTGGAGTGCAGTGA |
| | | GGCAATCTGAGGTCACTGCAACCTCCACCTCCCGGGTTCAAGCAATTCTCCTGCCT |
| | | CAGCCTCCCGATTAGCTGGGATCACAGGTGTGCACCACCATGCCCAGCTAATTATT |
| | | TATTTCTTTTGTATTTTTAGTAGAGACAGGGTTTCACCATGTTGGCCAGGCTGGTTT |
| | | CGAACTCCTGACCTCAGGTGATCCTCCTGCCTCGGCCTCCCAAAGTGCTGGGATTA |
| | | CAGGTGTGAGCCACCACACCTGACCCATAGGTCTTCAATAAATATTTAATGGAAGG |
| | | TTCCACAAGTCACCCTGTGATCAACAGTACCCGTATGGGACAAAGCTGCAAGGTCA |
| | | AGATGGTTCATTATGGCTGTGTTCACCATAGCAAACTGGAAACAATCTAGATATCC |
| | | AACAGTGAGGGTTAAGCAACATGGTGCATCTGTGGATAGAACGCCACCCAGCCGC |
| | | CCGGAGCAGGGACTGTCATTCAGGGAGGCTAAGGAGAGAGGCTTGCTTGGGATAT |
| | | AGAAAGATATCCTGACATTGGCCAGGCATGGTGGCTCACGCCTGTAATCCTGGCAC |
| | | TTTGGGAGGACGAAGCGAGTGGATCACTGAAGTCCAAGAGTTCGAGACCGGCCTG |
| | | CGAGACATGGCAAAACCCTGTCTCAAAAAAGAAAGAATGATGTCCTGACATGAAA |
| | | CAGCAGGCTACAAAACCACTGCATGCTGTGATCCCAATTTTGTGTTTTTCTTTCTAT |
| | | ATATGGATTAAAACAAAAATCCTAAAGGGAAATACGCCAAAATGTTGACAATGAC |
| | | TGTCTCCAGGTCAAAGGAGAGAGGTGGGATTGTGGGTGACTTTTAATGTGTATGAT |
| | | TGTCTGTATTTTACAGAATTTCTGCCATGACTGTGTATTTTGCATGACACATTTTAA |
| | | AAATAATAAACACTATTTTTAGAATAACAGAA |
| 6 | IL11 | ACTGCCGCGGCCCTGCTGCTCAGGGCACATGCCTCCCCTCCCCAGGCCGCG |
| | cDNA sequence | GCCCAGCTGACCCTCGGGGCTCCCCCGGCAGCGGACAGGGAAGGGTTAAA |
| | Isoform 2 | GGCCCCCGGCTCCCTGCCCCCTGCCCTGGGGAACCCCTGGCCCTGTGGGGA |
| | | CATGAACTAGGGACAAATTCCCAGCTGACGGGGACCACAACCTGGATTCC |
| | | CTGCCCACCCTGGCCATGAGTGCGGGGGCACTGGGAGCTCTACAGCTCCCA |
| | | GGTGTGCTGACAAGGCTGCGAGCGGACCTACTGTCCTACCTGCGGCACGTG |
| | | CAGTGGCTGCGCCGGGCAGGTGGCTCTTCCCTGAAGACCCTGGAGCCCGA |
| | | GCTGGGCACCCTGCAGGCCCGACTGGACCGGCTGCTGCGCCGGCTGCAGCT |
| | | CCTGATGTCCCGCCTGGCCCTGCCCCAGCCACCCCCGGACCCGCCGGCGCC |
| | | CCCGCTGGCGCCCCCCTCCTCAGCCTGGGGGGGCATCAGGGCCGCCCACGC |
| | | CATCCTGGGGGGGCTGCACCTGACACTTGACTGGGCCGTGAGGGGACTGCT |
| | | GCTGCTGAAGACTCGGCTGTGACCCGGGGCCCAAAGCCACCACCGTCCTTC |
| | | CAAAGCCAGATCTTATTTATTTATTTATTTCAGTACTGGGGGCGAAACAGC |
| | | CAGGTGATCCCCCCGCCATTATCTCCCCCTAGTTAGAGACAGTCCTTCCGT |
| | | GAGGCCTGGGGGGCATCTGTGCCTTATTTATACTTATTTATTTCAGGAGCA |
| | | GGGGTGGGAGGCAGGTGGACTCCTGGGTCCCCGAGGAGGAGGGGACTGGG |
| | | GTCCCGGATTCTTGGGTCTCCAAGAAGTCTGTCCACAGACTTCTGCCCTGG |
| | | CTCTTCCCCATCTAGGCCTGGGCAGGAACATATATTATTTATTTAAGCAATT |
| | | ACTTTTCATGTTGGGGTGGGGACGGAGGGGAAAGGGAAGCCTGGGTTTTT |
| | | GTACAAAAATGTGAGAAACCTTTGTGAGACAGAGAACAGGGAATTAAATG |
| | | TGTCATACATATCCACTTGAGGGCGATTTGTCTGAGAGCTGGGGCTGGATG |
| | | CTTGGGTAACTGGGGCAGGGCAGGTGGAGGGGAGACCTCCATTCAGGTGG |
| | | AGGTCCCGAGTGGGCGGGGCAGCGACTGGGAGATGGGTCGGTCACCCAGA |
| | | CAGCTCTGTGGAGGCAGGGTCTGAGCCTTGCCTGGGGCCCCGCACTGCATA |
| | | GGGCCTTTTGTTTGTTTTTTGAGATGGAGTCTCGCTCTGTTGCCTAGGCTGG |
| | | AGTGCAGTGAGGCAATCTGAGGTCACTGCAACCTCCACCTCCCGGGTTCAA |
| | | GCAATTCTCCTGCCTCAGCCTCCCGATTAGCTGGGATCACAGGTGTGCACC |
| | | ACCATGCCCAGCTAATTATTTATTTCTTTTGTATTTTTAGTAGAGACAGGGT |
| | | TTCACCATGTTGGCCAGGCTGGTTTCGAACTCCTGACCTCAGGTGATCCTCC |
| | | TGCCTCGGCCTCCCAAAGTGCTGGGATTACAGGTGTGAGCCACCACACCTG |
| | | ACCCATAGGTCTTCAATAAATATTTAATGGAAGGTTCCACAAGTCACCCTG |
| | | TGATCAACAGTACCCGTATGGGACAAAGCTGCAAGGTCAAGATGGTTCATT |
| | | ATGGCTGTGTTCACCATAGCAAACTGGAAACAATCTAGATATCCAACAGTG |
| | | AGGGTTAAGCAACATGGTGCATCTGTGGATAGAACGCCACCCAGCCGCCC |
| | | GGAGCAGGGACTGTCATTCAGGGAGGCTAAGGAGAGAGGCTTGCTTGGGA |
| | | TATAGAAAGATATCCTGACATTGGCCAGGCATGGTGGCTCACGCCTGTAAT |
| | | CCTGGCACTTTGGGAGGACGAAGCGAGTGGATCACTGAAGTCCAAGAGTT |
| | | CGAGACCGGCCTGCGAGACATGGCAAAACCCTGTCTCAAAAAAGAAAGAA |
| | | TGATGTCCTGACATGAAACAGCAGGCTACAAAACCACTGCATGCTGTGATC |
| | | CCAATTTTGTGTTTTTCTTTCTATATATGGATTAAAACAAAAATCCTAAAGG |
| | | GAAATACGCCAAAATGTTGACAATGACTGTCTCCAGGTCAAAGGAGAGAG |
| | | GTGGGATTGTGGGTGACTTTTAATGTGTATGATTGTCTGTATTTTACAGAAT |
| | | TTCTGCCATGACTGTGTATTTTGCATGACACATTTTAAAAATAATAAACACT |
| | | ATTTTTAGAATAACAGAAAAA |
| 7 | IL11RA | AGAGGGCGAGGGCGAGGGCAGAGGGCGCTGGCGGCAGCGGCCGC |
| | cDNA sequence | GGAAGATGAGCAGCAGCTGCTCAGGGCTGAGCAGGGTCCTGGTGG |
| | Isoform 3 | CCGTGGCTACAGCCCTGGTGTCTGCCTCCTCCCCCTGCCCCCAGGC |
| | | CTGGGGCCCCCCAGGGGTCCAGTATGGGCAGCCAGGCAGGTCCGT |
| | | GAAGCTGTGTTGTCCTGGAGTGACTGCCGGGGACCCAGTGTCCTGG |
| | | TTTCGGGATGGGGAGCCAAAGCTGCTCCAGGGACCTGACTCTGGG |
| | | CTAGGGCATGAACTGGTCCTGGCCCAGGCAGACAGCACTGATGAG |
| | | GGCACCTACATCTGCCAGACCCTGGATGGTGCACTTGGGGGCACA |
| | | GTGACCCTGCAGCTGGGCTACCCTCCAGCCCGCCCTGTTGTCTCCT |
| | | GCCAAGCAGCCGACTATGAGAACTTCTCTTGCACTTGGAGTCCCAG |
| | | CCAGATCAGCGGTTTACCCACCCGCTACCTCACCTCCTACAGGAAG |
| | | AAGACAGTCCTAGGAGCTGATAGCCAGAGGAGGAGTCCATCCACA |
| | | GGGCCCTGGCCATGCCCACAGGATCCCCTAGGGGCTGCCCGCTGTG |
| | | TTGTCCACGGGGCTGAGTTCTGGAGCCAGTACCGGATTAATGTGAC |
| | | TGAGGTGAACCCACTGGGTGCCAGCACACGCCTGCTGGATGTGAG |
| | | CTTGCAGAGCATCTTGCGCCCTGACCCACCCCAGGGCCTGCGGGTA |
| | | GAGTCAGTACCAGGTTACCCCCGACGCCTGCGAGCCAGCTGGACA |
| | | TACCCTGCCTCCTGGCCGTGCCAGCCCCACTTCCTGCTCAAGTTCC |
| | | GTTTGCAGTACCGTCCGGCGCAGCATCCAGCCTGGTCCACGGTGGA |
| | | GCCAGCTGGACTGGAGGAGGTGATCACAGATGCTGTGGCTGGGCT |
| | | GCCCCATGCTGTACGAGTCAGTGCCCGGGACTTTCTAGATGCTGGC |
| | | ACCTGGAGCACCTGGAGCCCGGAGGCCTGGGGAACTCCGAGCACT |
| | | GGGACCATACCAAAGGAGATACCAGCATGGGGCCAGCTACACACG |
| | | CAGCCAGAGGTGGAGCCTCAGGTGGACAGCCCTGCTCCTCCAAGG |
| | | CCCTCCCTCCAACCACACCCTCGGCTACTTGATCACAGGGACTCTG |
| | | TGGAGCAGGTAGCTGTGCTGGCGTCTTTGGGAATCCTTTCTTTCCT |
| | | GGGACTGGTGGCTGGGGCCCTGGCACTGGGGCTCTGGCTGAGGCT |
| | | GAGACGGGGTGGGAAGGATGGATCCCCAAAGCCTGGGTTCTTGGC |
| | | CTCAGTGATTCCAGTGGACAGGCGTCCAGGAGCTCCAAACCTGTA |
| | | GAGGACCCAGGAGGGCTTCGGCAGATTCCACCTATAATTCTGTCTT |
| | | GCTGGTGTGGATAGAAACCAGGCAGGACAGTAGATCCCTATGGTT |
| | | GGATCTCAGCTGGAAGTTCTGTTTGGAGCCCATTTCTGTGAGACCC |
| | | TGTATTTCAAATTTGCAGCTGAAAGGTGCTTGTACCTCTGATTTCAC |
| | | CCCAGAGTTGGAGTTCTGCTCAAGGAACGTGTGTAATGTGTACATC |
| | | TGTGTCCATGTGTGACCATGTGTCTGTGAGGCAGGGAACATGTATT |
| | | CTCTGCATGCATGTATGTAGGTGCCTGGGGAGTGTGTGTGGGTCCT |
| | | TGGCTCTTGGCCTTTCCCCTTGCAGGGGTTGTGCAGGTGTGAATAA |
| | | AGAGAATAAGGAAGTTCTTGGAGATTATACTCAGAAAAAAAAA |
| 8 | mIL-11RA1 | MSSSCSGLTRVLVAVATALVSSSSPCPQAWGPPGVQYGQPGRPVMLC |
| | AA sequence | CPGVSAGTPVSWFRDGDSRLLQGPDSGLGHRLVLAQVDSPDEGTYVC |
| | Isoform 1 | QTLDGVSGGMVTLKLGFPPARPEVSCQAVDYENFSCTWSPGQVSGLP |
| | | TRYLTSYRKKTLPGAESQRESPSTGPWPCPQDPLEASRCVVHGAEFWS |
| | | EYRINVTEVNPLGASTCLLDVRLQSILRPDPPQGLRVESVPGYPRRLHA |
| | | SWTYPASWRRQPHFLLKFRLQYRPAQHPAWSTVEPIGLEEVITDAVA |
| | | GLPHAVRVSARDFLDAGTWSAWSPEAWGTPSTGPLQDEIPDWSQGH |
| | | GQQLEAVVAQEDSPAPARPSLQPDPRPLDHRDPLEQVAVLASLGIFSC |
| | | LGLAVGALALGLWLRLRRSGKDGPQKPGLLAPMIPVEKLPGIPNLQRT |
| | | PENFS |
| 9 | mIL-11RA2 | MSSSCSGLTRVLVAVATALVSSSSPCPQAWGPPGVQYGQPGRPVMLC |
| | AA sequence | CPGVSAGTPVSWFRDGDSRLLQGPDSGLGHRLVLAQVDSPDEGTYVC |
| | Isoform 1 | QTLDGVSGGMVTLKLGFPPARPEVSCQAVDYENFSCTWSPGQVSGLP |
| | | TRYLTSYRKKTLPGAESQRESPSTGPWPCPQDPLEASRCVVHGAEFWS |
| | | EYRINVTEVNPLGASTCLLDVRLQSILRPDPPQGLRVESVPGYPRRLHA |
| | | SWTYPASWRRQPHFLLKFRLQYRPAQHPAWSTVEPIGLEEVITDTVAG |
| | | LPHAVRVSARDFLDAGTWSAWSPEAWGTPSTGLLQDEIPDWSQGHG |
| | | QQLEAVVAQEDSLAPARPSLQPDPRPLDHRDPLEQVAVLASLGIFSCL |
| | | GLAVGALALGLWLRLRRSGKEGPQKPGLLAPMIPVEKLPGIPNLQRTP |
| | | ENFS |
| 10 | mIl11RA1 | TATTGGCGCTGGGAGGAGCCGCCCCCACTGCCTGCGCTCAGCACTG |
| | cDNA sequence | GAGGGAGCAGAGGCCGGGGCAGAGGGTGAGGGCGGAGGGCGCTGG |
| | Isoform 2 | CGGCGGCTGCCGCAGAAGATGAGCAGCAGCTGCTCAGGGCTGACCA |
| | | GGGTCCTGGTGGCCGTGGCTACAGCCCTGGTGTCTTCCTCCTCCCCC |
| | | TGCCCCCAAGCTTGGGGTCCTCCAGGGGTCCAGTATGGACAACCTG |
| | | GCAGGCCCGTGATGCTGTGCTGCCCCGGAGTGAGTGCTGGGACTCC |
| | | AGTGTCCTGGTTTCGGGATGGAGATTCAAGGCTGCTCCAGGGACCT |
| | | GACTCTGGGTTAGGACACAGACTGGTCTTGGCCCAGGTGGACAGCC |
| | | CTGATGAAGGCACTTATGTCTGCCAGACCCTGGATGGTGTATCAGG |
| | | GGGCATGGTGACCCTGAAGCTGGGCTTTCCCCCAGCACGTCCTGAA |
| | | GTCTCCTGCCAAGCGGTAGACTATGAAAACTTCTCCTGTACTTGGAG |
| | | TCCAGGCCAGGTCAGCGGTTTGCCCACCCGCTACCTTACTTCCTACA |
| | | GGAAGAAGACGCTGCCAGGAGCTGAGAGTCAGAGGGAAAGTCCAT |
| | | CCACCGGGCCTTGGCCGTGTCCACAGGACCCTCTGGAGGCCTCCCG |
| | | ATGTGTGGTCCATGGGGCAGAGTTCTGGAGTGAGTACCGGATCAAT |
| | | GTGACCGAGGTGAACCCACTGGGTGCCAGCACGTGCCTACTGGATG |
| | | TGAGATTACAGAGCATCTTGCGTCCTGATCCACCCCAAGGACTGCG |
| | | GGTGGAATCCGTACCTGGTTACCCGAGACGCCTGCATGCCAGCTGG |
| | | ACATACCCTGCCTCCTGGCGTCGCCAACCCCACTTTCTGCTCAAGTT |
| | | CCGGTTGCAATACCGACCAGCACAGCATCCAGCCTGGTCCACGGTG |
| | | GAGCCCATTGGCTTGGAGGAAGTGATAACAGATGCTGTGGCTGGGC |
| | | TGCCACACGCGGTACGAGTCAGTGCCAGGGACTTTCTGGATGCTGG |
| | | CACCTGGAGCGCCTGGAGCCCAGAGGCCTGGGGTACTCCTAGCACT |
| | | GGTCCCCTGCAGGATGAGATACCTGATTGGAGCCAGGGACACGGAC |
| | | AGCAGCTAGAGGCAGTAGTAGCTCAGGAGGACAGCCCGGCTCCTGC |
| | | AAGGCCTTCCTTGCAGCCGGACCCAAGGCCACTTGATCACAGGGAC |
| | | CCCTTGGAGCAAGTAGCTGTGTTAGCGTCTCTGGGAATCTTCTCTTG |
| | | CCTTGGCCTGGCTGTTGGAGCTCTGGCACTGGGGCTCTGGCTGAGGC |
| | | TGAGACGGAGTGGGAAGGATGGACCGCAAAAACCTGGGCTCTTGGC |
| | | ACCCATGATCCCGGTGGAAAAGCTTCCAGGAATTCCAAACCTGCAG |
| | | AGGACCCCAGAGAACTTCAGCTGATTTCATCTGTAACCCGGTCAGA |
| | | CTTGGGTGGTTAAAAGGACAGGCAGAAAGAGGCGGGGCAGTGGAT |
| | | CCCTGTGGATGGAGGTCTCAGCTGAAAGTCTGAGCTCTTTTCTTTGA |
| | | CACCTATACTCCAAACTTGCTGCCGGCTGAAGGCTGTCTGGACTTCC |
| | | GATGTCCTGAGGTGGAAGTCCACCTGAGGAATGTGTACAGAAGTCT |
| | | GTGTTCCTGTGATCGTGTGTGTATGTGAGACAGGGAGCAAAAGTTCT |
| | | CTGCATGTGTGTACAGATGATTGGAGAGTGTGTGCGGTCTTGGGCTT |
| | | GGCCCTTCTGGGAAGTGTGAAGAGTTGAAATAAAAGAGACGGAAGT |
| | | TTTTGG |
| 11 | mIl11RA2 | GACTCAGTCCAGACCCCTTCCCCCCCGCCTGGGGTCAGGGCTGGACAGGGAGGAG |
| | cDNA sequence | AAGCGAGCTCCTTGCAAGACGAGTTGGGGACGAGATAGCTAGTGACCCTGGCCTT |
| | | GGCTGACCCGAAAGAGGCCTGGAGACATCTGTCCTCAAAGGATTGTCCACTTCCG |
| | | GTGACTCCCTGCTGCTGATAATAGGGCCCATGTTTCCCAGAAGGCCCGAGGTTATC |
| | | CCAAGACTGCTTCTCTCCCTTCACCTCCCATGGCTTCATTCCTGAAGAGCCTGTGA |
| | | TCACTTAGGGTCAGAAATAGACGGATGAGCAGCAGCTGCTCAGGGCTGACCAGGG |
| | | TCCTGGTGGCCGTGGCTACAGCCCTGGTGTCTTCCTCCTCCCCCTGCCCCCAAGCT |
| | | TGGGGTCCTCCAGGGGTCCAGTATGGACAACCTGGCAGGCCCGTGATGCTGTGCT |
| | | GCCCCGGAGTGAGTGCTGGGACTCCAGTGTCCTGGTTTCGGGATGGAGATTCAAG |
| | | GCTGCTCCAGGGACCTGACTCTGGGTTAGGACACAGACTGGTCTTGGCCCAGGTG |
| | | GACAGCCCTGATGAAGGCACTTATGTCTGCCAGACCCTGGATGGTGTATCAGGGG |
| | | GCATGGTGACCCTGAAGCTGGGCTTTCCCCCAGCACGTCCTGAAGTCTCTTGCCAA |
| | | GCGGTAGACTATGAAAACTTCTCCTGTACTTGGAGTCCAGGCCAGGTCAGCGGTTT |
| | | GCCCACCCGCTACCTTACTTCCTACAGGAAGAAGACGCTGCCAGGAGCTGAGAGT |
| | | CAGAGGGAAAGTCCATCCACCGGGCCTTGGCCGTGTCCACAGGACCCTCTGGAGG |
| | | CCTCCCGATGTGTGGTCCATGGGGCAGAGTTCTGGAGTGAGTACCGGATCAATGT |
| | | GACCGAGGTGAACCCACTGGGTGCCAGCACGTGCCTACTGGATGTGAGATTACAG |
| | | AGCATCTTGCGTCCCGATCCACCCCAAGGACTGCGGGTGGAATCCGTACCTGGTT |
| | | ACCCAAGACGCCTGCATGCCAGCTGGACATACCCTGCCTCCTGGCGTCGCCAACC |
| | | CCACTTTCTGCTCAAGTTCCGGTTGCAATACCGACCAGCACAGCATCCAGCCTGGT |
| | | CCACGGTGGAGCCCATTGGCTTGGAGGAAGTGATAACAGATACTGTGGCTGGGCT |
| | | GCCCCACGCGGTACGAGTCAGTGCCAGGGACTTTCTGGATGCTGGCACCTGGAGC |
| | | GCCTGGAGCCCAGAGGCCTGGGGTACTCCTAGCACTGGTCTCCTGCAGGATGAGA |
| | | TACCTGATTGGAGCCAGGGACATGGACAGCAGCTAGAGGCAGTAGTAGCTCAGG |
| | | AGGACAGCCTGGCTCCTGCAAGGCCTTCCTTGCAGCCAGACCCAAGGCCACTTGA |
| | | TCACAGGGATCCCTTGGAGCAAGTAGCTGTGTTAGCATCTCTGGGAATCTTCTCTT |
| | | GCCTTGGCCTGGCTGTTGGAGCCCTGGCACTGGGGCTCTGGCTGAGGCTGAGACG |
| | | GAGTGGGAAGGAAGGACCGCAAAAACCTGGGCTCTTGGCACCCATGATCCCGGTG |
| | | GAAAAGCTTCCAGGAATTCCAAACCTGCAGAGGACCCCAGAGAACTTCAGCTGAT |
| | | TTCATCTGTAACCCGGTCAGACTTGGGTGGTTAAAAGGACAGGCAGAAAGAGGCG |
| | | GGGCAGTGGATCCCTGTGGATGGAGGTCTCAGCTGGAAGTCTGAGCTCTTTTCTTT |
| | | GACACCTATACTCCAAACTTGCTGCCGGCTGAAGGCTGTCTGGACTTCTGATGTCC |
| | | TGAGGTGGAAGTCCACCTGAGGAATGTGTACAGAAGTCTGTGTTCCTGTGATCGT |
| | | GTGTGTATGTGAGACAGGGAGCAAAAGTTCTCTGCATGTGTGTACACATGATTGG |
| | | AGAGTGTGTGCGGTCTTGGGCTTGGCCCTTCTGGGGAGTGTGAAGAGTTGAAATA |
| | | AAAGAGACGGAAGTTTTTGGAGATTGTCGTCTAATGTTGCTTTATTAGTTTTGCTG |
| | | TTGGGGGTGTGAGTACATTTTCTTTTGTAGTACCAGATTTAAACTGGGTACAGTTC |
| | | CTTGGAGCCTTTTCCCTTGGCGTTCTAACCACCGAGCCAGGCTGCGGTTCTGGGGA |
| | | TGAACACAGACACTGCGCCGAGCCAGGTGAAGCCTGAGAAGGGAAAAGTCCAAA |
| | | GCTAGGCTCACATTTGGGGGATGAGCTTAGGTAGGGTTCAGGGGTTAGGGGTAAA |
| | | AGCCCGATGAAAATCCGGGTAGGTATTAGAGTTAGTAAAGGGGCTAGGGATGAAT |
| | | ATTGGGGGTTCGGACTGAGGGCAGAGTTAGAGGTATCAAAGTTGGGGGCAGAGCT |
| | | CACACGACAGCCTGGAGGTGACAGTCCCCATCAGCCTCCTGCAGTTCCATGTGGA |
| | | CAATCCTCCTCAGCAGCCTGCTTGGGAGTGGCTGTCTATAGAGCTGAGTACAGCA |
| | | GCTAGTGCTGGAGGGCAGAGGCAAGGCTGGAGAACAATAGGGCCACACTTCAGA |
| | | GAGGTCTGGACCCACAAGCTGCTCTGATTACATAGCTAGAGGTCCAACTTTCTGG |
| | | GCCCTGTTCCCTGTAGTCCCCTCGCTCAGCATTCATTCTATGGCCTCTGAGAACTTA |
| | | CCTGCTTCAGGAGCCGGGCTCAGAAGCAACAGTAACAGCGGGAGGCTGCTGGCG |
| | | GGGGAGAGCCCCTCCATCATGCTCACTCAG |
| 12 | hIL-6 | MLTLQTWLVQALFIFLTTESTGELLDPCGYISPESPVVQLHSNFTAVCVLKEKCMDYFHV |
| | receptor | NANYIVWKTNHFTIPKEQYTIINRTASSVTFTDIASLNIQLTCNILTFGQLEONVYGITI |
| | subunit | ISGLPPEKPKNLSCIVNEGKKMRCEWDGGRETHLETNFTLKSEWATHKFADCKAKRDTPT |
| | beta | SCTVDYSTVYFVNIEVWVEAENALGKVTSDHINFDPVYKVKPNPPHNLSVINSEELSSIL |
| | AA sequence | KLTWTNPSIKSVIILKYNIQYRTKDASTWSQIPPEDTASTRSSFTVQDLKPFTEYVFRIR |
| | | CMKEDGKGYWSDWSEEASGITYEDRPSKAPSFWYKIDPSHTQGYRTVQLVWKTLPPFEAN |
| | | GKILDYEVTLTRWKSHLQNYTVNATKLTVNLTNDRYLATLTVRNLVGKSDAAVLTIPACD |
| | | FQATHPVMDLKAFPKDNMLWVEWTTPRESVKKYILEWCVLSDKAPCITDWQQEDGTVHRT |
| | | YLRGNLAESKCYLITVTPVYADGPGSPESIKAYLKQAPPSKGPTVRTKKVGKNEAVLEWD |
| | | QLPVDVQNGFIRNYTIFYRTIIGNETAVNVDSSHTEYTLSSLTSDTLYMVRMAAYTDEGG |
| | | KDGPEFTFTTPKFAQGEIEAIVVPVCLAFLLTTLLGVLFCFNKRDLIKKHIWPNVPDPSK |
| | | SHIAQWSPHTPPRHNFNSKDQMYSDGNFTDVSVVEIEANDKKPFPEDLKSLDLFKKEKIN |
| | | TEGHSSGIGGSSCMSSSRPSISSSDENESSQNTSSTVQYSTVVHSGYRHQVPSVQVFSRS |
| | | ESTQPLLDSEERPEDLQLVDHVDGGDGILPRQQYFKQNCSQHESSPDISHFERSKQVSSV |
| | | NEEDFVRLKQQISDHISQSCGSGQMKMFQEVSAADAFGPGTEGQVERFETVGMEAATDEG |
| | | MPKSYLPQTVRQGGYMPQ |
| 13 | hIL-64 | MLTLQTWLVQALFIFLTTESTGELLDPCGYISPESPVVQLHSNFTAVCVLKEKCMDYFHV |
| | subunit | NANYIVWKTNHFTIPKEQYTIINRTASSVTFTDIASLNIQLTCNILTFGQLEQNVYGITI |
| | beta | ISGLPPEKPKNLSCIVNEGKKMRCEWDGGRETHLETNFTLKSEWATHKFADCKAKRDTPT |
| | Isoform 2; | SCTVDYSTVYFVNIEVWVEAENALGKVTSDHINFDPVYKVKPNPPHNLSVINSEELSSIL |
| | AA seq. | KLTWTNPSIKSVIILKYNIQYRTKDASTWSQIPPEDTASTRSSFTVQDLKPFTEYVFRIR |
| | | CMKEDGKGYWSDWSEEASGITYEDNIASF |
| 16 | mIL-11 | MNCVCRLVLVVLSLWPDRVVAPGPPAGSPRVSSDPRADLDSAVLLTRSLLADTRQLAAQM |
| | AA Seq. | RDKFPADGDHSLDSLPTLAMSAGTLGSLQLPGVLTRLRVDLMSYLRHVQWLRRAGGPSLK |
| | | TLEPELGALQARLERLLRRLOLLMSRLALPQAAPDQPVIPLGPPASAWGSIRAAHAILGG |
| | | LHLTLDWAVRGLLLLKTRL |
| 17 | Macaca IL-11 | MNCVCRLVLVVLSLWPDTAVAPGPPPGSPRASPDPRAELDSTVLLTRSLLEDTRQLTIQL |
| | AA Seq. | KDKFPADGDHNLDSLPTLAMSAGALGALQLPSVLTRLRADLLSYLRHVQWLRRAMGSSLK |
| | | TLEPELGTLQTRLDRLLRRLQLLMSRLALPQLPPDPPAPPLAPPSSTWGGIRAAHAILGG |
| | | LHLTLDWAVRGLLLLKTRL |
| 18 | rat IL-11 | MNCVCRLVLVVLSLWPDRVVAPGPPAGSPRVSSDPRADLDSAVLLTRSLLADTRQLAAQM |
| | AA Seq. | RDKFPADGDHNLDSLPTLAMSAGTLGSLQLPGVLTRLRVDLMSYFRHVQWLRRAAGPSLK |
| | | TLEPELGALQARLERLLRRLQLLMSRLALPQAAPDQPAVPLGPPASAWGSIRAAHAILGG |
| | | LHLTLDWAVRGLLLLKTRL |
| 22 | Macaca | MSSGCSGLSRVLVAVATALVSASSSCPQAWGPPGVQYGQPGRSVKLCCPGVTAGDPVSWFRDGE |
| | IL-11Ra | PRLLQG |
| | isoform X4; | PDSGLGHELVLTQADSTDEGTYICQTLDGALGGTVTLQLGYPPARPVVSCQAADYENFSCTWSP |
| | AA seq. | SQISGL |
| | | PTRYLTSYRKKTVLGADSQRRSPSTGPWPCPQDPLGAARCVVHGAEFWSQYRINVTEVNPLGAS |
| | | TRLLDV |
| | | SLQSILRPDPPQGLRVESVPGFPRRLRASWTYPASWPRQPHFLLKFRLQYRPAQHPAWSTVRPG |
| | | VEPAGL |
| | | EEMITDAVAGLPHAVRVSARDFLDAGTWSTWSPEAWGTPSTGTVPKEVPAWGQLHMQPEVEPQV |
| | | DSPAPP |
| | | SPSLQPHPRLLDHRDSVEQVAVLASLGILSFLGLMAGALALGLWSSKPIEDPGGLRQSPLIILS |
| | | CWCGWM |
| | | DSRSLWLGLSWKFCLEPISVRPCISNLPAERCLYL |
| 23 | Rat IL-11Ra | MSSSRSGLTRVLVAVATALVSSSTPCPQAWGPPGVQYGQPGRPVMLCCPGVNAGTPVSWF |
| | AA seq. | RDGDSRLLQGPDSGLGHRLVLAQVDSRDEGTYVCRTLDGVFGGMVTLKLGSPPARPEVSC |
| | | QAVDYENFSCTWSPGRVSGLPTRYLTSYRKKTLPGAESQRESPSTGPWPCPQDPLEASRC |
| | | VVHGAEFWSEYRINVTEVNPLGASTCLLDVRLQRILRPDPPQGLRVESVPGYPRRLHASW |
| | | TYPASWRRQPHFLLKFRLQYRPAQHPAWSTVEPIGLEELITDAVAGLPHAVRVSARDFLD |
| | | AGTWSAWSPEAWGTPSTGPLRDEVPDGSRGHEQKLEAAAQEDSPAPPSPSLOPDPRPLDH |
| | | RDPLEQVAVLASLGIFSFLGLAVGALALGLWLRLRRSGKDGPQKPGFLAPMIPGDKLPGI |
| | | PNLQRTPENFS |
| 26 | mIL-6ST | MSAPRIWLAQALLFFLTTESIGQLLEPCGYIYPEFPVVQRGSNFTAICVLKEACLQHYYV |
| | AA seq. | NASYIVWKTNHAAVPREQVTVINRTTSSVTFTDVVLPSVQLTCNILSFGQIEQNVYGVTM |
| | | LSGFPPDKPTNLTCIVNEGKNMLCQWDPGRETYLETNYTLKSEWATEKFPDCQSKHGTSC |
| | | MVSYMPTYYVNIEVWVEAENALGKVSSESINFDPVDKVKPTPPYNLSVTNSEELSSILKL |
| | | SWVSSGLGGLLDLKSDIQYRTKDASTWIQVPLEDTMSPRTSFTVQDLKPFTEYVFRIRSI |
| | | KDSGKGYWSDWSEEASGTTYEDRPSRPPSFWYKTNPSHGQEYRSVRLIWKALPLSEANGK |
| | | ILDYEVILTQSKSVSQTYTVTGTELTVNLTNDRYVASLAARNKVGKSAAAVLTIPSPHVT |
| | | AAYSVVNLKAFPKDNLLWVEWTPPPKPVSKYILEWCVLSENAPCVEDWQQEDATVNRTHL |
| | | RGRLLESKCYQITVTPVFATGPGGSESLKAYLKQAAPARGPTVRTKKVGKNEAVLAWDQI |
| | | PVDDQNGFIRNYSISYRTSVGKEMVVHVDSSHTEYTLSSLSSDTLYMVRMAAYTDEGGKD |
| | | GPEFTFTTPKFAQGEIEAIVVPVCLAFLLTTLLGVLFCFNKRDLIKKHIWPNVPDPSKSH |
| | | IAQWSPHTPPRHNFNSKDQMYSDGNFTDVSVVEIEANNKKPCPDDLKSVDLFKKEKVSTE |
| | | GHSSGIGGSSCMSSSRPSISSNEENESAQSTASTVQYSTVVHSGYRHQVPSVQVFSRSES |
| | | TQPLLDSEERPEDLQLVDSVDGGDEILPROSYFKQNCSQPEACPEISHFERSNQVLSGNE |
| | | EDFVRLKQQQVSDHISQPYGSEQRRLFQEGSTADALGTGADGQMERFESVGMETTIDEEI |
| | | PKSYLPOTVROGGYMPQ |
| 27 | Macaca IL6R | MKYPHKMLTLQTWVVQALFIFLTTESIGELLDPCGYISPESPVVQLHSNFTAVCVLKEKCMDYF |
| | subunit | HVNANY |
| | beta | IVWKTNHFTIPKEQYTIINRTASSVTFTDISSLNIQLTCNILTFGQLEQNVYGITIISGLPPEK |
| | isoform X1 | PKNLSC |
| | AA seq. | IVNEGKKMRCEWNRGRETHLETNFTLKSEWATHKFADCKAKRDTPTSCTVDYSTVYFVNIEVWV |
| | | EAENAL |
| | | GKVTSDHINFDPVYKVKPNPPHNLSVINSEELSSILKLTWTNPSIKSVIRLKYNIQYRTKDAST |
| | | WSQIPP |
| | | EDTASTRSSFTVQDLKPFTEYVFRICCMKEDGKGYWSDWSEEANGITYEDRPSKAPSFWYKIDP |
| | | SHAQGY |
| | | RTVQLMWKTLPPFEANGKILDYEVTLTRWKSHLQNYTVNDTKLTVNLTNDRYVATLTARNLVGK |
| | | SDAAVL |
| | | TIPACDFQATHPVMDLKAFPKDNMLWVEWTTPRESVKKYILEWCVLSDKAPCIADWQQEDGTVH |
| | | RTHLRG |
| | | NLAESKCYLITVTPVYADGPGSPESIKAYLKQAPPSKGPTVRTKKVGKNEAVLEWDQLPVDVQN |
| | | GFIRNY |
| | | TIFYRTIIGNETAVNVDSSHTEYTLSSLTSDTLYMVRMAAYTDEGGKDGPEFTFTTPKFAQGEI |
| | | EAIVVP |
| | | VCLAFLLTTLLGVLFCFNKRDLIKKHIWPNVPDPSKSHIAQWSPHTPPRHNFSSKDQMYSDGNF |
| | | TDVSVV |
| | | EIEANDKKPFPEDLKSLDLFKKEKINTEGHSSGIGGSSCMSSSRPSISSSDENESSQNTSSTVQ |
| | | YSTVVH |
| | | SGYRHQVPSVQVFSRSESTQPLLDSEERPEDLQLVDHVDGSDDILPRQQYFKQNCSQHESSPDI |
| | | SHFERS |
| | | KQVSSVNEEDFVRLKQQISDHISQSCGSGEMKMFQEVSAADPFGPGTEGQIERFETIGMEAAID |
| | | EGMPKS |
| | | YLPQTVRQGGYMPQ |
| 28 | rat IL6R | MSALRIWLMQALLIFLTTESIGQLVEPCGYIYPEFPVVQRGSNFTATCVLKEKCLQVYSV |
| | subunit | NATYIVWKTNHVAVPKEQVTVINRTASSVTFTDVVFQNVQLTCNILSFGQIEQNVYGITI |
| | beta | LSGYPPDIPTNLSCIVNEGKNMLCQLDPGRETYLETNYTLKSEWATEKFPDCRTKHGTSS |
| | AA seq. | CMMGYTPIYFVNIEVWVEAENALGNVSSEPINFDPVDKVKPSPPHNLSVTNSEELSSILK |
| | | LAWVNSGLDSILRLKSDIQYRTKDASTWIQVPLEDTVSPRTSFTVQDLKPFTEYVERIRS |
| | | IKENGKGYWSDWSEEASGTTYEDRPSKAPSFWYKVNANHPQEYRSARLIWKTLPLSEANG |
| | | KILDYEVVLTQSKSVSQTYTVNGTELIVNLTNNRYVASLAARNVVGKSPATVLTIPGSHF |
| | | KASHPVVDLKAFPKDNLLWVEWTPPSKPVNKYILEWCVLSENSPCIPDWQQEDGTVNRTH |
| | | LRGSLLESKCYLITVTPVFPGGPGSPESMKAYLKQAAPSKGPTVRTKKVGKNEAVLEWDH |
| | | LPVDVQNGFIRNYSISYRTSVGKEMVVRVDSSHTEYTLSSLSSDTLYMVHMAAYTEEGGK |
| | | DGPEFTFTTLKFAQGEIEAIVVPVCLAFLLTTLLGVLFCFNKRDLIKKHIWPNVPDPSKS |
| | | HIAQWSPHTPPRHNFNSKDQMYSDANFTDVSVVEIEANNKKPCPDDLKSLDLFKKEKIST |
| | | EGHSSGIGGSSCMSSSRPSISSSEENESAQSTASTVQYSTVVHSGYRHQVPSVQVFSRSE |
| | | STQPLLDSEERPEDLQLVDSVDSGDEILPRQQYFKQSCSQPGASPDVSHFGRSSQVPSGS |
| | | EEDFVRLKQQQVSDHISEPYGSEQRRLFQEGSVADALGTGTDGQIERFESVGMETAMDED |
| | | ISKSYLPQTVRQGGYMPQ |

Antibody Sequences

| | SEQ | |
|---|---|---|
| | ID | |
| Type | NO | Sequence |
| PRT | 32 | EVQLLESGGGLVQPGGSLRLSCAASGFTFSSYGMHWVRQAPGKGLEWVSV |
| | | ISYDGSYKYYADSVKGRFTISRDNSKNTLYLQMNSLRAEDTAVYYCAAGV |
| | | PDYWGQGTLVTVSS |
| PRT | 33 | SYGMH |
| PRT | 34 | VISYDGSYKYYADSVKG |
| PRT | 35 | GVPDY |
| PRT | 36 | QSVLTQPPSASGTPGQRVTISCTGSSSNIGAGYDVHWYQQLPGTAPKLLIYS |
| | | NNERPSGVPDRFSGSKSGTSASLAISGLRSEDEADYYCAAWDDSLNGPVFG |
| | | GGTKLTVL |
| PRT | 37 | TGSSSNIGAGYDVH |
| PRT | 38 | SNNERPS |
| PRT | 39 | AAWDDSLNGPV |
| PRT | 40 | EVQLLESGGGLVQPGGSLRLSCAASGFTFSSYGMHWVRQAPGKGLEWVSV |
| | | ISYDGSYKYYADSVKGRFTISRDNSKNTLYLQMNSLRAEDTAVYYCAAGV |
| | | PDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| PRT | 41 | QSVLTQPPSASGTPGQRVTISCTGSSSNIGAGYDVHWYQQLPGTAPKLLIYS |
| | | NNERPSGVPDRFSGSKSGTSASLAISGLRSEDEADYYCAAWDDSLNGPVFG |
| | | GGTKLTVLGQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| PRT | 42 | EVQLLESGGGLVQPGGSLRLSCAASGFTFSDYYMSWIRQAPGKGLEWVSGI |
| | | NWNGGSTGYADSVKGRFTISRDNSKNTLYLQMNSLRAEDTAVYYCATWD |
| | | GPFDYWGQGTLVTVSS |
| PRT | 43 | DYYMS |
| PRT | 44 | GINWNGGSTGYADSVKG |
| PRT | 45 | WDGPFDY |
| PRT | 46 | QSVLTQPPSASGTPGQRVTISCTGSSSNIGAGYDVHWYQQLPGTAPKLLIYY |
| | | DDLLPSGVPDRFSGSKSGTSASLAISGLRSEDEADYYCQSYDSSLGGSVFGG |
| | | GTKLTVL |
| PRT | 47 | TGSSSNIGAGYDVH |
| PRT | 48 | YDDLLPS |
| PRT | 49 | QSYDSSLGGSV |
| PRT | 50 | EVQLLESGGGLVQPGGSLRLSCAASGFTFSDYYMSWIRQAPGKGLEWVSGI |
| | | NWNGGSTGYADSVKGRFTISRDNSKNTLYLQMNSLRAEDTAVYYCATWD |
| | | GPFDYWGQGTLVTVSSAKTTPPSVYPLAPGSAAQTNSMVTLGCLVKGYFP |
| | | EPVTVTWNSGSLSSGVHTFPAVLQSDLYTLSSSVTVPSSTWPSETVTCNVAH |
| | | PASSTKVDKKIVPRDCGCKPCICTVPEVSSVFIFPPKPKDVLTITLTPKVTCV |
| | | VVDISKDDPEVQFSWFVDDVEVHTAQTQPREEQFNSTFRSVSELPIMHQDW |
| | | LNGKEFKCRVNSAAFPAPIEKTISKTKGRPKAPQVYTIPPPKEQMAKDKVSL |
| | | TCMITDFFPEDITVEWQWNGQPAENYKNTQPIMDTDGSYFVYSKLNVQKS |
| | | NWEAGNTFTCSVLHEGLHNHHTEKSLSHSPGK |
| PRT | 51 | QSVLTQPPSASGTPGQRVTISCTGSSSNIGAGYDVHWYQQLPGTAPKLLIYY |
| | | DDLLPSGVPDRFSGSKSGTSASLAISGLRSEDEADYYCQSYDSSLGGSVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 52 | EVQLLESGGGLVQPGGSLRLSCAASGFTFSSYGMHWVRQAPGKGLEWVSV |
| | | ISYDGSYKYYADSVKGRFTISRDNSKNTLYLQMNSLRAEDTAVYYCAAGV |
| | | PDYWGQGTLVTVSSAKTTPPSVYPLAPGSAAQTNSMVTLGCLVKGYFPEP |
| | | VTVTWNSGSLSSGVHTFPAVLQSDLYTLSSSVTVPSSTWPSETVTCNVAHP |
| | | ASSTKVDKKIVPRDCGCKPCICTVPEVSSVFIFPPKPKDVLTITLTPKVTCVV |
| | | VDISKDDPEVQFSWFVDDVEVHTAQTQPREEQFNSTFRSVSELPIMHQDWL |
| | | NGKEFKCRVNSAAFPAPIEKTISKTKGRPKAPQVYTIPPPKEQMAKDKVSLT |
| | | CMITDFFPEDITVEWQWNGQPAENYKNTQPIMDTDGSYFVYSKLNVQKSN |
| | | WEAGNTFTCSVLHEGLHNHHTEKSLSHSPGK |
| PRT | 53 | QSVLTQPPSASGTPGQRVTISCTGSSSNIGAGYDVHWYQQLPGTAPKLLIYS |
| | | NNERPSGVPDRFSGSKSGTSASLAISGLRSEDEADYYCAAWDDSLNGPVFG |
| | | GGTKLTVLGQPKSSPSVTLFPPSSEELETNKATLVCTITDFYPGVVTVDWKV |
| | | DGTPVTQGMETTQPSKQSNNKYMASSYLTLTARAWERHSSYSCQVTHEGH |
| | | TVEKSLSRADCS |
| PRT | 54 | EVQLLESGGGLVQPGGSLRLSCAASGFTFSHYDMNWLRQAPGKGLEWVG |
| | | VISYDGSNKFYADFVKGRFTISRDNSKNTLYLQMNSLRAEDTAVYYCARTP |
| | | GSHFDYWGQGTLVTVSS |
| PRT | 55 | HYDMN |
| PRT | 56 | VISYDGSNKFYADFVKG |
| PRT | 57 | TPGSHFDY |
| PRT | 58 | QSVLTQPPSASGTPGQRVTISCSGSSSNIGNNAVNWYQQLPGTAPKLLIYYD |
| | | DLLPSGVPDRFSGSKSGTSASLAISGLRSEDEADYYCSAWDDSLNGVVFGG |
| | | GTKLTVL |
| PRT | 59 | SGSSSNIGNNAVN |
| PRT | 60 | YDDLLPS |
| PRT | 61 | SAWDDSLNGVV |
| PRT | 62 | EVQLLESGGGLVQPGGSLRLSCAASGFTFSHYDMNWLRQAPGKGLEWVG |
| | | VISYDGSNKFYADFVKGRFTISRDNSKNTLYLQMNSLRAEDTAVYYCARTP |
| | | GSHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 63 | QSVLTQPPSASGTPGQRVTISCSGSSSNIGNNAVNWYQQLPGTAPKLLIYYD |
| | | DLLPSGVPDRFSGSKSGTSASLAISGLRSEDEADYYCSAWDDSLNGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 64 | EVQLLESGGGLVQPGGSLRLSCAASGFTFSIYAMSWVRQAPGKGLEWVSSI |
| | | SDTGDRTYYADSVKGRFTISRDNSKNTLYLQMNSLRAEDTAVYYCARSNW |
| | | GSLDYWGQGTLVTVSS |
| PRT | 65 | IYAMS |
| PRT | 66 | SISDTGDRTYYADSVKG |
| PRT | 67 | SNWGSLDY |
| PRT | 68 | QSVLTQPPSASGTPGQRVTISCSGSSSNIGSNYVYWYQQLPGTAPKLLIYYD |
| | | DLLPSGVPDRFSGSKSGTSASLAISGLRSEDEADYYCAAWDDSLSGRVFGG |
| | | GTKLTVL |
| PRT | 69 | SGSSSNIGSNYVY |
| PRT | 70 | YDDLLPS |
| PRT | 71 | AAWDDSLSGRV |
| PRT | 72 | EVQLLESGGGLVQPGGSLRLSCAASGFTFSIYAMSWVRQAPGKGLEWVSSI |
| | | SDTGDRTYYADSVKGRFTISRDNSKNTLYLQMNSLRAEDTAVYYCARSNW |
| | | GSLDYWGQGTLVTVSSAKTTPPSVYPLAPGSAAQTNSMVTLGCLVKGYFP |
| | | EPVTVTWNSGSLSSGVHTFPAVLQSDLYTLSSSVTVPSSTWPSETVTCNVAH |
| | | PASSTKVDKKIVPRDCGCKPCICTVPEVSSVFIFPPKPKDVLTITLTPKVTCV |
| | | VVDISKDDPEVQFSWFVDDVEVHTAQTQPREEQFNSTFRSVSELPIMHQDW |
| | | LNGKEFKCRVNSAAFPAPIEKTISKTKGRPKAPQVYTIPPPKEQMAKDKVSL |
| | | TCMITDFFPEDITVEWQWNGQPAENYKNTQPIMDTDGSYFVYSKLNVQKS |
| | | NWEAGNTFTCSVLHEGLHNHHTEKSLSHSPGK |
| PRT | 73 | QSVLTQPPSASGTPGQRVTISCSGSSSNIGSNYVYWYQQLPGTAPKLLIYYD |
| | | DLLPSGVPDRFSGSKSGTSASLAISGLRSEDEADYYCAAWDDSLSGRVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 74 | EVQLLESGGGLVQPGGSLRLSCAASGFTFSSYGMHWVRQAPGKGLEWVSV |
| | | ISYDGSYKYYADSVKGRFTISRDNSKNTLYLQMNSLRAEDTAVYYCAAGV |
| | | PDYWGQGTLVTVSSGGGGSGGGGSGGGGSQSVLTQPPSASGTPGQRVTISC |
| | | TGSSSNIGAGYDVHWYQQLPGTAPKLLIYSNNERPSGVPDRFSGSKSGTSAS |
| | | LAISGLRSEDEADYYCAAWDDSLNGPVFGGGTKLTVLGGGGSGGGGSGGG |
| | | GSGVECPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYK |
| | | CKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGNV |
| | | FSCSVMHEALHNHYTQKSLSLSPG |
| PRT | 75 | EVQLLESGGGLVQPGGSLRLSCAASGFTFSHYDMNWLRQAPGKGLEWVG |
| | | VISYDGSNKFYADFVKGRFTISRDNSKNTLYLQMNSLRAEDTAVYYCARTP |
| | | GSHFDYWGQGTLVTVSSGGGGSGGGGSGGGGSQSVLTQPPSASGTPGQRV |
| | | TISCSGSSSNIGNNAVNWYQQLPGTAPKLLIYYDDLLPSGVPDRFSGSKSGT |
| | | SASLAISGLRSEDEADYYCSAWDDSLNGVVFGGGTKLTVLGGGGSGGGGS |
| | | GGGGSGVECPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLSCAVK |
| | | GFYPSDIAVEWESNGQPENNYKTTPPVLDSDGSFFLVSKLTVDKSRWQQGN |
| | | VFSCSVMHEALHNHYTQKSLSLSPG |
| PRT | 76 | EVQLVQSGAEVKKPGASVKISCKASGYTFTDYNMDWVKQAPGQRLEWIG |
| | | DINPHNGGPIYNQKFTGRATLTVDKSASTAYMELSSLRSEDTAVYYCARGE |
| | | LGHWYFDVWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTYIC |
| | | NVNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| PRT | 77 | DIVLTQSPASLALSPGERATLSCRASKSVSTSGYSYIHWYQQKPGQPPRLLIY |
| | | LASNLDSGVPARFSGSGSGTDFTLNIHPLEEEDFATYYCQHSRDLPPTFGQG |
| | | TKLEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSS |
| | | PVTKSFNRGEC |
| PRT | 78 | EVQLVQSGAEVKKPGASVKISCKASGYTFTDYNMDWVKQAPGQRLEWIG |
| | | DINPHNGGPIYNQKFTGRATLTVDKSASTAYMELSSLRSEDTAVYYCARGE |
| | | LGHWYFDVWGQGTTVTVSSAKTTPPSVYPLAPGSAAQTNSMVTLGCLVK |
| | | GYFPEPVTVTWNSGSLSSGVHTFPAVLQSDLYTLSSSVTVPSSTWPSETVTC |
| | | NVAHPASSTKVDKKIVPRDCGCKPCICTVPEVSSVFIFPPKPKDVLTITLTPK |
| | | VTCVVVDISKDDPEVQFSWFVDDVEVHTAQTQPREEQFNSTFRSVSELPIM |
| | | HQDWLNGKEFKCRVNSAAFPAPIEKTISKTKGRPKAPQVYTIPPPKEQMAK |
| | | DKVSLTCMITDFFPEDITVEWQWNGQPAENYKNTQPIMDTDGSYFVYSKL |
| | | NVQKSNWEAGNTFTCSVLHEGLHNHHTEKSLSHSPGK |
| PRT | 79 | DIVLTQSPASLALSPGERATLSCRASKSVSTSGYSYIHWYQQKPGQPPRLLIY |
| | | LASNLDSGVPARFSGSGSGTDFTLNIHPLEEEDFATYYCQHSRDLPPTFGQG |
| | | TKLEIKRADAAPTVSIFPPSSEQLTSGGASVVCFLNNFYPKDINVKWKIDGSE |
| | | RQNGVLNSWTDQDSKDSTYSMSSTLTLTKDEYERHNSYTCEATHKTSTSPI |
| | | VKSFNRNEC |
| PRT | 80 | EVQLLESGGGLVQPGGSLRLSCAASGFTFSSYGMHWVRQAPGKGLEWVSV |
| | | ISYDGSYKYYADSVKGRFTISRDNSKNTLYLQMNSLRAEDTAVYYCAAGV |
| | | PDYWGQGTLVTVSSGGGGSGGGGSGGGGSQSVLTQPPSASGTPGQRVTISC |
| | | TGSSSNIGAGYDVHWYQQLPGTAPKLLIYSNNERPSGVPDRFSGSKSGTSAS |
| | | LAISGLRSEDEADYYCAAWDDSLNGPVFGGGTKLTVLGGGGSGGGGSGGG |
| | | GSGVECPPCPAPPVAGPSVFIFPPKPKDVLTITLTPKVTCVVVDISKDDPEVQ |
| | | FSWFVDDVEVHTAQTQPREEQFNSTFRSVSELPIMHQDWLNGKEFKCRVNS |
| | | AAFPAPIEKTISKTKGRPKAPQVYTIPPPKEQMAKDKVSLWCMITDFFPEDIT |
| | | VEWQWNGQPAENYKNTQPIMDTDGSYFVYSKLNVQKSNWEAGNTFTCSV |
| | | LHEGLHNHHTEKSLSHSPGK |
| PRT | 81 | EVQLLESGGGLVQPGGSLRLSCAASGFTFSHYDMNWLRQAPGKGLEWVG |
| | | VISYDGSNKFYADFVKGRFTISRDNSKNTLYLQMNSLRAEDTAVYYCARTP |
| | | GSHFDYWGQGTLVTVSSGGGGSGGGGSGGGGSQSVLTQPPSASGTPGQRV |
| | | TISCSGSSSNIGNNAVNWYQQLPGTAPKLLIYYDDLLPSGVPDRFSGSKSGT |
| | | SASLAISGLRSEDEADYYCSAWDDSLNGVVFGGGTKLTVLGGGGSGGGGS |
| | | GGGGSGVECPPCPAPPVAGPSVFIFPPKPKDVLTITLTPKVTCVVVDISKDDP |
| | | EVQFSWFVDDVEVHTAQTQPREEQFNSTFRSVSELPIMHQDWLNGKEFKC |
| | | RVNSAAFPAPIEKTISKTKGRPKAPQVYTIPPPKEQMAKDKVSLSCAITDFFP |
| | | EDITVEWQWNGQPAENYKNTQPIMDTDGSYFVVSKLNVQKSNWEAGNTF |
| | | TCSVLHEGLHNHHTEKSLSHSPGK |
| PRT | 82 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSS |
| PRT | 83 | SYGMH |
| PRT | 84 | VISYDGSYKYYADSVKG |
| PRT | 85 | GVPDY |
| PRT | 86 | QSVLTQPPSASGTPGQRVTISCSGSSSNIGSGYDVHWYQQLPGTAPKLLIYS |
| | | NNERPSGVPDRFSGSKSGTSASLAISGLRSEDEADYYCAAWDDSLSGPVFG |
| | | GGTKLTVL |
| PRT | 87 | SGSSSNIGSGYDVH |
| PRT | 88 | SNNERPS |
| PRT | 89 | AAWDDSLSGPV |
| PRT | 90 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 91 | QSVLTQPPSASGTPGQRVTISCSGSSSNIGSGYDVHWYQQLPGTAPKLLIYS |
| | | NNERPSGVPDRFSGSKSGTSASLAISGLRSEDEADYYCAAWDDSLSGPVFG |
| | | GGTKLTVLGQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| PRT | 92 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 93 | QSVLTQPPSASGTPGQRVTISCSGSSSNIGSDYDVHWYQQLPGTAPKLLIYS |
| | | NEERPSGVPDRFSGSKSGTSASLAISGLRSEDEADYYCSAWDSSLSGPVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 94 | QVQLVESGGGVVQPGRSLRLSCAASGFTFSHYDMHWVRQAPGKGLEWVA |
| | | VISYHGSVKFYADSVKGRFTISRDNSKNTLYLQMNSLRAEDTAVYYCARTP |
| | | GFHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 95 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 96 | QVQLVESGGGVVQPGRSLRLSCAASGFTFSHYDMHWVRQAPGKGLEWVA |
| | | VISYSGSVKFYADSVKGRFTISRDNSKNTLYLQMNSLRAEDTAVYYCARTP |
| | | GSHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 97 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 98 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 99 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 100 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 101 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 102 | QVQLVESGGGVVQPGRSLRLSCAASGFTFSHYDMHWVRQAPGKGLEWVA |
| | | VISYEGSVKFYADSVKGRFTISRDNSKNTLYLQMNSLRAEDTAVYYCARTP |
| | | GFHFDYWGQGTLVTVSSAKTTPPSVYPLAPGSAAQTNSMVTLGCLVKGYF |
| | | PEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTC |
| | | VVVDISKDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQMAKDKV |
| | | SLTCMITDFFPEDITVEWQWNGQPAENYKNTQPIMDTDGSYFVYSKLNVQ |
| | | KSNWEAGNTFTCSVLHEGLHNHHTEKSLSHSPGK |
| PRT | 103 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 104 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSAKTTPPSVYPLAPGSAAQTNSMVTLGCLVKGYFPE |
| | | PVTVTWNSGSLSSGVHTFPAVLQSDLYTLSSSVTVPSSTWPSETVTCNVAHP |
| | | ASSTKVDKKIVPRDCGCKPCICTVPEVSSVFIFPPKPKDVLTITLTPKVTCVV |
| | | VDISKDDPEVQFSWFVDDVEVHTAQTQPREEQFNSTFRSVSELPIMHQDWL |
| | | NGKEFKCRVNSAAFPAPIEKTISKTKGRPKAPQVYTIPPPKEQMAKDKVSLT |
| | | CMITDFFPEDITVEWQWNGQPAENYKNTQPIMDTDGSYFVYSKLNVQKSN |
| | | WEAGNTFTCSVLHEGLHNHHTEKSLSHSPGK |
| PRT | 105 | QSVLTQPPSASGTPGQRVTISCSGSSSNIGSDYDVHWYQQLPGTAPKLLIYS |
| | | NEERPSGVPDRFSGSKSGTSASLAISGLRSEDEADYYCSAWDSSLSGPVFGG |
| | | GTKLTVLGQPKSSPSVTLFPPSSEELETNKATLVCTITDFYPGVVTVDWKVD |
| | | GTPVTQGMETTQPSKQSNNKYMASSYLTLTARAWERHSSYSCQVTHEGHT |
| | | VEKSLSRADCS |
| PRT | 106 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSS |
| PRT | 107 | SYGMH |
| PRT | 108 | VISYDGSYKYYADSVKG |
| PRT | 109 | GVPDY |
| PRT | 110 | QSVLTQPPSASGTPGQRVTISCSGSSSNIGSDYDVHWYQQLPGTAPKLLIYS |
| | | NEERPSGVPDRFSGSKSGTSASLAISGLRSEDEADYYCSAWDSSLSGPVFGG |
| | | GTKLTVL |
| PRT | 111 | SGSSSNIGSDYDVH |
| PRT | 112 | SNEERPS |
| PRT | 113 | SAWDSSLSGPV |
| DNA | 114 | GAAGTTCAGCTGCTGGAATCTGGCGGCGGACTGGTTCAACCTGGCGGAT |
| | | CTCTGAGACTGAGCTGTGCCGCCAGCGGCTTCACCTTTAGCAGCTATGG |
| | | CATGCACTGGGTCCGACAGGCCCCTGGCAAAGGACTTGAATGGGTCGCC |
| | | GTGATCAGCTACGACGGCAGCTACAAGTACTACGCCGACAGCGTGAAG |
| | | GGCAGATTCACCATCAGCCGGGACAACAGCAAGAACACCCTGTACCTG |
| | | CAGATGAACAGCCTGAGAGCCGAGGACACCGCCGTGTACTATTGTGCTG |
| | | CTGGCGTGCCAGATTACTGGGGCCAGGGAACACTGGTCACAGTGTCATC |
| | | A |
| DNA | 115 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCGACT |
| | | ATGACGTGCACTGGTATCAGCAGCTGCCTGGCACAGCCCCTAAACTGCT |
| | | GATCTACAGCAACGAGGAACGGCCTAGCGGCGTGCCCGATAGATTTTCT |
| | | GGCAGCAAGAGCGGCACAAGCGCCAGCCTGGCTATCTCTGGACTGAGA |
| | | TCTGAGGACGAGGCCGACTACTACTGCAGCGCCTGGGATTCTTCTCTGT |
| | | CTGGCCCTGTTTTTGGCGGAGGCACCAAGCTGACAGTGCTA |
| PRT | 116 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKVEPKSCDKTHTCPPCPAPPVAGPSVFLFPPKPKDTLMISRTP |
| | | EVTCVVVGVSHEDPEVKFNWYVDGVEVHNAKTKPREEQYNSTYRVVSVL |
| | | TVLHQDWLNGKEYKCKVSNKQLPSPIEKTISKAKGQPREPQVYTLPPSRDE |
| | | LTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 117 | QSVLTQPPSASGTPGQRVTISCSGSSSNIGSDYDVHWYQQLPGTAPKLLIYS |
| | | NEERPSGVPDRFSGSKSGTSASLAISGLRSEDEADYYCSAWDSSLSGPVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 118 | QVQLVESGGGVVQPGRSLRLSCAASGFTFSHYDMHWVRQAPGKGLEWVA |
| | | VISYHGSVKFYADSVKGRFTISRDNSKNTLYLQMNSLRAEDTAVYYCARTP |
| | | GFHFDYWGQGTLVTVSSGGGGSGGGGSGGGGSQSVLTQPPSASGTPGQRV |
| | | TISCSGSSSNIGSNYVSWYQQLPGTAPKLLIYYDDLRPSGVPDRFSGSKSGTS |
| | | ASLAISGLRSEDEADYYCSAWDDSLSGVVFGGGTKLTVLGGGGSGGGGSG |
| | | GGGSGVECPPCPAPPVAGPSVFIFPPKPKDVLTITLTPKVTCVVVDISKDDPE |
| | | VQFSWFVDDVEVHTAQTQPREEQFNSTFRSVSELPIMHQDWLNGKEFKCR |
| | | VNSAAFPAPIEKTISKTKGRPKAPQVYTIPPCKEQMAKDKVSLWCMITDFFP |
| | | EDITVEWQWNGQPAENYKNTQPIMDTDGSYFVYSKLNVQKSNWEAGNTF |
| | | TCSVLHEGLHNHHTEKSLSHSPGK |
| PRT | 119 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSGGGGSGGGGSGGGGSQSVLTQPPSASGTPGQRVTIS |
| | | CSGSSSNIGSDYDVHWYQQLPGTAPKLLIYSNEERPSGVPDRFSGSKSGTSA |
| | | SLAISGLRSEDEADYYCSAWDSSLSGPVFGGGTKLTVLGGGGSGGGGSGGG |
| | | GSGVECPPCPAPPVAGPSVFIFPPKPKDVLTITLTPKVTCVVVDISKDDPEVQ |
| | | FSWFVDDVEVHTAQTQPREEQFNSTFRSVSELPIMHQDWLNGKEFKCRVNS |
| | | AAFPAPIEKTISKTKGRPKAPQVCTIPPPKEQMAKDKVSLSCAITDFFPEDITV |
| | | EWQWNGQPAENYKNTQPIMDTDGSYFVVSKLNVQKSNWEAGNTFTCSVL |
| | | HEGLHNHHTEKSLSHSPGK |
| PRT | 120 | QVQLVESGGGVVQPGRSLRLSCAASGFTFSHYDMHWVRQAPGKGLEWVA |
| | | VISYHGSVKFYADSVKGRFTISRDNSKNTLYLQMNSLRAEDTAVYYCARTP |
| | | GFHFDYWGQGTLVTVSS |
| PRT | 121 | HYDMH |
| PRT | 122 | VISYHGSVKFYADSVKG |
| PRT | 123 | TPGFHFDY |
| PRT | 124 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 125 | SGSSSNIGSNYVS |
| PRT | 126 | YDDLRPS |
| PRT | 127 | SAWDDSLSGVV |
| DNA | 128 | CAGGTTCAGCTGGTGGAATCTGGTGGCGGAGTTGTGCAGCCTGGCAGAA |
| | | GCCTGAGACTGTCTTGTGCCGCCAGCGGCTTCACCTTCAGCCACTACGA |
| | | TATGCACTGGGTCCGACAGGCCCCTGGCAAAGGACTTGAATGGGTCGCC |
| | | GTGATCAGCTACCACGGCAGCGTGAAGTTCTACGCCGACTCCGTGAAGG |
| | | GCAGATTCACCATCAGCCGGGACAACAGCAAGAACACCCTGTACCTGC |
| | | AGATGAACAGCCTGAGAGCCGAGGACACCGCCGTGTACTACTGTGCCA |
| | | GAACACCCGGCTTCCACTTCGACTATTGGGGCCAGGGAACCCTGGTCAC |
| | | AGTCTCTTCA |
| DNA | 129 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCAACT |
| | | ACGTGTCCTGGTATCAGCAGCTGCCTGGCACAGCCCCTAAACTGCTGAT |
| | | CTACTACGACGACCTGCGGCCTAGCGGCGTGCCAGATAGATTTTCTGGC |
| | | AGCAAGAGCGGCACCTCTGCCAGCCTGGCTATCTCTGGACTGAGAAGCG |
| | | AGGACGAGGCCGACTACTACTGTAGCGCCTGGGATGATAGCCTGTCCGG |
| | | CGTTGTGTTTGGCGGAGGCACAAAGCTGACAGTGCTA |
| PRT | 130 | QVQLVESGGGVVQPGRSLRLSCAASGFTFSHYDMHWVRQAPGKGLEWVA |
| | | VISYHGSVKFYADSVKGRFTISRDNSKNTLYLQMNSLRAEDTAVYYCARTP |
| | | GFHFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPPVAGPSVFLFPPKPKDTLMISRT |
| | | PEVTCVVVGVSHEDPEVKFNWYVDGVEVHNAKTKPREEQYNSTYRVVSV |
| | | LTVLHQDWLNGKEYKCKVSNKQLPSPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 131 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 132 | QVQLVESGGGVVQPGRSLRLSCAASGFTFSHYDMHWVRQAPGKGLEWVA |
| | | VISYHGSVKFYADSVKGRFTISRDNSKNTLYLQMNSLRAEDTAVYYCARTP |
| | | GFHFDYWGQGTLVTVSSGGGGSGGGGSGGGGSQSVLTQPPSASGTPGQRV |
| | | TISCSGSSSNIGSNYVSWYQQLPGTAPKLLIYYDDLRPSGVPDRFSGSKSGTS |
| | | ASLAISGLRSEDEADYYCSAWDDSLSGVVFGGGTKLTVLGGGGSGGGGSG |
| | | GGGSGVECPPCPAPPVAGPSVFLFPPKPKDTLMISRTPEVTCVVVGVSHEDP |
| | | EVKFNWYVDGVEVHNAKTKPREEQYNSTYRVVSVLTVLHQDWLNGKEY |
| | | KCKVSNKQLPSPIEKTISKAKGQPREPQVYTLPPCRDELTKNQVSLWCLVK |
| | | GFYPSDIAVEWESNGQPENNYKTTPPVLDSDGSFFLYSKLTVDKSRWQQGN |
| | | VFSCSVMHEALHNHYTQKSLSLSPGK |
| PRT | 133 | EVQLLESGGGLVQPGGSLRLSCAASGFTFSSYGMHWVRQAPGKGLEWVA |
| | | VISYDGSYKYYADSVKGRFTISRDNSKNTLYLQMNSLRAEDTAVYYCAAG |
| | | VPDYWGQGTLVTVSSGGGGSGGGGSGGGGSQSVLTQPPSASGTPGQRVTIS |
| | | CSGSSSNIGSDYDVHWYQQLPGTAPKLLIYSNEERPSGVPDRFSGSKSGTSA |
| | | SLAISGLRSEDEADYYCSAWDSSLSGPVFGGGTKLTVLGGGGSGGGGSGGG |
| | | GSGVECPPCPAPPVAGPSVFLFPPKPKDTLMISRTPEVTCVVVGVSHEDPEV |
| | | KFNWYVDGVEVHNAKTKPREEQYNSTYRVVSVLTVLHQDWLNGKEYKC |
| | | KVSNKQLPSPIEKTISKAKGQPREPQVCTLPPSRDELTKNQVSLSCAVKGFY |
| | | PSDIAVEWESNGQPENNYKTTPPVLDSDGSFFLVSKLTVDKSRWQQGNVFS |
| | | CSVMHEALHNHYTQKSLSLSPGK |
| DNA | 134 | CAGGTGCAGCTGGTGGAATCTGGTGGCGGAGTTGTGCAGCCTGGCAGAT |
| | | CCCTGAGACTGTCTTGTGCCGCCTCCGGCTTCACCTTCAGCCACTACGAT |
| | | ATGCACTGGGTCCGACAGGCCCCTGGCAAAGGATTGGAATGGGTCGCC |
| | | GTGATCTCCTACCACGGCTCCGTGAAGTTCTACGCCGACAGCGTGAAGG |
| | | GCAGATTCACCATCTCTCGGGACAACTCCAAGAACACCCTGTACCTGCA |
| | | GATGAACTCCCTGAGAGCCGAGGACACCGCCGTGTACTACTGTGCTAGA |
| | | ACCCCTGGCTTCCACTTCGACTATTGGGGCCAGGGCACACTGGTCACAG |
| | | TTTCTAGCGGAGGCGGAGGAAGTGGCGGCGGAGGATCTGGCGGTGGTG |
| | | GTTCTCAGTCTGTGCTGACCCAACCTCCTTCCGCTTCTGGCACACCTGGC |
| | | CAGAGAGTGACCATCTCTTGCTCCGGCTCCTCCTCCAACATCGGCTCCA |
| | | ATTACGTGTCCTGGTATCAGCAGCTGCCCGGCACAGCTCCCAAACTGCT |
| | | GATCTACTACGACGACCTGCGGCCTTCTGGCGTGCCCGATAGATTCTCC |
| | | GGCTCTAAGTCTGGCACCTCTGCCAGCCTGGCTATCTCCGGACTGAGAT |
| | | CTGAGGACGAGGCCGACTACTACTGCTCTGCCTGGGACGATTCTCTGTC |
| | | CGGCGTTGTGTTTGGCGGAGGCACCAAACTGACAGTGCTCGGAGGTGGT |
| | | GGCTCTGGTGGCGGAGGAAGTGGCGGAGGCGGTTCTGGTGTTGAATGTC |
| | | CTCCATGTCCTGCTCCTCCAGTGGCTGGCCCTTCCGTGTTTCTGTTCCCTC |
| | | CAAAGCCTAAGGACACCCTGATGATCTCTCGGACCCCTGAAGTGACCTG |
| | | TGTGGTCGTGGGAGTGTCTCACGAGGATCCCGAAGTGAAGTTCAATTGG |
| | | TACGTGGACGGCGTGGAAGTGCACAACGCCAAGACCAAGCCTAGAGAG |
| | | GAACAGTACAACTCCACCTACAGAGTGGTGTCCGTGCTGACCGTGCTGC |
| | | ACCAGGATTGGCTGAACGGCAAAGAGTACAAGTGCAAGGTGTCCAACA |
| | | AGCAGCTGCCCTCTCCAATCGAAAAGACCATCTCCAAGGCCAAGGGAC |
| | | AGCCCAGAGAACCCCAGGTGTACACACTGCCTCCATGCAGAGATGAGCT |
| | | GACCAAGAACCAGGTGTCCCTGTGGTGTCTGGTCAAGGGCTTCTACCCC |
| | | TCCGATATCGCCGTGGAATGGGAGTCTAATGGCCAGCCTGAGAACAACT |
| | | ACAAGACCACACCTCCTGTGCTGGACTCCGACGGCTCATTCTTCCTGTAC |
| | | TCCAAGCTGACAGTGGACAAGTCCAGATGGCAGCAGGGCAACGTGTTCT |
| | | CCTGCTCCGTGATGCACGAGGCCCTGCACAATCACTACACCCAGAAGTC |
| | | CCTGTCTCTGTCCCCTGGCAAG |
| DNA | 135 | GAAGTGCAGCTGCTGGAATCTGGCGGAGGATTGGTTCAGCCTGGCGGCT |
| | | CTCTGAGACTGTCTTGTGCCGCTTCCGGCTTCACCTTCTCCAGCTACGGA |
| | | ATGCACTGGGTCCGACAGGCCCCTGGCAAAGGATTGGAATGGGTCGCC |
| | | GTGATCTCCTACGACGGCTCCTACAAGTACTACGCCGACTCCGTGAAGG |
| | | GCAGATTCACCATCTCTCGGGACAACTCCAAGAACACCCTGTACCTGCA |
| | | GATGAACTCCCTGAGAGCCGAGGACACCGCCGTGTACTATTGTGCTGCT |
| | | GGCGTGCCAGATTACTGGGGCCAGGGAACACTGGTCACAGTGTCTAGCG |
| | | GCGGAGGTGGAAGCGGAGGCGGAGGTTCTGGTGGTGGTGGATCTCAGT |
| | | CTGTGCTGACCCAGCCTCCTTCTGCTTCTGGCACACCTGGCCAGAGAGT |
| | | GACCATCTCTTGCTCCGGCTCCTCCTCCAACATCGGCTCTGACTACGACG |
| | | TGCACTGGTATCAGCAGCTCCCTGGCACAGCTCCCAAACTGCTGATCTA |
| | | CTCCAACGAGGAACGGCCTAGCGGCGTGCCCGATAGATTCTCCGGATCT |
| | | AAGTCCGGCACCTCTGCCAGCCTGGCTATCTCTGGCCTGAGATCTGAGG |
| | | ACGAGGCCGACTACTACTGCTCCGCCTGGGACTCTTCTCTGTCTGGCCCT |
| | | GTTTTTGGCGGAGGCACCAAGCTGACAGTGCTCGGAGGTGGTGGCTCTG |
| | | GTGGCGGAGGAAGTGGCGGAGGCGGTTCTGGTGTTGAATGTCCTCCATG |
| | | TCCTGCTCCTCCAGTGGCTGGCCCTTCCGTGTTTCTGTTCCCTCCAAAGC |
| | | CTAAGGACACCCTGATGATCTCTCGGACCCCTGAAGTGACCTGTGTGGT |
| | | CGTGGGAGTGTCTCACGAGGATCCCGAAGTGAAGTTCAATTGGTACGTG |
| | | GACGGCGTGGAAGTGCACAACGCCAAGACCAAGCCTAGAGAGGAACAG |
| | | TACAACTCCACCTACAGAGTGGTGTCCGTGCTGACCGTGCTGCACCAGG |
| | | ATTGGCTGAACGGCAAAGAGTACAAGTGCAAGGTGTCCAACAAGCAGC |
| | | TGCCCTCTCCAATCGAAAAGACCATCTCCAAGGCCAAGGGACAGCCCCG |
| | | GGAACCTCAAGTCTGTACCTTGCCTCCTAGCCGGGACGAGCTGACCAAG |
| | | AATCAGGTGTCCCTGTCCTGTGCCGTGAAGGGCTTCTACCCTTCCGATAT |
| | | CGCCGTGGAATGGGAGTCCAATGGCCAGCCTGAGAACAACTACAAGAC |
| | | CACACCTCCTGTGCTGGACTCCGACGGCTCATTCTTCCTGGTGTCCAAGC |
| | | TGACAGTGGACAAGTCCAGATGGCAGCAGGGCAACGTGTTCTCCTGCTC |
| | | CGTGATGCACGAGGCCCTGCACAATCACTACACCCAGAAGTCCCTGAGC |
| | | CTGTCTCCTGGCAAG |
| PRT | 136 | QVQLVESGGGVVQPGRSLRLSCAASGFTFSHYDMHWVRQAPGKGLEWVA |
| | | VISYSGSVKFYADSVKGRFTISRDNSKNTLYLQMNSLRAEDTAVYYCARTP |
| | | GSHFDYWGQGTLVTVSS |
| PRT | 137 | HYDMH |
| PRT | 138 | VISYSGSVKFYADSVKG |
| PRT | 139 | TPGSHFDY |
| PRT | 140 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 141 | SGSSSNIGSNYVN |
| PRT | 142 | YDDLRPS |
| PRT | 143 | SAWDDSLSGVV |
| DNA | 144 | CAGGTTCAGCTGGTGGAATCTGGTGGCGGAGTTGTGCAGCCTGGCAGAA |
| | | GCCTGAGACTGTCTTGTGCCGCCAGCGGCTTCACCTTCAGCCACTACGA |
| | | TATGCACTGGGTCCGACAGGCCCCTGGCAAAGGACTTGAATGGGTCGCC |
| | | GTGATCAGCTACAGCGGCAGCGTGAAGTTCTACGCCGACTCCGTGAAGG |
| | | GCAGATTCACCATCAGCCGGGACAACAGCAAGAACACCCTGTACCTGC |
| | | AGATGAACAGCCTGAGAGCCGAGGACACCGCCGTGTACTACTGTGCCA |
| | | GAACACCTGGCAGCCACTTCGACTATTGGGGCCAGGGAACACTGGTCAC |
| | | CGTTAGCTCA |
| DNA | 145 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCAACT |
| | | ACGTGAACTGGTATCAGCAGCTGCCCGGCACAGCCCCTAAACTGCTGAT |
| | | CTACTACGACGACCTGCGGCCTAGCGGCGTGCCAGATAGATTTTCTGGC |
| | | AGCAAGAGCGGCACCTCTGCCAGCCTGGCTATCTCTGGACTGAGAAGCG |
| | | AGGACGAGGCCGACTACTACTGTAGCGCCTGGGATGATAGCCTGTCCGG |
| | | CGTTGTGTTTGGCGGAGGCACAAAGCTGACAGTGCTA |
| PRT | 146 | QVQLVESGGGVVQPGRSLRLSCAASGFTFSHYDMHWVRQAPGKGLEWVA |
| | | VISYSGSVKFYADSVKGRFTISRDNSKNTLYLQMNSLRAEDTAVYYCARTP |
| | | GSHFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPPVAGPSVFLFPPKPKDTLMISRT |
| | | PEVTCVVVGVSHEDPEVKFNWYVDGVEVHNAKTKPREEQYNSTYRVVSV |
| | | LTVLHQDWLNGKEYKCKVSNKQLPSPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 147 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 148 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSS |
| PRT | 149 | HYDMH |
| PRT | 150 | VISYEGSNKFYADSVKG |
| PRT | 151 | TPGFHFDY |
| PRT | 152 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 153 | SGSSSNIGSNYVS |
| PRT | 154 | YDDLRPS |
| PRT | 155 | SAWDDSLSGVV |
| DNA | 156 | CAGGTTCAGCTGGTGGAATCTGGTGGCGGAGTTGTGCAGCCTGGCAGAA |
| | | GCCTGAGACTGTCTTGTGCCGCCAGCGGCTTCACCTTCAGCCACTACGA |
| | | TATGCACTGGGTCCGACAGGCCCCTGGCAAAGGACTTGAATGGGTCGCC |
| | | GTGATCAGCTACGAGGGCAGCAACAAGTTCTACGCCGACAGCGTGAAG |
| | | GGCAGATTCACCATCAGCCGGGACAACAGCAAGAACACCCTGTACCTG |
| | | CAGATGAACAGCCTGAGAGCCGAGGACACCGCCGTGTACTACTGTGCC |
| | | AGAACACCCGGCTTCCACTTCGACTATTGGGGCCAGGGAACCCTGGTCA |
| | | CAGTCTCTTCA |
| DNA | 157 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCAACT |
| | | ACGTGTCCTGGTATCAGCAGCTGCCTGGCACAGCCCCTAAACTGCTGAT |
| | | CTACTACGACGACCTGCGGCCTAGCGGCGTGCCAGATAGATTTTCTGGC |
| | | AGCAAGAGCGGCACCTCTGCCAGCCTGGCTATCTCTGGACTGAGAAGCG |
| | | AGGACGAGGCCGACTACTACTGTAGCGCCTGGGATGATAGCCTGTCCGG |
| | | CGTTGTGTTTGGCGGAGGCACAAAGCTGACAGTGCTA |
| PRT | 158 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPPVAGPSVFLFPPKPKDTLMISRT |
| | | PEVTCVVVGVSHEDPEVKFNWYVDGVEVHNAKTKPREEQYNSTYRVVSV |
| | | LTVLHQDWLNGKEYKCKVSNKQLPSPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 159 | QSVLTQPPSASGTPGQRVTISCSGSSSNIGSNYVSWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 160 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSS |
| PRT | 161 | HYDMH |
| PRT | 162 | VISYEGSNKFYADSVKG |
| PRT | 163 | TPGFHFDY |
| PRT | 164 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 165 | SGSSSNIGSNYVN |
| PRT | 166 | YDDLRPS |
| PRT | 167 | SAWDDSLSGVV |
| DNA | 168 | CAGGTTCAGCTGGTGGAATCTGGTGGCGGAGTTGTGCAGCCTGGCAGAA |
| | | GCCTGAGACTGTCTTGTGCCGCCAGCGGCTTCACCTTCAGCCACTACGA |
| | | TATGCACTGGGTCCGACAGGCCCCTGGCAAAGGACTTGAATGGGTCGCC |
| | | GTGATCAGCTACGAGGGCAGCAACAAGTTCTACGCCGACAGCGTGAAG |
| | | GGCAGATTCACCATCAGCCGGGACAACAGCAAGAACACCCTGTACCTG |
| | | CAGATGAACAGCCTGAGAGCCGAGGACACCGCCGTGTACTACTGTGCC |
| | | AGAACACCCGGCTTCCACTTCGACTATTGGGGCCAGGGAACCCTGGTCA |
| | | CAGTCTCTTCA |
| DNA | 169 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCAACT |
| | | ACGTGAACTGGTATCAGCAGCTGCCCGGCACAGCCCCTAAACTGCTGAT |
| | | CTACTACGACGACCTGCGGCCTAGCGGCGTGCCAGATAGATTTTCTGGC |
| | | AGCAAGAGCGGCACCTCTGCCAGCCTGGCTATCTCTGGACTGAGAAGCG |
| | | AGGACGAGGCCGACTACTACTGTAGCGCCTGGGATGATAGCCTGTCCGG |
| | | CGTTGTGTTTGGCGGAGGCACAAAGCTGACAGTGCTA |
| PRT | 170 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GFHFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPPVAGPSVFLFPPKPKDTLMISRT |
| | | PEVTCVVVGVSHEDPEVKFNWYVDGVEVHNAKTKPREEQYNSTYRVVSV |
| | | LTVLHQDWLNGKEYKCKVSNKQLPSPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 171 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 172 | QVQLVESGGGVVQPGRSLRLSCAASGFTFSHYDMHWVRQAPGKGLEWVA |
| | | VISYEGSVKFYADSVKGRFTISRDNSKNTLYLQMNSLRAEDTAVYYCARTP |
| | | GFHFDYWGQGTLVTVSS |
| PRT | 173 | HYDMH |
| PRT | 174 | VISYEGSVKFYADSVKG |
| PRT | 175 | TPGFHFDY |
| PRT | 176 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 177 | SGSSSNIGSNYVN |
| PRT | 178 | YDDLRPS |
| PRT | 179 | SAWDDSLSGVV |
| DNA | 180 | CAGGTTCAGCTGGTGGAATCTGGTGGCGGAGTTGTGCAGCCTGGCAGAA |
| | | GCCTGAGACTGTCTTGTGCCGCCAGCGGCTTCACCTTCAGCCACTACGA |
| | | TATGCACTGGGTCCGACAGGCCCCTGGCAAAGGACTTGAATGGGTCGCC |
| | | GTGATCAGCTACGAGGGCAGCGTGAAGTTCTACGCCGACTCCGTGAAGG |
| | | GCAGATTCACCATCAGCCGGGACAACAGCAAGAACACCCTGTACCTGC |
| | | AGATGAACAGCCTGAGAGCCGAGGACACCGCCGTGTACTACTGTGCCA |
| | | GAACACCCGGCTTCCACTTCGACTATTGGGGCCAGGGAACCCTGGTCAC |
| | | AGTCTCTTCA |
| DNA | 181 | CAGTCTGTTCTGACACAGCCTCCTAGCGCCTCTGGCACACCTGGACAGA |
| | | GAGTGACCATCAGCTGTAGCGGCAGCAGCTCCAACATCGGCAGCAACT |
| | | ACGTGAACTGGTATCAGCAGCTGCCCGGCACAGCCCCTAAACTGCTGAT |
| | | CTACTACGACGACCTGCGGCCTAGCGGCGTGCCAGATAGATTTTCTGGC |
| | | AGCAAGAGCGGCACCTCTGCCAGCCTGGCTATCTCTGGACTGAGAAGCG |
| | | AGGACGAGGCCGACTACTACTGTAGCGCCTGGGATGATAGCCTGTCCGG |
| | | CGTTGTGTTTGGCGGAGGCACAAAGCTGACAGTGCTA |
| PRT | 182 | QVQLVESGGGVVQPGRSLRLSCAASGFTFSHYDMHWVRQAPGKGLEWVA |
| | | VISYEGSVKFYADSVKGRFTISRDNSKNTLYLQMNSLRAEDTAVYYCARTP |
| | | GFHFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPPVAGPSVFLFPPKPKDTLMISRT |
| | | PEVTCVVVGVSHEDPEVKFNWYVDGVEVHNAKTKPREEQYNSTYRVVSV |
| | | LTVLHQDWLNGKEYKCKVSNKQLPSPIEKTISKAKGQPREPQVYTLPPSRD |
| | | ELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| PRT | 183 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 184 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GSHFDYWGQGTLVTVSS |
| PRT | 185 | HYDMH |
| PRT | 186 | VISYEGSNKFYADSVKG |
| PRT | 187 | TPGSHFDY |
| PRT | 188 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVL |
| PRT | 189 | SGSSSNIGSNYVN |
| PRT | 190 | YDDLRPS |
| PRT | 191 | SAWDDSLSGVV |
| PRT | 192 | QVQLVESGGGVVQPGRSLRLSCAASGFTFSHYDMHWVRQAPGKGLEWVA |
| | | VISYEGSNKFYADSVKGRFTISRDNSKNTLYLQMNSLRAEDTAVYYCARTP |
| | | GSHFDYWGQGTLVTVSSASTKGPSVFPLAPCSRSTSESTAALGCLVKDYFPE |
| | | PVTVSWNSGALTSGVHTFPAVLQSSGLYSLSSVVTVPSSSLGTKTYTCNVD |
| | | HKPSNTKVDKRVESKYGPPCPPCPAPEFEGGPSVFLFPPKPKDTLMISRTPEV |
| | | TCVVVDVSQEDPEVQFNWYVDGVEVHNAKTKPREEQFNSTYRVVSVLTV |
| | | LHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRL |
| | | TVDKSRWQEGNVFSCSVMHEALHNHYTQKSLSLSLGK |
| PRT | 193 | QSVLTQPPSASGTPGQRVTISCSGSSSNIGSNYVNWYQQLPGTAPKLLIYYD |
| | | DLRPSGVPDRFSGSKSGTSASLAISGLRSEDEADYYCSAWDDSLSGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 194 | EVQLLESGGGLVQPGGSLRLSCAASGFTFSHYDMNWLRQAPGKGLEWVG |
| | | VISYDGSNKFYADFVKGRFTISRDNSKNTLYLQMNSLRAEDTAVYYCARTP |
| | | GSHFDYWGQGTLVTVSSASTKGPSVFPLAPCSRSTSESTAALGCLVKDYFPE |
| | | PVTVSWNSGALTSGVHTFPAVLQSSGLYSLSSVVTVPSSSLGTKTYTCNVD |
| | | HKPSNTKVDKRVESKYGPPCPPCPAPEFLGGPSVFLFPPKPKDTLMISRTPEV |
| | | TCVVVDVSQEDPEVQFNWYVDGVEVHNAKTKPREEQFNSTYRVVSVLTV |
| | | LHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRL |
| | | TVDKSRWQEGNVFSCSVMHEALHNHYTQKSLSLSLGK |
| PRT | 195 | QSVLTQPPSASGTPGQRVTISCSGSSSNIGNNAVNWYQQLPGTAPKLLIYYD |
| | | DLLPSGVPDRFSGSKSGTSASLAISGLRSEDEADYYCSAWDDSLNGVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| PRT | 196 | EVQLLESGGGLVQPGGSLRLSCAASGFTFSIYAMSWVRQAPGKGLEWVSSI |
| | | SDTGDRTYYADSVKGRFTISRDNSKNTLYLQMNSLRAEDTAVYYCARSNW |
| | | GSLDYWGQGTLVTVSSASTKGPSVFPLAPCSRSTSESTAALGCLVKDYFPEP |
| | | VTVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTPEVT |
| | | CVVVDVSQEDPEVQFNWYVDGVEVHNAKTKPREEQFNSTYRVVSVLTVL |
| | | HQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLT |
| | | VDKSRWQEGNVFSCSVMHEALHNHYTQKSLSLSLGK |
| PRT | 197 | QSVLTQPPSASGTPGQRVTISCSGSSSNIGSNYVYWYQQLPGTAPKLLIYYD |
| | | DLLPSGVPDRFSGSKSGTSASLAISGLRSEDEADYYCAAWDDSLSGRVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |

### REFERENCES

The following articles are referred to in this specification. For enablement purposes of the present invention, the content thereof is incorporated herein by reference.
Adamson G D, Kennedy S H, Hummelshoj L. Creating solutions in endometriosis: global collaboration through the World Endometriosis Research Foundation. Journal of Endometriosis 2010; 2: 3-6
Blind, M. & Blank, M. (2015). Mol Ther Nucleic Acids. 2015 January; 4(1): e223.
Bockhorn et al. (2013). Nat. Commun. 4, 1393
Bolt et al., Eur J Immunol. 1993 February; 23(2):403-11
Burney R O, Lathi R B. Menstrual bleeding from an endometriotic lesion, Fertil. Steril., 91 (2009), pp. 1926-1927.
Cork B A, Tuckernman E M, Li T C, and Laird S M. Expression of interleukin (IL)-11 receptor by the human endometrium in vivo and effects of IL-11, IL-6 and LIF on the production of MMP and cytokines by endometrial cells in vitro, Molecular Human Reproduction, 2002; 8(9):841-848
Dimitriadis E, Stoikos S, Stafford-Bell M, Clark I, Paiva P et al., Journal of Reproductive Immunology 69 (2006) 53-64.
DiVasta A D, Laufer M R. The use of gonadotropin releasing hormone (GnRH) analogues in adolescent and young patients with endometriosis. Curr Opin Obstet Gynecol 2013; 25: 287-92.
Durben et al., 2015, Mol Ther. 2015 April; 23(4):648-55
El-Hemaidi I, Gharaibeh A, Shehata H. Menorrhagia and bleeding disorders. Curr Opin Obstet Gynecol 2007; 19(6): 513-20.
Fraser I S, Langham S, Uhl-Hochgraeber K. Health-related quality of life and economic burden of abnormal uterine bleeding. Expert Rev Obstet Gynecol 2009; 4(2): 179-89.
Gazvani, M R, Bates M, Vince G, Christmas, S, Lewis-Jones I. Peritoneal fluid concentrations of interleukin-11 in women with endometriosis. Fertility And Sterility Vol. 74, No. 6, December 2000.
Giudice, L C. Clinical practice. Endometriosis. N Engl J Med. 2010; 362:2389-98.
Giudice L C, Kao L C. Endometriosis. Lancet 2004; 364:1789-99.
Hallberg L, Nilsson L. Determination of Menstrual Blood Loss. Scand J Clin Lab Invest (1964) 16: 244-248.
Hallberg L, Hogdahl A M, Nilsson L, Rybo G. Menstrual blood loss—a population study. Variation at different ages and attempts to define normality. Acta Obstet Gynecol Scand 1966; 45(3): 320-51.
Harmegnies D, Wang X M, Vandenbussche P, Leon A, Vusio P. et al. Characterization of a potent human interleukin-11 agonist. Biochem J. 2003 Oct. 1; 375(Pt 1):23-32.
Janssen E B, Rijkers A C, Hoppenbrouwers K, Meuleman C, D'Hooghe T M. Prevalence of endometriosis diagnosed by laparoscopy in adolescents with dysmenorrhea or chronic pelvic pain: a systematic review. Human Reproduction Update (2013) 19 (5): 570-82.
Jinek, M. et al (2012): Science, 337, 816-821
Ju H, Jones M, Mishra G. The Prevalence and Risk Factors of Dysmenorrhea. Epidemiologic Reviews 2014: Volume 36, Issue 1, 1 Jan. 2014, 104-113.
Kadir R A, Davies J. Hemostatic disorders in women. J Thromb Haemost 2013, 11 (Suppl.1): 170-9.
Kjerulff K H, Erickson B A, Langenberg P W. Am J. Public Health. 1996; 86:195-9.
Laux-Biehlmann A, d'Hooghe T, Zollner T M. Menstruation pulls the trigger for inflammation and pain in endometriosis. Trends Pharmacol Sci. 2015 May; 36(5):270-6.
Leabman et al., MAbs November-December 2013; 5(6):896-903
Lethaby A, Duckitt K, Farquhar C. Non-steroidal anti-inflammatory drugs for heavy menstrual bleeding. The Cochrane Database of Systematic Reviews 2013 1 (1): CD000400.
Lokau et al., 2016 Cell Reports 14, 1761-1773
Lokau J, Agthe M, Flynn C M, Garbers C. Proteolytic control of Interleukin-11 and Interleukin-6 biology. BBA—Molecular Cell Research 1864 (2017) 2105-2117.
Luo X, Ding L, Xu J, Williams R S, Chegini N. Leiomyoma and myometrial gene expression profiles and their responses to gonadotropin-releasing hormone analog therapy. Endocrinology. 2005 March; 146(3):1074-96. Epub 2004 Dec. 16.
Marjoribanks J, Ayeleke R O, Farquhar C, Proctor M. Nonsteroidal anti-inflammatory drugs for dysmenorrhea. The Cochrane Database of Systematic Reviews 2015 (7): CD001751.
Menning A, Walter A, Rudolph M, Gashaw I, Fritzemeier K-H, Roese L. Granulocytes and Vascularization Regulate uterine Bleeding and Tissue Remodeling in a Mouse Menstruation Model PLoS ONE (2012) 7(8): e41800.
Mikolajczyk M, Wirstlein P, Skrzypczak J. Leukaemia inhibitory factor and interleukin 11 levels in uterine flushings of infertile patients with endometriosis. Human Reproduction 2006 Vol. 21, No. 12, 3054-3058.
Murbach T S, Hirka G, Szakonyind I P, Gericke N, Endres J R. A toxicological safety assessment of a standardized extract of Sceletium tortuosum (Zembrin®) in rats. Food and Chemical Toxicology 74 (2014) 190-199
Nordan R P, Pumphrey J G, Rudikoff S, Purification and NH2-terminal sequence of a plasmacytoma growth factor derived from the murine macrophage cell line P388D1. (1987) J. Immunol. 139:813-7.
Oganesyan et al., Acta Crystallogr D Biol Crystallogr. 2008 June; 64
Olive D L. Endometriosis: does surgery make a difference? OB/GYN Management 2002; 14. 56-58 and 68-70
Osayande A S, Mehulic S. Diagnosis and initial management of dysmenorrhea. American Family Physician. 2014: 89 (5): 341-6.
Paul S R, Bennett F, Calvetti J A, Kelleher K, Wood C R et al. Molecular cloning of a cDNA encoding interleukin 11, a stromal cell-derived lymphopoietic and hematopoietic cytokine. Proc Natl Acad Sci USA. 1990 October; 87(19):7512-6.
Power J, French R, Cowan F. Subdermal implantable contraceptives versus other forms of reversible contraceptives or other implants as effective methods of preventing pregnancy. Power J, ed. The Cochrane Database of Systematic Reviews 2007 (3): CD001326.
Ragni M V, Jankowitz R C, Jaworski K, Merricks E P, Kloos M T and Nichols T C, Phase II prospective open-label trial of recombinant interleukin-11 in women with mild von Willebrand disease and refractory menorrhagia, Thromb Haemost.; 2011; 106(4): 641-645.
Shapley M, Jordan K, Croft P R. An epidemiological survey of symptoms of menstrual loss in the community. Br J Gen Pract 2004; 54(502): 359-63.
Sachedina A, Todd N. Dysmenorrhea, Endometriosis and Chronic Pelvic Pain in Adolescents. J Clin Res Pediatr Endocrinol 2020: 12(Suppl 1):7-17
Schlothauer et al., Protein Eng Des Sel 2016 October; 29(10):457-466)
Sumida K, Ohno Y, Ohtake J, Kaneumi S, Kishikawa T, Takahashi N, Taketomi A, Kitamura H. IL-11 induces differentiation of myeloid-derived suppressor cells through activation of STAT3 signaling pathway. Scientific Reports 12015; 5:13650
Sinaii, N, Plumb K, Cotton L, Lambert A, Kennedy S et al. Differences in characteristics among 1,000 women with endometriosis based on extent of disease. Fertil Steril. 2008; 89:538-45.
Tam et al., Antibodies (Basel) 2017 Sep. 1; 6(3):12)
Tao and Morrison, J Immunol. 1989 Oct. 15; 143(8):2595-601
Thiel, K. & Giangrande, P. (2010): (2010). Therapeutic delivery. 1. 849-861.
Walker et al., Biochem J. 1989 Apr. 15; 259(2):347-53
Wong C L, Farquhar C, Roberts H, Proctor M. Oral contraceptive pill for primary dysmenorrhea. The Cochrane Database of Systematic Reviews 2009 (4): CD002120.
Wright J/Solange W. The Washington Manual Obstetrics and Gynecology Survival Guide. Lippincott Williams and Wilkins, 2003. ISBN 0-7817-4363-X.
Xu et al., Cell Immunol 2000 Feb. 25; 200(1):16-26
Yin, H. et al (2016). Nature Biotechnology, 34, 328-333.
Zetsche, B. et al. (2015): Cell, 22, 163(3): 759-771.
Zhou et al., MAbs January-December 2020; 12(1):1814583
WO9603143
WO 99/59608
WO 2018/109174
WO 2005/098041A2
WO2018109170,
WO2017103108,
WO2019238882,
WO2019238884
US 2014/0219919
EP2794658

What is claimed is:

1. An isolated antibody or antigen-binding fragment thereof capable of binding to IL-11 and inhibiting IL-11 mediated signaling, wherein said isolated antibody or antigen-binding fragment thereof is capable of reducing blood loss during abnormal uterine bleeding.

2. The isolated antibody or antigen-binding fragment thereof capable of binding to IL-11 and inhibiting IL-11 mediated signaling, according to claim 1, wherein said isolated antibody or antigen-binding fragment thereof binds to human IL-11 with a dissociation constant (K_{D})≤1.0 E-08 M, ≤1.0 E-09 M, ≤5.0 E-10 M, ≤1.0 E-10 M, ≤5.0 E-11 M, ≤2.5 E-11 M or ≤1.0 E-11 M.

3. The isolated antibody or antigen-binding fragment thereof capable of binding to IL-11 and inhibiting IL-11 mediated signaling, according to claim 1, wherein said isolated antibody or antigen-binding fragment thereof inhibits human IL-11 mediated signaling with an IC₅₀ of ≤100 nM, ≤50 nM, ≤25 nM, ≤10 nM, ≤1 nM, ≤0.5 nM or ≤0.1 nM.

4. The isolated antibody or antigen-binding fragment thereof capable of binding to IL-11 and inhibiting IL-11 mediated signaling, according to claim 1, wherein said isolated antibody or antigen-binding fragment thereof inhibits the interaction of IL-11 with IL-11Ra; and wherein said isolated antibody or antigen-binding fragment thereof inhibits the formation of IL-11/IL-11Ra/gp130 complex.

5. The isolated antibody or antigen-binding fragment thereof capable of binding to IL-11 and inhibiting IL-11 mediated signaling, according to claim 1, wherein said isolated antibody or antigen-binding fragment thereof inhibits the formation of IL-11/IL-11Ra/gp130 complex; and wherein said isolated antibody or antigen-binding fragment thereof does not inhibit the interaction of IL-11 with IL-11Ra.

6. The isolated antibody or antigen-binding fragment thereof according to claim 1, wherein said isolated antibody or antigen-binding fragment thereof is a bispecific antibody or antigen-binding fragment thereof capable of binding to two different IL-11 epitopes.

7. The isolated antibody or antigen-binding fragment thereof according to claim 6, wherein said isolated antibody or antigen-binding fragment thereof inhibits the binding of IL-11 to IL-11Ra; and wherein said isolated antibody or antigen-binding fragment thereof inhibits the formation of IL-11/IL-11Ra/gp130 complex.

8. The isolated antibody or antigen-binding fragment thereof according to claim 1, wherein said human IL-11 is of the sequence of SEQ ID NO: 1, aa 22-199; wherein said human IL-11Ra is of the sequence of SEQ ID NO: 3; and/or wherein said human gp130 is sequence SEQ ID 12.

9. The isolated antibody or antigen-binding fragment thereof according to claim 1, wherein said isolated antibody or antigen-binding fragment thereof does not bind to IL-2Ra.

10. The isolated antibody or antigen-binding fragment thereof according to claim 1, comprising:
i) a heavy chain antigen-binding region that comprises an H-CDR1 comprising SEQ ID NO: 33, an H-CDR2 comprising SEQ ID NO: 34, and an H-CDR3 comprising SEQ ID NO: 35 and a light chain antigen-binding region that comprises an L-CDR1 comprising SEQ ID NO: 37, an L-CDR2 comprising SEQ ID NO: 38, and an L-CDR3 comprising SEQ ID NO:39; or
ii) a heavy chain antigen-binding region that comprises an H-CDR1 comprising SEQ ID NO: 43, an H-CDR2 comprising SEQ ID NO: 44, and an H-CDR3 comprising SEQ ID NO: 45 and a light chain antigen-binding region that comprises an L-CDR1 comprising SEQ ID NO: 47, an L-CDR2 comprising SEQ ID NO: 48, and an L-CDR3 comprising SEQ ID NO:49; or
iii) a heavy chain antigen-binding region that comprises an H-CDR1 comprising SEQ ID NO: 55, an H-CDR2 comprising SEQ ID NO: 56, and an H-CDR3 comprising SEQ ID NO: 57 and a light chain antigen-binding region that comprises an L-CDR1 comprising SEQ ID NO: 59, an L-CDR2 comprising SEQ ID NO: 60, and an L-CDR3 comprising SEQ ID NO:61; or
iv) a heavy chain antigen-binding region that comprises an H-CDR1 comprising SEQ ID NO: 65, an H-CDR2 comprising SEQ ID NO: 66, and an H-CDR3 comprising SEQ ID NO: 67 and a light chain antigen-binding region that comprises an L-CDR1 comprising SEQ ID NO: 69, an L-CDR2 comprising SEQ ID NO: 70, and an L-CDR3 comprising SEQ ID NO:71; or
v) a heavy chain antigen-binding region that comprises an H-CDR1 comprising SEQ ID NO: 83, an H-CDR2 comprising SEQ ID NO: 84, and an H-CDR3 comprising SEQ ID NO: 85 and a light chain antigen-binding region that comprises an L-CDR1 comprising SEQ ID NO: 87, an L-CDR2 comprising SEQ ID NO: 88, and an L-CDR3 comprising SEQ ID NO:89; or
vi) a heavy chain antigen-binding region that comprises an H-CDR1 comprising SEQ ID NO: 107, an H-CDR2 comprising SEQ ID NO: 108, and an H-CDR3 comprising SEQ ID NO: 109 and a light chain antigen-binding region that comprises an L-CDR1 comprising SEQ ID NO: 111, an L-CDR2 comprising SEQ ID NO: 112, and an L-CDR3 comprising SEQ ID NO:113; or
vii) a heavy chain antigen-binding region that comprises an H-CDR1 comprising SEQ ID NO: 121, an H-CDR2 comprising SEQ ID NO: 122, and an H-CDR3 comprising SEQ ID NO: 123 and a light chain antigen-binding region that comprises an L-CDR1 comprising SEQ ID NO: 125, an L-CDR2 comprising SEQ ID NO: 126, and an L-CDR3 comprising SEQ ID NO:127; or
viii) a heavy chain antigen-binding region that comprises an H-CDR1 comprising SEQ ID NO: 137, an H-CDR2 comprising SEQ ID NO: 138, and an H-CDR3 comprising SEQ ID NO: 139 and a light chain antigen-binding region that comprises an L-CDR1 comprising SEQ ID NO: 141, an L-CDR2 comprising SEQ ID NO: 142, and an L-CDR3 comprising SEQ ID NO:143; or
ix) a heavy chain antigen-binding region that comprises an H-CDR1 comprising SEQ ID NO: 149, an H-CDR2 comprising SEQ ID NO: 150, and an H-CDR3 comprising SEQ ID NO: 151 and a light chain antigen-binding region that comprises an L-CDR1 comprising SEQ ID NO: 153, an L-CDR2 comprising SEQ ID NO: 154, and an L-CDR3 comprising SEQ ID NO:155; or
x) a heavy chain antigen-binding region that comprises an H-CDR1 comprising SEQ ID NO: 161, an H-CDR2 comprising SEQ ID NO: 162, and an H-CDR3 comprising SEQ ID NO: 163 and a light chain antigen-binding region that comprises an L-CDR1 comprising SEQ ID NO: 165, an L-CDR2 comprising SEQ ID NO: 166, and an L-CDR3 comprising SEQ ID NO:167; or
xi) a heavy chain antigen-binding region that comprises an H-CDR1 comprising SEQ ID NO: 173, an H-CDR2 comprising SEQ ID NO: 174, and an H-CDR3 comprising SEQ ID NO: 175 and a light chain antigen-binding region that comprises an L-CDR1 comprising SEQ ID NO: 177, an L-CDR2 comprising SEQ ID NO: 178, and an L-CDR3 comprising SEQ ID NO:179; or
xii) a heavy chain antigen-binding region that comprises an H-CDR1 comprising SEQ ID NO: 185, an H-CDR2 comprising SEQ ID NO: 186, and an H-CDR3 comprising SEQ ID NO: 187 and a light chain antigen-binding region that comprises an L-CDR1 comprising SEQ ID NO: 189, an L-CDR2 comprising SEQ ID NO: 190, and an L-CDR3 comprising SEQ ID NO:191.

11. The isolated antibody or antigen-binding fragment thereof according to claim 1, comprising;
xiii) a variable heavy chain domain comprising SEQ ID NO: 32 and a variable light chain domain comprising SEQ ID NO: 36; or
xiv) a variable heavy chain domain comprising SEQ ID NO: 42 and a variable light chain domain comprising SEQ ID NO: 46; or
xv) a variable heavy chain domain comprising SEQ ID NO: 54 and a variable light chain domain comprising SEQ ID NO: 58; or
xvi) a variable heavy chain domain comprising SEQ ID NO: 64 and a variable light chain domain comprising SEQ ID NO: 68; or
xvii) a variable heavy chain domain comprising SEQ ID NO: 82 and a variable light chain domain comprising SEQ ID NO: 86; or
xviii) a variable heavy chain domain comprising SEQ ID NO: 106 and a variable light chain domain comprising SEQ ID NO: 110; or
xix) a variable heavy chain domain comprising SEQ ID NO: 120 and a variable light chain domain comprising SEQ ID NO: 124; or
xx) a variable heavy chain domain comprising SEQ ID NO: 136 and a variable light chain domain comprising SEQ ID NO: 140; or
xxi) a variable heavy chain domain comprising SEQ ID NO: 148 and a variable light chain domain comprising SEQ ID NO: 152; or
xxii) a variable heavy chain domain comprising SEQ ID NO: 160 and a variable light chain domain comprising SEQ ID NO: 164; or
xxiii) a variable heavy chain domain comprising SEQ ID NO: 172 and a variable light chain domain comprising SEQ ID NO: 176; or
xxiv) a variable heavy chain domain comprising SEQ ID NO: 184 and a variable light chain domain comprising SEQ ID NO: 188.

12. The isolated antibody according to claim 1, wherein the isolated antibody is an IgG antibody.

13. The isolated antibody according to claim 1, wherein the antibody comprises:
xxv) a heavy chain comprising SEQ ID NO: 40 and a light chain comprising SEQ ID NO: 41; or
xxvi) a heavy chain comprising SEQ ID NO: 90 and a light chain comprising SEQ ID NO: 91; or
xxvii) a heavy chain comprising SEQ ID NO: 92 and a light chain comprising SEQ ID NO: 33; or
xxviii) a heavy chain comprising SEQ ID NO: 116 and a light chain comprising SEQ ID NO: 117; or
xxix) a heavy chain comprising SEQ ID NO: 130 and a light chain comprising SEQ ID NO: 131; or
xxx) a heavy chain comprising SEQ ID NO: 146 and a light chain comprising SEQ ID NO: 147; or
xxxi) a heavy chain comprising SEQ ID NO: 158 and a light chain comprising SEQ ID NO: 159; or
xxxii) a heavy chain comprising SEQ ID NO: 170 and a light chain comprising SEQ ID NO: 171; or
xxxiii) a heavy chain comprising SEQ ID NO: 182 and a light chain comprising SEQ ID NO: 183; or
xxxiv) a heavy chain comprising SEQ ID NO: 192 and a light chain comprising SEQ ID NO: 193; or
xxxv) a heavy chain comprising SEQ ID NO: 194 and a light chain comprising SEQ ID NO: 195; or
xxxvi) a heavy chain comprising SEQ ID NO: 196 and a light chain comprising SEQ ID NO: 197.

14. The isolated antibody or antigen-binding fragment thereof according to claim 6, comprising:
xxxvii) a first chain comprising a heavy chain antigen-binding region that comprises an H-CDR1 comprising SEQ ID NO: 33, an H-CDR2 comprising SEQ ID NO: 34, and an H-CDR3 comprising SEQ ID NO: 35 and a light chain antigen-binding region that comprises an L-CDR1 comprising SEQ ID NO: 37, an L-CDR2 comprising SEQ ID NO: 38, and an L-CDR3 comprising SEQ ID NO:39, and a second chain comprising a heavy chain antigen-binding region that comprises an H-CDR1 comprising SEQ ID NO: 55, an H-CDR2 comprising SEQ ID NO: 56, and an H-CDR3 comprising SEQ ID NO: 57 and a light chain antigen-binding region that comprises an L-CDR1 comprising SEQ ID NO: 59, an L-CDR2 comprising SEQ ID NO: 60, and an L-CDR3 comprising SEQ ID NO:61; or
xxxviii) a first chain comprising a heavy chain antigen-binding region that comprises an H-CDR1 comprising SEQ ID NO: 121, an H-CDR2 comprising SEQ ID NO: 122, and an H-CDR3 comprising SEQ ID NO: 123 and a light chain antigen-binding region that comprises an L-CDR1 comprising SEQ ID NO: 125, an L-CDR2 comprising SEQ ID NO: 126, and an L-CDR3 comprising SEQ ID NO:127, and a second chain comprising a heavy chain antigen-binding region that comprises an H-CDR1 comprising SEQ ID NO: 107, an H-CDR2 comprising SEQ ID NO: 108, and an H-CDR3 comprising SEQ ID NO: 109 and a light chain antigen-binding region that comprises an L-CDR1 comprising SEQ ID NO: 111, an L-CDR2 comprising SEQ ID NO: 112, and an L-CDR3 comprising SEQ ID NO:113.

15. The isolated antibody or antigen-binding fragment thereof according to claim 6, comprising:
xxxix) a first chain comprising a variable heavy chain domain comprising SEQ ID NO: 32 and a variable light chain domain comprising SEQ ID NO: 36 and a second chain comprising a variable heavy chain domain comprising SEQ ID NO: 54 and a variable light chain domain comprising SEQ ID NO: 58; or
xl) a first chain comprising a variable heavy chain domain comprising SEQ ID NO: 120 and a variable light chain domain comprising SEQ ID NO: 124 and a second chain comprising a variable heavy chain domain comprising SEQ ID NO: 106 and a variable light chain domain comprising SEQ ID NO: 110.

16. The isolated antibody according to claim 6, comprising:
xli) a first chain comprising SEQ ID NO: 74 and a second chain comprising SEQ ID NO: 75; or
xlii) a first chain comprising SEQ ID NO: 132 and a second chain comprising SEQ ID NO: 133.

17. The isolated antibody or antigen-binding fragment of claim 1, wherein the antigen-binding fragment is an scFv, Fab, Fab′ fragment, or a F(ab′)2 fragment.

18. The isolated antibody or antigen-binding fragment according to claim 1, wherein the antibody is a monoclonal antibody or antigen-binding fragment.

19. The isolated antibody or antigen-binding fragment according to claim 1, wherein the isolated antibody or antigen-binding fragment is a human, humanized, or chimeric antibody or antigen-binding fragment.

20. An isolated antibody or antigen-binding fragment thereof that competes with the isolated antibody or antigen-binding fragment according to claim 1 for binding to IL-11.

21. An antibody conjugate, comprising the isolated antibody or antigen binding fragment according to claim 1.

22. An isolated nucleic acid sequence that encodes the antibody or antigen-binding fragment according to claim 1.

23. A vector comprising a nucleic acid sequence according to claim 22.

24. An isolated cell expressing the antibody or antigen-binding fragment according to claim 1 and/or comprising a nucleic acid encoding the antibody or antigen-binding fragment according to claim 1 or a vector comprising a nucleic acid encoding the antigen or antigen-binding fragment according to claim 1.

25. The isolated cell according to claim 24, wherein said cell is a prokaryotic or a eukaryotic cell.

26. A method of producing the isolated antibody or antigen-binding fragment according to claim 1 comprising culturing a cell expressing the antibody or antigen-binding fragment and optionally purifying the antibody or antigen-binding fragment.

27. A pharmaceutical composition comprising the isolated antibody or antigen-binding fragment according to claim 1 or an antibody conjugate thereof and optionally one or more pharmaceutically acceptable excipients.

28. (canceled)

29. A diagnostic agent comprising the isolated antibody or antigen-binding fragment according to claim 1 or an antibody conjugate thereof.

30. A method for treating or preventing abnormal uterine bleeding (AUB), dysmenorrhea, leiomyoma, or endometriosis comprising administration of the isolated antibody or antigen-binding fragment according to claim 1 or a conjugate thereof or a pharmaceutical composition comprising the antibody or antigen-binding fragment or conjugate thereof.

31. The method of claim 30, wherein the abnormal uterine bleeding is heavy menstrual bleeding prolonged bleeding, or altered bleeding pattern.

32. The method of claim 30, wherein the abnormal uterine bleeding is heavy menstrual bleeding and wherein heavy menstrual bleeding is secondary to leiomyoma or endometriosis.

33. The method of claim 30, wherein the abnormal uterine bleeding is associated with dysmenorrhea.

34. The method of claim 30, wherein the abnormal uterine bleeding is associated with dysmenorrhea secondary to leiomyoma or endometriosis.

35.-36. (canceled)

37. A kit comprising the isolated antibody or antigen-binding fragment according to claim 1 or a conjugate thereof, or a pharmaceutical composition comprising the antibody or antigen-binding fragment or conjugate thereof and instructions for use.


SEQUENCE LISTING (197 sequences)

SEQ ID NO: 1 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 2 - protein, 120 aa, Homo sapiens
MSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 3 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 4 - protein, 390 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLW

SEQ ID NO: 5 - DNA, 2378 bp, Homo sapiens
actgccgcggccctgctgctcagggcacatgcctcccctccccaggccgcggcccagctgaccctcggggctcccccggcagcggacagggaagggttaaaggcccccggctccctgccccctgccctggggaacccctggccctgtggggacatgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaataacagaa

SEQ ID NO: 6 - DNA, 2208 bp, Homo sapiens
actgccgcggccctgctgctcagggcacatgcctcccctccccaggccgcggcccagctgaccctcggggctcccccggcagcggacagggaagggttaaaggcccccggctccctgccccctgccctggggaacccctggccctgtggggacatgaactagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaataacagaaaaa

SEQ ID NO: 7 - DNA, 1728 bp, Homo sapiens
agagggcgagggcgagggcagagggcgctggcggcagcggccgcggaagatgagcagcagctgctcagggctgagcagggtcctggtggccgtggctacagccctggtgtctgcctcctccccctgcccccaggcctggggccccccaggggtccagtatgggcagccaggcaggtccgtgaagctgtgttgtcctggagtgactgccggggacccagtgtcctggtttcgggatggggagccaaagctgctccagggacctgactctgggctagggcatgaactggtcctggcccaggcagacagcactgatgagggcacctacatctgccagaccctggatggtgcacttgggggcacagtgaccctgcagctgggctaccctccagcccgccctgttgtctcctgccaagcagccgactatgagaacttctcttgcacttggagtcccagccagatcagcggtttacccacccgctacctcacctcctacaggaagaagacagtcctaggagctgatagccagaggaggagtccatccacagggccctggccatgcccacaggatcccctaggggctgcccgctgtgttgtccacggggctgagttctggagccagtaccggattaatgtgactgaggtgaacccactgggtgccagcacacgcctgctggatgtgagcttgcagagcatcttgcgccctgacccaccccagggcctgcgggtagagtcagtaccaggttacccccgacgcctgcgagccagctggacataccctgcctcctggccgtgccagccccacttcctgctcaagttccgtttgcagtaccgtccggcgcagcatccagcctggtccacggtggagccagctggactggaggaggtgatcacagatgctgtggctgggctgccccatgctgtacgagtcagtgcccgggactttctagatgctggcacctggagcacctggagcccggaggcctggggaactccgagcactgggaccataccaaaggagataccagcatggggccagctacacacgcagccagaggtggagcctcaggtggacagccctgctcctccaaggccctccctccaaccacaccctcggctacttgatcacagggactctgtggagcaggtagctgtgctggcgtctttgggaatcctttctttcctgggactggtggctggggccctggcactggggctctggctgaggctgagacggggtgggaaggatggatccccaaagcctgggttcttggcctcagtgattccagtggacaggcgtccaggagctccaaacctgtagaggacccaggagggcttcggcagattccacctataattctgtcttgctggtgtggatagaaaccaggcaggacagtagatccctatggttggatctcagctggaagttctgtttggagcccatttctgtgagaccctgtatttcaaatttgcagctgaaaggtgcttgtacctctgatttcaccccagagttggagttctgctcaaggaacgtgtgtaatgtgtacatctgtgtccatgtgtgaccatgtgtctgtgaggcagggaacatgtattctctgcatgcatgtatgtaggtgcctggggagtgtgtgtgggtccttggctcttggcctttccccttgcaggggttgtgcaggtgtgaataaagagaataaggaagttcttggagattatactcagaaaaaaaaa

SEQ ID NO: 8 - protein, 432 aa, Mus musculus
MSSSCSGLTRVLVAVATALVSSSSPCPQAWGPPGVQYGQPGRPVMLCCPGVSAGTPVSWFRDGDSRLLQGPDSGLGHRLVLAQVDSPDEGTYVCQTLDGVSGGMVTLKLGFPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPSTGPLQDEIPDWSQGHGQQLEAVVAQEDSPAPARPSLQPDPRPLDHRDPLEQVAVLASLGIFSCLGLAVGALALGLWLRLRRSGKDGPQKPGLLAPMIPVEKLPGIPNLQRTPENFS

SEQ ID NO: 9 - protein, 432 aa, Mus musculus
MSSSCSGLTRVLVAVATALVSSSSPCPQAWGPPGVQYGQPGRPVMLCCPGVSAGTPVSWFRDGDSRLLQGPDSGLGHRLVLAQVDSPDEGTYVCQTLDGVSGGMVTLKLGFPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDTVAGLPHAVRVSARDFLDAGTWSAWSPEAWGTPSTGLLQDEIPDWSQGHGQQLEAVVAQEDSLAPARPSLQPDPRPLDHRDPLEQVAVLASLGIFSCLGLAVGALALGLWLRLRRSGKEGPQKPGLLAPMIPVEKLPGIPNLQRTPENFS

SEQ ID NO: 10 - DNA, 1761 bp, Mus musculus
tattggcgctgggaggagccgcccccactgcctgcgctcagcactggagggagcagaggccggggcagagggtgagggcggagggcgctggcggcggctgccgcagaagatgagcagcagctgctcagggctgaccagggtcctggtggccgtggctacagccctggtgtcttcctcctccccctgcccccaagcttggggtcctccaggggtccagtatggacaacctggcaggcccgtgatgctgtgctgccccggagtgagtgctgggactccagtgtcctggtttcgggatggagattcaaggctgctccagggacctgactctgggttaggacacagactggtcttggcccaggtggacagccctgatgaaggcacttatgtctgccagaccctggatggtgtatcagggggcatggtgaccctgaagctgggctttcccccagcacgtcctgaagtctcctgccaagcggtagactatgaaaacttctcctgtacttggagtccaggccaggtcagcggtttgcccacccgctaccttacttcctacaggaagaagacgctgccaggagctgagagtcagagggaaagtccatccaccgggccttggccgtgtccacaggaccctctggaggcctcccgatgtgtggtccatggggcagagttctggagtgagtaccggatcaatgtgaccgaggtgaacccactgggtgccagcacgtgcctactggatgtgagattacagagcatcttgcgtcctgatccaccccaaggactgcgggtggaatccgtacctggttacccgagacgcctgcatgccagctggacataccctgcctcctggcgtcgccaaccccactttctgctcaagttccggttgcaataccgaccagcacagcatccagcctggtccacggtggagcccattggcttggaggaagtgataacagatgctgtggctgggctgccacacgcggtacgagtcagtgccagggactttctggatgctggcacctggagcgcctggagcccagaggcctggggtactcctagcactggtcccctgcaggatgagatacctgattggagccagggacacggacagcagctagaggcagtagtagctcaggaggacagcccggctcctgcaaggccttccttgcagccggacccaaggccacttgatcacagggaccccttggagcaagtagctgtgttagcgtctctgggaatcttctcttgccttggcctggctgttggagctctggcactggggctctggctgaggctgagacggagtgggaaggatggaccgcaaaaacctgggctcttggcacccatgatcccggtggaaaagcttccaggaattccaaacctgcagaggaccccagagaacttcagctgatttcatctgtaacccggtcagacttgggtggttaaaaggacaggcagaaagaggcggggcagtggatccctgtggatggaggtctcagctgaaagtctgagctcttttctttgacacctatactccaaacttgctgccggctgaaggctgtctggacttccgatgtcctgaggtggaagtccacctgaggaatgtgtacagaagtctgtgttcctgtgatcgtgtgtgtatgtgagacagggagcaaaagttctctgcatgtgtgtacagatgattggagagtgtgtgcggtcttgggcttggcccttctgggaagtgtgaagagttgaaataaaagagacggaagtttttgg

SEQ ID NO: 11 - DNA, 2680 bp, Mus musculus
gactcagtccagaccccttcccccccgcctggggtcagggctggacagggaggagaagcgagctccttgcaagacgagttggggacgagatagctagtgaccctggccttggctgacccgaaagaggcctggagacatctgtcctcaaaggattgtccacttccggtgactccctgctgctgataatagggcccatgtttcccagaaggcccgaggttatcccaagactgcttctctcccttcacctcccatggcttcattcctgaagagcctgtgatcacttagggtcagaaatagacggatgagcagcagctgctcagggctgaccagggtcctggtggccgtggctacagccctggtgtcttcctcctccccctgcccccaagcttggggtcctccaggggtccagtatggacaacctggcaggcccgtgatgctgtgctgccccggagtgagtgctgggactccagtgtcctggtttcgggatggagattcaaggctgctccagggacctgactctgggttaggacacagactggtcttggcccaggtggacagccctgatgaaggcacttatgtctgccagaccctggatggtgtatcagggggcatggtgaccctgaagctgggctttcccccagcacgtcctgaagtctcttgccaagcggtagactatgaaaacttctcctgtacttggagtccaggccaggtcagcggtttgcccacccgctaccttacttcctacaggaagaagacgctgccaggagctgagagtcagagggaaagtccatccaccgggccttggccgtgtccacaggaccctctggaggcctcccgatgtgtggtccatggggcagagttctggagtgagtaccggatcaatgtgaccgaggtgaacccactgggtgccagcacgtgcctactggatgtgagattacagagcatcttgcgtcccgatccaccccaaggactgcgggtggaatccgtacctggttacccaagacgcctgcatgccagctggacataccctgcctcctggcgtcgccaaccccactttctgctcaagttccggttgcaataccgaccagcacagcatccagcctggtccacggtggagcccattggcttggaggaagtgataacagatactgtggctgggctgccccacgcggtacgagtcagtgccagggactttctggatgctggcacctggagcgcctggagcccagaggcctggggtactcctagcactggtctcctgcaggatgagatacctgattggagccagggacatggacagcagctagaggcagtagtagctcaggaggacagcctggctcctgcaaggccttccttgcagccagacccaaggccacttgatcacagggatcccttggagcaagtagctgtgttagcatctctgggaatcttctcttgccttggcctggctgttggagccctggcactggggctctggctgaggctgagacggagtgggaaggaaggaccgcaaaaacctgggctcttggcacccatgatcccggtggaaaagcttccaggaattccaaacctgcagaggaccccagagaacttcagctgatttcatctgtaacccggtcagacttgggtggttaaaaggacaggcagaaagaggcggggcagtggatccctgtggatggaggtctcagctggaagtctgagctcttttctttgacacctatactccaaacttgctgccggctgaaggctgtctggacttctgatgtcctgaggtggaagtccacctgaggaatgtgtacagaagtctgtgttcctgtgatcgtgtgtgtatgtgagacagggagcaaaagttctctgcatgtgtgtacacatgattggagagtgtgtgcggtcttgggcttggcccttctggggagtgtgaagagttgaaataaaagagacggaagtttttggagattgtcgtctaatgttgctttattagttttgctgttgggggtgtgagtacattttcttttgtagtaccagatttaaactgggtacagttccttggagccttttcccttggcgttctaaccaccgagccaggctgcggttctggggatgaacacagacactgcgccgagccaggtgaagcctgagaagggaaaagtccaaagctaggctcacatttgggggatgagcttaggtagggttcaggggttaggggtaaaagcccgatgaaaatccgggtaggtattagagttagtaaaggggctagggatgaatattgggggttcggactgagggcagagttagaggtatcaaagttgggggcagagctcacacgacagcctggaggtgacagtccccatcagcctcctgcagttccatgtggacaatcctcctcagcagcctgcttgggagtggctgtctatagagctgagtacagcagctagtgctggagggcagaggcaaggctggagaacaatagggccacacttcagagaggtctggacccacaagctgctctgattacatagctagaggtccaactttctgggccctgttccctgtagtcccctcgctcagcattcattctatggcctctgagaacttacctgcttcaggagccgggctcagaagcaacagtaacagcgggaggctgctggcgggggagagcccctccatcatgctcactcag

SEQ ID NO: 12 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 13 - protein, 329 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDNIASF

SEQ ID NO: 14 - DNA, 9027 bp, Homo sapiens
ggatctgacagtgttccggagccggggcgagcagccaaaaggcccgcggagtcgcgctgggccgccccggcgcagctgaaccgggggccgcgcctgccaggccgacgggtctggcccagcctggcgccaaggggttcgtgcgctgtggagacgcggagggtcgaggcggcgcggcctgagtgaaacccaatggaaaaagcatgacatttagaagtagaagacttagcttcaaatccctactccttcacttactaattttgtgatttggaaatatccgcgcaagatgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatctagcaaccctaacagtaagaaatcttgttggcaaatcagatgcagctgttttaactatccctgcctgtgactttcaagctactcaccctgtaatggatcttaaagcattccccaaagataacatgctttgggtggaatggactactccaagggaatctgtaaagaaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtgaaggactagtagttcctgctacaacttcagcagtacctataaagtaaagctaaaatgattttatctgtgaattcagattttaaaaagtcttcactctctgaagatgatcatttgcccttaaggacaaaaatgaactgaagtttcacatgagctatttccattccagaatatctgggattctactttaagcactacataaactgactttatcctcagactagctgaatgattttgtgctgtttcaggatgtttgcactgaagaaaaacagaaagcttatctgaaatttataaaactttttgttttgctacatagaaaacagaaggtatttgaataataagcagtgatatgcttagtgagcacagctatactgattttgattagaatagtcatcagagtggcttagggacagttaatataaaagaggagcaaggtgtagaccatcatctacttctgctaaaataacttaaaaagaggtccataggccataactacatgagcccagcttttgtaatctgacaaaaaaatgaggagcagcttcgtgtatatcagtgtacacggtattccttaggtcccttccattggtagtgatgctgcgagttattactggagaaaaggaattctagagctttaacttggcagattaaaagtactcattttttattcatcaataattagtaatctcactagttttcaaaaatttgcatattattgacaacctctttgaagatgcatttcacaaactcaacagagtgccatgataagagctagggatcccccaaactatctcaagcatctaaaaaattgccatttttaaaggcttaaattgtagtagtaaaggggaaaacaggaagtagtagtaaaggggaaaaaaaaccaataaagcatctaaaaaattggcatgttaaaaggcttaaattgctaatgtgtgtatatatatatatatatatacacacacatatcattgacttttcttaagacttcagagtactgggtagatgaacactttatacagtatatatcttcagcttaaatttgttttgagtattttttttatttttaaataagtaggcaaagatttaaatttttttatttttagtaaatgtttgaggcacactaagacaacttgggcaatatttgccaaaacaaaacagaaccccaaaaaatgtacatcttgttcttagcaaatatcattattgtagagacacttaataaagagatggtattttaatgtctgcagttctgaggtagggtggaacttagttctacattgtgatttaggaatttttaaaaccttttttcttcaagggagaagtgacccaggcctcgagtttagtgctaaagccgctagtgtacttatgctgtcccctaaccaccacgtgcgatatggaagcagatgctaaatataggggttttcttagaaagtaagaggaaattagcaagcgttattagtgattgactactgctatcaagtgaattcaaaggaaacaggtttttatgccatatttaagttacagaaaccaggcatgcttagaatagtttctagaggttattggagaatagaaagctaagaaaacttggtatacatttacaatggaaatataattacactttttactctcagaatattgttcacattagacttcctgtttatcttttatattcttgcatttatataatgcctcatcctttcaaagttctttcacatattatatgatcttctttatgaaaaaaatagatgtttcattctgatatattcagtttcccactttaggcaaaagtagattaatagaatgacgaattcaaagtagatgaggaaaatcaggcacagagaagtaaaggtagggatagacccaaatttacacaacaagataatgacatctccagcttttaagttgatcatcaaaggctgggctggatttgtcttgctgtatgtgtcaggaaatttatacctattacattttccattttctcaaaatttaagtcacatgactaatatttagctgcaactttcctcataacaaatagtgtcatgaagaatgttgtagtgtgaagtttgtacatttcagggtcagatatacaatatgaactcttaatctacaggaatgagaatggaggatcattgaaggccatgatataaacaaatttgcatgttgaagcctgtataaaacatggtacagtgagtgaatatacccccatccccaagaacactttatacatattaaatggatatatgattactgtgcaaaaattcattctggaaatgaacatatatttgagcactaatatgtaatgtacacctgccctaaggagaaaataaattataaaactttttacattcaaaattactttcccaagcatgtcttagaataatctatgtgttgatgcatgtaaattgtactttaggtaggcaaagaaatctggttatttatgtaaaaactagtctaataaagttagttagtggctttatcactttaaatctttagtgtccaaaagtggtgtttaaagtaatagcacatcagaaaaccttgtctggacaaaactagttcactcactgcttctgcacctgcagttgctccctttagggttataaaataatgacccaaatgttacatgtgttgatattataacttgtcagttactgatgtctgtggtatcctaccctcatctctgaaagggataatactgaataattattagaaaactataaaacttcacactttgtaccattaaaacctaaaattttaatcttgtccttttttactatggatcagtcggcactcgggaacagcagcaaggaaaaaaagcaaatttcattcacatgttctgtgttcatacctcttctctacctaattgttcatttaaatttcagccttattccttgataagggattttaccacatgaagtcatccagtgaccctagctcttattgtgaagttagtggagtatacttagaaatgttacaactttaaaatgttacaaaacattcattaaagctcatatttaaagtagagcatctagtttgagaaatagaaatcaattattaaagatgtcttttttctacccatttaactagttaaaaccatgacatgtaaatgtagaagtagaataatcatagaattccctaaaatatttctgtttactaacatatattgaccaagtacatcaagcaggagagatcttccttcattctgttatagtccacatcattctaattttgctcagttgttattaagagcatattcctaaaccatacacttttgtttcaataaagttttattttgttgagatgaataaaataacaaagttataagctgcataagacaaaagttcaattgttcaaaaaaaatttactgggatagctttctattacaggtattgttagattatattgtgctgataagattactttctaaaaaatttgtacttttctgtaaattaaaagaatatggagtcataaaatggcaagtgttttaggattagcctaaaattggacattgtcattgatttcaaagaaggtatgaactagcagtcttacagcctaattcttctttggactggtccttggcagcagttccttttcagactcgataaacagaattcagatgatgtaagtcaaaacaaaactttacaaagccaagcgtattatcttttgcattaacctatttttttccatcatacatgctactagtatgtgcattagcatgatattctcatatacattgcattaaaaattaaaaggtggcagctcagggtgagctcttctgttgctcatttgttcctaaatttttaagggctttttctcagtcaatagtttgtacaaactggttagtttaacttcattacccatttcattaaagttgatgggtcgtgtgatgagatgcatttaaggccgatagtgatagatgttttttttatttcttgaacacaggctttgtctgaatgatgttcttttatctcttgaacacaagctttgaatgataactacaggttttaagtgctgttacattaataccataatgtgatgtgttagaaacaaagggatatttcaaaggtagatatttgaaaattctctagtctcaatatgtatgtgtattgaatatactctaaaaataaatgtgcaatttgctagtaggacaatgcagtgactgactagcattaggtatgtttcttttatatcctagctatgtcccactttcttctaagtgcaatcctttcatgttcacttgctgttttaccccatctactctaacttcatttggaaggcttgtctagagtatagcatgtatttttacctttgcagtgaattgcatgtgctaattgtaaccacagctatttttatgttgacataactccaaatgttatattaaatgttctattatatattagctctaatcccttaagtaaattttaagaaataaatacttgttcaaattttttttctgtatgtggttactatcatctgactatgcatatttgtaacagcatttatcattagtggtgttagctaaataagcatcttagtgtaaatgagatgcttcgtgtgggttttgtgacattttaaatgacataatggaatgtgatttaaaagaaaaccagtacactatcttggtcttaataacatagaatggagatggcaaatttatccactagttttccagatttactatttaatagctgaggtctgaaatcgtagcatcctccctcctagtggacattaaaaaaaaaaaaaaaaaaaaaaacctacttggttgtcaagagcccaagtatggaggtgctgcgccatcttgtggcctgtctgtgcccaccctgcactctgctggagtctccatccttgttgcagtgagacttgaagttcaagattgatacatggcatcctcctgctacttcttgaggttactaagtagtatatgaaactaatcagtcagcaagtccacctggaaggaaaagaaaatctcaactattaatgtgccttcacattgtgattttgtctaaaaaaatgtagtgagtcaaaaaacccacaagccagccaacagtaactccttcacatatataccagagtttatagaaataacatgtcagctttgggctatgtgctcctttgtttaaaatcttctatttggttatggcttgtataggctcaagcctgatttctttaaggtgtggtggctcatcttatcctaatgtgtatgatagatacagtccatcctgctttggaaaagattatgtaactccttgagagcatactctttctctagcccaaaggcagtgagagagttttcttgttcaggattgcttaactttccatttaagctttttctttttaaattaatacaaacttctacactttcaaaatacgaaatatattacaactgcgtataggctcttccatacttaagtccagtgcttgggcaagttaatggagtgaaagactacaagcaaagaggaactgaggtagaaaaagaagaatgtgtgaaagcagcaggaagctcagccaactcgaaagcagggtgaacagcttgagtcctgttgctgctgatcggggttggctcttggacaacttagtaagatcatggaaaggctgcttgggttctccatagaaaagttctgtctccatcaagggaggaaaatgtacctttcaactcaaaattcaatatttgtttttaaatatagctattttccccaaccgctaaagattttcaacagatacgaagccagagcttagttttagaaacctgtggacattcaaacctgattctttattccctgtgactatggttatgtcattttacatgtcaaaaaagtgtatctagaattgtcatttcttatttttgagctttttttagtgagaattatcccctcacttaaatggctttttatttaaacatctgtgcattctgtatgaaattgtagtctttctgggataacatggtgagctatatggtggtaatccacacacacaaaaataaaagccaaaaaaaaaccaaaa

SEQ ID NO: 15 - DNA, 8844 bp, Homo sapiens
ggatctgacagtgttccggagccggggcgagcagccaaaaggcccgcggagtcgcgctgggccgccccggcgcagctgaaccgggggccgcgcctgccaggccgacgggtctggcccagcctggcgccaaggggttcgtgcgctgtggagacgcggagggtcgaggcggcgcggcctgagtgaaacccaatggaaaaagcatgacatttagaagtagaagacttagcttcaaatccctactccttcacttactaattttgtgatttggaaatatccgcgcaagatgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatctagcaaccctaacagtaagaaatcttgttggcaaatcagatgcagctgttttaactatccctgcctgtgactttcaagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtgaaggactagtagttcctgctacaacttcagcagtacctataaagtaaagctaaaatgattttatctgtgaattcagattttaaaaagtcttcactctctgaagatgatcatttgcccttaaggacaaaaatgaactgaagtttcacatgagctatttccattccagaatatctgggattctactttaagcactacataaactgactttatcctcagactagctgaatgattttgtgctgtttcaggatgtttgcactgaagaaaaacagaaagcttatctgaaatttataaaactttttgttttgctacatagaaaacagaaggtatttgaataataagcagtgatatgcttagtgagcacagctatactgattttgattagaatagtcatcagagtggcttagggacagttaatataaaagaggagcaaggtgtagaccatcatctacttctgctaaaataacttaaaaagaggtccataggccataactacatgagcccagcttttgtaatctgacaaaaaaatgaggagcagcttcgtgtatatcagtgtacacggtattccttaggtcccttccattggtagtgatgctgcgagttattactggagaaaaggaattctagagctttaacttggcagattaaaagtactcattttttattcatcaataattagtaatctcactagttttcaaaaatttgcatattattgacaacctctttgaagatgcatttcacaaactcaacagagtgccatgataagagctagggatcccccaaactatctcaagcatctaaaaaattgccatttttaaaggcttaaattgtagtagtaaaggggaaaacaggaagtagtagtaaaggggaaaaaaaaccaataaagcatctaaaaaattggcatgttaaaaggcttaaattgctaatgtgtgtatatatatatatatatatacacacacatatcattgacttttcttaagacttcagagtactgggtagatgaacactttatacagtatatatcttcagcttaaatttgttttgagtattttttttatttttaaataagtaggcaaagatttaaatttttttatttttagtaaatgtttgaggcacactaagacaacttgggcaatatttgccaaaacaaaacagaaccccaaaaaatgtacatcttgttcttagcaaatatcattattgtagagacacttaataaagagatggtattttaatgtctgcagttctgaggtagggtggaacttagttctacattgtgatttaggaatttttaaaaccttttttcttcaagggagaagtgacccaggcctcgagtttagtgctaaagccgctagtgtacttatgctgtcccctaaccaccacgtgcgatatggaagcagatgctaaatataggggttttcttagaaagtaagaggaaattagcaagcgttattagtgattgactactgctatcaagtgaattcaaaggaaacaggtttttatgccatatttaagttacagaaaccaggcatgcttagaatagtttctagaggttattggagaatagaaagctaagaaaacttggtatacatttacaatggaaatataattacactttttactctcagaatattgttcacattagacttcctgtttatcttttatattcttgcatttatataatgcctcatcctttcaaagttctttcacatattatatgatcttctttatgaaaaaaatagatgtttcattctgatatattcagtttcccactttaggcaaaagtagattaatagaatgacgaattcaaagtagatgaggaaaatcaggcacagagaagtaaaggtagggatagacccaaatttacacaacaagataatgacatctccagcttttaagttgatcatcaaaggctgggctggatttgtcttgctgtatgtgtcaggaaatttatacctattacattttccattttctcaaaatttaagtcacatgactaatatttagctgcaactttcctcataacaaatagtgtcatgaagaatgttgtagtgtgaagtttgtacatttcagggtcagatatacaatatgaactcttaatctacaggaatgagaatggaggatcattgaaggccatgatataaacaaatttgcatgttgaagcctgtataaaacatggtacagtgagtgaatatacccccatccccaagaacactttatacatattaaatggatatatgattactgtgcaaaaattcattctggaaatgaacatatatttgagcactaatatgtaatgtacacctgccctaaggagaaaataaattataaaactttttacattcaaaattactttcccaagcatgtcttagaataatctatgtgttgatgcatgtaaattgtactttaggtaggcaaagaaatctggttatttatgtaaaaactagtctaataaagttagttagtggctttatcactttaaatctttagtgtccaaaagtggtgtttaaagtaatagcacatcagaaaaccttgtctggacaaaactagttcactcactgcttctgcacctgcagttgctccctttagggttataaaataatgacccaaatgttacatgtgttgatattataacttgtcagttactgatgtctgtggtatcctaccctcatctctgaaagggataatactgaataattattagaaaactataaaacttcacactttgtaccattaaaacctaaaattttaatcttgtccttttttactatggatcagtcggcactcgggaacagcagcaaggaaaaaaagcaaatttcattcacatgttctgtgttcatacctcttctctacctaattgttcatttaaatttcagccttattccttgataagggattttaccacatgaagtcatccagtgaccctagctcttattgtgaagttagtggagtatacttagaaatgttacaactttaaaatgttacaaaacattcattaaagctcatatttaaagtagagcatctagtttgagaaatagaaatcaattattaaagatgtcttttttctacccatttaactagttaaaaccatgacatgtaaatgtagaagtagaataatcatagaattccctaaaatatttctgtttactaacatatattgaccaagtacatcaagcaggagagatcttccttcattctgttatagtccacatcattctaattttgctcagttgttattaagagcatattcctaaaccatacacttttgtttcaataaagttttattttgttgagatgaataaaataacaaagttataagctgcataagacaaaagttcaattgttcaaaaaaaatttactgggatagctttctattacaggtattgttagattatattgtgctgataagattactttctaaaaaatttgtacttttctgtaaattaaaagaatatggagtcataaaatggcaagtgttttaggattagcctaaaattggacattgtcattgatttcaaagaaggtatgaactagcagtcttacagcctaattcttctttggactggtccttggcagcagttccttttcagactcgataaacagaattcagatgatgtaagtcaaaacaaaactttacaaagccaagcgtattatcttttgcattaacctatttttttccatcatacatgctactagtatgtgcattagcatgatattctcatatacattgcattaaaaattaaaaggtggcagctcagggtgagctcttctgttgctcatttgttcctaaatttttaagggctttttctcagtcaatagtttgtacaaactggttagtttaacttcattacccatttcattaaagttgatgggtcgtgtgatgagatgcatttaaggccgatagtgatagatgttttttttatttcttgaacacaggctttgtctgaatgatgttcttttatctcttgaacacaagctttgaatgataactacaggttttaagtgctgttacattaataccataatgtgatgtgttagaaacaaagggatatttcaaaggtagatatttgaaaattctctagtctcaatatgtatgtgtattgaatatactctaaaaataaatgtgcaatttgctagtaggacaatgcagtgactgactagcattaggtatgtttcttttatatcctagctatgtcccactttcttctaagtgcaatcctttcatgttcacttgctgttttaccccatctactctaacttcatttggaaggcttgtctagagtatagcatgtatttttacctttgcagtgaattgcatgtgctaattgtaaccacagctatttttatgttgacataactccaaatgttatattaaatgttctattatatattagctctaatcccttaagtaaattttaagaaataaatacttgttcaaattttttttctgtatgtggttactatcatctgactatgcatatttgtaacagcatttatcattagtggtgttagctaaataagcatcttagtgtaaatgagatgcttcgtgtgggttttgtgacattttaaatgacataatggaatgtgatttaaaagaaaaccagtacactatcttggtcttaataacatagaatggagatggcaaatttatccactagttttccagatttactatttaatagctgaggtctgaaatcgtagcatcctccctcctagtggacattaaaaaaaaaaaaaaaaaaaaaaacctacttggttgtcaagagcccaagtatggaggtgctgcgccatcttgtggcctgtctgtgcccaccctgcactctgctggagtctccatccttgttgcagtgagacttgaagttcaagattgatacatggcatcctcctgctacttcttgaggttactaagtagtatatgaaactaatcagtcagcaagtccacctggaaggaaaagaaaatctcaactattaatgtgccttcacattgtgattttgtctaaaaaaatgtagtgagtcaaaaaacccacaagccagccaacagtaactccttcacatatataccagagtttatagaaataacatgtcagctttgggctatgtgctcctttgtttaaaatcttctatttggttatggcttgtataggctcaagcctgatttctttaaggtgtggtggctcatcttatcctaatgtgtatgatagatacagtccatcctgctttggaaaagattatgtaactccttgagagcatactctttctctagcccaaaggcagtgagagagttttcttgttcaggattgcttaactttccatttaagctttttctttttaaattaatacaaacttctacactttcaaaatacgaaatatattacaactgcgtataggctcttccatacttaagtccagtgcttgggcaagttaatggagtgaaagactacaagcaaagaggaactgaggtagaaaaagaagaatgtgtgaaagcagcaggaagctcagccaactcgaaagcagggtgaacagcttgagtcctgttgctgctgatcggggttggctcttggacaacttagtaagatcatggaaaggctgcttgggttctccatagaaaagttctgtctccatcaagggaggaaaatgtacctttcaactcaaaattcaatatttgtttttaaatatagctattttccccaaccgctaaagattttcaacagatacgaagccagagcttagttttagaaacctgtggacattcaaacctgattctttattccctgtgactatggttatgtcattttacatgtcaaaaaagtgtatctagaattgtcatttcttatttttgagctttttttagtgagaattatcccctcacttaaatggctttttatttaaacatctgtgcattctgtatgaaattgtagtctttctgggataacatggtgagctatatggtggtaatccacacacacaaaaataaaagccaaaaaaaaaccaaaa

SEQ ID NO: 16 - protein, 199 aa, Mus musculus
MNCVCRLVLVVLSLWPDRVVAPGPPAGSPRVSSDPRADLDSAVLLTRSLLADTRQLAAQMRDKFPADGDHSLDSLPTLAMSAGTLGSLQLPGVLTRLRVDLMSYLRHVQWLRRAGGPSLKTLEPELGALQARLERLLRRLQLLMSRLALPQAAPDQPVIPLGPPASAWGSIRAAHAILGGLHLTLDWAVRGLLLLKTRL

SEQ ID NO: 17 - protein, 199 aa, Macaca fascicularis
MNCVCRLVLVVLSLWPDTAVAPGPPPGSPRASPDPRAELDSTVLLTRSLLEDTRQLTIQLKDKFPADGDHNLDSLPTLAMSAGALGALQLPSVLTRLRADLLSYLRHVQWLRRAMGSSLKTLEPELGTLQTRLDRLLRRLQLLMSRLALPQLPPDPPAPPLAPPSSTWGGIRAAHAILGGLHLTLDWAVRGLLLLKTRL

SEQ ID NO: 18 - protein, 199 aa, Rattus norvegicus
MNCVCRLVLVVLSLWPDRVVAPGPPAGSPRVSSDPRADLDSAVLLTRSLLADTRQLAAQMRDKFPADGDHNLDSLPTLAMSAGTLGSLQLPGVLTRLRVDLMSYFRHVQWLRRAAGPSLKTLEPELGALQARLERLLRRLQLLMSRLALPQAAPDQPAVPLGPPASAWGSIRAAHAILGGLHLTLDWAVRGLLLLKTRL

SEQ ID NO: 19 - DNA, 1949 bp, Mus musculus
tgcctggcccttgctgctcacactcacaaacctcccctccccaggccgaggcccagctgaccccctgggtcccccggcagcggacagggaagggttaaagcccccccccccggctccctgccccctgccctggggaacccctggcctgtggggacatgaactgtgtttgtcgcctggtcctggtggtgctgagcctctggccagatagagtcgttgcccctgggccaccagctggctcccctcgagtctcttcagaccctcgagcagatctggacagcgctgttctcctaacccgatccctcctggcagacacacggcaactagctgcacagatgagagacaaattcccagctgacggagatcacagtctggactccctgcccaccttggccatgagcgctgggacattgggatctttgcagcttcctggtgtgctgacaaggcttcgagtagacttgatgtcctacctccggcatgtacaatggctgcgccgtgcaggtggtccttccctaaagactctggagccagagctgggtgccctgcaagcccgactggaacggctactccgccgtttacagctcttgatgtctcgcctggccttgccccaggcagccccagaccaacctgtgatccccctgggccctcctgcctcagcctggggaagcatccgggcagctcatgccatcctaggagggctgcacctgaccttggactgggccgtgcggggcctgctgttgttaaagactcgactgtgactcgggactgaaaaccaccatcgataccgccctttaaaaccagatcttatttatttatttattttggtacttggaggggggcatgatgatcaggggcatgccacaccccaaacaaccgccccacacccagctagacagtctttccagtagacctgggtgagggggatcacctgtggcttatttatacttatttatttaaacaattggggggtgggtgggtgcatctgagaccctgaagagcagggaactgagatcctgggttctggggtctctatgaaatctgttcacagtgtcttcctcacttcctacattatttattaaacgattactttttatattaagaggaggaaggagggaagccgggcgtggtggcgcatgcctttaatcccagcactcgggaggcagaggcaggcagatttctgagttcgaggccagcctggtctaagagtgagttcgtgagttccaggacagccagggctacacagagaaaccctgtctcgaaaaaccaaaaaaaaaaaaaaaaaaaaagaggaggaaggagggggaaaaaggaagcctggggttttataccaaaatgtgagtttttttttgtgagtcagagaagtgagaggatttaaatacatatctatttagaggttattagcaggagtatgccgaaaggatcggagtctaaactggtaccctagcatttcaaaggtcctcaacagatacttaacaaaaagttccagagcatcaccttataactaatagcacctgtatggtacagagctgtaaggtacaaggttttttgtttcagcaaaattcacgatgaccaatgggaaacaatctaggctttctggggggcgggcaggggatggatgaataaagctagggatacatccgcatgatgaagttctagctcgccaaccataaaaaggcaagttccaggcgaagactgctttagatattgaacgtctctgcgacccaggagcagcagatggtagaactactctccctatacatgagtatggactgtttggtttttcaaaatggaaacaccatgatgttgacgatagctgcctcagggctgaaaacaagcaatggcacagtgggtgactgttactgtttgtgtttgcattttccagagttttcaccatgactgtattttgcaggacacatttataaacattaataaacactatttttagaaaaaaa

SEQ ID NO: 20 - DNA, 2550 bp, Macaca fascicularis
cgaccacccccccctttcccttttcaacttttccaacttttccttccgtgccctcctccgagcgcggcggcgtgagccctgcaaggcagccgctccgtgtgaatggaaaaggcaggcagggagggtgagtcaggatgtgtcaggccgccctcccctgccgcctgccccccgcccgcccgccccagccccctatataaccccccaggcgtccacactccctcactgcctcggccctgctgctgacgggcacatgcctcccctccccaggccgcggcccagctgacccccggggctcccccggcagcggacagggaagggttaaaggcccccggctccctgccccctgccctggggaacccctggccctgcggggacatgaactgtgtttgccgcctggtcctggtcgtgctgagcctgtggccagatacagctgttgcccctgggccaccacctggctcccctcgagcttccccagaccctcgggccgagctggacagcaccgtgctcctgacccgctctctcctggaggacacgcggcagctgactatacagctgaaggacaaattcccagctgacggggaccacaacctggattccctgcccaccctggccatgagcgcgggggcactgggagctctacagctcccgagtgtgctgacaaggctgcgagcggacctactgtcctacctgcggcatgtgcagtggctgcgtcgggcaatgggctcttccctgaagaccctggagcctgagctgggcaccctgcagacccggctggaccggctgctgcgccggctgcagctcctgatgtcccgcctggccctgccccagctgcccccagacccgccggcgcccccgctggcgcccccctcctcaacctgggggggcatcagggccgcccacgccatcctgggggggctgcacctgacacttgactgggccgtgagggggctactgctgctgaagactcggctgtgacccgaggcccagagccaccaccgtccttccaaagccacatcttatttatttatttatttcggtactgggggcgaaacagccaggtgatccccctgcctttatctccccctagttagagacagtccttccgtgaggctggggggcatctgtgccttatttatacttatttatttcaggagcgggggtgggctcctgggtccccgaggaggagggagttggggtcccggattcttgtgtccacagacttctgccctggctcctcccgctcgaggcctgggcaggaatacatactatttatttaagcaattacttttcatgttggggtggggagggaggggaaagggaagcctgggtttttgtacaaaaatgtgagaaacctttgtgagacgaagaacaaggaattaaatgtgtcatacatatccacttgagggcgacttgtctgagagctggggctggatgctcgggtaactggggcagggcaggtggaggtcccgagtgggcggggcagggactgggagatgggtcagtcacccagacagctctggggaggcagagtttgagcctcgcctggggccccacactgcacagggctctttgttttttgagatggagactggctctgttgcctaggttggagtgcagtggcgcaatctaaactcactgcaacctccacctcccgggtttaatcgattctcctgcctcagcctcccgattagctgggatcacaggcgtgcaccaccacgcccggctaattatttatttcttttgtattttcagtagagacaaggtttcaccatgttgaccaggctggtttcgaactcctgacctcaggtgatcctcctgccttggcctcccaaagtgctgggattacaggtgtgagccaccgcacctgacctatataggtcttcaataaatatttaatggaaggttccagaagtcaccctgcgattaacggtacccgtatgtgacaaagctgcaaggtcaagatggttcattatggctgtgttgaccgtagcaaattggaaacaatccagatatccaacaggtgagggttaagcaacatggtgcatcggtggatggaatgccacccggccacccagagcagggactagcattcagggaggctaaggagagaggcctgcttcggatatagaaagatagcctgacattggccaggcatggtggtcacgcctgtaatcctagcactctgggaggacgaagcaagtggatcactgaagtccaatagtttgagaccagcctgggagacgtggcgaaaccctgtctcaaaagagaaaaaacgatgtcctgacgtgaaacagctacaaaaccactgcatgatgcgattccaatttttgtgtttttctttctatatatggattaaaaaaaaaaaaaaaaccctaaagggaaataagccaaatgttgacaaggactgtctccaggtcaaaggagagaggtgggattgtgggtgacttttgatgtttatgattgtctgtattttacagaatttctgccatgactgtgtattttgcatgacatattttaaaaataataaacactatttttagaagaacagaa

SEQ ID NO: 21 - DNA, 1675 bp, Rattus norvegicus
gccgaggcccagctgaccccctgggctcccccggcagcggacagggaagggttaaaggccccctccccccggctccctgccccctgccctggggaacccctggcctgtggggacatgaactgtgtttgtcacctggtcctggtggtgctgagcctctggccagatagagtcgttgcccctgggccaccagctggctcccctcgagtgtcttcagaccctcgtgcagatctggatagcgctgtcctcttgaccaggtccctcctggcagacacacggcaactagctgcacagatgagagacaaattcccagctgatggagaccacaatctggactccctacctaccttggccatgagcgctgggacactgggatctttgcagcttcctggagtgctgacaaggcttcgagtagacttaatgtcctacttccgacatgtacagtggttgcgccgggcagctggtccttccctaaagactctggagccagagctgggtgccctgcaagcccgactggaacggctacttcgtcgcttacagctcttgatgtctcgcctagccttgccccaggcagccccggaccaacctgcggtccctctgggccctcctgcctcggcctggggaagcatccgggcagctcatgccatcctaggagggctgcacctgaccttggactgggccgtgcggggcctgctgttgttaaagactcggctgtaactcaggactgaaagccaccatcgacatcgtcctttaaagccagatcttatttatttatttattttggtacttggagcgggggagcatgatgaacaggggcatgccacaccccaaacagccacacccagctagataaagtccttccaggagacctgggtgagggggctcacctgtggcttatttatacttatttatttaaagaattggggtgggtgcatctgagaccctgaggagcagggaactgagatcctgggtcctgggtctttacgaagtctattcacagtgtcttcctcacttttttattatttattaaacaattactttttatattaggaggatggagggggggaaaggaagcctggggtttttataccaaaatgtgattttttttttgtgacacagagaagtggggtatttaactattaatgtggggtatataactatttagaggttattagcaggaatattccaaagggatctgagtgtaaaccggtaccctagcacttcaaaggtcctcaacagatacttagcaaacggaccgaaacacctcatgtctaacagcacctgcatggtacagagatagcaaggttcaaggtgtttcagcaagagtcacgcaccctggcaaatgggaaacaacttaggttttcggggggggggggggggcaggggacggatgaatctagttagggaagttccaggcaaagacttctttagatgttgaacgtccctgtgccccgagagcagcagaggatagaacctctctttctatacatggatatagattttttttgtttttcaaaatggaaacaccaaaatgttgacaacagctgcctcagggttgaaaacaagagatggcactgtgggtgactgttattgtttgtgcttgcattttccagagtttccaccatgactgtattttgcatgacacgtttacaaacaataataaacactatttttagaa

SEQ ID NO: 22 - protein, 455 aa, Macaca fascicularis
MSSGCSGLSRVLVAVATALVSASSSCPQAWGPPGVQYGQPGRSVKLCCPGVTAGDPVSWFRDGEPRLLQGPDSGLGHELVLTQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGFPRRLRASWTYPASWPRQPHFLLKFRLQYRPAQHPAWSTVRPGVEPAGLEEMITDAVAGLPHAVRVSARDFLDAGTWSTWSPEAWGTPSTGTVPKEVPAWGQLHMQPEVEPQVDSPAPPSPSLQPHPRLLDHRDSVEQVAVLASLGILSFLGLMAGALALGLWSSKPIEDPGGLRQSPLIILSCWCGWMDSRSLWLGLSWKFCLEPISVRPCISNLPAERCLYL

SEQ ID NO: 23 - protein, 431 aa, Rattus norvegicus
MSSSRSGLTRVLVAVATALVSSSTPCPQAWGPPGVQYGQPGRPVMLCCPGVNAGTPVSWFRDGDSRLLQGPDSGLGHRLVLAQVDSRDEGTYVCRTLDGVFGGMVTLKLGSPPARPEVSCQAVDYENFSCTWSPGRVSGLPTRYLTSYRKKTLPGAESQRESPSTGPWPCPQDPLEASRCVVHGAEFWSEYRINVTEVNPLGASTCLLDVRLQRILRPDPPQGLRVESVPGYPRRLHASWTYPASWRRQPHFLLKFRLQYRPAQHPAWSTVEPIGLEELITDAVAGLPHAVRVSARDFLDAGTWSAWSPEAWGTPSTGPLRDEVPDGSRGHEQKLEAAAQEDSPAPPSPSLQPDPRPLDHRDPLEQVAVLASLGIFSFLGLAVGALALGLWLRLRRSGKDGPQKPGFLAPMIPGDKLPGIPNLQRTPENFS

SEQ ID NO: 24 - DNA, 3389 bp, Macaca fascicularis
agagggcgagggcagagggcgctggcggcagcggccgcggaaggtgaggcctggtgtagacgccaaagtcttgtcacgagtgggcggggcagggtgatgtatgatctgaaaggccgagagtgactgacgactgtcagtgtgtgtgtgcctgtgagtctgtgtctgtgtgacagtatcattgtgtgcgattctgagtgtcacttggtgcagctgtcagctaatgtgactgcgtgtgtcagtgtgcatggagctggggctgcatgtggctgtgcaagtgtgggattatgtgtgtatctgtcaatgcacatttttggtgggactgggttgtgtgtttgtgtgagagggactgtgaccacattagggaatgtgttttgggaaggtctctgtggggtttgagaccatgtctgccagcatgagagtgggtggctggctttgtgtgtgccaatgtgtgacttcagcagtagcagagcaaaagtgaggtttgtgactatgtgtgtgtgctgggtcctaacttaaaccgtgtgggagtacagctgtattcatgtattgggggacctacttcaagagcccataggactgacctttctcttaagtgtccagcatggacggacacacccacaacacacgtggcttcccctccttcttcctctgctcaagaatccccagcatcaggtctactgctccttactttctgtttccctgcttgactcagtttcccttacatgccgttttctctccctgttgctgtcttactagggatatctctgcgtgtttcttcatagctttgttcccctggtctctgtgtggtgcatttccagttttgcctctcctgtctctttgtttctccctgtgcccctatgcctgccttccctgctgatcttgatctctttggctagttgggggcctgtaacagtgatgagatgcaggtctgccactgctaagagctccctgagagagggggcttagaccgtgatgaggagttgcttttaggcggtgctgtgagccctggtagggggctgtgtatgtgtgtgactgcgtgagtttatgaatatgtgtaagtgtatctgagtctgaggctgtgtggctgtgcctgtgtgactgtgagtgaccgcatgtgagagtgtgtaagtctgagggtaactgtgggtcttgtgtatctggcaccaagggaccaaaggcatttcctggtgctggggcccaaagagtggagggggaggtgagaaatggctggaacttccaggggagggggaactgcactctctggcctctccttatatcccccaacttggggcctgctttcccctggaagcacagccagctgtgccctaaggaaacatttggtgaggagtgaaaagaggaggagtttatatctgatttttcatttgattgtgtccacgtgtgttcaaaggcatgcccttttgttaactgtgcgtcatgagtccatgtgtgaatgtgactatgtctggctgtgtgaaaacacagggcatctctcactgacagggacataagtattagccaccctcttttccctcttgctggccttggctgtgtctgtggcttcctttccagacttgggggaggcctcttcccttcacagggccttgggtgggagggaaggagggaggaggcagaagcggggagggggcctggctaggggcggcggagctccagggcttagagggggcggggcagggaaggggaaggaggggatggctttggggtcccagaactgagtggagtaggagacaggggctgtagctggtgagaagtcctggaggccatggacactctgctgctgggatcaccgagatgagcagcggctgctcagggctgagcagggtcctggtggccgtggctacagccctggtgtctgcctcctcctcctgcccccaggcctggggccccccaggggtccagtatgggcagcccggcaggtccgtgaagctgtgttgtcctggagtgactgctggggacccagtgtcctggtttcgggacggggagccaaggctgctccagggacctgactctgggctagggcatgaactggtcctgacccaggcagacagcactgatgagggcacctacatctgccagacgctggatggtgcacttgggggcacagtgaccctgcagctgggctaccctccagcccgccctgttgtctcctgccaagcagctgactatgagaacttctcttgcacttggagtcccagccagatcagcggtttacccacccgctacctcacctcctacaggaagaagacagttctaggagctgatagccagaggaggagtccatccacagggccctggccatgcccacaggatcccttaggtgctgcccgctgtgttgtccacggggctgagttctggagccagtaccggattaatgtgactgaggtgaacccactgggcgccagcacacgcctgctggatgtgagcttgcagagcatcttgcgccctgacccaccccagggcctgcgggtagagtcggtaccaggtttcccccgacgcctgcgagccagctggacataccctgcctcctggccgcgccagccccatttcctgctcaagttccgtttgcagtaccgtccggcacagcatccagcctggtccacggtgaggcctggagtggagccagctggactggaggagatgatcacagatgctgtggctgggctgccccatgccgtacgagtcagtgcccgggactttctggatgctggcacctggagcacctggagcccggaggcctggggaactccgagcactgggaccgtaccaaaggaggtaccagcttggggccagctacacatgcagccagaggtggagcctcaggtggacagccctgctcctccaagcccctccctccaaccacaccctcggctacttgatcacagggactctgtggagcaggtagctgtgctggcgtctttgggaatcctttctttcctgggactgatggctggagccctggcactggggctctggagctccaaacctatagaggacccaggagggcttcggcagagtccacttataattctgtcttgctggtgtggatggatggacagtagatccctatggttgggtctcagctggaagttctgtttggagcccatttctgtgagaccctgtatttcaaatttgccagctgaaaggtgcctgtacctctgatttcaccccagagttggagttctgctcgaggaatgtgtgtaatgtgtacatctgtgtccatgtgtgaccatgtgtctgtgaggcagggaacatatattctctgcatgcatgtatgtaggtgcctggggagtgtgtgtgggtccttggctcttggcctttccccttgcaggggttgcgcaggtgtgaataaagagaataaggaagttcttgga

SEQ ID NO: 25 - DNA, 1713 bp, Rattus norvegicus
ctcctgtaggagggagtcttggaggccatgagcactcagtcactgtgattttcaagatgagcagcagccgctcagggctgaccagggtcctggtggccgtggctacagccctggtgtcttcctccaccccctgcccccaagcttggggtcctccaggggtccagtatgggcagcctggcaggcccgtgatgctgtgctgccccggagtgaatgctgggactccggtgtcctggtttcgggatggggactcgaggctgctccagggacctgactctggactaggacacagactggtcttggcccaggtggacagccgtgacgagggcacttacgtctgtcggacgctggatggtgtatttgggggcatggtgaccctgaagctgggctcccccccagctcggccggaggtctcctgccaagcagtagactatgaaaacttttcttgtacttggagtccaggccgggtcagcggtttgcccacccgctaccttacttcctacaggaagaagaccctgccaggagctgagagtcagagggagagtccgtctaccgggccttggccatgcccacaagaccccctggaggcttcccgatgtgtggtccacggggcagagttctggagtgaataccggatcaatgtgactgaggtgaacccactgggcgccagcacgtgcctactggatgtgagattacagaggatcttacgtcctgatccaccccaagggctgcgggtggaatcagtacctggctacccgagacgcctgcatgccagctggacataccctgcctcctggcgtcgccagccccacttcctgctcaagttccggttgcaataccgtccagcacagcatccagcctggtccacggtggagcccattggcttggaggagttgataacagatgccgtggctgggctgccccatgcggtacgagtcagcgccagggactttctggatgctggcacctggagtgcttggagcccagaggcctggggtactcctagcaccggtcccctgcgggatgaggtacctgatgggagccggggacacgaacagaagctagaggcagcagctcaggaggacagccccgctcctccaagcccttccttgcagccagacccaaggccacttgatcacagggaccccttggagcaagtggctgtgttagcatctctgggaatcttctctttccttggcctggctgttggagccctggctctggggctctggctgaggctgaggcggagtgggaaggacgggcctcaaaagcctggcttcttggcaccaatgatcccaggggacaagcttccaggaatcccaaacctgcagaggaccccagagaacttcagctgatctcatctgcaaccctgtcagactggggtggttaaatggacaggcaggaggaggcaggacagcggatccctatggatggaggtctcagttcgaagtctggagcacttttctttgacacctgaactccaaacttgctgccagctgctcactctctggacctctgatgccgtccttgaggtggaagtccgcctgaggagcgtgtatcgaagtctgtgtccctgtgactgtgtgtgtgtgtgatggggagagcacgttctccgtatgtgtgtgtatagatgattggagagtgtgtgtgtggtcttgggcttggctctcctggggagcatggagcgtgaaataaagaggcggacgtttttggaaa

SEQ ID NO: 26 - protein, 917 aa, Mus musculus
MSAPRIWLAQALLFFLTTESIGQLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANNKKPCPDDLKSVDLFKKEKVSTEGHSSGIGGSSCMSSSRPSISSNEENESAQSTASTVQYSTVVHSGYRHQVPSVQVFSRSESTQPLLDSEERPEDLQLVDSVDGGDEILPRQSYFKQNCSQPEACPEISHFERSNQVLSGNEEDFVRLKQQQVSDHISQPYGSEQRRLFQEGSTADALGTGADGQMERFESVGMETTIDEEIPKSYLPQTVRQGGYMPQ

SEQ ID NO: 27 - protein, 924 aa, Macaca fascicularis
MKYPHKMLTLQTWVVQALFIFLTTESIGELLDPCGYISPESPVVQLHSNFTAVCVLKEKCMDYFHVNANYIVWKTNHFTIPKEQYTIINRTASSVTFTDISSLNIQLTCNILTFGQLEQNVYGITIISGLPPEKPKNLSCIVNEGKKMRCEWNRGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIRLKYNIQYRTKDASTWSQIPPEDTASTRSSFTVQDLKPFTEYVFRICCMKEDGKGYWSDWSEEANGITYEDRPSKAPSFWYKIDPSHAQGYRTVQLMWKTLPPFEANGKILDYEVTLTRWKSHLQNYTVNDTKLTVNLTNDRYVATLTARNLVGKSDAAVLTIPACDFQATHPVMDLKAFPKDNMLWVEWTTPRESVKKYILEWCVLSDKAPCIADWQQEDGTVHRTHLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFSSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGSDDILPRQQYFKQNCSQHESSPDISHFERSKQVSSVNEEDFVRLKQQISDHISQSCGSGEMKMFQEVSAADPFGPGTEGQIERFETIGMEAAIDEGMPKSYLPQTVRQGGYMPQ

SEQ ID NO: 28 - protein, 918 aa, Rattus norvegicus
MSALRIWLMQALLIFLTTESIGQLVEPCGYIYPEFPVVQRGSNFTATCVLKEKCLQVYSVNATYIVWKTNHVAVPKEQVTVINRTASSVTFTDVVFQNVQLTCNILSFGQIEQNVYGITILSGYPPDIPTNLSCIVNEGKNMLCQLDPGRETYLETNYTLKSEWATEKFPDCRTKHGTSSCMMGYTPIYFVNIEVWVEAENALGNVSSEPINFDPVDKVKPSPPHNLSVTNSEELSSILKLAWVNSGLDSILRLKSDIQYRTKDASTWIQVPLEDTVSPRTSFTVQDLKPFTEYVFRIRSIKENGKGYWSDWSEEASGTTYEDRPSKAPSFWYKVNANHPQEYRSARLIWKTLPLSEANGKILDYEVVLTQSKSVSQTYTVNGTELIVNLTNNRYVASLAARNVVGKSPATVLTIPGSHFKASHPVVDLKAFPKDNLLWVEWTPPSKPVNKYILEWCVLSENSPCIPDWQQEDGTVNRTHLRGSLLESKCYLITVTPVFPGGPGSPESMKAYLKQAAPSKGPTVRTKKVGKNEAVLEWDHLPVDVQNGFIRNYSISYRTSVGKEMVVRVDSSHTEYTLSSLSSDTLYMVHMAAYTEEGGKDGPEFTFTTLKFAQGEIEAIVVPVCLAFLLTTLLGVLFCFNKRDLIKKHIWPNVPDPSKSHIAQWSPHTPPRHNFNSKDQMYSDANFTDVSVVEIEANNKKPCPDDLKSLDLFKKEKISTEGHSSGIGGSSCMSSSRPSISSSEENESAQSTASTVQYSTVVHSGYRHQVPSVQVFSRSESTQPLLDSEERPEDLQLVDSVDSGDEILPRQQYFKQSCSQPGASPDVSHFGRSSQVPSGSEEDFVRLKQQQVSDHISEPYGSEQRRLFQEGSVADALGTGTDGQIERFESVGMETAMDEDISKSYLPQTVRQGGYMPQ

SEQ ID NO: 29 - DNA, 5452 bp, Mus musculus
ggggccgtggggggcggagctaggagagcccgggtcagcggcggacggagggtggcgtgtgcggtcgggaggccagagcttcgagccatccgggctgcggctccgctgagggaccggtggtgtgggccgggatccgcggggctgagggacgctcaggagcggcgaggctcgcgtccggaaggaagacccgatagaaggaacatgacatctagaggcagcgaacttgtttccgattcatgctttatcatttcttaatttcgtatgttgggaacatccctgcaagatgtcagcaccaaggatttggctagcgcaagctttgctttttttcctcaccactgaatctataggtcaacttttggaaccgtgtggttacatctaccctgaatttccagttgtccagcgcggctcgaacttcactgccatttgtgtgctgaaggaggcgtgtctgcagcattactacgtgaatgccagctacatcgtgtggaagaccaaccatgctgctgttcccagggagcaggtcactgtcatcaacagaaccacgtccagtgtcacgttcacagacgtggtcctcccgagcgtgcagctcacctgcaacatcctgtcctttgggcagatcgagcagaatgtgtatggagtcaccatgctttcaggctttcctccagataaacctacaaatttgacttgcattgtgaatgaggggaagaatatgctgtgccagtgggaccccggaagggagacttaccttgaaacaaactacactttgaaatcagagtgggcaacagagaagtttcctgattgccagtcaaagcatggcacttcatgtatggtcagctacatgcccacctattatgtcaacattgaagtctgggtggaagcagagaatgcccttgggaaggtctcctcagagtctatcaattttgaccccgtggataaagtgaaacccaccccaccatataatttatcagtgaccaactcagaagaattatccagtatattaaagctatcatgggtcagttcagggctgggcggtcttttagatctaaagtctgacatccaatataggaccaaagatgcctcaacttggatccaggtccctcttgaagatacaatgtctcctcgaacttccttcactgtgcaggacctcaagccttttacagaatatgtgtttaggatccggtccattaaggacagtgggaagggctactggagtgactggagtgaggaggctagtgggaccacatacgaagacagaccatccagaccaccaagtttctggtataagacaaatccatcccatgggcaggaatatagatctgtacggctcatatggaaggcactgcctctttctgaagccaatgggaaaatcttggattatgaagtgattcttacgcagtcaaagtccgtctcacaaacgtacacagtcactggcacagagctgaccgtgaatctcaccaatgaccgctatgtcgcgtctctagcagcaagaaacaaggtgggcaaatcagctgcagctgtcctcaccatccccagcccccacgtcacagctgcttattctgtagtgaatcttaaagcatttccaaaagataacctgctctgggtggaatggacacctccacctaaacccgtgagcaagtacatcttagagtggtgtgtgttgtcagagaacgcaccctgtgttgaagactggcagcaggaagacgctaccgtgaatcggacccacttgagaggacgcctcctggagagcaagtgctatcaaatcacagtaactcccgtattcgccacggggcccggaggctctgagtccttgaaggcgtacctcaaacaagccgctcctgccagaggaccgactgttcggacaaagaaagtggggaaaaatgaagctgtcttagcgtgggaccagattcctgtggacgaccagaatggcttcattagaaactactccatatcttacagaaccagcgtgggaaaggagatggttgtgcatgtggattcttctcacacggagtacacgctgtcctctctgagtagtgatacgttgtacatggtccgaatggccgcgtacacagatgaaggtgggaaagatgggccggaattcacttttacaacaccaaagttcgctcaaggagaaatagaagccatagtcgtgcctgtgtgcttagccttcctcctgacaaccctgctgggcgtcttgttctgctttaacaaacgagacctaattaaaaaacacatctggcctaatgttcctgatccttccaagagtcatattgcccagtggtcacctcacacccccccaaggcacaattttaactccaaagatcaaatgtactcggacggcaatttcactgatgtaagcgttgtggaaatagaagcaaacaacaagaagccttgtccagatgacctgaagtccgtggacctgttcaagaaggagaaagtgagtacagaagggcacagcagtggcatcgggggctcttcatgcatgtcctcctccaggcccagcatctccagcaacgaggagaatgagtctgctcagagcaccgccagcacggtgcagtactccactgtggtgcacagcggctacaggcaccaggtcccgtccgtgcaagtgttctcaaggtccgagtccacccagcccctgctagactcggaggagcggccagaagacctgcagctggtggatagtgtagacggtggggatgagatcttgcccaggcaaccgtatttcaagcagaactgcagtcagcctgaagcctgtccagagatttcacattttgaaaggtcaaaccaggttttgtccggcaatgaggaggattttgtcagactgaagcagcagcaggtttcagatcacatttctcagccctatggatccgagcaacggaggctgtttcaggaaggctctacagcggatgctcttggcacgggggctgatggacagatggagagatttgaatctgttggaatggagaccacaattgatgaagaaattcccaaaagttacttgccacagactgtaagacaaggtggctacatgccgcagtgaaggactggctcctgaacttcagcaggaactgcaaaataaagctaaagacgagtggcttcagatgagaaacagtcctcactccctgaagataggcattgcctctaaggacaaagtcacacctgggccgtctccattccagagtagctggaattctccttcaagcactacatgaactgactttatcctctatagctggaggactctgtgctctttcagaatgtttgcactgccgaaaacaaagtgtgtctgagtttataaagctgttggtttgctacatagaaaacgggggatttaaatgagccacaatgtcatggcatcgttcagctgggctggttttgatgagactgaccatccagatgccagcgagaaggccagcagcatcatttaaaagtgccctgtggcacagtgtccagattatgtcatctagaagacgaaagaatggaggggtggctcgtttctgggtctgtcagagaagcatggcctgctccttgctgttggtatcgatgccggttactactgaggaagaggataaactttaaagagaagcaacatagaagcacccgctttcttatagtcacctacagcctcccaagaaactgcttattattgaaaatgtctttccgagtcaatttcatccaccctatagtggaccaggctggcctcaaactcagagatctgtctgcctctgcctctgcctctgcctccagagtaccgggatgaaaggcagtgcaccaccactgctccacttacgaagattgctgtttttaaagacttaagttgttgacatttttctgaacacacataaccacaccagggacagatgattctagagactggttgaggaagcactttatccagaatgtcttcaactttaaattactttaattgactccaccttgttttgattttcaagaatagcagggtgtttgttgaagggtggcatttcccagagcctcatgcagtcccagaacacacatcccaaccatgtcagcgaccaccacaatgacagaagtcctcctacagagctgaggaagctggaagggtcctgctagtttgcagggtgggctttgaacccacagccctgtgcatgctagacattcactctaccgctgagctgctcctccgcccggctcattatgtcatctaggctctcgtgatcctcctggcccagcttcctgactgtctgggactgtggatgtgtgctgtcacacacccaaccaggtctttttaatatctgccatttatgaggtagagtttttacacgacttaggactatcttaaacctattccttctagttgctttattaaggaatagcctaggcatccagtgtttagcactaaagccattaatgtatttgtgcctaacttcccagtgtgacatggaggtgggggctaaaggtagacactgggcagacaggaattggctgagcgccgttagaggctgatcactaagtgtgctcacaggaaaagggttgaagtttcagagcatgggcacccagatcatttatagcggttaccacacagtgcctaggaggagttgatatgcacttagttggatgcagtttcaccttttactctcagaaggttgtttatattctacttgctgatgtgttatcatttatatttttatactgggtagtccttttctgtgttctttcacatgtataatcctcgtgagactgtacatgctttgctctggtatattcagttgcctccttggcaaaaccagcctggaattgccaaaatgccggaagtgaagggagatggtgaaagtcgggcacaggaaggccaggtcaggggcagagagaccagaattcacactacaaggctggcaccattgccattcaagttggacgtcaaagcctgatcccagttaggactgagttttctatgtatatatctgccaggaagttaacacttgctgaatttctcatcttctcacagtaagcagtctagttatgggactctatccatgttcagctgtgacttccctcatatcagacagtggtgctgtgaacgttgcagtgtgaagtctgtactggctagggatggtagcccagcgtgagttcaggcatgaacatagaggccactgaaggaagagcctcctggaagcaagttggcacgttaaatcccagacagaaagtgtcccagggagccagggcctccgccagcagggagggggtgtgctcactgcaggaacgttcatttagaagatgtaccatggagcacctccctttgctctaagagaaaacccatcttaaagttcatcttgaatacagacttcgctcgagcatgttttagaataatctggatgctgtaccttgtaccttaggtagcagagaaagtaatctggcgtctttctgtaaaaccgtctaataaagctagttctgcccgtgggctcgtattctttcgtgt

SEQ ID NO: 30 - DNA, 7509 bp, Macaca fascicularis
ggggagcgccccgccccgccgcgggactggggcggcgcgctacctctgcggagaaggatccgcgagcgttccggagccggggcgagcagccaaaaggcccgcggagccgcgctgggccgccccggtgcagctgaaccgggggccgcgcctgccaggccgacggctctggcccagcctggcgccaagggtttcgtgcgctgtggagacgcggagggtcgaggcggcgcggcctgagtgaaacccaatggaaaaagcatgacatttagaagcagaagacttagcttcaaatccctactccttcacttagtgattttgtgatttgggtatcttttaaggagacatattatgaaatatccgcacaagatgttgacgttgcagacttgggtagtgcaagccttgtttatttttctcaccactgaatcaataggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcctccagtgtcacctttacagatatatcttcattaaatattcagctcacttgcaacatccttacatttggacagcttgaacagaatgtttatggaatcacaataatttcaggcttgcctccagaaaaacctaaaaatttgagttgcattgtgaatgagggaaagaaaatgaggtgtgagtggaatcgtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataagactaaaatataacattcaatataggaccaaagatgcctcaacttggagccagattccccctgaagatacagcatccacccgatcttcattcactgtccaagaccttaaaccttttacagaatatgtgtttaggatttgctgtatgaaggaagatggtaagggatactggagtgactggagtgaagaagcaaatgggatcacctatgaagatagaccatctaaagcaccaagtttctggtataaaatagatccatcccatgctcaaggatatagaactgtacaactcatgtggaagacattgcctccttttgaagccaatggaaaaatcttggattatgaagtgactctcacaagatggaaatcacatttacaaaattatacagttaatgacacaaaactgacagtaaatctcacaaatgatcgctatgtagcaaccctaacagcaaggaatcttgttggcaaatcagatgcagctgttttaactatccctgcctgtgactttcaagctactcaccctgtaatggatcttaaagcattccccaaagataacatgctttgggtagaatggactactccaagggaatctgtaaagaaatatatacttgagtggtgtgtgttatcagataaagcgccctgtatcgcagactggcaacaggaagatggtactgtgcatcgcacccatttaagagggaacttagcagagagcaaatgctatttgataacagttactccagtatatgctgacggaccaggaagccctgaatccataaaggcataccttaaacaagctccaccttccaaaggacctactgttcggacaaagaaagtagggaaaaacgaagctgtcttagagtgggaccaacttcctgttgatgttcagaatggatttatcagaaattatactatattttatagaaccatcattggaaatgaaaccgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcgttcctattgacaactcttctgggagtgctgttctgctttaataagcgagacctaattaaaaaacacatctggcctaatgttccagatccttcaaagagtcatattgcccagtggtcacctcacactcctccaaggcacaactttagttcaaaagatcaaatgtattcagatggcaatttcactgatgtaagtgttgtggaaatagaagcaaatgacaaaaaaccttttccagaagatctgaaatcattggacctgttcaaaaaggaaaaaattaatactgaaggacacagcagtggtattgggggttcttcatgcatgtcatcttctaggccaagcatttctagcagtgatgaaaatgaatcttcacaaaacacttcgagcactgtccagtattctactgtggtgcacagtggctacagacaccaagttccatccgtgcaagtcttctcaagatccgagtctacccagcccttgttagattcagaggagcggccagaagatctacaattagtagatcatgtagatggcagtgatgacattttgcccaggcaacagtatttcaaacagaactgcagtcagcatgaatccagtcccgatatttcacattttgaaaggtcaaagcaagtttcatcagtcaatgaggaagattttgttagacttaaacaacagatttcagatcatatttcacaatcctgtggatctggggaaatgaaaatgtttcaggaagtttctgcagcagatccttttggtccaggtactgagggacaaatagaaagatttgaaacaattgggatggaggctgccattgatgaaggaatgcctaaaagttacttaccacagactgtacggcaaggtggctacatgcctcagtgaaggactagtagttcctgctgcaacttcagcagtacctataaagtaaagctaaaattattttatctgtgaattcagatttcaaaaagtcttcactctctgaagatgatcatttgcccttaaggacaaaaatgaactgaagtttcacatgagctatttccattccagaatatctgggattctactttaagcactacataaactgacttcatcctcagactagctgaatgattttgtgctgtttcaggatgtttgcactgaagaaaaatagaaagcttatctgaaatttataaaactttttgttttgctacataaaaaacaaggtatttaaataataagcagtgatatgcttagtgagcacagctatactgattttgatgagtcatcaaagtggcttagggacagttaatataaaagattagaggagcaaggtgtagaccatctacttcagctaaaataacttaaaaagagatccataggccataactatatgagcccagcttttgtaatctgccaaaaaaaaatgagcagcctcgtgtatatcaatgtacacaatattccttaggtcccttccattggtagtgatgctgccagttattactggagaaaaggaattctagagctttaacttggcaggttaaaagtactcattttttattcatcaataattagtaatctcactagttttcaaaaatttgcatattattgacaacctctttgaagaactatttcacaaactcaacagagtgccatgataagagctagggatcccccaaactatctcaagcatctaaaaaattgccatttttaaaggcttaaattgtagtaataaagggggaaaaagtggtagtaaagaggaaaaaaaaaaacaaagcatctaaaaaaatggcatttttaaaggcttaaattgctgatgtttatatatatatacacacacatatatacacacatatcattgaccttaagacttcagagaattgggtagatgaagcactttatatagtatatatcttcagcttaaatttgtttagagtactttttaaaatttttaagtaggtaaagatttaaaactttttatttttagtgaatgtttgaagcaaactaagatgacttgggcaatatttaccaaaacaaaacagaaccccaaaaaatgtacatcttgatcttagcaaatatccttattgtagagacagtaaagagatggtattttaatatctgcagttctgaggtagggtggaacttagttctacattgtgatttaggaatttttaaaaccctttttcttcaagggagaagtgacccaggcctcaagtttaatgctaaagccactagtgtacttatgctgtcccctaaccaccacttggcattatggagacagaggctaaatataggggctttcttaagaaagtaagaggaaattagcaagcattattagtgattgactactgctatcaagtgaattcaaaggaaacagatttttaatgccatatttaagttacagaaaccaggcatgcttagaatagtttctagaggttattggagaatagaaagctaagaaaacttggtatacatttacaattgaaatataattacactttttactctcagaatattattcacattaggcttcctgtttcttttatattcttgcatttatataacacctgatcctttcaaagttctttcacatattatatgatcgtctttatgaaaaaaaatgcttttttctgatatattcagtttcccacttgagacaaaattagattaatagagtcactaattcaaagtagatgaggaaaatcaggcacagagaagtaaaggtagagatagacccaaatttacacaacaagataatgatattgccaccgtttaagttggtcatcaaaggctgggctggatttgtcttgctatatgtgtcaggaaatttatacctattacattttccattttctcaaaataagtcacatgactgtaatgtttagctgcaacttataacaaatagtgtcatgaagaatgttttagtgtgaggtttgtacatttcagagtcagttatacaatgtgaactcttgatctatgggaatgagaatggaggatcattgaaggccatgatacaaacaaatgtacatgttaaagcctgtataaaacatggtacagtgagtgaatacccccatccccaagaacacttgatacatattaaatgggtatatgattactgtccagaaatgcattccggagatgaacattcatttgagcactaatatataatgtacatcttctgccctaaggagaaaataaattataaaacttgttttacattcaaaattactttcccgagcatgtcttagaataatctgtgtgttgatgcatgtaaattgtactttaggtaggcaaagaaacaaatttggttatttatgtaaaaactaatctaataaagttagtgactttatcactttaaatctttagagtctaaaagtggtgtttaaagtaacactagcacatcagagaaccttgtctggacaaaactagttcactcactgcttctgcaactgcagttgctcccttcagggttatagaaaaaggacccaaatgttacatgtgttgatattataacttgtcagttactgatgtctgtagtatcctaccctcacctctgaaagggataatactgaataattattagaaaactataaaacttcacactttgtaccattaagacctaaaattttaatcctatccttttttatgatggatcagtcagcactcgaacagcagcaaggaaaaaaagcaaatttcattcacatattctgtgttcatctctcttctctacctaattgttcatttaatttcagccttgttccttgataagggattttaccacatgaagtcatccagtgtccctagctcttactgtgaagttagtggagtataattacaaatgttacaacttaaaaatgttacaaaacatttatcaaagctcatatttagagtatctagtttgagaaatagaaatcaattattaaagatgtcttttttctacccaatttaactagttaaaaccatgacatgtaaatgtagaagtagaataatcatacagaattccctaaaatgtttctgtttactaacattgagcaagtacatcaagtagagagatcttccttcattctgttatagtccacatcattctaattttgctcagttattattaagagcatattcctaaaccatacacttttatttcaataaagttttattttgttgagatgaataaaatatcaaagttataagctacataagacaaaagttcaattgttcaaacaaatttactgggatagctttctattatagggattgttagattatattgctctggtaagattactttctaaaaagtttgtacttttctgtaaaataaaagaatatggaatcataaaatggcacgtgttttagggttagcctaaaattgggcattgtcatcgatttcaaagaaggtatgaactagcaggcttacagcctaatctttggactggtccttggcagcagttccttttcagactcaaacagaattcagatagatgtaagtcaaaacaaaacttcacaaagccaagcatattatcttttgcattaacctatttctccatcatacatgatactagtatgtgcattagcatgatattctcatatacattatattaaaaattaaaaggtggcagctcagggtgagctcttctgttattcatttgttcctaaatttttaagggctttttctcagtcaatagtttgtacaaactggttagtttaacttcattatccatttctttaaagttgatggatcgtgataagatgcatttaaggccaatagtgatagattttttttatctcttgaacacaagctttgtctgaatgatactcttatctcttaaacacaagctttgaatgataactacaggttttaagtgctgttacattaataccataatgtgatgtgttagaaacaaagggatatttcaaaggtaggtatttgaaaattctctagtctcaatgtatatgtgtattgaatatactctaaaaataaatgtgcaatttgctagtaggacaatgcagtgattagcattaggtatgtctcttttatatcctagctgtgtcccactttcttctaagtgcaatcctttcatgttcacttgctgttttattccatctactcttaactgcatttggaaggcttgtctagagtatagcatgtatttttacctttgcagtgaattgcatgtgctaattgtaaccacagctatttttatgttgacataactccaaatgttatattaaatgttctattacatatcagctctaatcccttaagtaaagtttaagaaataaattcttgttcaaattgtt

SEQ ID NO: 31 - DNA, 7454 bp, Rattus norvegicus
gagcatcccttgcaagatgtccgcactaaggatctggctaatgcaagctttgcttattttcctcactactgagtctataggtcaacttgtggaaccatgtggttatatctaccctgagtttccagttgtccagcgtggctctaacttcactgccacttgtgtgctaaaggagaagtgtctgcaggtgtactccgtgaatgccacttacatcgtgtggaagacaaaccatgttgccgttcctaaggagcaggtcacagtcatcaacagaacggcatccagtgtcacattcacagacgtggtctttcagaacgtccagctcacctgcaatatcctgtcatttggacagatcgagcagaatgtgtatggaatcaccatactttcaggctatcctccagatatacctacaaatttgagttgcattgtgaatgaggggaagaacatgctgtgtcagtgggacccaggaagggagacataccttgaaacaaactacactttgaagtcagagtgggcaacagagaagtttcctgattgtcgaacaaagcatggcacgagctcctgcatgatgggctatacccccatctattttgtcaacatcgaggtctgggtggaggccgagaatgcccttgggaatgtctcctcagagcctatcaattttgaccccgtggataaagtgaaacccagcccacctcataatttgtcagtgaccaactcagaagaattatccagtatattaaaactagcatgggtcaattcaggtttggacagtattttaaggctgaagtcggacatccaatataggaccaaagatgcctcaacttggattcaggtccctcttgaagatacagtgtctccccggacttcgttcactgttcaggatctcaagccttttacagaatatgtgtttaggattcggtccattaaggagaatgggaagggctactggagtgactggagtgaggaggcgagcgggaccacatatgaagacagaccatccaaagcgccaagtttctggtataaggtaaatgcaaaccatccacaggagtataggtctgcacggctcatatggaagacattgcccctttctgaagccaatgggaagatcttggattatgaagtggttcttacacagtccaagtcagtttcgcaaacttacacagttaacggcacagagttgatagtaaacctcaccaataaccgctatgtagcgtctctagcagcaagaaatgtggtcggcaagtcccctgcaaccgtcctcaccatccccggctcccacttcaaagcttctcaccccgtagtggatcttaaagcatttccaaaagataacctgctctgggtagaatggacaccgccatctaaacctgtgaacaaatacatactagagtggtgtgtgttgtcagagaactcaccctgcatcccagactggcagcaagaagatggcactgtgaatcggacccacttaagaggaagcctactggagagcaagtgctatctgatcacagtaaccccagtgtttcccggtgggcctggaagccctgagtccatgaaggcgtacctcaaacaagcagctccttctaaaggaccgactgttcggacaaagaaagtggggaaaaatgaagctgtcttagagtgggaccatcttcctgttgatgtccagaacggattcattagaaactactccatatcttatagaaccagtgttggaaaagaaatggttgtgcgtgtggattcttctcacacagaatacacactgtcctctttgagtagcgatacactgtacatggtccacatggcagcatacacagaagaaggtgggaaggatgggccggaattcacttttacaacactaaagtttgctcaaggagaaatcgaagccatagtcgtgcctgtgtgcttagctttcctcctgacaacgctgctgggagtcttgttctgctttaataaacgagacctaattaaaaaacacatctggccgaatgtcccagatccttcaaagagtcatattgcccagtggtcacctcacacccccccaaggcacaattttaactccaaagatcagatgtactcagatgccaatttcactgacgtaagcgttgtggaaatagaagcaaacaacaaaaagccttgtccagatgacctgaaatccttggacctgttcaagaaggagaaaataagtacagaagggcacagcagtggcattggggggtcctcgtgcatgtcctcttctaggcccagcatctccagcagtgaggagaacgagtctgctcagagcaccgcaagcacggtgcagtactccaccgtggtgcacagcggctacaggcaccaggtgccgtcagtgcaagtgttctcaaggtctgagtccacccagcccctgctagactccgaggagcggccagaggacctgcagctggtggatagtgtggacagcggtgacgagatcttacccaggcaacagtatttcaaacaaagctgcagtcagcctggagccagtccagatgtttcacacttcgaaaggtcaagccaggttccgtcaggcagtgaggaggattttgtcagactgaaacagcagcaggtttcagatcacatttcagagccctatggctccgagcaacggaggctgtttcaggaaggttctgtggcagatgctcttggcacagggactgatggacagatagagagattcgaatctgttggaatggagacagcgatggatgaagacatttccaaaagttacttgccacagactgtaagacaaggtggctacatgccacagtgagggaccggctcctgcgggacctcagcgggactgcgaagtaaagctaaaatgctcgtgacttcagatgggaaataatcttcagtccctgatgataaccattgcctttaaggacaaagtcacaactgggccatctccattccagagtatctgggattctccttcaagcactacatgaactgactttatcctctgaatagctggaggactctgcgctgtttcagaatgtttgcactgctgaaaactaagtgtgtctgagtttgtaaagcttttgttttgctccatagaaagtggggaatttaaatgagccgagccgagccatggcgtcgctcagctgtgctggtggtgagactgaccattcagatgccgggagcaaaggtctaaccctcctcagccagcagcttcatgtcagagttccccgggcagactgcccagattgtgtcacctatcaaaaaaaaaaaaaaaagagtggagggggcagggtggctcatttctgggtttgttattagagaatcttggcttgtttggtgccgttggtatcgatgcttccagttattactgaagaaaaggacgaactttcaagagaagcagcatagaagaacccattaccttatattcacctaaaacctcacagaaacctgcttattagtgataacatttttcaaagtcagtttcaccaacactctagtggaccgggctggcctcaaactcacagagatttgtctgcctcagcctccggagtactggggttaaaggcatgtaccaccaccaccacttgacttaaaaatttgctgtttttaaagacttaagttgctgacatttttctgaacgtatataaacacaccagggacagatgattccagatgttggtggaggaagcactttatccagaatgtcttcaactgtacattactttaattgactccaccttgtttttgtttttaagactagcaaggtgtttgctgaagggtggcattgcccagaggctcaggcgagtcccagagctcacgcaccgatgtctgtgaccaccaccgtggcagaagtcctcctacaaagctgacgaagccgtgagggttttgttagtttgctgggcggggattgagcccacagccttgtacatgctagacactaccactgagctcacctacacatctcactatgttatgtaggctcaagcgatcctcctgtctcagcttcctgagtgttgggacttcgggtgtgcgctgccacacacccgaccaggtagttttaatatctgccatttatgaggtagagttttacacaggatttaggactatcttaaacctgttccttaggttgctttgttaaggaatagcctggacatccggtgtttagcactaaagccattatgtatgcgcctaactttccagtgacatgtcctgggtgggcaggagttgactgagcgccattagtggttgattaccaccaagtgtgcgcacaggaaaaggattaaagtttcagaacatggcccttaatcattcatagccgctgccccacggtggctgaggaggagttgatgtgcactgagctggatgtggttttgccgttcactcagaggactgtttatactctacttgtgatgtgttatcgttatatttttatactaggtaatcctgttctgagtcctttcacgtgtataatcctcatgggactgtacgtgctttgctctggtattcagttgccttcttggcaaaaccagcctagaattgctaaaatgccaggattgaagttagatgatgaaagtcgggcacaggaaggccaggtcagggtcagagagaccagaattcacactacaaggctggcaccatcaccattcaagttgggcatggaagcctgggcccggcaggactgagctccctatgtatgtgtatctgccaggaaattaacacttgctaaatttcccatcctctcacagcgagcagtgtacctgtgcgctgctgtgaacgttgtagtgtgaagtctgtcctggggtagggagttaccagcgtgaggtcagggatgaacacagctcactgaagacgagcctggtggaagagagtttgccattgtgcccactgcatgaacattccctcgggggatgtatcatggagcacctctctttgctctaagaaagagtcttagaattcatcttaaatacagactttgctacagcatgtttaagaataatctggatgcacgtaacttgtattttaggtaggcagagaaactaatctggctgctttctgtaaaaccagtctaataaagctaattctatacatgttcttttcttctctcgtgtgtaaaactgtagtttaaggcaatcagaaaacaacctatcttgtggccaaaactagttcactgtagtttaaggcaatcagaaaacaacctatcttgtggccaaaactagttcactgcttctgatgcagttgcctgcttaggagtcatagacaaagaaggtttaatgtaatcaatattaccaaacagagttcccatcttttactcaccaaacaaagtcagtccttcgaaaagcataagtcagtactggcggacacagagaagccaaagtaggaaaagtgcaaccccatcctggttctcctgtctacagttcattcagcttctgcatagttcctgaactgtagggttttttgcttttcgggttttttttgttttgttttggggtttgtttgtttgttttgtttttggggattttgggggggtgtttttggttttttaagatagggtttctctgtgtggccttggctgtcctggatctgccagcctggtgtgccaccactgtctggtgagaccatagttcttactgtgaacttaaagggctgaagaggtagaagggcttatgggacggactgcagcttctaattttttctcaaccctactttaataatggttcttaaatgctttaaccttccttctagcccaccacccattaggggtagtggaaaagagaggatacaggtgaagtggacctgtttagaaatggttctttggagcaaatcctgtctgcactgtcaggaaatcagcagttcggttcactggtcagcagtggcagccccactcgagaacacacttcctggatgcaccagcagtccagttcagtagtgtcgggagagcagcagcggtggcccgacctagcaggagtggccaggcctcagcctcagcacaagtcagcagaaagaaccagaagttctcagctgtgcctctcagtgaaatgaaaatatccaagactgttgagtcctatatatactgcctccaaatatcatgtgtccgtgggtgtggtctccccacgtgtcttgcctcagtatgtgagtctgtatcagctgacatgactgccaatcgacccaggtcaaggaattcgcaagaagccaccagaacacacgttttttggtgtttctctttatggagtcctgacaaatggcgcacaatgatacatgtgaggcaaaccaatatgtgtgttattacaaagaatccttcatcatgtgtcctttcgaatgctttctgtagcaaaatatcctttcacctgtgtctgctttagcaaaacaccgtccaacacaactgactttccaaagaaccttaagtttccattccacaggacactgtcaaagccaggactgctaacgtcccattagacatcgcactgtttttaaagctgtcttctttttcctgcccacagaattagagctacagcattcctcagtgtttgtcatagacatgtattaaccaggtagaacaagccaggaggttttagactattaagtttactctcctgcttcatgttactgagtttcctttaaaagcaagcttttaacacaacttctggttagtagagattttcatgacatgagtagaatcagtgagctgcaaaaaaaagtgtaaaggtgaattgttaaaaataaccccttgggctagctctgctctaggcactgttgttacattacccaggtaagattacgttttaaaatgtgcctttctatgagttagaaaacacactatatggcaaactatataattaattcatagacaaagtcttctcaagccagatgtactatctctcaggttcatggtttgttctgtcacatgcaggttagcctatgctcactccggtaaagctaaaacggtgatggctgtaggtgttactcgctctaacttttttagggctttttgtcaccagtaagctcccacagactggtcagttttttgtttgttttttgtttgtttgttttaattagttgtcaacaccagtgagggggtcagcatgaaaacgcaccaggccggttgggtagagattcttagtcctgtgatcaccagcttagtctggtaaccacagactgcaaatgctgttacgtaactagaaacatgggacgtatcagaacaaggggatgctacacaggtgtttgtgtaatatacagtgcaccttgctgctagtgtgacagtaggcattccctgtggccccttatgcctttgctctcctgtactccacgtgagccctgttctctcaactctgccccacccacccatgcagcttcacatgggaagcttgcccgtgttagcgctgtagtaagttgcatgtgctgagtataaccaaagctatcatttttatgttgaccctgtcataaatgtcttactatatcagctctaatcctttttaaataaatttgagaaagaaact

SEQ ID NO: 32 - protein, 114 aa, Artificial Sequence
  Heavy chain variable
EVQLLESGGGLVQPGGSLRLSCAASGFTFSSYGMHWVRQAPGKGLEWVSVISYDGSYKYYADSVKGRFTISRDNSKNTLYLQMNSLRAEDTAVYYCAAGVPDYWGQGTLVTVSS

SEQ ID NO: 33 - protein, 5 aa, Artificial Sequence
  Heavy chain CDR1
SYGMH

SEQ ID NO: 34 - protein, 17 aa, Artificial Sequence
  Heavy chain CDR2
VISYDGSYKYYADSVKG

SEQ ID NO: 35 - protein, 5 aa, Artificial Sequence
  Heavy chain CDR3
GVPDY

SEQ ID NO: 36 - protein, 111 aa, Artificial Sequence
  Light chain variable
QSVLTQPPSASGTPGQRVTISCTGSSSNIGAGYDVHWYQQLPGTAPKLLIYSNNERPSGVPDRFSGSKSGTSASLAISGLRSEDEADYYCAAWDDSLNGPVFGGGTKLTVL

SEQ ID NO: 37 - protein, 14 aa, Artificial Sequence
  Light chain CDR1
TGSSSNIGAGYDVH

SEQ ID NO: 38 - protein, 7 aa, Artificial Sequence
  Light chain CDR2
SNNERPS

SEQ ID NO: 39 - protein, 11 aa, Artificial Sequence
  Light chain CDR3
AAWDDSLNGPV

SEQ ID NO: 40 - protein, 443 aa, Artificial Sequence
  TPP-16478 heavy chain full
EVQLLESGGGLVQPGGSLRLSCAASGFTFSSYGMHWVRQAPGKGLEWVSVISYDGSYKYYADSVKGRFTISRDNSKNTLYLQMNSLRAEDTAVYYCAAGVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 217 aa, Artificial Sequence
  TPP-16478 light chain full
QSVLTQPPSASGTPGQRVTISCTGSSSNIGAGYDVHWYQQLPGTAPKLLIYSNNERPSGVPDRFSGSKSGTSASLAISGLRSEDEADYYCAAWDDSLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 116 aa, Artificial Sequence
  Heavy chain variable
EVQLLESGGGLVQPGGSLRLSCAASGFTFSDYYMSWIRQAPGKGLEWVSGINWNGGSTGYADSVKGRFTISRDNSKNTLYLQMNSLRAEDTAVYYCATWDGPFDYWGQGTLVTVSS

SEQ ID NO: 43 - protein, 5 aa, Artificial Sequence
  Heavy chain CDR1
DYYMS

SEQ ID NO: 44 - protein, 17 aa, Artificial Sequence
  Heacy chain CDR2
GINWNGGSTGYADSVKG

SEQ ID NO: 45 - protein, 7 aa, Artificial Sequence
  Heavy chain CDR3
WDGPFDY

SEQ ID NO: 46 - protein, 111 aa, Artificial Sequence
  Light chain variable
QSVLTQPPSASGTPGQRVTISCTGSSSNIGAGYDVHWYQQLPGTAPKLLIYYDDLLPSGVPDRFSGSKSGTSASLAISGLRSEDEADYYCQSYDSSLGGSVFGGGTKLTVL

SEQ ID NO: 47 - protein, 14 aa, Artificial Sequence
  Light chain CDR1
TGSSSNIGAGYDVH

SEQ ID NO: 48 - protein, 7 aa, Artificial Sequence
  Light chain CDR2
YDDLLPS

SEQ ID NO: 49 - protein, 11 aa, Artificial Sequence
  Light chain CDR3
QSYDSSLGGSV

SEQ ID NO: 50 - protein, 440 aa, Artificial Sequence
  TPP-18063 heavy chain full
EVQLLESGGGLVQPGGSLRLSCAASGFTFSDYYMSWIRQAPGKGLEWVSGINWNGGSTGYADSVKGRFTISRDNSKNTLYLQMNSLRAEDTAVYYCATWDGPFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 51 - protein, 217 aa, Artificial Sequence
  TPP-18063 light chain full
QSVLTQPPSASGTPGQRVTISCTGSSSNIGAGYDVHWYQQLPGTAPKLLIYYDDLLPSGVPDRFSGSKSGTSASLAISGLRSEDEADYYCQSYDSSLGGSVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 52 - protein, 438 aa, Artificial Sequence
  TPP-18068 heavy chain full
EVQLLESGGGLVQPGGSLRLSCAASGFTFSSYGMHWVRQAPGKGLEWVSVISYDGSYKYYADSVKGRFTISRDNSKNTLYLQMNSLRAEDTAVYYCAAGVPDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 217 aa, Artificial Sequence
  TPP-18068 light chain full
QSVLTQPPSASGTPGQRVTISCTGSSSNIGAGYDVHWYQQLPGTAPKLLIYSNNERPSGVPDRFSGSKSGTSASLAISGLRSEDEADYYCAAWDDSLNGPVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 54 - protein, 117 aa, Artificial Sequence
  Heavy chain variable
EVQLLESGGGLVQPGGSLRLSCAASGFTFSHYDMNWLRQAPGKGLEWVGVISYDGSNKFYADFVKGRFTISRDNSKNTLYLQMNSLRAEDTAVYYCARTPGSHFDYWGQGTLVTVSS

SEQ ID NO: 55 - protein, 5 aa, Artificial Sequence
  Heavy chain CDR1
HYDMN

SEQ ID NO: 56 - protein, 17 aa, Artificial Sequence
  Heavy chain CDR2
VISYDGSNKFYADFVKG

SEQ ID NO: 57 - protein, 8 aa, Artificial Sequence
  Heavy chain CDR3
TPGSHFDY

SEQ ID NO: 58 - protein, 110 aa, Artificial Sequence
  Light chain variable
QSVLTQPPSASGTPGQRVTISCSGSSSNIGNNAVNWYQQLPGTAPKLLIYYDDLLPSGVPDRFSGSKSGTSASLAISGLRSEDEADYYCSAWDDSLNGVVFGGGTKLTVL

SEQ ID NO: 59 - protein, 13 aa, Artificial Sequence
  Light chain CDR1
SGSSSNIGNNAVN

SEQ ID NO: 60 - protein, 7 aa, Artificial Sequence
  Light chain CDR2
YDDLLPS

SEQ ID NO: 61 - protein, 11 aa, Artificial Sequence
  Light chain CDR3
SAWDDSLNGVV

SEQ ID NO: 62 - protein, 441 aa, Artificial Sequence
  TPP-18087 heavy chain full
EVQLLESGGGLVQPGGSLRLSCAASGFTFSHYDMNWLRQAPGKGLEWVGVISYDGSNKFYADFVKGRFTISRDNSKNTLYLQMNSLRAEDTAVYYCARTPGSHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 63 - protein, 216 aa, Artificial Sequence
  TPP-18087 light chain full
QSVLTQPPSASGTPGQRVTISCSGSSSNIGNNAVNWYQQLPGTAPKLLIYYDDLLPSGVPDRFSGSKSGTSASLAISGLRSEDEADYYCSAWDDSLNGVVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 64 - protein, 117 aa, Artificial Sequence
  Heavy chain variable
EVQLLESGGGLVQPGGSLRLSCAASGFTFSIYAMSWVRQAPGKGLEWVSSISDTGDRTYYADSVKGRFTISRDNSKNTLYLQMNSLRAEDTAVYYCARSNWGSLDYWGQGTLVTVSS

SEQ ID NO: 65 - protein, 5 aa, Artificial Sequence
  Heavy chain CDR1
IYAMS

SEQ ID NO: 66 - protein, 17 aa, Artificial Sequence
  Heavy chain CDR2
SISDTGDRTYYADSVKG

SEQ ID NO: 67 - protein, 8 aa, Artificial Sequence
  Heavy chain CDR3
SNWGSLDY

SEQ ID NO: 68 - protein, 110 aa, Artificial Sequence
  Light chain variable
QSVLTQPPSASGTPGQRVTISCSGSSSNIGSNYVYWYQQLPGTAPKLLIYYDDLLPSGVPDRFSGSKSGTSASLAISGLRSEDEADYYCAAWDDSLSGRVFGGGTKLTVL

SEQ ID NO: 69 - protein, 13 aa, Artificial Sequence
  Light chain CDR1
SGSSSNIGSNYVY

SEQ ID NO: 70 - protein, 7 aa, Artificial Sequence
  Light chain CDR2
YDDLLPS

SEQ ID NO: 71 - protein, 11 aa, Artificial Sequence
  Light chain CDR3
AAWDDSLSGRV

SEQ ID NO: 72 - protein, 441 aa, Artificial Sequence
  TPP-19528 heavy chain full
EVQLLESGGGLVQPGGSLRLSCAASGFTFSIYAMSWVRQAPGKGLEWVSSISDTGDRTYYADSVKGRFTISRDNSKNTLYLQMNSLRAEDTAVYYCARSNWGSLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 73 - protein, 216 aa, Artificial Sequence
  TPP-19528 light chain full
QSVLTQPPSASGTPGQRVTISCSGSSSNIGSNYVYWYQQLPGTAPKLLIYYDDLLPSGVPDRFSGSKSGTSASLAISGLRSEDEADYYCAAWDDSLSGRVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 74 - protein, 479 aa, Artificial Sequence
  TPP-20489 Chain 1
EVQLLESGGGLVQPGGSLRLSCAASGFTFSSYGMHWVRQAPGKGLEWVSVISYDGSYKYYADSVKGRFTISRDNSKNTLYLQMNSLRAEDTAVYYCAAGVPDYWGQGTLVTVSSGGGGSGGGGSGGGGSQSVLTQPPSASGTPGQRVTISCTGSSSNIGAGYDVHWYQQLPGTAPKLLIYSNNERPSGVPDRFSGSKSGTSASLAISGLRSEDEADYYCAAWDDSLNGPVFGGGTKLTVLGGGGSGGGGSGGGGSGVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 481 aa, Artificial Sequence
  TPP-20489 chain 2
EVQLLESGGGLVQPGGSLRLSCAASGFTFSHYDMNWLRQAPGKGLEWVGVISYDGSNKFYADFVKGRFTISRDNSKNTLYLQMNSLRAEDTAVYYCARTPGSHFDYWGQGTLVTVSSGGGGSGGGGSGGGGSQSVLTQPPSASGTPGQRVTISCSGSSSNIGNNAVNWYQQLPGTAPKLLIYYDDLLPSGVPDRFSGSKSGTSASLAISGLRSEDEADYYCSAWDDSLNGVVFGGGTKLTVLGGGGSGGGGSGGGGSGVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 76 - protein, 449 aa, Artificial Sequence
  TPP-23552 competitor AB heavy chain full
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 218 aa, Artificial Sequence
  TPP-23552 competitor AB light chain
DIVLTQSPASLALSPGERATLSCRASKSVSTSGYSYIHWYQQKPGQPPRLLIYLASNLDSGVPARFSGSGSGTDFTLNIHPLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 443 aa, Artificial Sequence
  TPP-23580 competitor AB heavy chain full
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 79 - protein, 218 aa, Artificial Sequence
  TPP-23580 competitor AB light chain full
DIVLTQSPASLALSPGERATLSCRASKSVSTSGYSYIHWYQQKPGQPPRLLIYLASNLDSGVPARFSGSGSGTDFTLNIHPLEEEDFATYYCQHSRDLPPTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 80 - protein, 479 aa, Artificial Sequence
  TPP-26195 Chain 1
EVQLLESGGGLVQPGGSLRLSCAASGFTFSSYGMHWVRQAPGKGLEWVSVISYDGSYKYYADSVKGRFTISRDNSKNTLYLQMNSLRAEDTAVYYCAAGVPDYWGQGTLVTVSSGGGGSGGGGSGGGGSQSVLTQPPSASGTPGQRVTISCTGSSSNIGAGYDVHWYQQLPGTAPKLLIYSNNERPSGVPDRFSGSKSGTSASLAISGLRSEDEADYYCAAWDDSLNGPVFGGGTKLTVLGGGGSGGGGSGGGGSGVECPPCPAPPVAGPSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLWCMITDFFPEDITVEWQWNGQPAENYKNTQPIMDTDGSYFVYSKLNVQKSNWEAGNTFTCSVLHEGLHNHHTEKSLSHSPGK

SEQ ID NO: 81 - protein, 481 aa, Artificial Sequence
  TPP-26195 Chain 2
EVQLLESGGGLVQPGGSLRLSCAASGFTFSHYDMNWLRQAPGKGLEWVGVISYDGSNKFYADFVKGRFTISRDNSKNTLYLQMNSLRAEDTAVYYCARTPGSHFDYWGQGTLVTVSSGGGGSGGGGSGGGGSQSVLTQPPSASGTPGQRVTISCSGSSSNIGNNAVNWYQQLPGTAPKLLIYYDDLLPSGVPDRFSGSKSGTSASLAISGLRSEDEADYYCSAWDDSLNGVVFGGGTKLTVLGGGGSGGGGSGGGGSGVECPPCPAPPVAGPSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLSCAITDFFPEDITVEWQWNGQPAENYKNTQPIMDTDGSYFVVSKLNVQKSNWEAGNTFTCSVLHEGLHNHHTEKSLSHSPGK

SEQ ID NO: 82 - protein, 114 aa, Artificial Sequence
  Heavy chain variable
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSS

SEQ ID NO: 83 - protein, 5 aa, Artificial Sequence
  Heavy chain CDR1
SYGMH

SEQ ID NO: 84 - protein, 17 aa, Artificial Sequence
  Heavy chain CDR2
VISYDGSYKYYADSVKG

SEQ ID NO: 85 - protein, 5 aa, Artificial Sequence
  Heavy chain CDR3
GVPDY

SEQ ID NO: 86 - protein, 111 aa, Artificial Sequence
  Light chain variable
QSVLTQPPSASGTPGQRVTISCSGSSSNIGSGYDVHWYQQLPGTAPKLLIYSNNERPSGVPDRFSGSKSGTSASLAISGLRSEDEADYYCAAWDDSLSGPVFGGGTKLTVL

SEQ ID NO: 87 - protein, 14 aa, Artificial Sequence
  Light chain CDR1
SGSSSNIGSGYDVH

SEQ ID NO: 88 - protein, 7 aa, Artificial Sequence
  Light chain CDR2
SNNERPS

SEQ ID NO: 89 - protein, 11 aa, Artificial Sequence
  Light chain CDR3
AAWDDSLSGPV

SEQ ID NO: 90 - protein, 444 aa, Artificial Sequence
  TPP-27159 heavy chain full
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 217 aa, Artificial Sequence
  TPP-27159 light chain full
QSVLTQPPSASGTPGQRVTISCSGSSSNIGSGYDVHWYQQLPGTAPKLLIYSNNERPSGVPDRFSGSKSGTSASLAISGLRSEDEADYYCAAWDDSLSGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 92 - protein, 444 aa, Artificial Sequence; identical to SEQ ID NO: 90
  TPP-29386 heavy chain full

SEQ ID NO: 93 - protein, 217 aa, Artificial Sequence
  TPP-29386 light chain full
QSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 94 - protein, 441 aa, Artificial Sequence
  TPP-29519 heavy chain full
QVQLVESGGGVVQPGRSLRLSCAASGFTFSHYDMHWVRQAPGKGLEWVAVISYHGSVKFYADSVKGRFTISRDNSKNTLYLQMNSLRAEDTAVYYCARTPGFHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 95 - protein, 216 aa, Artificial Sequence
  TPP-29519 light chain full
QSVLTQPPSASGTPGQRVTISCSGSSSNIGSNYVSWYQQLPGTAPKLLIYYDDLRPSGVPDRFSGSKSGTSASLAISGLRSEDEADYYCSAWDDSLSGVVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 96 - protein, 441 aa, Artificial Sequence
  TPP-29520 heavy chain full
QVQLVESGGGVVQPGRSLRLSCAASGFTFSHYDMHWVRQAPGKGLEWVAVISYSGSVKFYADSVKGRFTISRDNSKNTLYLQMNSLRAEDTAVYYCARTPGSHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 97 - protein, 216 aa, Artificial Sequence
  TPP-29520 light chain full
QSVLTQPPSASGTPGQRVTISCSGSSSNIGSNYVNWYQQLPGTAPKLLIYYDDLRPSGVPDRFSGSKSGTSASLAISGLRSEDEADYYCSAWDDSLSGVVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 98 - protein, 441 aa, Artificial Sequence
  TPP-29521 heavy chain full
QVQLVESGGGVVQPGRSLRLSCAASGFTFSHYDMHWVRQAPGKGLEWVAVISYEGSNKFYADSVKGRFTISRDNSKNTLYLQMNSLRAEDTAVYYCARTPGFHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 99 - protein, 216 aa, Artificial Sequence; identical to SEQ ID NO: 95
  TPP-29521 light chain full

SEQ ID NO: 100 - protein, 441 aa, Artificial Sequence; identical to SEQ ID NO: 98
  TPP-29522 heavy chain full

SEQ ID NO: 101 - protein, 216 aa, Artificial Sequence; identical to SEQ ID NO: 97
  TPP-29522 light chain full

SEQ ID NO: 102 - protein, 441 aa, Artificial Sequence
  TPP-29523 heavy chain full
QVQLVESGGGVVQPGRSLRLSCAASGFTFSHYDMHWVRQAPGKGLEWVAVISYEGSVKFYADSVKGRFTISRDNSKNTLYLQMNSLRAEDTAVYYCARTPGFHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 103 - protein, 216 aa, Artificial Sequence; identical to SEQ ID NO: 97
  TPP-29523 light chain full

SEQ ID NO: 104 - protein, 438 aa, Artificial Sequence
  TPP-29528 heavy chain full
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 105 - protein, 217 aa, Artificial Sequence
  TPP-29528 light chain full
QSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 106 - protein, 114 aa, Artificial Sequence; identical to SEQ ID NO: 82
  Heavy chain variable

SEQ ID NO: 107 - protein, 5 aa, Artificial Sequence
  Heavy chain CDR1
SYGMH

SEQ ID NO: 108 - protein, 17 aa, Artificial Sequence
  Heavy chain CDR2
VISYDGSYKYYADSVKG

SEQ ID NO: 109 - protein, 5 aa, Artificial Sequence
  Heavy chain CDR3
GVPDY

SEQ ID NO: 110 - protein, 111 aa, Artificial Sequence
  Light chain variable
QSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVL

SEQ ID NO: 111 - protein, 14 aa, Artificial Sequence
  Light chain CDR1
SGSSSNIGSDYDVH

SEQ ID NO: 112 - protein, 7 aa, Artificial Sequence
  Light chain CDR2
SNEERPS

SEQ ID NO: 113 - protein, 11 aa, Artificial Sequence
  Light chain CDR3
SAWDSSLSGPV

SEQ ID NO: 114 - DNA, 342 bp, Artificial Sequence
  TPP-29536 heavy chain full
gaagttcagctgctggaatctggcggcggactggttcaacctggcggatctctgagactgagctgtgccgccagcggcttcacctttagcagctatggcatgcactgggtccgacaggcccctggcaaaggacttgaatgggtcgccgtgatcagctacgacggcagctacaagtactacgccgacagcgtgaagggcagattcaccatcagccgggacaacagcaagaacaccctgtacctgcagatgaacagcctgagagccgaggacaccgccgtgtactattgtgctgctggcgtgccagattactggggccagggaacactggtcacagtgtcatca

SEQ ID NO: 115 - DNA, 333 bp, Artificial Sequence
  TPP-29536 light chain full
cagtctgttctgacacagcctcctagcgcctctggcacacctggacagagagtgaccatcagctgtagcggcagcagctccaacatcggcagcgactatgacgtgcactggtatcagcagctgcctggcacagcccctaaactgctgatctacagcaacgaggaacggcctagcggcgtgcccgatagattttctggcagcaagagcggcacaagcgccagcctggctatctctggactgagatctgaggacgaggccgactactactgcagcgcctgggattcttctctgtctggccctgtttttggcggaggcaccaagctgacagtgcta

SEQ ID NO: 116 - protein, 443 aa, Artificial Sequence
  TPP-29536 heavy chain full
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 217 aa, Artificial Sequence; identical to SEQ ID NO: 93
  TPP-29536 light chain full

SEQ ID NO: 118 - protein, 481 aa, Artificial Sequence
  TPP-29603 Chain 1
QVQLVESGGGVVQPGRSLRLSCAASGFTFSHYDMHWVRQAPGKGLEWVAVISYHGSVKFYADSVKGRFTISRDNSKNTLYLQMNSLRAEDTAVYYCARTPGFHFDYWGQGTLVTVSSGGGGSGGGGSGGGGSQSVLTQPPSASGTPGQRVTISCSGSSSNIGSNYVSWYQQLPGTAPKLLIYYDDLRPSGVPDRFSGSKSGTSASLAISGLRSEDEADYYCSAWDDSLSGVVFGGGTKLTVLGGGGSGGGGSGGGGSGVECPPCPAPPVAGPSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPCKEQMAKDKVSLWCMITDFFPEDITVEWQWNGQPAENYKNTQPIMDTDGSYFVYSKLNVQKSNWEAGNTFTCSVLHEGLHNHHTEKSLSHSPGK

SEQ ID NO: 119 - protein, 479 aa, Artificial Sequence
  TPP-29603 Chain 2
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSSGGGGSGGGGSGGGGSQSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVLGGGGSGGGGSGGGGSGVECPPCPAPPVAGPSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVCTIPPPKEQMAKDKVSLSCAITDFFPEDITVEWQWNGQPAENYKNTQPIMDTDGSYFVVSKLNVQKSNWEAGNTFTCSVLHEGLHNHHTEKSLSHSPGK

SEQ ID NO: 120 - protein, 117 aa, Artificial Sequence
  Heavy chain variable
QVQLVESGGGVVQPGRSLRLSCAASGFTFSHYDMHWVRQAPGKGLEWVAVISYHGSVKFYADSVKGRFTISRDNSKNTLYLQMNSLRAEDTAVYYCARTPGFHFDYWGQGTLVTVSS

SEQ ID NO: 121 - protein, 5 aa, Artificial Sequence
  Heavy chain CDR1
HYDMH

SEQ ID NO: 122 - protein, 17 aa, Artificial Sequence
  Heavy chain CDR2
VISYHGSVKFYADSVKG

SEQ ID NO: 123 - protein, 8 aa, Artificial Sequence
  Heavy chain CDR3
TPGFHFDY

SEQ ID NO: 124 - protein, 110 aa, Artificial Sequence
  Light chain variable
QSVLTQPPSASGTPGQRVTISCSGSSSNIGSNYVSWYQQLPGTAPKLLIYYDDLRPSGVPDRFSGSKSGTSASLAISGLRSEDEADYYCSAWDDSLSGVVFGGGTKLTVL

SEQ ID NO: 125 - protein, 13 aa, Artificial Sequence
  Light chain CDR1
SGSSSNIGSNYVS

SEQ ID NO: 126 - protein, 7 aa, Artificial Sequence
  Light chain CDR2
YDDLRPS

SEQ ID NO: 127 - protein, 11 aa, Artificial Sequence
  Light chain CDR3
SAWDDSLSGVV

SEQ ID NO: 128 - DNA, 351 bp, Artificial Sequence
  TPP-29680 heavy chain full
caggttcagctggtggaatctggtggcggagttgtgcagcctggcagaagcctgagactgtcttgtgccgccagcggcttcaccttcagccactacgatatgcactgggtccgacaggcccctggcaaaggacttgaatgggtcgccgtgatcagctaccacggcagcgtgaagttctacgccgactccgtgaagggcagattcaccatcagccgggacaacagcaagaacaccctgtacctgcagatgaacagcctgagagccgaggacaccgccgtgtactactgtgccagaacacccggcttccacttcgactattggggccagggaaccctggtcacagtctcttca

SEQ ID NO: 129 - DNA, 330 bp, Artificial Sequence
  TPP-29680 light chain full
cagtctgttctgacacagcctcctagcgcctctggcacacctggacagagagtgaccatcagctgtagcggcagcagctccaacatcggcagcaactacgtgtcctggtatcagcagctgcctggcacagcccctaaactgctgatctactacgacgacctgcggcctagcggcgtgccagatagattttctggcagcaagagcggcacctctgccagcctggctatctctggactgagaagcgaggacgaggccgactactactgtagcgcctgggatgatagcctgtccggcgttgtgtttggcggaggcacaaagctgacagtgcta

SEQ ID NO: 130 - protein, 446 aa, Artificial Sequence
  TPP-29680 heavy chain full
QVQLVESGGGVVQPGRSLRLSCAASGFTFSHYDMHWVRQAPGKGLEWVAVISYHGSVKFYADSVKGRFTISRDNSKNTLYLQMNSLRAEDTAVYYCARTPGFHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 216 aa, Artificial Sequence
  TPP-29680 light chain full
QSVLTQPPSASGTPGQRVTISCSGSSSNIGSNYVSWYQQLPGTAPKLLIYYDDLRPSGVPDRFSGSKSGTSASLAISGLRSEDEADYYCSAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 132 - protein, 481 aa, Artificial Sequence
  TPP-29697 Chain 1
QVQLVESGGGVVQPGRSLRLSCAASGFTFSHYDMHWVRQAPGKGLEWVAVISYHGSVKFYADSVKGRFTISRDNSKNTLYLQMNSLRAEDTAVYYCARTPGFHFDYWGQGTLVTVSSGGGGSGGGGSGGGGSQSVLTQPPSASGTPGQRVTISCSGSSSNIGSNYVSWYQQLPGTAPKLLIYYDDLRPSGVPDRFSGSKSGTSASLAISGLRSEDEADYYCSAWDDSLSGVVFGGGTKLTVLGGGGSGGGGSGGGGSGVECPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 479 aa, Artificial Sequence
  TPP-29697 Chain 2
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSSGGGGSGGGGSGGGGSQSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVLGGGGSGGGGSGGGGSGVECPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 134 - DNA, 1443 bp, Artificial Sequence
  TPP-29697 chain 1
caggtgcagctggtggaatctggtggcggagttgtgcagcctggcagatccctgagactgtcttgtgccgcctccggcttcaccttcagccactacgatatgcactgggtccgacaggcccctggcaaaggattggaatgggtcgccgtgatctcctaccacggctccgtgaagttctacgccgacagcgtgaagggcagattcaccatctctcgggacaactccaagaacaccctgtacctgcagatgaactccctgagagccgaggacaccgccgtgtactactgtgctagaacccctggcttccacttcgactattggggccagggcacactggtcacagtttctagcggaggcggaggaagtggcggcggaggatctggcggtggtggttctcagtctgtgctgacccaacctccttccgcttctggcacacctggccagagagtgaccatctcttgctccggctcctcctccaacatcggctccaattacgtgtcctggtatcagcagctgcccggcacagctcccaaactgctgatctactacgacgacctgcggccttctggcgtgcccgatagattctccggctctaagtctggcacctctgccagcctggctatctccggactgagatctgaggacgaggccgactactactgctctgcctgggacgattctctgtccggcgttgtgtttggcggaggcaccaaactgacagtgctcggaggtggtggctctggtggcggaggaagtggcggaggcggttctggtgttgaatgtcctccatgtcctgctcctccagtggctggcccttccgtgtttctgttccctccaaagcctaaggacaccctgatgatctctcggacccctgaagtgacctgtgtggtcgtgggagtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagcagctgccctctccaatcgaaaagaccatctccaaggccaagggacagcccagagaaccccaggtgtacacactgcctccatgcagagatgagctgaccaagaaccaggtgtccctgtggtgtctggtcaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 135 - DNA, 1437 bp, Artificial Sequence
  TPP-29697 chain 2
gaagtgcagctgctggaatctggcggaggattggttcagcctggcggctctctgagactgtcttgtgccgcttccggcttcaccttctccagctacggaatgcactgggtccgacaggcccctggcaaaggattggaatgggtcgccgtgatctcctacgacggctcctacaagtactacgccgactccgtgaagggcagattcaccatctctcgggacaactccaagaacaccctgtacctgcagatgaactccctgagagccgaggacaccgccgtgtactattgtgctgctggcgtgccagattactggggccagggaacactggtcacagtgtctagcggcggaggtggaagcggaggcggaggttctggtggtggtggatctcagtctgtgctgacccagcctccttctgcttctggcacacctggccagagagtgaccatctcttgctccggctcctcctccaacatcggctctgactacgacgtgcactggtatcagcagctccctggcacagctcccaaactgctgatctactccaacgaggaacggcctagcggcgtgcccgatagattctccggatctaagtccggcacctctgccagcctggctatctctggcctgagatctgaggacgaggccgactactactgctccgcctgggactcttctctgtctggccctgtttttggcggaggcaccaagctgacagtgctcggaggtggtggctctggtggcggaggaagtggcggaggcggttctggtgttgaatgtcctccatgtcctgctcctccagtggctggcccttccgtgtttctgttccctccaaagcctaaggacaccctgatgatctctcggacccctgaagtgacctgtgtggtcgtgggagtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagcagctgccctctccaatcgaaaagaccatctccaaggccaagggacagccccgggaacctcaagtctgtaccttgcctcctagccgggacgagctgaccaagaatcaggtgtccctgtcctgtgccgtgaagggcttctacccttccgatatcgccgtggaatgggagtccaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggcaag

SEQ ID NO: 136 - protein, 117 aa, Artificial Sequence
  Heavy chain variable
QVQLVESGGGVVQPGRSLRLSCAASGFTFSHYDMHWVRQAPGKGLEWVAVISYSGSVKFYADSVKGRFTISRDNSKNTLYLQMNSLRAEDTAVYYCARTPGSHFDYWGQGTLVTVSS

SEQ ID NO: 137 - protein, 5 aa, Artificial Sequence
  Heavy chain CDR1
HYDMH

SEQ ID NO: 138 - protein, 17 aa, Artificial Sequence
  Heavy chain CDR2
VISYSGSVKFYADSVKG

SEQ ID NO: 139 - protein, 8 aa, Artificial Sequence
  Heavy chain CDR3
TPGSHFDY

SEQ ID NO: 140 - protein, 110 aa, Artificial Sequence
  Light chain variable
QSVLTQPPSASGTPGQRVTISCSGSSSNIGSNYVNWYQQLPGTAPKLLIYYDDLRPSGVPDRFSGSKSGTSASLAISGLRSEDEADYYCSAWDDSLSGVVFGGGTKLTVL

SEQ ID NO: 141 - protein, 13 aa, Artificial Sequence
  Light chain CDR1
SGSSSNIGSNYVN

SEQ ID NO: 142 - protein, 7 aa, Artificial Sequence
  Light chain CDR2
YDDLRPS

SEQ ID NO: 143 - protein, 11 aa, Artificial Sequence
  Light chain CDR3
SAWDDSLSGVV

SEQ ID NO: 144 - DNA, 351 bp, Artificial Sequence
  TPP-30000 heavy chain full
caggttcagctggtggaatctggtggcggagttgtgcagcctggcagaagcctgagactgtcttgtgccgccagcggcttcaccttcagccactacgatatgcactgggtccgacaggcccctggcaaaggacttgaatgggtcgccgtgatcagctacagcggcagcgtgaagttctacgccgactccgtgaagggcagattcaccatcagccgggacaacagcaagaacaccctgtacctgcagatgaacagcctgagagccgaggacaccgccgtgtactactgtgccagaacacctggcagccacttcgactattggggccagggaacactggtcaccgttagctca

SEQ ID NO: 145 - DNA, 330 bp, Artificial Sequence
  TPP-30000 light chain full
cagtctgttctgacacagcctcctagcgcctctggcacacctggacagagagtgaccatcagctgtagcggcagcagctccaacatcggcagcaactacgtgaactggtatcagcagctgcccggcacagcccctaaactgctgatctactacgacgacctgcggcctagcggcgtgccagatagattttctggcagcaagagcggcacctctgccagcctggctatctctggactgagaagcgaggacgaggccgactactactgtagcgcctgggatgatagcctgtccggcgttgtgtttggcggaggcacaaagctgacagtgcta

SEQ ID NO: 146 - protein, 446 aa, Artificial Sequence
  TPP-30000 heavy chain full
QVQLVESGGGVVQPGRSLRLSCAASGFTFSHYDMHWVRQAPGKGLEWVAVISYSGSVKFYADSVKGRFTISRDNSKNTLYLQMNSLRAEDTAVYYCARTPGSHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 216 aa, Artificial Sequence
  TPP-30000 light chain full
QSVLTQPPSASGTPGQRVTISCSGSSSNIGSNYVNWYQQLPGTAPKLLIYYDDLRPSGVPDRFSGSKSGTSASLAISGLRSEDEADYYCSAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 148 - protein, 117 aa, Artificial Sequence
  Heavy chain variable
QVQLVESGGGVVQPGRSLRLSCAASGFTFSHYDMHWVRQAPGKGLEWVAVISYEGSNKFYADSVKGRFTISRDNSKNTLYLQMNSLRAEDTAVYYCARTPGFHFDYWGQGTLVTVSS

SEQ ID NO: 149 - protein, 5 aa, Artificial Sequence
  Heavy chain CDR1
HYDMH

SEQ ID NO: 150 - protein, 17 aa, Artificial Sequence
  Heavy chain CDR2
VISYEGSNKFYADSVKG

SEQ ID NO: 151 - protein, 8 aa, Artificial Sequence
  Heavy chain CDR3
TPGFHFDY

SEQ ID NO: 152 - protein, 110 aa, Artificial Sequence; identical to SEQ ID NO: 124
  Light chain variable

SEQ ID NO: 153 - protein, 13 aa, Artificial Sequence
  Light chain CDR1
SGSSSNIGSNYVS

SEQ ID NO: 154 - protein, 7 aa, Artificial Sequence
  Light chain CDR2
YDDLRPS

SEQ ID NO: 155 - protein, 11 aa, Artificial Sequence
  Light chain CDR3
SAWDDSLSGVV

SEQ ID NO: 156 - DNA, 351 bp, Artificial Sequence
  TPP-30001 heavy chain full
caggttcagctggtggaatctggtggcggagttgtgcagcctggcagaagcctgagactgtcttgtgccgccagcggcttcaccttcagccactacgatatgcactgggtccgacaggcccctggcaaaggacttgaatgggtcgccgtgatcagctacgagggcagcaacaagttctacgccgacagcgtgaagggcagattcaccatcagccgggacaacagcaagaacaccctgtacctgcagatgaacagcctgagagccgaggacaccgccgtgtactactgtgccagaacacccggcttccacttcgactattggggccagggaaccctggtcacagtctcttca

SEQ ID NO: 157 - DNA, 330 bp, Artificial Sequence; identical to SEQ ID NO: 129
  TPP-30001 light chain full

SEQ ID NO: 158 - protein, 446 aa, Artificial Sequence
  TPP-30001 heavy chain full
QVQLVESGGGVVQPGRSLRLSCAASGFTFSHYDMHWVRQAPGKGLEWVAVISYEGSNKFYADSVKGRFTISRDNSKNTLYLQMNSLRAEDTAVYYCARTPGFHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 216 aa, Artificial Sequence; identical to SEQ ID NO: 131
  TPP-30001 light chain full

SEQ ID NO: 160 - protein, 117 aa, Artificial Sequence; identical to SEQ ID NO: 148
  Heavy chain variable

SEQ ID NO: 161 - protein, 5 aa, Artificial Sequence
  Heavy chain CDR1
HYDMH

SEQ ID NO: 162 - protein, 17 aa, Artificial Sequence
  Heavy chain CDR2
VISYEGSNKFYADSVKG

SEQ ID NO: 163 - protein, 8 aa, Artificial Sequence
  Heavy chain CDR3
TPGFHFDY

SEQ ID NO: 164 - protein, 110 aa, Artificial Sequence; identical to SEQ ID NO: 140
  Light chain variable

SEQ ID NO: 165 - protein, 13 aa, Artificial Sequence
  Light chain CDR1
SGSSSNIGSNYVN

SEQ ID NO: 166 - protein, 7 aa, Artificial Sequence
  Light chain CDR2
YDDLRPS

SEQ ID NO: 167 - protein, 11 aa, Artificial Sequence
  Light chain CDR3
SAWDDSLSGVV

SEQ ID NO: 168 - DNA, 351 bp, Artificial Sequence; identical to SEQ ID NO: 156
  TPP-30002 heavy chain full

SEQ ID NO: 169 - DNA, 330 bp, Artificial Sequence; identical to SEQ ID NO: 145
  TPP-30002 light chain full

SEQ ID NO: 170 - protein, 446 aa, Artificial Sequence; identical to SEQ ID NO: 158
  TPP-30002 heavy chain full

SEQ ID NO: 171 - protein, 216 aa, Artificial Sequence; identical to SEQ ID NO: 147
  TPP-30002 light chain full

SEQ ID NO: 172 - protein, 117 aa, Artificial Sequence
  Heavy chain variable
QVQLVESGGGVVQPGRSLRLSCAASGFTFSHYDMHWVRQAPGKGLEWVAVISYEGSVKFYADSVKGRFTISRDNSKNTLYLQMNSLRAEDTAVYYCARTPGFHFDYWGQGTLVTVSS

SEQ ID NO: 173 - protein, 5 aa, Artificial Sequence
  Heavy chain CDR1
HYDMH

SEQ ID NO: 174 - protein, 17 aa, Artificial Sequence
  Heavy chain CDR2
VISYEGSVKFYADSVKG

SEQ ID NO: 175 - protein, 8 aa, Artificial Sequence
  Heavy chain CDR3
TPGFHFDY

SEQ ID NO: 176 - protein, 110 aa, Artificial Sequence; identical to SEQ ID NO: 140
  Light chain variable

SEQ ID NO: 177 - protein, 13 aa, Artificial Sequence
  Light chain CDR1
SGSSSNIGSNYVN

SEQ ID NO: 178 - protein, 7 aa, Artificial Sequence
  Light chain CDR2
YDDLRPS

SEQ ID NO: 179 - protein, 11 aa, Artificial Sequence
  Light chain CDR3
SAWDDSLSGVV

SEQ ID NO: 180 - DNA, 351 bp, Artificial Sequence
  TPP-30003 heavy chain
caggttcagctggtggaatctggtggcggagttgtgcagcctggcagaagcctgagactgtcttgtgccgccagcggcttcaccttcagccactacgatatgcactgggtccgacaggcccctggcaaaggacttgaatgggtcgccgtgatcagctacgagggcagcgtgaagttctacgccgactccgtgaagggcagattcaccatcagccgggacaacagcaagaacaccctgtacctgcagatgaacagcctgagagccgaggacaccgccgtgtactactgtgccagaacacccggcttccacttcgactattggggccagggaaccctggtcacagtctcttca

SEQ ID NO: 181 - DNA, 330 bp, Artificial Sequence; identical to SEQ ID NO: 145
  TPP-30003 light chain

SEQ ID NO: 182 - protein, 446 aa, Artificial Sequence
  TPP-30003 heavy chain full
QVQLVESGGGVVQPGRSLRLSCAASGFTFSHYDMHWVRQAPGKGLEWVAVISYEGSVKFYADSVKGRFTISRDNSKNTLYLQMNSLRAEDTAVYYCARTPGFHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 216 aa, Artificial Sequence; identical to SEQ ID NO: 147
  TPP-30003 light chain full

SEQ ID NO: 184 - protein, 117 aa, Artificial Sequence
  Heavy chain variable
QVQLVESGGGVVQPGRSLRLSCAASGFTFSHYDMHWVRQAPGKGLEWVAVISYEGSNKFYADSVKGRFTISRDNSKNTLYLQMNSLRAEDTAVYYCARTPGSHFDYWGQGTLVTVSS

SEQ ID NO: 185 - protein, 5 aa, Artificial Sequence
  Heavy chain CDR1
HYDMH

SEQ ID NO: 186 - protein, 17 aa, Artificial Sequence
  Heavy chain CDR2
VISYEGSNKFYADSVKG

SEQ ID NO: 187 - protein, 8 aa, Artificial Sequence
  Heavy chain CDR3
TPGSHFDY

SEQ ID NO: 188 - protein, 110 aa, Artificial Sequence; identical to SEQ ID NO: 140
  Light chain variable

SEQ ID NO: 189 - protein, 13 aa, Artificial Sequence
  Light chain CDR1
SGSSSNIGSNYVN

SEQ ID NO: 190 - protein, 7 aa, Artificial Sequence
  Light chain CDR2
YDDLRPS

SEQ ID NO: 191 - protein, 11 aa, Artificial Sequence
  Light chain CDR3
SAWDDSLSGVV

SEQ ID NO: 192 - protein, 444 aa, Artificial Sequence
  TPP-31325 heavy chain full
QVQLVESGGGVVQPGRSLRLSCAASGFTFSHYDMHWVRQAPGKGLEWVAVISYEGSNKFYADSVKGRFTISRDNSKNTLYLQMNSLRAEDTAVYYCARTPGSHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 193 - protein, 216 aa, Artificial Sequence; identical to SEQ ID NO: 147
  TPP-31325 light chain full

SEQ ID NO: 194 - protein, 444 aa, Artificial Sequence
  TPP-31391 heavy chain full
EVQLLESGGGLVQPGGSLRLSCAASGFTFSHYDMNWLRQAPGKGLEWVGVISYDGSNKFYADFVKGRFTISRDNSKNTLYLQMNSLRAEDTAVYYCARTPGSHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - protein, 216 aa, Artificial Sequence
  TPP-31385 light chain full
QSVLTQPPSASGTPGQRVTISCSGSSSNIGNNAVNWYQQLPGTAPKLLIYYDDLLPSGVPDRFSGSKSGTSASLAISGLRSEDEADYYCSAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 196 - protein, 444 aa, Artificial Sequence
  TPP-31391 heavy chain full
EVQLLESGGGLVQPGGSLRLSCAASGFTFSIYAMSWVRQAPGKGLEWVSSISDTGDRTYYADSVKGRFTISRDNSKNTLYLQMNSLRAEDTAVYYCARSNWGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - protein, 216 aa, Artificial Sequence
  TPP-31391 light chain full
QSVLTQPPSASGTPGQRVTISCSGSSSNIGSNYVYWYQQLPGTAPKLLIYYDDLLPSGVPDRFSGSKSGTSASLAISGLRSEDEADYYCAAWDDSLSGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS